(19) United States
(12) Patent Application Publication
(10) Pub. No.: US 2025/0333407 A1
(43) Pub. Date: Oct. 30, 2025

(54) Compounds and Their Use for Treatment of Hemoglobinopathies

(71) Applicant: Bristol-Myers Squibb Company, Princeton, NJ (US)

(72) Inventors: Thomas J. Cummins, San Diego, CA (US); Giorgio Tamo, Biel (CH); Hannah L. Powers, San Diego, CA (US); Scott Arne Johnson, San Diego, CA (US); Shuang Liu, San Diego, CA (US)

(73) Assignee: Bristol-Myers Squibb Company, Princeton, NJ (US)

(21) Appl. No.: 19/191,220

(22) Filed: Apr. 28, 2025

Related U.S. Application Data
(60) Provisional application No. 63/639,781, filed on Apr. 29, 2024.

(51) Int. Cl. C07D 417/14; A61K 31/4545; A61K 31/4709; A61K 31/536; A61K 31/538; A61K 31/5415; C07D 401/14; C07D 413/14; C07D 471/04

(52) U.S. Cl. CPC C07D 417/14; A61K 31/4545; A61K 31/4709; A61K 31/536; A61K 31/538; A61K 31/5415; C07D 401/14; C07D 413/14; C07D 471/04

(57) ABSTRACT

Disclosed herein are compounds and methods for the prevention and/or treatment of hemoglobinopathies. Also provided herein are such compounds for use in such methods. Also disclosed herein are pharmaceutical compositions comprising such compounds for use in such methods. In certain embodiments, the compounds are of the following structural formula:

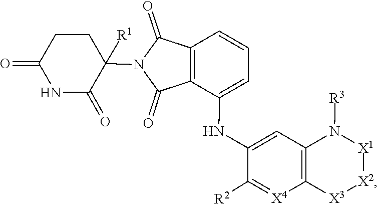

wherein values for the variables (e.g., X¹, X², X³, X⁴, R¹, R², R³) are as described herein.

### CROSS-REFERENCE TO RELATED APPLICATION

This application claims the benefit of U.S. Provisional Application No. 63/639,781, filed Apr. 29, 2024, which is incorporated herein by reference in its entirety for any purpose.

### FIELD

Disclosed herein are compounds and methods for the prevention and/or treatment of hemoglobinopathies. Also provided herein are such compounds for use in such methods. Also disclosed herein are pharmaceutical compositions comprising such compounds for use in such methods.

### BACKGROUND

Hemoglobin is an iron-containing metalloprotein present in red blood cells. Hemoglobin transports oxygen to various tissues throughout the body. Fetal hemoglobin (HbF) is present in fetal red blood cells and is involved the transport of oxygen from the mother to the fetus. After birth, a “fetal switch” occurs, at which point, erythroid precursors switch from producing predominantly fetal hemoglobin to making predominantly adult hemoglobin. Adult hemoglobin and fetal hemoglobin are tetramers containing two alpha- and two beta-globin subunits (α2β2) or two alpha- and two gamma-globin subunits (α2γ2), respectively. Fetal hemoglobin binds to oxygen more strongly than adult hemoglobin.

Hemoglobinopathies can arise, for example, when there is either abnormal adult hemoglobin expression or structural abnormalities in the adult hemoglobin protein. For example, sickle cell disease, including sickle cell anemia, arises when a person has a point mutation that occurs in the beta-globin gene, resulting in the formation of hemoglobin S (HbS), a hemoglobin composed of two normal alpha-globin chains and two beta-globin type mutated chains. The presence of HbS causes the red blood cells to have an abnormal shape, assuming a sickle form when exposed to decreased amounts of oxygen, which impedes blood flow and can lead to hemolysis. Patients with sickle cell disease suffer from a variety of symptoms including pain, anema, bacterial infections, increased risk of stroke, and reduced life expectancy.

Another example of a hemoglobinopathy is beta-thalassemia. Beta-thalassemias occur when the beta chain of hemoglobin is reduced or absent. Without treatment, patients with severe forms of beta-thalassemia can suffer from numerous health complications such as poor growth, skeletal abnormalities, and heart failure. Although beta-thalassemia patients may be treated via blood transfusions, transfusions carry the risk of iron overload and resulting complications with the spleen, liver, and heart.

Reactivation of functional HbF expression in adult hematopoietic cells has the potential of great clinical benefit in patients with hemoglobin disorders such as sickle cell disease and beta-thalassemia. Hydroxyurea is a current standard of care for sickle cell disease that works via induction of fetal hemoglobin, but it is limited by variable clinical responses, myelotoxicity and the risk of carcinogenesis. Thus, alternative, and more effective, treatments for hemoglobinopathies are needed.

### SUMMARY

Provided herein are compounds of structural formula (I):

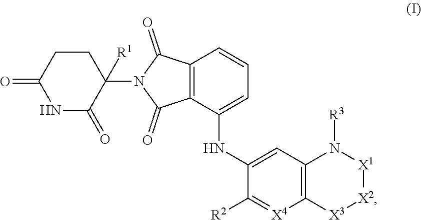

and pharmaceutically acceptable salts, tautomers, isotopologues, and stereoisomers thereof, wherein values for the variables (e.g., X¹, X², X³, X⁴, R¹, R², R³) are as described herein.

Also provided herein are pharmaceutical compositions comprising a compound of the disclosure, e.g., a compound of structural formula (I), or a substructure thereof, such as a compound of structural formula II, III, or IV, or of Table 1, or a pharmaceutically acceptable salt, tautomer, isotopologue, or stereoisomer thereof, and a pharmaceutically acceptable carrier, excipient or vehicle.

Also provided herein are methods of inducing HbF expression in a cell and/or decreasing WIZ expression and/or ZBTB7A expression in a cell (e.g., a cell expressing WIZ and/or ZBTB7A), comprising contacting the cell with a compound of the disclosure, e.g., a compound of structural formula (I), or a substructure thereof, such as a compound of structural formula II, III, or IV, or of Table 1, or a pharmaceutically acceptable salt, tautomer, isotopologue, or stereoisomer thereof.

Also provided herein are methods of treating a hemoglobinopathy in a subject in need thereof, comprising administering to the subject an effective amount of a compound of the disclosure, e.g., a compound of structural formula (I), or a substructure thereof, such as a compound of structural formula II, III, or IV, or of Table 1, or a pharmaceutically acceptable salt, tautomer, isotopologue, or stereoisomer thereof, or a pharmaceutical composition thereof.

Also provided herein are uses of a compound of the disclosure, e.g., a compound of structural formula (I), or a substructure thereof, such as a compound of structural formula II, III, or IV, or of Table 1, or a pharmaceutically acceptable salt, tautomer, isotopologue, or stereoisomer thereof, or a pharmaceutical composition thereof, e.g., for inducing HbF expression; decreasing WIZ expression; decreasing ZBTB7A expression; treating or preventing a hemoglobinopathy.

Also provided herein is a compound of the disclosure, e.g., a compound of structural formula (I), or a substructure thereof, such as a compound of structural formula II, III, or IV, or of Table 1, or a pharmaceutically acceptable salt, tautomer, isotopologue, or stereoisomer thereof, or a pharmaceutical composition thereof, for use as a medicament, e.g., for inducing HbF expression; decreasing WIZ expression; decreasing ZBTB7A expression; treating a hemoglobinopathy.

Also provided herein is a compound of the disclosure, e.g., a compound of structural formula (I), or a substructure thereof, such as a compound of structural formula II, III, or IV, or of Table 1, or a pharmaceutically acceptable salt, tautomer, isotopologue, or stereoisomer thereof, for manufacture of a medicament, e.g., for a use described herein, such as inducing HbF expression; decreasing WIZ expression; decreasing ZBTB7A expression; treating a hemoglobinopathy.

Also provided herein is a compound of the disclosure, e.g., a compound of structural formula (I), or a substructure thereof, such as a compound of structural formula II, III, or IV, or of Table 1, or a pharmaceutically acceptable salt, tautomer, isotopologue, or stereoisomer thereof, or a pharmaceutical composition thereof, for use as described herein (e.g., inducing HbF expression; decreasing WIZ expression; decreasing ZBTB7A expression; treating a hemoglobinopathy).

The present embodiments can be understood more fully by reference to the detailed description and examples, which are intended to exemplify non-limiting embodiments.

### DETAILED DESCRIPTION

### Definitions

As used herein, the terms “comprising” and “including” can be used interchangeably. The terms “comprising” and “including” are to be interpreted as specifying the presence of the stated features or components as referred to, but do not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein and unless otherwise specified, an “alkyl” group is a saturated, straight chain or branched, non-cyclic hydrocarbon having from 1 to 10 carbon atoms, typically from 1 to 8 carbons or, in some embodiments, from 1 to 6, 1 to 4, 1 to 3, or 2 to 6 carbon atoms. Representative straight chain alkyl groups include methyl, ethyl, n-propyl, n-butyl, n-pentyl and n-hexyl; while branched alkyls include isopropyl, sec-butyl, isobutyl, tert-butyl, isopentyl, neopentyl, tert-pentyl, 2-methylpentyl, 3-methylpentyl, 4-methylpentyl, 2,3-dimethylbutyl, and the like.

As used herein and unless otherwise specified, an “alkenyl” group is an unsaturated, straight chain or branched, non-cyclic hydrocarbon having from 1 to 10 carbon atoms, typically from 1 to 8 carbons or, in some embodiments, from 1 to 6, 1 to 4, 1 to 3, or 2 to 6 carbon atoms, that contains one or more carbon-carbon double bonds. Examples of alkenyl groups include, but are not limited to, vinyl, allyl, —CH═CH(CH₃), —CH═C(CH₃)₂, —C(CH₃)=CH₂, —C(CH₃)═CH(CH₃), and —C(CH₂CH₃)=CH₂, among others.

As used herein an unless specified otherwise, an “alkynyl” group is an unsaturated, straight chain or branched, non-cyclic hydrocarbon having from 1 to 10 carbon atoms, typically from 1 to 8 carbons or, in some embodiments, from 1 to 6, 1 to 4, 1 to 3, or 2 to 6 carbon atoms, that contains one or more carbon-carbon triple bonds. Examples of alkynyl groups include, but are not limited to, —C≡CH, —C≡C(CH₃), —C≡C(CH₂CH₃), —CH₂C≡CH, —CH₂C≡C(CH₃) and —CH₂C≡C(CH₂CH₃), among others.

Alkyl, alkenyl, and alkynyl groups can be substituted or unsubstituted. When the alkyl groups described herein are said to be “substituted,” they may be substituted with any substituent or substituents as those found in the exemplified compounds and embodiments disclosed herein, as well as halogen; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In some embodiments, one or more hydrogens, such as one, two, three, four, or five hydrogens, in an alkyl, alkenyl, or alkynyl group may be replaced with halogen.

As used herein and unless otherwise specified, a “cycloalkyl” group is a saturated, or partially saturated cyclic hydrocarbon having from 3 to 10 carbon atoms in a single ring or multiple condensed, spiro, or bridged rings, which can be optionally substituted. In some embodiments, a cycloalkyl group has 3 to 8 ring carbon atoms, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like. In some embodiments, a cycloalkyl is saturated.

As used herein and unless otherwise specified, an “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryl groups include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein and unless otherwise specified, a “heteroaryl” group is an aromatic ring system having one to four heteroatoms as ring atoms in a heteroaromatic ring system, wherein the remainder of the ring atoms are carbon atoms. In some embodiments, heteroaryl groups contain 3 to 6 ring atoms, and in others from 5 to 9, 6 to 9, 5 to 10, or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indol-2-onyl), isoindolin-1-onyl, azaindolyl, pyrrolopyridyl (e.g., 1H-pyrrolo[2,3-b]pyridyl), indazolyl, benzimidazolyl (e.g., 1H-benzo[d]imidazolyl), azabenzimidazolyl, imidazopyridyl (e.g., 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl, 3,4-dihydroisoquinolin-1(2H)-onyl, tetrahydroquinolinyl, quinoxalinyl, and quinazolinyl groups. A heteroaryl group can be substituted or unsubstituted. The phrase “heteroaryl groups” also includes groups containing fused rings, such as fused heteroaromatic-aliphatic or fused heteroaromatic-heteroaliphatic ring systems.

As used herein and unless otherwise specified, a “heterocyclyl” or “heterocycloalkyl” is a non-aromatic cycloalkyl in which one to four of the ring carbon atoms are independently replaced with a heteroatom. Suitable heteroatoms include oxygen, sulfur and nitrogen. In some embodiments, heterocyclyl groups include 3 to 10 ring members, whereas other such groups have 3 to 5, 3 to 6, or 3 to 8 ring members. A heterocyclyl group can be substituted or unsubstituted. When a heterocyclyl is substituted, a substituent can be bonded to the heterocyclyl at any ring atom (i.e., at any carbon atom or heteroatom of the heterocyclic ring). The phrase includes fused, spiro, and bridged polycyclic ring systems containing a heteroatom such as, but not limited to, quinuclidyl. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one.

As used herein and unless otherwise specified, a “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the aryl group is fused to a cycloalkyl group such as indan-4-yl ethyl.

As used herein and unless otherwise specified, a “heterocyclylalkyl” group is a radical of the formula: -alkyl-heterocyclyl, wherein alkyl and heterocyclyl are defined above. A “heteroarylalkyl” group is a radical of the formula: -alkyl-heteroaryl, wherein alkyl and heteroaryl are defined above. Substituted heterocyclylalkyl groups may be substituted at the alkyl, the heterocyclyl, or both the alkyl and the heterocyclyl portions of the group. Representative heterocylylalkyl groups include but are not limited to morpholin-4-yl ethyl, morpholin-4-yl propyl, furan-2-yl methyl, furan-3-yl methyl, pyridin-3-yl methyl, tetrahydrofuran-2-yl ethyl, and indol-2-yl propyl.

As used herein and unless otherwise specified, a “halogen” is fluorine, chlorine, bromine or iodine.

As used herein and unless otherwise specified, a “hydroxyalkyl” group is an alkyl group as described above substituted with one or more hydroxy groups.

As used herein and unless otherwise specified, an “alkoxy” group is —O-(alkyl), wherein alkyl is defined above. An “alkylthio” group is —S-(alkyl), wherein alkyl is defined above.

As used herein and unless otherwise specified, an “alkoxyalkyl” group is -(alkyl)-O-(alkyl), wherein alkyl is defined above.

As used herein and unless otherwise specified, a “cycloalkyloxy” group is —O-(cycloalkyl), wherein cycloalkyl is defined above.

As used herein and unless otherwise specified, an “aryloxy” group is —O-(aryl), wherein aryl is defined above.

As used herein and unless otherwise specified, a “heterocyclyloxy” group is —O-(heterocyclyl), wherein heterocyclyl is defined above. A “heteroaryloxy” group is —O-(heteroaryl), wherein heteroaryl is defined above.

As used herein and unless otherwise specified, an “amino” group is a radical of the formula: —NH₂, —NH(R^{#}), or —N(R^{#})₂, wherein each R⁴ is independently an alkyl, cycloalkyl, cycloalkylalkyl, aryl, aralkyl, heterocyclyl (e.g., heteroaryl or heterocycloalkyl), or heterocyclylalkyl (e.g., heteroarylalkyl or heterocycloalkylalkyl) group defined above, each of which is independently substituted or unsubstituted.

In one embodiment, an “amino” group is an “alkylamino” group, which is a radical of the formula: —NH-alkyl or —N(alkyl)₂, wherein each alkyl is independently defined above. The term “cycloalkylamino”, “arylamino”, “heterocyclylamino”, “heteroarylamino”, “heterocycloalkylamino”, or the like, mirrors the above description for “alkylamino” where the term “alkyl” is replaced with “cycloalkyl”, “aryl”, “heterocyclyl”, “heteroaryl”, “heterocycloalkyl”, or the like, respectively.

As used herein and unless otherwise specified, a “carboxy” group is a radical of the formula: —C(O)OH.

As used herein and unless otherwise specified, an “acyl” group is a radical of the formula: —C(O)(R^{#}) or —C(O)H, wherein R^{#} is defined above. A “formyl” group is a radical of the formula: —C(O)H.

As used herein and unless otherwise specified, an “amido” group is a radical of the formula: —C(O)—NH₂, —C(O)—NH(R^{#}), —C(O)—N(R^{#})₂, —NH—C(O)H, —NH—C(O)—(R^{#}), —N(R^{#})—C(O)H, or —N(R^{#})—C(O)—(R^{#}), wherein each R^{#} is independently defined above.

In one embodiment, an “amido” group is an “aminocarbonyl” group, which is a radical of the formula: —C(O)—NH₂, —C(O)—NH(R^{#}), —C(O)—N(R^{#})₂, wherein each R^{#} is independently defined above.

In one embodiment, an “amido” group is an “acylamino” group, which is a radical of the formula: —NH—C(O)H, —NH—C(O)—(R^{#}), —N(R)—C(O)H, or —N(R^{#})—C(O)—(R^{#}), wherein each R^{#} is independently defined above.

As used herein and unless otherwise specified, a “sulfonylamino” group is a radical of the formula: —NHSO₂(R^{#}) or —N(R^{#})SO₂(R^{#}), wherein each R^{#} is defined above.

As used herein and unless otherwise specified, an “ester” group is a radical of the formula: —C(O)—O—(R^{#}) or —O—C(O)—(R^{#}), wherein R^{#} is defined above.

In one embodiment, an “ester” group is an “alkoxycarbonyl” group, which is a radical of the formula: —C(O)—O-(alkyl), wherein alkyl is defined above. The term “cycloalkyloxycarbonyl”, “aryloxycarbonyl”, “heterocyclyloxycarbonyl”, “heteroaryloxycarbonyl”, “heterocycloalkyloxycarbonyl”, or the like, mirrors the above description for “alkoxycarbonyl” where the term “alkoxy” is replaced with “cycloalkyloxy”, “aryloxy”, “heterocyclyloxy”, “heteroaryloxy”, “heterocycloalkyloxy”, or the like, respectively.

As used herein and unless otherwise specified, a “carbamate” group is a radical of the formula: —O—C(O)—NH₂, —O—C(O)—NH(R^{#}), —O—C(O)—N(R^{#})₂, —NH—C(O)—O—(R^{#}), or —N(R^{#})—C(O)—O—(R^{#}), wherein each R^{#} is independently defined above.

As used herein and unless otherwise specified, a “urea” group is a radical of the formula: —NH(CO)NH₂, —NHC(O)NH(R^{#}), —NHC(O)N(R^{#})₂, —N(R^{#})C(O)NH₂, —N(R^{#})C(O)NH(R^{#}), or —N(R^{#})C(O)N(R^{#})₂, wherein each R^{#} is independently defined above.

As used herein and unless otherwise specified, a “sulfinyl” group is a radical of the formula: —S(O)R^{#}, wherein R^{#} is defined above.

As used herein and unless otherwise specified, a “sulfonyl” group is a radical of the formula: —S(O)₂R^{#}, wherein R^{#} is defined above.

As used herein and unless otherwise specified, an “aminosulfonyl” group is a radical of the formula: —SO₂NH₂, —SO₂NH(R^{#}), or —SO₂N(R^{#})₂, wherein each R^{#} is independently defined above.

When the groups described herein, with the exception of alkyl, alkenyl, and alkynyl groups, are said to be “substituted,” they may be substituted with any appropriate substituent or substituents. Illustrative examples of substituents are those found in the exemplary compounds and embodiments disclosed herein, as well as halogen; alkyl, alkenyl, alkynyl, cycloalkyl, aryl, heterocyclyl, heteroaryl, cycloalkylalkyl, aralkyl, heterocyclylalkyl, heteroarylalkyl, optionally further substituted; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; oxo (═O); oxide (e.g., a nitrogen atom substituted with an oxide is called N-oxide); amino, alkylamino, cycloalkylamino, arylamino, heterocyclylamino, heteroarylamino, imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In some embodiments, one or more hydrogens, such as one, two, three, four, or five hydrogens, in a substituent may be replaced with halogen. In some embodiments, substitution replaces a hydrogen atom with alkyl, alkoxy, aryloxy, halogen, or haloalkyl.

As used herein, the term “hemoglobinopathy” or “hemoglobinopathies” means any disease or disorder that affects red blood cells. Hemoglobinopathies include, but are not limited to, sickle cell disease and anemias.

As used herein, the term “HbF” means fetal hemoglobin.

As used herein, the term “gene therapy” means the treatment of a disease or condition by the transfer or contact of genetic material into a cell.

As used herein, the term “CRISPR” means clustered regularly interspaced short palindromic repeats.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds described herein include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride and mesylate salts. Other salts are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. Compounds can have chiral centers and can occur as racemates, individual enantiomers or diastereomers, and mixtures thereof. All such isomeric forms are included within the embodiments disclosed herein, including mixtures thereof.

The use of stereomerically pure forms of compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted that compounds can include E and Z isomers, or cis and trans isomers. All such isomeric forms are included within the embodiments disclosed herein, including mixtures thereof.

“Tautomers” refers to isomeric forms of a compound that are in equilibrium with each other through the migration of a proton. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

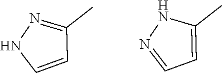

As readily understood by one skilled in the art, a wide variety of functional groups and other structures may exhibit tautomerism and all tautomers of compounds are within the scope of the present disclosure.

It should also be noted that compounds can contain unnatural proportions of atomic isotopes at least one of the atoms. For example, compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of compounds described herein, whether radioactive or not, are intended to be encompassed within the scope of the embodiments provided herein. In some embodiments, there are provided isotopologues of the compounds, for example, the isotopologues are carbon-13, or nitrogen-15 enriched. As used herein, “deuterated”, means a compound wherein at least one hydrogen (H) has been replaced by deuterium (indicated by D or ²H), that is, the compound is enriched in deuterium in at least one position. In some embodiments, an isotopologue is a deuterated isotopologue of a specified compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder, disorder or condition is a hemoglobinopathy.

The term “effective amount” means an amount capable of treating or preventing a disorder, disease or condition, or symptoms thereof, disclosed herein. In one embodiment, the disorder, disorder or condition is a hemoglobinopathy.

The term “subject” or “patient” includes humans.

The term “combination” or administration “in combination” includes administration as a mixture, simultaneous administration using separate formulations, and consecutive administration in any order.

Unless otherwise defined, the technical and scientific terms used herein have the same meaning as commonly understood by one of ordinary skill in the art to which this application belongs.

### COMPOUNDS

Provided herein is a compound of structural formula (I):

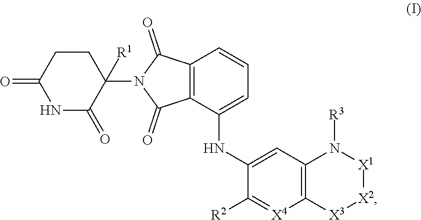

or a pharmaceutically acceptable salt, tautomer, isotopologue, and/or stereoisomer thereof, wherein:
X¹ is C(═O), C(═S), S(═O)₂, or C(R¹⁰)₂;
   each R¹⁰ is independently H or (C₁-C₃)alkyl;
X² is C(R²⁰)₂ and X³ is O or C(R³⁰)₂; or X² is O and X³ is C(R³⁰)₂ or absent;
   each R²⁰ is independently H or (C₁-C₃)alkyl;
   each R³⁰ is independently H or (C₁-C₃)alkyl;
X⁴ is C(R⁴⁰) or N;
   R⁴⁰ is H or F;
R¹ is H or methyl;
R² is five- to ten-membered heteroaryl, (C₆-C₁₀)aryl, three- to eight-membered heterocyclyl, or (C₃-C₈)cycloalkyl, and is optionally substituted with (R⁴)ₓ;
R³ is (C₁-C₃)alkyl, (C₁-C₃)alkenyl, or (C₃-C₆)cycloalkyl;
each R⁴ is independently cyano, halo, (C₁-C₃)alkyl, halo(C₁-C₃)alkyl, —O—(C₁-C₃)alkyl, (C₃-C₆)cycloalkyl, —C(O)(C₁-C₃)alkyl, —C(O)(C₃-C₆)cycloalkyl, —NH₂, —N(H)(C₁-C₃)alkyl, or —N((C₁-C₃)alkyl)₂; and
x is 1, 2, 3, or 4.

In some embodiments, the compound is not

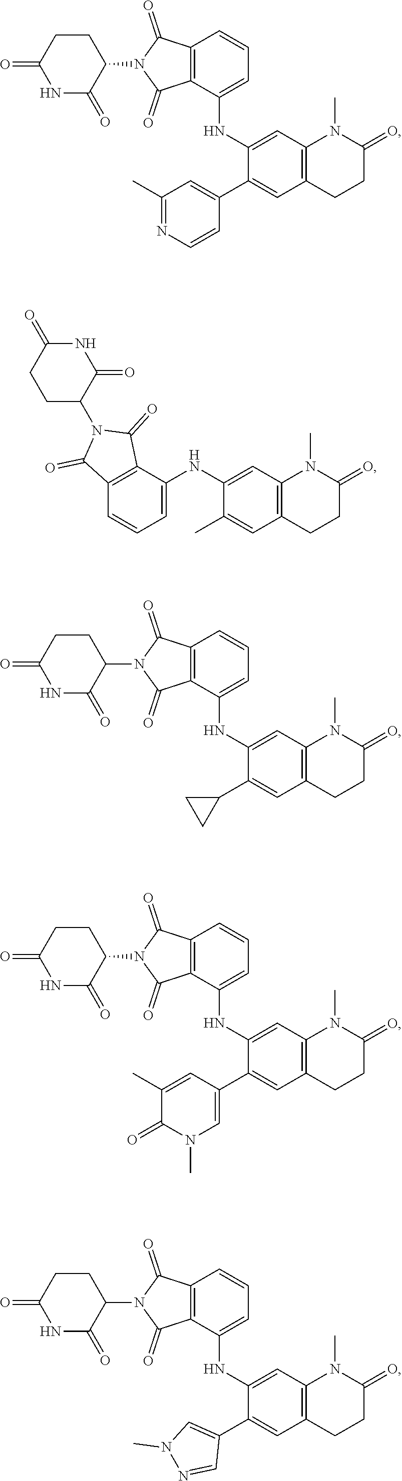 [SMILES: Cc1cc(-c2cc3c(cc2Nc2cccc4c2C(=O)N([C@H]2CCC(=O)NC2=O)C4=O)N(C)C(=O)CC3)ccn1 ; Cc1cc2c(cc1Nc1cccc3c1C(=O)N(C1CCC(=O)NC1=O)C3=O)N(C)C(=O)CC2 ; CN1C(=O)CCc2cc(C3CC3)c(Nc3cccc4c3C(=O)N(C3CCC(=O)NC3=O)C4=O)cc21 ; Cc1cc(-c2cc3c(cc2Nc2cccc4c2C(=O)N([C@H]2CCC(=O)NC2=O)C4=O)N(C)C(=O)CC3)cn(C)c1=O ; CN1C(=O)CCc2cc(-c3cnn(C)c3)c(Nc3cccc4c3C(=O)N(C3CCC(=O)NC3=O)C4=O)cc21]

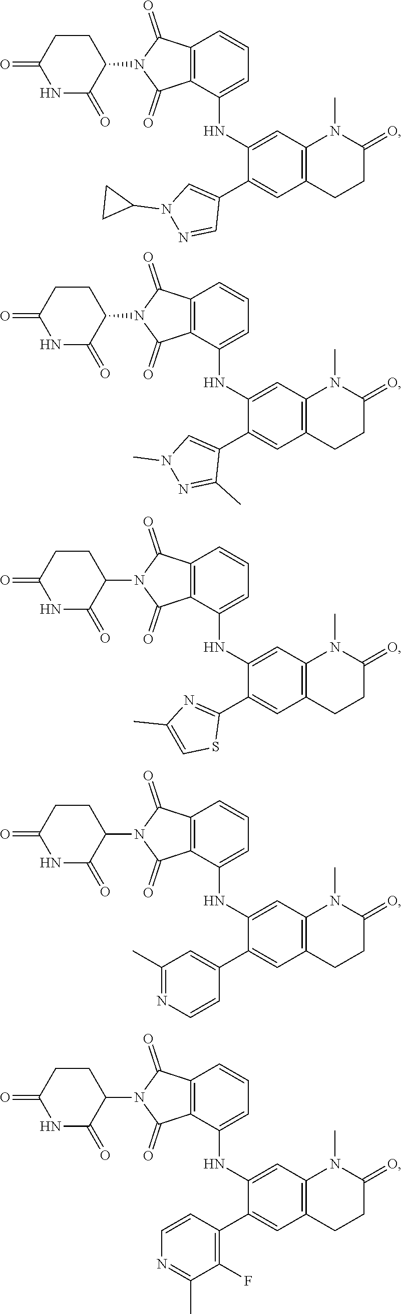

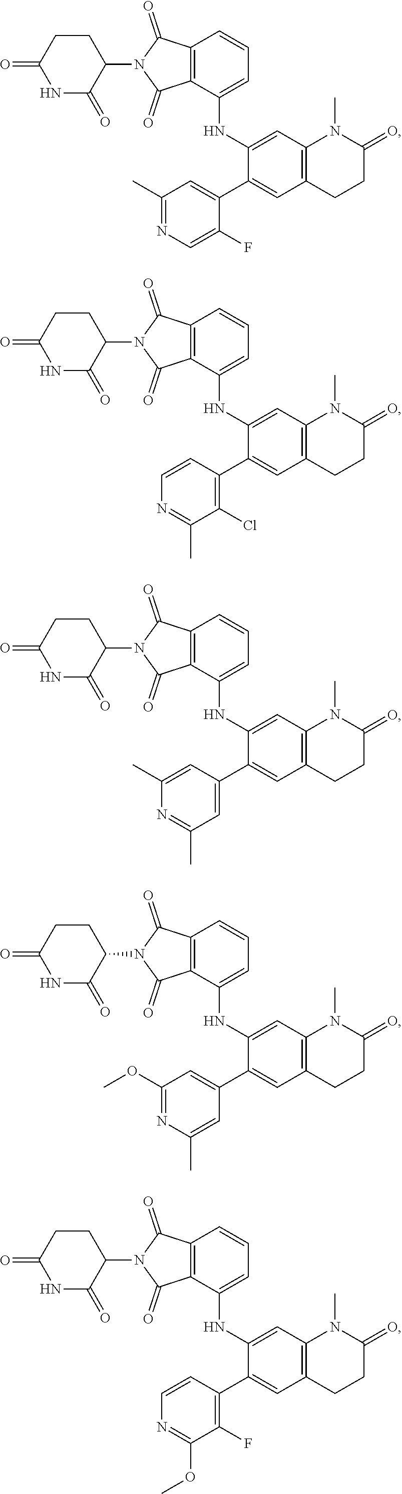 [SMILES: Cc1cc(-c2cc3c(cc2Nc2cccc4c2C(=O)N(C2CCC(=O)NC2=O)C4=O)N(C)C(=O)CC3)c(F)cn1 ; Cc1nccc(-c2cc3c(cc2Nc2cccc4c2C(=O)N(C2CCC(=O)NC2=O)C4=O)N(C)C(=O)CC3)c1Cl ; Cc1cc(-c2cc3c(cc2Nc2cccc4c2C(=O)N(C2CCC(=O)NC2=O)C4=O)N(C)C(=O)CC3)cc(C)n1 ; COc1cc(-c2cc3c(cc2Nc2cccc4c2C(=O)N([C@H]2CCC(=O)NC2=O)C4=O)N(C)C(=O)CC3)cc(C)n1 ; COc1nccc(-c2cc3c(cc2Nc2cccc4c2C(=O)N(C2CCC(=O)NC2=O)C4=O)N(C)C(=O)CC3)c1F]

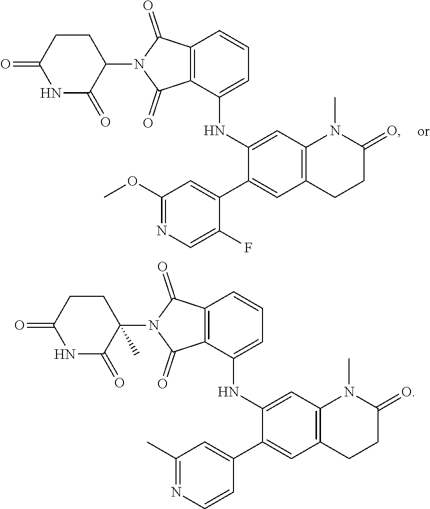

In some embodiments, when X¹ is C(═O), and X² and X³ are each C(H)₂, and X⁴ is C(H), then R³ is not methyl.

Also provided herein is a compound of structural formula (II):

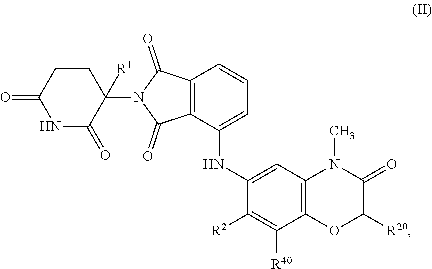

or a pharmaceutically acceptable salt, tautomer, isotopologue, and/or stereoisomer thereof, wherein values for the variables (e.g., R¹, R², R²⁰, R⁴⁰) are as described with respect to structural formula (I).

In some embodiments, X¹ is C(═O), C(═S), S(═O)₂, C(H)₂, or C(H)(CH₃). In some embodiments, X¹ is C(═O). In some embodiments, X¹ is C(═O) or C(═S). In some embodiments, X¹ is S(═O)₂. In some embodiments, X¹ is C(H)₂, C(CH₃)₂, or C(H)(CH₃).

In some embodiments, X² is C(R²⁰)₂. In some embodiments, X² is C(H)₂, C(H)(CH₃) or C(CH₃)₂. In alternative embodiments, X² is O. In some embodiments, X² is O, C(H)₂, C(H)(CH₃) or C(CH₃)₂.

In some embodiments, X³ is O. In alternative embodiments, X³ is C(R³⁰)₂. In some embodiments, X³ is C(H)₂, C(H)(CH₃), or C(CH₃)₂, or absent. In some embodiments, X³ is C(H)₂, C(H)(CH₃), or C(CH₃)₂. In some embodiments, X³ is O, C(H)₂, C(H)(CH₃), or C(CH₃)₂, or absent. In some embodiments, X³ is O, C(H)₂, C(H)(CH₃), or C(CH₃)₂. In some embodiments, X³ is absent.

In some embodiments, X² is C(R²⁰)₂ and X³ is O or C(R³⁰)₂. In some embodiments, X² is C(R²⁰)₂ and X³ is 0. In some embodiments, X² is C(H)₂, C(H)(CH₃), or C(CH₃)₂, and X³ is 0. In some embodiments, X² is C(R²⁰)₂ and X³ is C(R³⁰)₂. In some embodiments, X² is C(H)₂, C(H)(CH₃), or C(CH₃)₂, and X³ is C(R³⁰)₂. In some embodiments, X² is C(R²⁰)₂ and X³ is C(H)₂, C(H)(CH₃), or C(CH₃)₂. In some embodiments, X² is C(H)₂, C(H)(CH₃), or C(CH₃)₂, and X³ is C(H)₂, C(H)(CH₃), or C(CH₃)₂. In some embodiments, X² is C(R²⁰)₂ and X³ is O, C(H)₂, C(H)(CH₃), or C(CH₃)₂. In some embodiments, X² is C(H)₂, C(H)(CH₃), or C(CH₃)₂, and X³ is O, C(H)₂, C(H)(CH₃), or C(CH₃)₂.

In some embodiments, X² is O and X³ is C(R³⁰)₂ or absent. In some embodiments, X² is O and X³ is C(R³⁰)₂. In some embodiments, X² is O and X³ is C(H)₂, C(H)(CH₃), or C(CH₃)₂, or absent. In some embodiments, X² is O and X³ is C(H)₂, C(H)(CH₃), or C(CH₃)₂. In some embodiments, X² is O and X³ is absent.

In some embodiments, X⁴ is C(R⁴⁰). In some embodiments, X⁴ is C(H). In some embodiments, X⁴ is C(F). In some embodiments, X⁴ is N.

In some embodiments, R¹ is H. In alternative embodiments, R¹ is methyl.

In some embodiments, R² is five- to ten-membered heteroaryl or (C₆-C₁₀)aryl, and is optionally substituted with (R⁴)ₓ. In some embodiments, R² is five- or six-membered heteroaryl or phenyl, and is optionally substituted with (R⁴)ₓ. In some embodiments, R² is six-membered heteroaryl or phenyl, and is optionally substituted with (R⁴)ₓ. In some embodiments, R² is pyridinyl or phenyl, and is optionally substituted with (R⁴)ₓ.

In some embodiments, R² is five- to ten-membered heteroaryl optionally substituted with (R⁴)ₓ. In some embodiments, R² is five- or six-membered heteroaryl optionally substituted with (R⁴)ₓ. In some embodiments, R² is six-membered heteroaryl optionally substituted with (R⁴)ₓ. In some embodiments, R² is pyridinyl optionally substituted with (R⁴)ₓ.

In some embodiments, R² is (C₆-C₁₀)aryl optionally substituted with (R⁴)ₓ. In some embodiments, R² is phenyl optionally substituted with (R⁴)ₓ.

In some embodiments, R² is three- to eight-membered heterocyclyl or (C₃-C₈)cycloalkyl, and is optionally substituted with (R⁴)ₓ. In some embodiments, R² is six-membered heterocyclyl or (C₆)cycloalkyl, and is optionally substituted with (R⁴)ₓ. In some embodiments, R² is piperidinyl or cyclohexyl, and is optionally substituted with (R⁴)ₓ.

In some embodiments, R² is three- to eight-membered heterocyclyl optionally substituted with (R⁴)ₓ. In some embodiments, R² is six-membered heterocyclyl optionally substituted with (R⁴)ₓ. In some embodiments, R² is piperidinyl optionally substituted with (R⁴)ₓ.

In some embodiments, R² is (C₃-C₈)cycloalkyl optionally substituted with (R⁴)ₓ. In some embodiments, R² is (C₆)cycloalkyl optionally substituted with (R⁴)ₓ. In some embodiments, R² is cyclohexyl optionally substituted with (R⁴)ₓ.

In some embodiments, R² is pyridinyl, phenyl, piperidinyl, or cyclohexyl, and is optionally substituted with (R⁴)ₓ.

In some embodiments, R² is

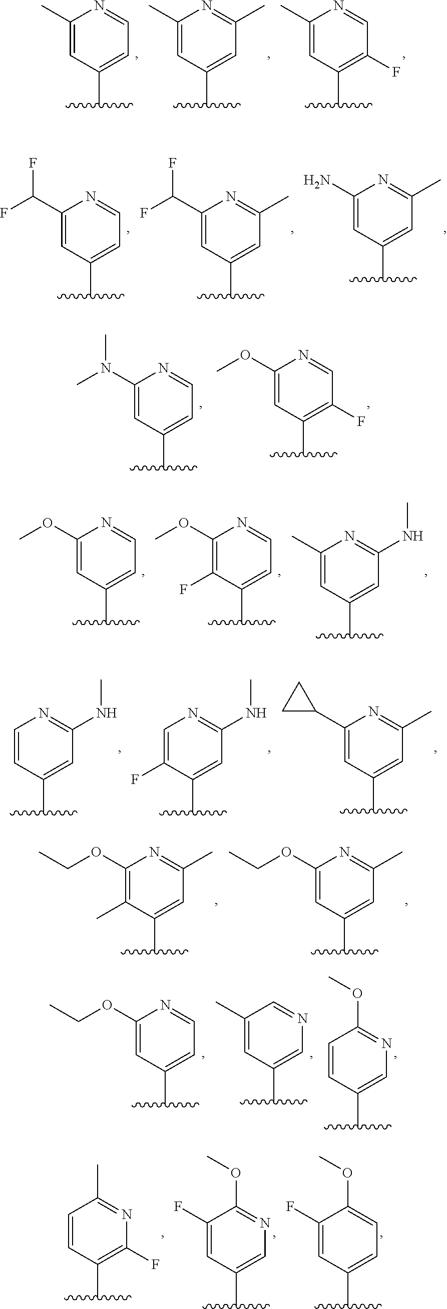

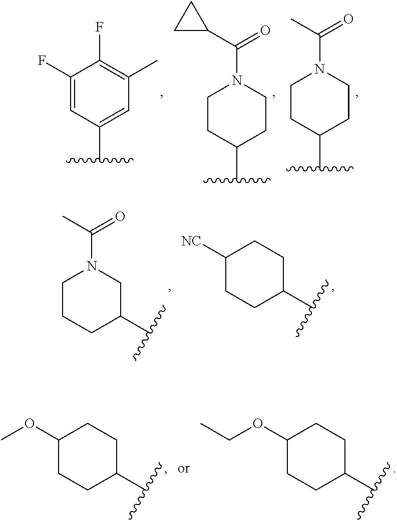

In some embodiments, R³ is (C₁-C₃)alkyl or (C₁-C₃)alkenyl. In some embodiments, R³ is (C₁-C₃)alkyl or (C₃-C₆)cycloalkyl. In some embodiments, R³ is (C₁-C₃)alkyl. In some embodiments, R³ is methyl, ethyl, vinyl, or cyclopropyl. In some embodiments, R³ is methyl.

In some embodiments, each R⁴ is independently cyano, fluoro, methyl, difluoromethyl, methoxy, ethoxy, cyclopropyl, acetyl, cyclopropanecarbonyl, —NH₂, —N(H)CH₃, or —N(CH₃)₂. In some embodiments, each R⁴ is independently cyano or —O—(C₁-C₃)alkyl. In some embodiments, each R⁴ is independently cyano, methoxy, or ethoxy.

In some embodiments, each R¹⁰ is H. In some embodiments, each R¹⁰ is independently (C₁-C₃)alkyl. In some embodiments, each R¹⁰ is methyl. In some embodiments, one R¹⁰ is H and one R¹⁰ is (C₁-C₃)alkyl. In some embodiments, one R¹⁰ is H and one R¹⁰ is methyl.

In some embodiments, each R²⁰ is H. In some embodiments, each R²⁰ is independently (C₁-C₃)alkyl. In some embodiments, each R²⁰ is methyl. In some embodiments, one R²⁰ is H and one R²⁰ is (C₁-C₃)alkyl. In some embodiments, one R²⁰ is H and one R²⁰ is methyl.

In some embodiments, each R³⁰ is H. In some embodiments, each R³⁰ is independently (C₁-C₃)alkyl. In some embodiments, each R³⁰ is methyl. In some embodiments, one R³⁰ is H and one R³⁰ is (C₁-C₃)alkyl. In some embodiments, one R³⁰ is H and one R³⁰ is methyl.

In some embodiments, R⁴⁰ is H. In some embodiments, R⁴⁰ is F.

In some embodiments, x is 1, 2, or 3. In some embodiments, x is 1 or 2. In some embodiments, x is 1.

Also provided herein is a compound of structural formula (III):

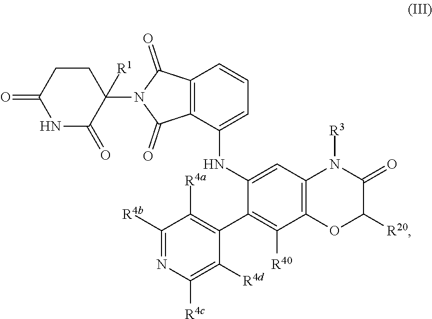

or a pharmaceutically acceptable salt, tautomer, isotopologue, and/or stereoisomer thereof, wherein R^{4a}, R^{4b}, R^{4c}, and R^{4d} are each independently H, cyano, halo, (C₁-C₃)alkyl, halo(C₁-C₃)alkyl, —O—(C₁-C₃)alkyl, (C₃-C₆)cycloalkyl, —C(O)(C₁-C₃)alkyl, —C(O)(C₃-C₆)cycloalkyl, —NH₂, —N(H)(C₁-C₃)alkyl, or —N((C₁-C₃)alkyl)₂; and values and alternative values for the remaining variables (e.g., R¹, R³, R²⁰, R⁴⁰) are as described herein.

In some embodiments, R^{4a}, R^{4b}, R^{4c}, and R^{4d} are each independently H, cyano, fluoro, methyl, difluoromethyl, methoxy, ethoxy, cyclopropyl, acetyl, cyclopropanecarbonyl, —NH₂, —N(H)CH₃, or —N(CH₃)₂.

In some embodiments, R^{4a} is H, halo, or (C₁-C₃)alkyl. In some embodiments, R^{4a} is H, F, or methyl.

In some embodiments, R^{4b} is (C₁-C₃)alkyl, (C₃-C₆)cycloalkyl, —O—(C₁-C₃)alkyl, —NH₂, —N(H)(C₁-C₃)alkyl, or —N((C₁-C₃)alkyl)₂. In some embodiments, R^{4b} is methyl, cyclopropyl, methoxy, ethoxy, —NH₂, —N(H)CH₃, or —N(CH₃)₂.

In some embodiments, R^{4c} is H, halo, (C₁-C₃)alkyl, or halo(C₁-C₃)alkyl. In some embodiments, R^{4c} is H, F, methyl, or difluoromethyl.

In some embodiments, R^{4d} is H or halo. In some embodiments, R^{4d} is H or F.

In some embodiments,

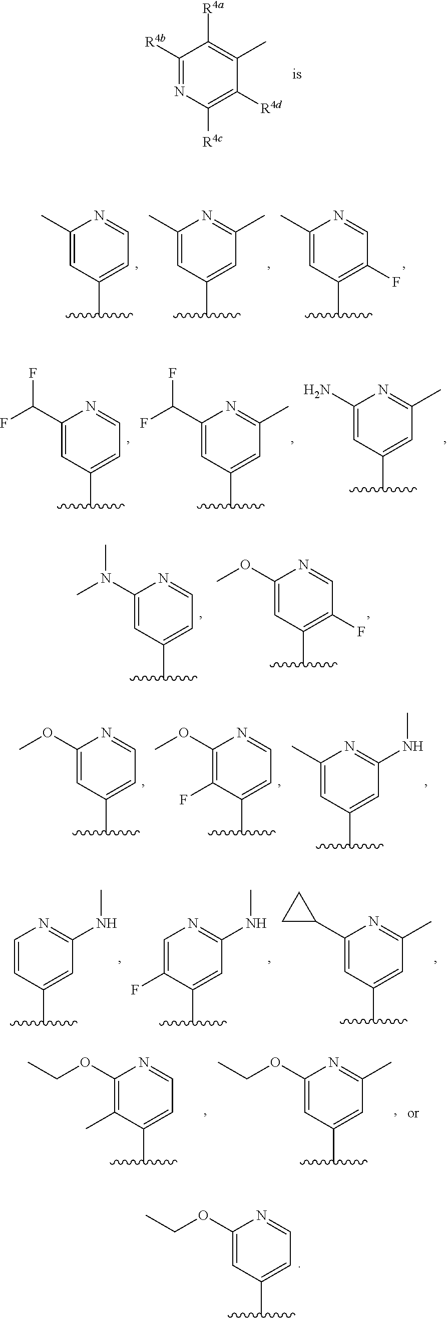

Also provided herein is a compound of structural formula (IV):

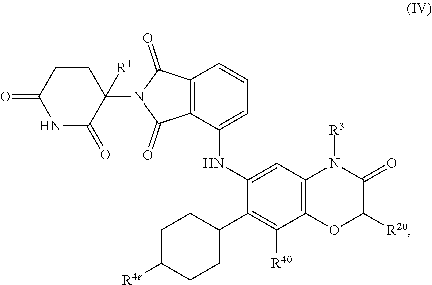

or a pharmaceutically acceptable salt, tautomer, isotopologue, and/or stereoisomer thereof, wherein R^{4e} is H, cyano, halo, (C₁-C₃)alkyl, halo(C₁-C₃)alkyl, —O—(C₁-C₃)alkyl, (C₃-C₆)cycloalkyl, —C(O)(C₁-C₃)alkyl, —C(O)(C₃-C₆)cycloalkyl, —NH₂, —N(H)(C₁-C₃)alkyl, or —N((C₁-C₃)alkyl)₂; and values and alternative values for the remaining variables (e.g., R¹, R³, R²⁰, R⁴⁰) are as described herein.

In some embodiments, R^{4e} is cyano or —O—(C₁-C₃)alkyl. In some embodiments, R^{4e} is cyano, methoxy, or ethoxy.

Representative compounds of structural formulas (I), (II), (III), and (IV) are set forth in Table 1. In some embodiments, provided herein is one or more compounds of Table 1, or a pharmaceutically acceptable salt, tautomer, isotopologue, and/or stereoisomer thereof.

### Methods of Use

In some embodiments, the present disclosure provides methods of inducing HbF expression in a cell, comprising contacting the cell with a compound of the present disclosure (e.g., a compound of structural formula (I), (II), (III), or (IV), of Table 1), or a pharmaceutically acceptable salt, tautomer, isotopologue, or stereoisomer thereof.

In some embodiments, the present disclosure provides methods of decreasing WIZ expression in a cell (e.g., a cell expressing WIZ), comprising contacting the cell with a compound of the present disclosure (e.g., a compound of structural formula (I), (II), (III), or (IV), of Table 1), or a pharmaceutically acceptable salt, tautomer, isotopologue, or stereoisomer thereof.

In some embodiments, the present disclosure provides methods of decreasing ZBTB7A expression in a cell (e.g., a cell expressing ZBTB7A), comprising contacting the cell with with a compound of the present disclosure (e.g., a compound of structural formula (I), (II), (III), or (IV), of Table 1), or a pharmaceutically acceptable salt, tautomer, isotopologue, or stereoisomer thereof.

In some embodiments, the present disclosure provides methods of inducing HbF expression in a cell, and/or decreasing ZBTB7A expression in a cell, and/or decreasing WIZ expression in a cell, comprising contacting a cell with with a compound of the present disclosure (e.g., a compound of structural formula (I), (II), (III), or (IV), of Table 1), or a pharmaceutically acceptable salt, tautomer, isotopologue, or stereoisomer thereof.

In some embodiments, the cell is in vitro. In some embodiments, the cell is ex vivo. In some embodiments, the cell is in vivo. In some embodiments, the cell is in a subject (e.g., a subject in need thereof, such as a subject affected by a hemoglobinopathy).

In some embodiments, the present disclosure provides methods of inducing HbF expression in a subject (e.g., a subject in need thereof, such as a subject affected by a hemoglobinopathy), comprising administering to the subject a compound of the present disclosure (e.g., a compound of structural formula (I), (II), (III), or (IV), of Table 1), or a pharmaceutically acceptable salt, tautomer, isotopologue, or stereoisomer thereof, e.g., an effective amount of a compound of the present disclosure, or a pharmaceutically acceptable salt, tautomer, isotopologue, or stereoisomer thereof.

In some embodiments, the present disclosure provides methods of decreasing WIZ expression in a subject (e.g., a subject in need thereof, such as a subject affected by a hemoglobinopathy), comprising administering to the subject a compound of the present disclosure (e.g., a compound of structural formula (I), (II), (III), or (IV), of Table 1), or a pharmaceutically acceptable salt, tautomer, isotopologue, or stereoisomer thereof, e.g., an effective amount of a compound of the present disclosure, or a pharmaceutically acceptable salt, tautomer, isotopologue, or stereoisomer thereof.

In some embodiments, the present disclosure provides methods of decreasing ZBTB7A expression in a subject (e.g., a subject in need thereof, such as a subject affected by a hemoglobinopathy), comprising administering to the subject a compound of the present disclosure (e.g., a compound of structural formula (I), (II), (III), or (IV), of Table 1), or a pharmaceutically acceptable salt, tautomer, isotopologue, or stereoisomer thereof, e.g., an effective amount of a compound of the present disclosure, or a pharmaceutically acceptable salt, tautomer, isotopologue, or stereoisomer thereof.

In some embodiments, the present disclosure provides methods of treating a hemoglobinopathy, comprising administering to a subject in need thereof a compound of the present disclosure (e.g., a compound of structural formula (I), (II), (III), or (IV), of Table 1), or a pharmaceutically acceptable salt, tautomer, isotopologue, or stereoisomer thereof (e.g., an effective amount of a compound of the present disclosure, or a pharmaceutically acceptable salt, tautomer, isotopologue, or stereoisomer thereof).

In some embodiments, the present disclosure provides use of a compound of the present disclosure (e.g., a compound of structural formula (I), (II), (III), or (IV), of Table 1), or a pharmaceutically acceptable salt, tautomer, isotopologue, or stereoisomer thereof for the treatment of a hemoglobinopathy.

In certain embodiments, the hemoglobinopathy is anemia. In certain embodiments, the hemoglobinopathy is sickle cell disease. In certain embodiments, the hemoglobinopathy is thalassemia. In certain embodiments, the hemoglobinopathy is alpha-thalassemia. In certain embodiments, the hemoglobinopathy is beta-thalassemia.

In certain embodiments, provided herein are methods of treating a hemoglobinopathy, comprising administering to a subject in need thereof a compound of the present disclosure (e.g., a compound of structural formula (I), (II), (III), or (IV), of Table 1), or a pharmaceutically acceptable salt, tautomer, isotopologue, or stereoisomer thereof (e.g., an effective amount of a compound of the present disclosure), or a pharmaceutically acceptable salt, tautomer, isotopologue, or stereoisomer thereof in combination with an additional active agent and/or therapy. In certain embodiments, the additional active agent and/or therapy is an active agent selected from the group consisting of luspatercept, voxelotor, crizanlizumab-tmca, hydroxyurea, L-glutamine, etavopivat, mitapivat, osivelotor, and inclacumab. In certain embodiments, the additional active agent and/or therapy is a therapy selected from the group consisting of a blood transfusion, a stem cell and/or bone marrow transplant, and/or a gene therapy (e.g., CRISPR therapy).

In certain embodiments, the additional active agent and/or therapy comprises luspatercept. In certain embodiments, the additional active agent and/or therapy comprises voxelotor. In certain embodiments, the additional active agent and/or therapy comprises crizanlizumab-tmca. In certain embodiments, the additional active agent and/or therapy comprises hydroxyurea. In certain embodiments, the additional active agent and/or therapy comprises L-glutamine. In certain embodiments, the additional active agent and/or therapy comprises etavopivat. In certain embodiments, the additional active agent and/or therapy comprises mitapivat. In certain embodiments, the additional active agent and/or therapy comprises osivelotor. In certain embodiments, the additional active agent and/or therapy comprises inclacumab. In certain embodiments, the additional active agent and/or therapy comprises or further comprises a blood transfusion. In certain embodiments, the additional active agent and/or therapy comprises or further comprises a stem cell transplant. In certain embodiments, the additional active agent and/or therapy comprises or further comprises a bone marrow transplant. In certain embodiments, the additional active agent and/or therapy comprises or further comprises a gene therapy. In further embodiments, the gene therapy is a CRISPR therapy.

In certain embodiments, the additional active agent and/or therapy is luspatercept. In certain embodiments, the additional active agent and/or therapy is voxelotor. In certain embodiments, the additional active agent and/or therapy is crizanlizumab-tmca. In certain embodiments, the additional active agent and/or therapy is hydroxyurea. In certain embodiments, the additional active agent and/or therapy is L-glutamine. In certain embodiments, the additional active agent and/or therapy is etavopivat. In certain embodiments, the additional active agent and/or therapy is mitapivat. In certain embodiments, the additional active agent and/or therapy is osivelotor. In certain embodiments, the additional active agent and/or therapy is inclacumab. In certain embodiments, the additional active agent and/or therapy is a blood transfusion. In certain embodiments, the additional active agent and/or therapy is a stem cell transplant. In certain embodiments, the additional active agent and/or therapy is a bone marrow transplant. In certain embodiments, the additional active agent and/or therapy is a gene therapy. In further embodiments, the gene therapy is a CRISPR therapy.

In certain embodiments, provided herein are methods of treating a hemoglobinopathy (e.g., any of the hemoglobinopathies disclosed herein), comprising administering to a subject in need thereof a compound of the present disclosure (e.g., a compound of structural formula (I), (II), (III), or (IV), of Table 1), or a pharmaceutically acceptable salt, tautomer, isotopologue, or stereoisomer thereof (e.g., an effective amount of a compound of the present disclosure, or a pharmaceutically acceptable salt, tautomer, isotopologue, or stereoisomer thereof) in combination with an additional active agent. In particular embodiments, the additional active agent is selected from the group consisting of luspatercept, voxelotor, crizanlizumab-tmca, hydroxyurea, L-glutamine, etavopivat, mitapivat, osivelotor and inclacumab.

In certain embodiments, provided herein are methods of treating a hemoglobinopathy (e.g., any of the hemoglobinopathies disclosed herein), comprising administering to a subject in need thereof a compound of the present disclosure (e.g., a compound of structural formula (I), (II), (III), or (IV), of Table 1), or a pharmaceutically acceptable salt, tautomer, isotopologue, or stereoisomer thereof (e.g., an effective amount of a compound of the present disclosure, or a pharmaceutically acceptable salt, tautomer, isotopologue, or stereoisomer thereof) in combination with an additional therapy. In particular embodiments, the additional therapy is selected from the group consisting of a blood transfusion, a stem cell and/or bone marrow transplant, and/or a gene therapy. In certain embodiments, the gene therapy is a CRISPR therapy.

### Pharmaceutical Compositions and Routes of Administration

Provided herein are pharmaceutical compositions comprising a compound of the present disclosure (e.g., a compound of structural formula (I), (II), (III), or (IV), of Table 1), or a pharmaceutically acceptable salt, tautomer, isotopologue, or stereoisomer thereof, (e.g., an effective amount of a compound of the present disclosure or a pharmaceutically acceptable salt, tautomer, isotopologue, or stereoisomer thereof, and a pharmaceutically acceptable carrier, excipient or vehicle.

The compounds described herein can be administered to a subject enterally (for example, orally, rectally), topically, or parenterally (for example, intravenously, intramuscularly, subcutaneously), in the conventional form of preparations, such as capsules, microcapsules, tablets, granules, powder, troches, pills, suppositories, injections, suspensions, syrups, patches, creams, lotions, ointments, gels, sprays, solutions and emulsions.

Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrrolidone or aluminum stearate), a dispersing agent (e.g., hydroxypropylmethylcellulose), a diluent (e.g., water), a cosolvent (e.g., propylene glocyl/glycofurol), a buffer, a copolymer (e.g., poly(lactic-co-glycolic acid, i.e PLGA), and base wax (e.g., cocoa butter, white petrolatum or polyethylene glycol). The effective amount of the compound in a pharmaceutical composition may be at a level that will exert the desired effect; for example, about 0.005 mg/kg of a subject's body weight to about 20 mg/kg of a subject's body weight in unit dosage for both oral and parenteral administration.

The dose of a compound to be administered to a subject is rather widely variable and can be subject to the judgment of a health-care practitioner. In general, the compounds described herein can be administered one to four times a day in a dose of about 0.5 mg/kg of a subject's body weight to about 20 mg/kg of a subject's body weight in a subject, but the above dosage may be properly varied depending, for example, on the age, body weight, and medical condition of the subject and/or the route of administration. In one embodiment, the dose is about 0.1 mg/kg of a subject's body weight to about 3 mg/kg of a subject's body weight, about 0.5 mg/kg of a subject's body weight to about 2 mg/kg of a subject's body weight, about 1 mg/kg of a subject's body weight to about 2 mg/kg of a subject's body weight or about 1.5 mg/kg of a subject's body weight to about 2 mg/kg of a subject's body weight. In one embodiment, the dose is about 1 mg/kg of a subject's body weight to about 3 mg/kg of a subject's body weight. In one embodiment, the dose is about 0.5 mg/kg of a subject's body weight to about 1 mg/kg of a subject's body weight. In one embodiment, the dose is about 1 mg/kg of a subject's body weight to about 2 mg/kg of a subject's body weight. In one embodiment, the dose is about 1.5, 1.6, 1.7, 1.8, 1.9, 2.0, 2.1, 2.2, 2.3, 2.4, 2.5, 2.6, 2.7, 2.8, 2.9, 3.0 mg/kg of a subject's body weight. In one embodiment, one dose is given per day. In any given case, the amount of the compound administered will depend on such factors as the solubility of the active component, the formulation used, and the route of administration. In one embodiment, application of a topical concentration provides intracellular exposures or concentrations of about 0.01-10 μM.

In some embodiments of the methods described herein, about 1 mg/day to about 1200 mg/day of a compound of the present disclosure (e.g., a compound of structural formula (I), (II), (III), or (IV), of Table 1), or a pharmaceutically acceptable salt, tautomer, isotopologue, or stereoisomer thereof, is administered to a subject, e.g., a subject affected by a hemoglobinopathy. In some embodiments, about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a compound of the present disclosure, or a pharmaceutically acceptable salt, tautomer, isotopologue, or stereoisomer thereof, is administered to a subject, e.g., a subject affected by a hemoglobinopathy. In some embodiments, about 0.375 mg/day to about 750 mg/day of a compound of the present disclosure, or a pharmaceutically acceptable salt, tautomer, isotopologue, or stereoisomer thereof, is administered to a subject, e.g., a subject affected by a hemoglobinopathy. In some embodiments, about 3.75 mg/day to about 75 mg/day of a compound of the present disclosure, or a pharmaceutically acceptable salt, tautomer, isotopologue, or stereoisomer thereof, is administered to a subject, e.g., a subject affected a hemoglobinopathy.

Also provided herein are unit dosage formulations of a compound of the disclosure (e.g., a compound of structural formula (I), (II), (III), or (IV), of Table 1), or a pharmaceutically acceptable salt, tautomer, isotopologue, or stereoisomer thereof. In some embodiments, the unit dosage formulation comprises between about 1 mg and 200 mg, about 35 mg and about 1400 mg, about 125 mg and about 1000 mg, about 250 mg and about 1000 mg, or about 500 mg and about 1000 mg of a compound of the disclosure, or a pharmaceutically acceptable salt, tautomer, isotopologue, or stereoisomer thereof. In some embodiments, the unit dosage formulation comprises between about 1 mg and 200 mg of a compound of the disclosure, or a pharmaceutically acceptable salt, tautomer, isotopologue, or stereoisomer thereof. In some embodiments, the unit dosage formulation comprises between about 35 mg and about 1,400 mg of a compound of the disclosure, or a pharmaceutically acceptable salt, tautomer, isotopologue, or stereoisomer thereof. In some embodiments, the unit dosage formulation comprises between about 125 mg and about 1,000 mg of a compound of the disclosure, or a pharmaceutically acceptable salt, tautomer, isotopologue, or stereoisomer thereof. In some embodiments, the unit dosage formulation comprises between about 250 mg and about 1,000 mg of a compound of the disclosure, or a pharmaceutically acceptable salt, tautomer, isotopologue, or stereoisomer thereof. In some embodiments, the unit dosage formulation comprises between about 500 mg and about 1,000 mg of a compound of the disclosure, or a pharmaceutically acceptable salt, tautomer, isotopologue, or stereoisomer thereof. In a particular embodiment, provided herein are unit dosage formulations comprising about 100 mg or 400 mg of a compound of the disclosure, or a pharmaceutically acceptable salt, tautomer, isotopologue, or stereoisomer thereof.

In another embodiment, provided herein are unit dosage formulations that comprise 1 mg, 5 mg, 10 mg, 15 mg, 20 mg, 30 mg, 35 mg, 40 mg, 50 mg, 70 mg, 100 mg, 125 mg, 130 mg, 140 mg, 175 mg, 200 mg, 250 mg, 280 mg, 350 mg, 500 mg, 560 mg, 700 mg, 750 mg, 1000 mg or 1400 mg of a compound of the disclosure (e.g., a compound of structural formula (I), (II), (III), or (IV), of Table 1), or a pharmaceutically acceptable salt, tautomer, isotopologue, or stereoisomer thereof. In some embodiments, the unit dosage formulations comprise 1 mg of a compound of the disclosure, or a pharmaceutically acceptable salt, tautomer, isotopologue, or stereoisomer thereof. In some embodiments, the unit dosage formulations comprise 5 mg of a compound of the disclosure, or a pharmaceutically acceptable salt, tautomer, isotopologue, or stereoisomer thereof. In some embodiments, the unit dosage formulations comprise 10 mg of a compound of the disclosure, or a pharmaceutically acceptable salt, tautomer, isotopologue, or stereoisomer thereof. In some embodiments, the unit dosage formulations comprise 15 mg of a compound of the disclosure, or a pharmaceutically acceptable salt, tautomer, isotopologue, or stereoisomer thereof. In some embodiments, the unit dosage formulations comprise 20 mg of a compound of the disclosure, or a pharmaceutically acceptable salt, tautomer, isotopologue, or stereoisomer thereof. In some embodiments, the unit dosage formulations comprise 25 mg of a compound of the disclosure, or a pharmaceutically acceptable salt, tautomer, isotopologue, or stereoisomer thereof. In some embodiments, the unit dosage formulations comprise 30 mg of a compound of the disclosure, or a pharmaceutically acceptable salt, tautomer, isotopologue, or stereoisomer thereof. In some embodiments, the unit dosage formulations comprise 35 mg of a compound of the disclosure, or a pharmaceutically acceptable salt, tautomer, isotopologue, or stereoisomer thereof. In some embodiments, the unit dosage formulations comprise 40 mg of a compound of the disclosure, or a pharmaceutically acceptable salt, tautomer, isotopologue, or stereoisomer thereof. In some embodiments, the unit dosage formulations comprise 50 mg of a compound of the disclosure, or a pharmaceutically acceptable salt, tautomer, isotopologue, or stereoisomer thereof. In some embodiments, the unit dosage formulations comprise 70 mg of a compound of the disclosure, or a pharmaceutically acceptable salt, tautomer, isotopologue, or stereoisomer thereof. In some embodiments, the unit dosage formulations comprise 100 mg of a compound of the disclosure, or a pharmaceutically acceptable salt, tautomer, isotopologue, or stereoisomer thereof. In some embodiments, the unit dosage formulations comprise 125 mg of a compound of the disclosure, or a pharmaceutically acceptable salt, tautomer, isotopologue, or stereoisomer thereof. In some embodiments, the unit dosage formulations comprise 130 mg of a compound of the disclosure, or a pharmaceutically acceptable salt, tautomer, isotopologue, or stereoisomer thereof. In some embodiments, the unit dosage formulations comprise 140 mg of a compound of the disclosure, or a pharmaceutically acceptable salt, tautomer, isotopologue, or stereoisomer thereof. In some embodiments, the unit dosage formulations comprise 175 mg of a compound of the disclosure, or a pharmaceutically acceptable salt, tautomer, isotopologue, or stereoisomer thereof. In some embodiments, the unit dosage formulations comprise 200 mg of a compound of the disclosure, or a pharmaceutically acceptable salt, tautomer, isotopologue, or stereoisomer thereof. In some embodiments, the unit dosage formulations comprise 250 mg of a compound of the disclosure, or a pharmaceutically acceptable salt, tautomer, isotopologue, or stereoisomer thereof. In some embodiments, the unit dosage formulations comprise 280 mg of a compound of the disclosure, or a pharmaceutically acceptable salt, tautomer, isotopologue, or stereoisomer thereof. In some embodiments, the unit dosage formulations comprise 350 mg of a compound of the disclosure, or a pharmaceutically acceptable salt, tautomer, isotopologue, or stereoisomer thereof. In some embodiments, the unit dosage formulations comprise 500 mg of a compound of the disclosure, or a pharmaceutically acceptable salt, tautomer, isotopologue, or stereoisomer thereof. In some embodiments, the unit dosage formulations comprise 560 mg of a compound of the disclosure, or a pharmaceutically acceptable salt, tautomer, isotopologue, or stereoisomer thereof. In some embodiments, the unit dosage formulations comprise 700 mg of a compound of the disclosure, or a pharmaceutically acceptable salt, tautomer, isotopologue, or stereoisomer thereof. In some embodiments, the unit dosage formulations comprise 750 mg of a compound of the disclosure, or a pharmaceutically acceptable salt, tautomer, isotopologue, or stereoisomer thereof. In some embodiments, the unit dosage formulations comprise 1,000 mg of a compound of the disclosure, or a pharmaceutically acceptable salt, tautomer, isotopologue, or stereoisomer thereof. In some embodiments, the unit dosage formulations comprise 1,400 mg of a compound of the disclosure, or a pharmaceutically acceptable salt, tautomer, isotopologue, or stereoisomer thereof.

A compound of the disclosure, or a pharmaceutically acceptable salt, tautomer, isotopologue, or stereoisomer thereof can be administered once, twice, three, four or more times daily. In a particular embodiment, daily doses of 600 mg or less are administered as a once daily dose and daily doses of more than 600 mg are administered twice daily in an amount equal to one half of the total daily dose.

A compound of the disclosure, or a pharmaceutically acceptable salt, tautomer, isotopologue, or stereoisomer thereof can be administered orally. In one embodiment, when administered orally, a compound of the disclosure, or a pharmaceutically acceptable salt, tautomer, isotopologue, or stereoisomer thereof is administered with a meal and water. In another embodiment, the compound of the disclosure, or a pharmaceutically acceptable salt, tautomer, isotopologue, or stereoisomer thereof is dispersed in water or juice (e.g., apple juice or orange juice) and administered orally as a suspension.

The compound of the disclosure, or a pharmaceutically acceptable salt, tautomer, isotopologue, or stereoisomer thereof can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, topically to the ears, nose, eyes, or skin, or by local ocular (i.e., subconjunctival, intravitreal, retrobulbar, intracameral). The mode of administration is left to the discretion of the healthcare practitioner, and can depend in-part upon the site of the medical condition.

In one embodiment, provided herein are capsules containing a compound the disclosure, or a pharmaceutically acceptable salt, tautomer, isotopologue, or stereoisomer thereof without an additional carrier, excipient or vehicle.

Compositions can be in the form of tablets, chewable tablets, capsules, solutions, parenteral solutions, troches, suppositories, suspensions, gels, intra-ruminal devices (e.g., for prolonged prophylaxis or controlled release), implants, topical pour-ons, transdermal delivery gels, spot-ons, implants (including devices, gels, liquids (e.g., PLGA), and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of the disclosure, or a pharmaceutically acceptable salt, tautomer, isotopologue, or stereoisomer thereof, with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

The effect of a compound of the disclosure, or a pharmaceutically acceptable salt, tautomer, isotopologue, or stereoisomer thereof can be delayed or prolonged by proper formulation. For example, a slowly soluble pellet of a compound of the disclosure, or a pharmaceutically acceptable salt, tautomer, isotopologue, or stereoisomer thereof can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, e.g., by dissolving or suspending a compound of the disclosure, or a pharmaceutically acceptable salt, tautomer, isotopologue, or stereoisomer thereof in oily or emulsified vehicles, or adding amounts of PLGA, that allow it to disperse slowly in the serum.

### EXAMPLES

The following Examples are presented by way of illustration, not limitation. Compounds are named using the automatic name generating tool provided in Chemdraw Ultra 20.1 (Cambridgesoft), which generates systematic names for chemical structures, with support for the Cahn-Ingold-Prelog rules for stereochemistry. One skilled in the art can modify the procedures set forth in the illustrative examples to arrive at the desired products.

Provided herein are compounds as described in the below table:

TABLE 1

| | | | Observed | |
|---|---|---|---|---|
| Com- | | | LCMS | 1H NMR Data (400 MHz, |
| pound | | | ESI+ | DMSO-d6-unless indicated |
| No. | Structure | Name | (ESI−) | otherwise), δ |
| 1 | 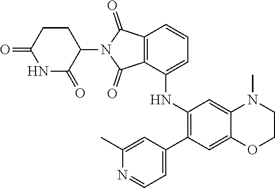 [SMILES: Cc1cc(-c2cc3c(cc2Nc2cccc4c2C(=O)N(C2CCC(=O)NC2=O)C4=O)N(C)CCO3)ccn1] | 2-(2,6-dioxopiperidin-3-yl)-4- ((4-methyl-7-(2-methylpyridin-4- yl)-3,4-dihydro-2H- benzo[b][1,4]oxazin-6- yl)amino)isoindoline-1,3-dione | m/z: 512.2 [M + 1]⁺ | 11.16-11.08 (m, 1H), 8.58-8.48 (m, 1H), 8.38-8.32 (m, 1H), 7.94-7.87 (m, 1H), 7.86-7.79 (m, 1H), 7.46-7.39 (m, 1H), 7.07 (s, 2H), 6.80-6.75 (m, 1H), 6.72- 6.68 (m, 1H), 5.13-5.07 (m, 1H), 4.33-4.27 (m, 2H), 3.43-3.39 (m, 2H), 2.95-2.92 (m, 3H), 2.80-2.70 (m, 1H), 2.65-2.63 (m, 1H), 2.60-2.59 (m, 1H), 2.57 (s, 3H), 2.08-2.03 (m, 1H) |
| 2 | 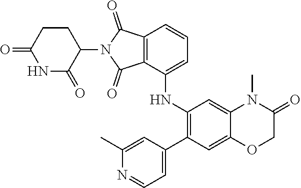 [SMILES: Cc1cc(-c2cc3c(cc2Nc2cccc4c2C(=O)N(C2CCC(=O)NC2=O)C4=O)N(C)C(=O)CO3)ccn1] | 2-(2,6-dioxopiperidin-3-yl)-4- ((4-methyl-7-(2-methylpyridin-4- yl)-3-oxo-3,4-dihydro-2H- benzo[b][1,4]oxazin-6- yl)amino)isoindoline-1,3-dione | m/z: 526.2 [M + 1]⁺ | 11.09 (s, 1H), 8.34 (d, J = 5.2 Hz, 1H), 8.27 (s, 1H), 7.47-7.40 (m, 1H), 7.30 (d, J = 14.8 Hz, 2H), 7.22 (dd, J = 1.2, 5.2 Hz, 1H), 7.15 (s, 1H), 7.09 (d, J = 7.2 Hz, 1H), 6.88-6.80 (m, 1H), 5.08 (dd, J = 5.2, 12.8 Hz, 1H), 4.75 (s, 2H), 3.30 (s, 3H), 2.96-2.82 (m, 1H), 2.63-2.53 (m, 2H), 2.39 (s, 3H), 2.09-1.97 (m, 1H) |
| 3 | 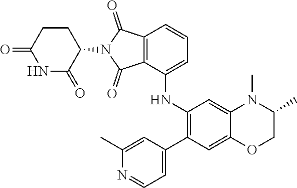 [SMILES: Cc1cc(-c2cc3c(cc2Nc2cccc4c2C(=O)N([C@H]2CCC(=O)NC2=O)C4=O)N(C)[C@H](C)CO3)ccn1] | 4-(((R)-3,4-dimethyl-7-(2- methylpyridin-4-yl)-3,4-dihydro- 2H-benzo[b][1,4]oxazin-6- yl)amino)-2-((S)-2,6- dioxopiperidin-3-yl)isoindoline- 1,3-dione | m/z: 526.2 [M + 1]⁺ | 11.11 (s, 1H), 8.26 (d, J = 5.2 Hz, 1H), 8.12 (s, 1H), 7.41 (dd, J = 7.2, 8.4 Hz, 1H), 7.26 (s, 1H), 7.17 (d, J = 5.2 Hz, 1H), 7.04 (d, J = 7.2 Hz, 1H), 6.86 (s, 1H), 6.77 (d, J = 8.4 Hz, 1H), 6.66 (s, 1H), 5.08 (dd, J = 5.2, 12.8 Hz, 1H), 4.17-4.06 (m, 2H), 3.56-3.48 (m, 1H), 2.94-2.87 (m, 1H), 2.86 (s, 3H), 2.64-2.57 (m 1H), 2.56- 2.53 (m 1H), 2.35 (s, 3H), 2.09- 1.99 (m, 1H), 1.16 (d, J = 6.4 Hz, 3H). |
| 4 | 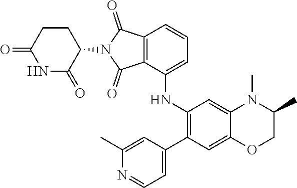 [SMILES: Cc1cc(-c2cc3c(cc2Nc2cccc4c2C(=O)N([C@H]2CCC(=O)NC2=O)C4=O)N(C)[C@@H](C)CO3)ccn1] | 4-(((S)-3,4-dimethyl-7-(2- methylpyridin-4-yl)-3,4-dihydro- 2H-benzo[b][1,4]oxazin-6- yl)amino)-2-((S)-2,6- dioxopiperidin-3-yl)isoindoline- 1,3-dione | m/z: 526.3 [M + 1]⁺ | 11.11 (s, 1H), 8.26 (d, J = 5.2 Hz, 1H), 8.12 (s, 1H), 7.44-7.38 (m, 1H), 7.25 (s, 1H), 7.16 (dd, J = 1.2, 5.1 Hz, 1H), 7.04 (d, J = 7.2 Hz, 1H), 6.86 (s, 1H), 6.80-6.75 (m, 1H), 6.66 (s, 1H), 5.08 (dd, J = 5.2, 12.8 Hz, 1H), 4.17-4.06 (m, 2H), 3.56-3.48 (m, 1H), 2.94- 2.87 (m, 1H), 2.86 (s, 3H), 2.64- 2.57 (m 1H), 2.54-2.52 (m 1H), 2.35 (s, 3H), 2.08-1.98 (m, 1H), 1.16 (d, J = 6.4 Hz, 3H). |
| 5 | 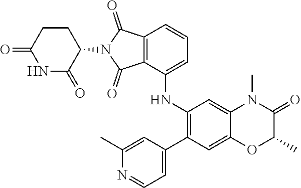 [SMILES: Cc1cc(-c2cc3c(cc2Nc2cccc4c2C(=O)N([C@H]2CCC(=O)NC2=O)C4=O)N(C)C(=O)[C@H](C)O3)ccn1] | 4-(((S)-2,4-dimethyl-7-(2- methylpyridin-4-yl)-3-oxo-3,4- dihydro-2H-benzo[b][1,4]oxazin- 6-yl)amino)-2-((S)-2,6- dioxopiperidin-3-yl)isoindoline- 1,3-dione | m/z: 540.3 [M + 1]⁺ | 11.12-11.09 (m, 1H), 8.33 (d, J = 5.2 Hz, 1H), 8.27 (s, 1H), 7.49- 7.39 (m, 1H), 7.32 (s, 1H), 7.28 (s, 1H), 7.23 (d, J = 4.0 Hz, 1H), 7.16 (s, 1H), 7.09 (d, J = 7.2 Hz, 1H), 6.85 (d, J = 8.8 Hz, 1H), 5.04 (s, 1H), 4.87-4.80 (m, 1H), 3.30 (s, 3H), 2.64-2.57 (m, 1H), 2.44 (dd, J = 11.2, 2.4 Hz, 3H), 2.39 (s, 3H), 1.49 (d, J = 7.2 Hz, 3H), 1.40-1.37 (m, 2H). |
| 6 | 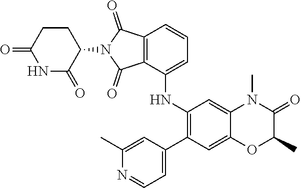 [SMILES: Cc1cc(-c2cc3c(cc2Nc2cccc4c2C(=O)N([C@H]2CCC(=O)NC2=O)C4=O)N(C)C(=O)[C@@H](C)O3)ccn1] | 4-(((R)-2,4-dimethyl-7-(2- methylpyridin-4-yl)-3-oxo-3,4- dihydro-2H-benzo[b][1,4]oxazin- 6-yl)amino)-2-((S)-2,6- dioxopiperidin-3-yl)isoindoline- 1,3-dione | m/z 540.3 [M + 1]⁺ | 11.11 (s, 1H), 8.34 (d, J = 5.2 Hz, 1H), 8.28 (s, 1H), 7.43 (t, J = 8.0 Hz, 1H), 7.34 (s, 1H), 7.28 (s, 1H), 7.25 (d, J = 5.2 Hz, 1H), 7.17 (s, 1H), 7.09 (d, J = 7.2 Hz, 1H), 6.88- 6.81 (m, 1H), 5.08 (dd, J = 12.4, 5.2 Hz, 1H), 4.88-4.80 (m, 1H), 3.31-3.31 (m, 3H), 2.67 (s, 1H), 2.64-2.59 (m, 1H), 2.57 (s, 1H), 2.47 (d, J = 2.4 Hz, 1H), 2.39 (s, 3H), 1.49 (d, J = 6.4 Hz, 3H). |
| 7 | 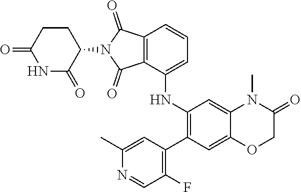 [SMILES: Cc1cc(-c2cc3c(cc2Nc2cccc4c2C(=O)N([C@H]2CCC(=O)NC2=O)C4=O)N(C)C(=O)CO3)c(F)cn1] | (S)-2-(2,6-dioxopiperidin-3-yl)- 4-((7-(5-fluoro-2-methylpyridin- 4-yl)-4-methyl-3-oxo-3,4- dihydro-2H-benzo[b][1,4]oxazin- 6-yl)amino)isoindoline-1,3-dione | m/z 544.3 [M + 1]⁺ | 11.11 (s, 1H), 8.37 (d, J = 1.2 Hz, 1H), 8.10 (s, 1H), 7.48 (dd, J = 7.6, 8.4 Hz, 1H), 7.35-7.30 (m, 2H), 7.16 (s, 1H), 7.12 (d, J = 7.2 Hz, 1H), 6.96 (d, J = 8.8 Hz, 1H), 5.07 (dd, J = 5.2, 12.8 Hz, 1H), 4.76 (s, 2H), 3.30 (s, 3H), 2.94- 2.81 (m, 1H), 2.64-2.51 (m, 2H), 2.40 (s, 3H), 2.07-1.99 (m, 1H) |
| 8 | 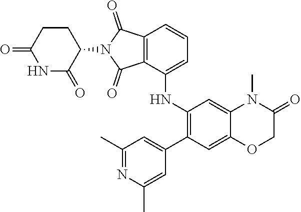 [SMILES: Cc1cc(-c2cc3c(cc2Nc2cccc4c2C(=O)N([C@H]2CCC(=O)NC2=O)C4=O)N(C)C(=O)CO3)cc(C)n1] | (S)-4-((7-(2,6-dimethylpyridin-4- yl)-4-methyl-3-oxo-3,4-dihydro- 2H-benzo[b][1,4]oxazin-6- yl)amino)-2-(2,6-dioxopiperidin- 3-yl)isoindoline-1,3-dione | m/z: 540.2 [M + 1]⁺ | 11.11 (s, 1H), 8.28 (s, 1H), 7.47- 7.41 (m, 1H), 7.27 (s, 1H), 7.13 (s, 1H), 7.10 (d, J = 7.2 Hz, 1H), 7.08 (s, 2H), 6.91-6.85 (m, 1H), 5.09 (dd, J = 5.6, 12.8 Hz, 1H), 4.75 (s, 2H), 3.31-3.30 (m, 3H), 2.96-2.83 (m, 1H), 2.64-2.57 (m, 1H), 2.56-2.53 (m, 1H), 2.33 (s, 6H), 2.08-1.99 (m, 1H) |
| 9 | 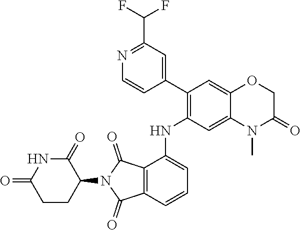 [SMILES: CN1C(=O)COc2cc(-c3ccnc(C(F)F)c3)c(Nc3cccc4c3C(=O)N([C@H]3CCC(=O)NC3=O)C4=O)cc21] | (S)-4-((7-(2- (difluoromethyl)pyridin-4-yl)-4- methyl-3-oxo-3,4-dihydro-2H- benzo[b][1,4]oxazin-6- yl)amino)-2-(2,6-dioxopiperidin- 3-yl)isoindoline-1,3-dione | m/z 562.2 [M + 1]⁺ | 11.12 (s, 1H), 8.58 (d, J = 5.2 Hz, 1H), 8.39 (s, 1H), 7.74 (s, 1H), 7.63 (d, J = 4.8 Hz, 1H), 7.42-7.36 (m, 1H), 7.32-7.28 (m, 1H), 7.25 (s, 1H), 7.07 (d, J = 7.2 Hz, 1H), 6.99-6.70 (m, 2H), 5.08 (dd, J = 5.2, 12.8 Hz, 1H), 4.77 (s, 2H), 3.30 (s, 3H), 2.95-2.82 (m, 1H), 2.64-2.53 (m, 2H), 2.05-1.95 (m, 1H) |
| 10 | 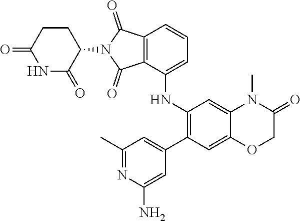 [SMILES: Cc1cc(-c2cc3c(cc2Nc2cccc4c2C(=O)N([C@H]2CCC(=O)NC2=O)C4=O)N(C)C(=O)CO3)cc(N)n1] | (S)-4-((7-(2-amino-6- methylpyridin-4-yl)-4-methyl-3- oxo-3,4-dihydro-2H- benzo[b][1,4]oxazin-6- yl)amino)-2-(2,6-dioxopiperidin- 3-yl)isoindoline-1,3-dione | m/z: 541.3 [M + 1]⁺ | 11.11 (s, 1H), 8.12 (s, 1H), 7.54- 7.49 (m, 1H), 7.26 (s, 1H), 7.16 (d, J = 7.2 Hz, 1H), 7.11-7.06 (m, 1H), 7.01 (s, 1H), 6.38 (s, 1H), 6.22 (s, 1H), 5.84-5.79 (m, 1H), 5.09 (dd, J = 12.8, 5.2 Hz, 1H), 4.74 (s, 2H), 3.30 (s, 3H), 2.95-2.84 (m, 1H), 2.65-2.57 (m, 2H), 2.16 (s, 3H), 2.07-2.00 (m, 1H). |
| 11 | 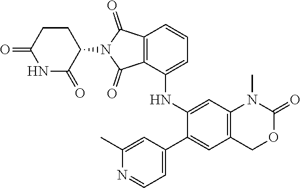 [SMILES: Cc1cc(-c2cc3c(cc2Nc2cccc4c2C(=O)N([C@H]2CCC(=O)NC2=O)C4=O)N(C)C(=O)OC3)ccn1] | (S)-2-(2,6-dioxopiperidin-3-yl)- 4-((1-methyl-6-(2-methylpyridin- 4-yl)-2-oxo-1,4-dihydro-2H- benzo[d][1,3]oxazin-7- yl)amino)isoindoline-1,3-dione | m/z 526.2 [M + 1]⁺ | 11.11 (s, 1H), 8.39 (s, 1H), 8.37 (d, J = 5.2 Hz, 1H), 7.48 (dd, J = 7.2, 8.4 Hz, 1H), 7.41 (s, 1H), 7.30 (s, 1H), 7.23-7.19 (m, 2H), 7.16 (d, J = 7.2 Hz, 1H), 7.08 (d, J = 8.4 Hz, 1H), 5.32 (s, 2H), 5.08 (dd, J = 5.2, 12.8 Hz, 1H), 3.30 (s, 3H), 2.93-2.83 (m, 1H), 2.64- 2.54 (m, 2H), 2.41 (s, 3H), 2.06- 1.99 (m, 1H) |
| 12 | 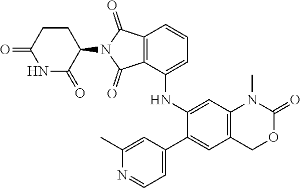 [SMILES: Cc1cc(-c2cc3c(cc2Nc2cccc4c2C(=O)N([C@@H]2CCC(=O)NC2=O)C4=O)N(C)C(=O)OC3)ccn1] | (R)-2-(2,6-dioxopiperidin-3-yl)- 4-((1-methyl-6-(2-methylpyridin- 4-yl)-2-oxo-1,4-dihydro-2H- benzo[d][1,3]oxazin-7- yl)amino)isoindoline-1,3-dione | m/z 526.3 [M + 1]⁺ | 11.11 (s, 1H), 8.39 (s, 1H), 8.37 (d, J = 5.2 Hz, 1H), 7.48 (dd, J = 7.2, 8.4 Hz, 1H), 7.41 (s, 1H), 7.31 (s, 1H), 7.23-7.20 (m, 2H), 7.16 (d, J = 7.2 Hz, 1H), 7.08 (d, J = 8.4 Hz, 1H), 5.32 (s, 2H), 5.08 (dd, J = 5.2, 12.8 Hz, 1H), 3.30 (s, 3H), 2.93-2.83 (m, 1H), 2.63- 2.55 (m, 2H), 2.41 (s, 3H), 2.07- 2.00 (m, 1H) |
| 13 | 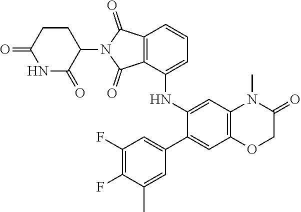 [SMILES: Cc1cc(-c2cc3c(cc2Nc2cccc4c2C(=O)N(C2CCC(=O)NC2=O)C4=O)N(C)C(=O)CO3)cc(F)c1F] | 4-((7-(3,4-difluoro-5- methylphenyl)-4-methyl-3-oxo- 3,4-dihydro-2H- benzo[b][1,4]oxazin-6- yl)amino)-2-(2,6-dioxopiperidin- 3-yl)isoindoline-1,3-dione | m/z: 560.9 [M + 1]⁺ (558.8) | 11.12 (s, 1H), 8.26 (s, 1H), 7.44 (dd, J = 8.4, 7.3 Hz, 1H), 7.30-7.39 (m, 1H), 7.26 (s, 1H), 7.18 (br d, J = 6.3 Hz, 1H), 7.12 (s, 1H), 7.10 (d, J = 6.9 Hz, 1H), 6.82 (d, J = 8.6 Hz, 1H), 5.09 (dd, J = 12.8, 5.4 Hz, 1H), 4.75 (s, 2H), 3.30 (s, 3H), 2.77-2.96 (m, 1H), 2.53-2.69 (m, 2H), 2.16-2.25 (m, 3H), 1.98-2.11 (m, 1H) |
| 14 | 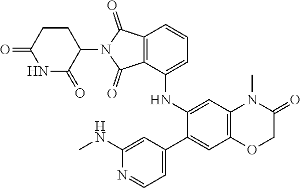 [SMILES: CNc1cc(-c2cc3c(cc2Nc2cccc4c2C(=O)N(C2CCC(=O)NC2=O)C4=O)N(C)C(=O)CO3)ccn1] | 2-(2,6-dioxopiperidin-3-yl)-4- ((4-methyl-7-(2- (methylamino)pyridin-4-yl)-3- oxo-3,4-dihydro-2H- benzo[b][1,4]oxazin-6- yl)amino)isoindoline-1,3-dione | m/z: 541.5 [M + 1]⁺ | 12.74 (br s, 1H), 11.11 (s, 1H), 8.12-8.17 (m, 2H), 7.90 (d, J = 5.3 Hz, 1H), 7.49 (dd, J = 8.4, 7.3 Hz, 1H), 7.27 (s, 1H), 7.13 (d, J = 7.0 Hz, 1H), 7.06 (s, 1H), 6.97 (d, J = 8.5 Hz, 1H), 6.52 (dd, J = 5.3, 1.4 Hz, 1H), 6.40-6.46 (m, 2H), 5.08 (dd, J = 12.8, 5.4 Hz, 1H), 4.75 (s, 2H), 3.28-3.30 (m, 3H), 2.83-2.94 (m, 1H), 2.65-2.73 (m, 3H), 1.99-2.10 (m, 1H) |
| 15 | 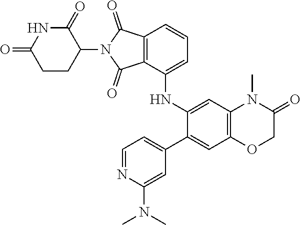 [SMILES: CN(C)c1cc(-c2cc3c(cc2Nc2cccc4c2C(=O)N(C2CCC(=O)NC2=O)C4=O)N(C)C(=O)CO3)ccn1] | 4-((7-(2-(dimethylamino)pyridin- 4-yl)-4-methyl-3-oxo-3,4- dihydro-2H-benzo[b][1,4]oxazin- 6-yl)amino)-2-(2,6- dioxopiperidin-3-yl)isoindoline- 1,3-dione | m/z: 555.5 [M + 1]⁺ | 8.25 (s, 1H), 7.99-8.01 (d, J = 8.0 Hz, 1H), 7.44-7.48 (t, J = 16.0 Hz, 1H), 7.28 (s, 1H), 7.15 (s, 1H), 7.10-7.12 (d, J = 8.0 Hz, 1H), 6.88- 6.89 (d, J = 4.0 Hz, 1H), 6.66 (s, 1H), 6.58-6.59 (d, J = 4.0 Hz, 1H), 5.06-5.11 (dd, J = 12.0 Hz, 8.0 Hz, 1H), 4.75 (s, 2H), 3.30 (s, 3H), 2.68-2.93 (m, 7H), 2.54-2.67 (m, 2H), 2.02-2.06 (m, 1H) |
| 16 | 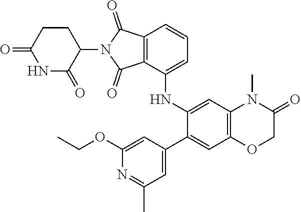 [SMILES: CCOc1cc(-c2cc3c(cc2Nc2cccc4c2C(=O)N(C2CCC(=O)NC2=O)C4=O)N(C)C(=O)CO3)cc(C)n1] | 2-(2,6-dioxopiperidin-3-yl)-4- ((7-(2-ethoxy-6-methylpyridin-4- yl)-4-methyl-3-oxo-3,4-dihydro- 2H-benzo[b][1,4]oxazin-6- yl)amino)isoindoline-1,3-dione | m/z: 570.2 [M + 1]⁺ (568.2) | 11.11 (s, 1H), 8.26 (s, 1H), 7.46 (dd, J = 8.4, 7.3 Hz, 1H), 7.26 (s, 1H), 7.12 (s, 1H), 7.11 (d, J = 7.3 Hz, 1H), 6.83-6.91 (m, 2H), 6.61 (s, 1H), 5.09 (dd, J = 12.7, 5.4 Hz, 1H), 4.75 (s, 2H), 4.21 (q, J = 7.0 Hz, 2H), 3.30 (s, 3H), 2.84-2.95 (m, 1H), 2.57-2.65 (m, 2H), 2.29 (s, 3H), 2.00-2.11 (m, 1H), 1.25 (t, J = 7.1 Hz, 3H) |
| 17 | 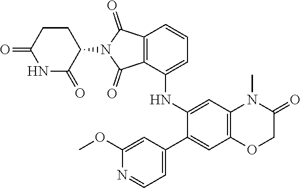 [SMILES: COc1cc(-c2cc3c(cc2Nc2cccc4c2C(=O)N([C@H]2CCC(=O)NC2=O)C4=O)N(C)C(=O)CO3)ccn1] | (S)-2-(2,6-dioxopiperidin-3-yl)- 4-((7-(2-methoxypyridin-4-yl)-4- methyl-3-oxo-3,4-dihydro-2H- benzo[b][1,4]oxazin-6- yl)amino)isoindoline-1,3-dione | m/z: 542.2 [M + 1]⁺ | 11.11 (s, 1H), 8.25 (s, 1H), 8.07 (d, J = 5.2 Hz, 1H), 7.47-7.42 (m, 1H), 7.26 (s, 1H), 7.14 (s, 1H), 7.10 (d, J = 7.2 Hz, 1H), 7.04 (dd, J = 12.8, 5.6 Hz, 1H), 6.87 (s, 1H), 6.82 (d, J = 8.8 Hz, 1H), 5.08 (dd, J = 5.6, 12.8 Hz, 1H), 4.75 (s, 2H), 3.79 (s, 3H), 3.28 (s, 3H), 2.89-2.84 (m, 1H), 2.63-2.52 (m, 2H), 2.09-1.97 (m, 1H) |
| 18 | 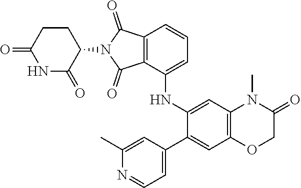 [SMILES: Cc1cc(-c2cc3c(cc2Nc2cccc4c2C(=O)N([C@H]2CCC(=O)NC2=O)C4=O)N(C)C(=O)CO3)ccn1] | (S)-2-(2,6-dioxopiperidin-3-yl)- 4-((4-methyl-7-(2-methylpyridin- 4-yl)-3-oxo-3,4-dihydro-2H- benzo[b][1,4]oxazin-6- yl)amino)isoindoline-1,3-dione | m/z 526.3 [M + 1]⁺ | 11.11 (s, 1H), 8.34 (d, J = 5.2 Hz, 1H), 8.27 (s, 1H), 7.48-7.40 (m, 1H), 7.32 (s, 1H), 7.28 (s, 1H), 7.22 (d, J = 5.2 Hz, 1H), 7.15 (s, 1H), 7.09 (d, J = 7.2 Hz, 1H), 6.83 (d, J = 8.8 Hz, 1H), 5.08 (dd, J = 5.2, 12.8 Hz, 1H), 4.75 (s, 2H), 3.30 (s, 3H), 2.96-2.81 (m, 1H), 2.64-2.52 (m, 2H), 2.39 (s, 3H), 2.10-1.96 (m, 1H) |
| 19 | 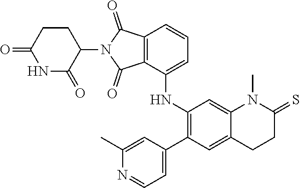 [SMILES: Cc1cc(-c2cc3c(cc2Nc2cccc4c2C(=O)N(C2CCC(=O)NC2=O)C4=O)N(C)C(=S)CC3)ccn1] | 2-(2,6-dioxopiperidin-3-yl)-4- ((1-methyl-6-(2-methylpyridin-4- yl)-2-thioxo-1,2,3,4- tetrahydroquinolin-7- yl)amino)isoindoline-1,3-dione | m/z: 540.2 [M + 1]⁺ | 11.12 (s, 1H), 8.58 (s, 1H), 8.50 (s, 1H), 7.82-7.75 (m, 1H), 7.71- 7.66 (m, 1H), 7.53 (s, 1H), 7.49- 7.45 (m, 2H), 7.17 (d, J = 7.2 Hz, 1H), 6.96 (d, J = 8.4 Hz, 1H), 5.09 (dd, J = 12.8, 5.2 Hz, 1H), 3.85 (s, 3H), 3.19-3.15 (m, 2H), 2.94-2.90 (m, 1H), 2.89-2.86 (m, 2H), 2.62-2.57 (m, 2H), 2.56 (s, 3H), 2.07-2.01 (m, 1H). |
| 20 | 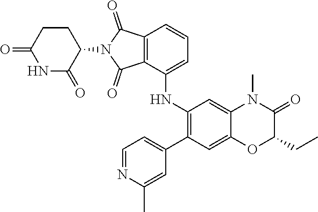 [SMILES: CC[C@@H]1Oc2cc(-c3ccnc(C)c3)c(Nc3cccc4c3C(=O)N([C@H]3CCC(=O)NC3=O)C4=O)cc2N(C)C1=O] | 2-((S)-2,6-dioxopiperidin-3-yl)- 4-(((S)-2-ethyl-4-methyl-7-(2- methylpyridin-4-yl)-3-oxo-3,4- dihydro-2H-benzo[b][1,4]oxazin- 6-yl)amino)isoindoline-1,3-dione | m/z 554.3 [M + 1]⁺ | 11.21 (s, 1H), 8.44 (d, J = 5.2 Hz, 1H), 8.37 (s, 1H), 7.57-7.51 (m, 1H), 7.44 (s, 1H), 7.39-7.32 (m, 2H), 7.28 (s, 1H), 7.20 (d, J = 7.2 Hz, 1H), 6.96 (d, J = 8.4 Hz, 1H), 5.21-5.17 (m, 1H), 4.79-4.76 (m, 1H), 3.41 (s, 3H), 3.05-2.95 (m, 1H), 2.76-2.65 (m, 2H), 2.61 (s, 3H), 2.20-2.12 (m, 1H), 2.03- 1.88 (m, 2H), 1.14 (t, J = 7.2 Hz, 3H). |
| 21 | 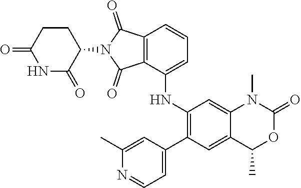 [SMILES: Cc1cc(-c2cc3c(cc2Nc2cccc4c2C(=O)N([C@H]2CCC(=O)NC2=O)C4=O)N(C)C(=O)O[C@@H]3C)ccn1] | 4-(((R)-1,4-dimethyl-6-(2- methylpyridin-4-yl)-2-oxo-1,4- dihydro-2H-benzo[d][1,3]oxazin- 7-yl)amino)-2-((S)-2,6- dioxopiperidin-3-yl)isoindoline- 1,3-dione | m/z 540.6 [M + 1]+ | (ACETONITRILE-d3) 8.95 (br s, 1H), 8.48-8.33 (m, 1H), 7.99 (s, 1H), 7.48 (dd, J = 8.3, 7.3 Hz, 1H), 7.32-7.24 (m, 2H), 7.23-7.14 (m, 4H), 5.52-5.40 (m, 1H), 4.99- 4.85 (m, 1H), 3.38-3.26 (m, 3H), 2.81-2.59 (m, 3H), 2.49- 2.42 (m, 3H), 2.12-2.06 (m, 1H), 1.72-1.62 (m, 3H) |
| 22 | 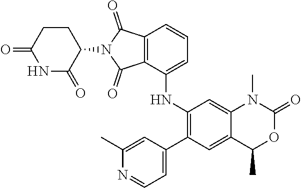 [SMILES: Cc1cc(-c2cc3c(cc2Nc2cccc4c2C(=O)N([C@H]2CCC(=O)NC2=O)C4=O)N(C)C(=O)O[C@H]3C)ccn1] | 4-(((S)-1,4-dimethyl-6-(2- methylpyridin-4-yl)-2-oxo-1,4- dihydro-2H-benzo[d][1,3]oxazin- 7-yl)amino)-2-((S)-2,6- dioxopiperidin-3-yl)isoindoline- 1,3-dione | m/z 540.6 [M + 1]+ | (ACETONITRILE-d3) 8.95 (br s, 1H), 8.44-8.30 (m, 1H), 7.99 (s, 1H), 7.59-7.41 (m, 1H), 7.30- 7.25 (m, 2H), 7.22-7.14 (m, 4H), 5.50-5.40 (m, 1H), 4.99-4.88 (m, 1H), 3.36-3.29 (m, 3H), 2.82- 2.58 (m, 3H), 2.48-2.42 (m, 3H), 2.12-2.06 (m, 1H), 1.71- 1.64 (m, 3H) |
| 23 | 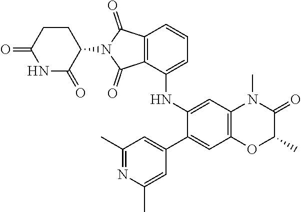 [SMILES: Cc1cc(-c2cc3c(cc2Nc2cccc4c2C(=O)N([C@H]2CCC(=O)NC2=O)C4=O)N(C)C(=O)[C@H](C)O3)cc(C)n1] | 4-(((S)-7-(2,6-dimethylpyridin-4- yl)-2,4-dimethyl-3-oxo-3,4- dihydro-2H-benzo[b][1,4]oxazin- 6-yl)amino)-2-((S)-2,6- dioxopiperidin-3-yl)isoindoline- 1,3-dione | m/z: 554.3 [M + 1]⁺ | 11.12 (s, 1H), 8.28 (s, 1H), 7.44- 7.42 (m, 1H), 7.27 (s, 1H), 7.14 (s, 1H), 7.12-7.07 (m, 3H), 6.88 (d, J = 8.4 Hz, 1H), 5.09 (dd, J = 5.6, 12.8 Hz, 1H), 4.83 (q, J = 6.8 Hz, 1H), 3.31 (s, 3H), 2.89 (s, 1H), 2.73-2.54 (m, 2H), 2.33 (s, 6H), 2.08-2.00 (m, 1H), 1.49 (d, J = 6.8 Hz, 3H). |
| 24 | 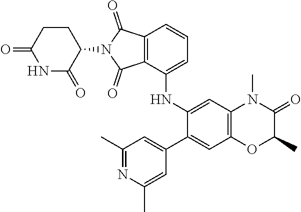 [SMILES: Cc1cc(-c2cc3c(cc2Nc2cccc4c2C(=O)N([C@H]2CCC(=O)NC2=O)C4=O)N(C)C(=O)[C@@H](C)O3)cc(C)n1] | 4-(((R)-7-(2,6-dimethylpyridin- 4-yl)-2,4-dimethyl-3-oxo-3,4- dihydro-2H-benzo[b][1,4]oxazin- 6-yl)amino)-2-((S)-2,6- dioxopiperidin-3-yl)isoindoline- 1,3-dione | m/z: 554.3 [M + 1]⁺ | 11.12 (s, 1H), 8.28 (s, 1H), 7.53- 7.41 (m, 1H), 7.27 (s, 1H), 7.14 (s, 1H), 7.12-7.08 (m, 3H), 6.89 (d, J = 8.4 Hz, 1H), 5.09 (dd, J = 5.6, 12.8 Hz, 1H), 4.87-4.79 (m, 1H), 3.31 (s, 3H), 2.95-2.83 (m, 1H), 2.69-2.52 (m, 2H), 2.33 (s, 6H), 2.07-1.99 (m, 1H), 1.49 (d, J = 6.8 Hz, 3H). |
| 25 | 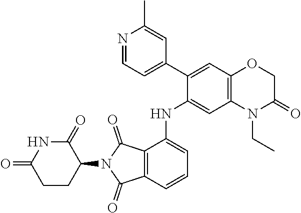 [SMILES: CCN1C(=O)COc2cc(-c3ccnc(C)c3)c(Nc3cccc4c3C(=O)N([C@H]3CCC(=O)NC3=O)C4=O)cc21] | (S)-2-(2,6-dioxopiperidin-3-yl)- 4-((4-ethyl-7-(2-methylpyridin-4- yl)-3-oxo-3,4-dihydro-2H- benzo[b][1,4]oxazin-6- yl)amino)isoindoline-1,3-dione | m/z: 540.3 [M + 1]⁺ | 11.11 (s, 1H), 8.34-8.29 (m, 2H), 7.46-7.38 (m, 1H), 7.31 (s, 2H), 7.22 (d, J = 5.2 Hz, 1H), 7.16 (s, 1H), 7.08 (d, J = 7.2 Hz, 1H), 6.76 (d, J = 8.4 Hz, 1H), 5.08 (dd, J = 5.2, 12.8 Hz, 1H), 4.73 (s, 2H), 3.98-3.93 (m, 2H), 2.94- 2.84 (m, 1H), 2.69-2.53 (m, 2H), 2.38 (s, 3H), 2.08-1.99 (m, 1H), 1.16 (t, J = 7.2 Hz, 3H) |
| 26 | 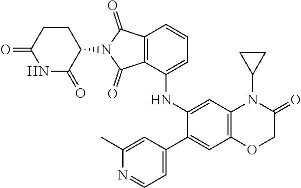 [SMILES: Cc1cc(-c2cc3c(cc2Nc2cccc4c2C(=O)N([C@H]2CCC(=O)NC2=O)C4=O)N(C2CC2)C(=O)CO3)ccn1] | (S)-4-((4-cyclopropyl-7-(2- methylpyridin-4-yl)-3-oxo-3,4- dihydro-2H-benzo[b][1,4]oxazin- 6-yl)amino)-2-(2,6- dioxopiperidin-3-yl)isoindoline- 1,3-dione | m/z: 552.3 [M + 1]⁺ | 11.11 (s, 1H), 8.35 (d, J = 5.2 Hz, 1H), 8.29 (s, 1H), 7.50-746 (m, 1H), 7.43 (s, 1H), 7.31 (s, 1H), 7.23-7.21 (m, 1H), 7.14-7.10 (m, 2H), 6.94 (d, J = 8.4 Hz, 1H), 5.10-5.05 (m, 1H), 4.68 (s, 2H), 2.93-2.83 (m, 1H), 2.78-2.72 (m, 1H), 2.62-2.52 (m, 2H), 2.40 (s, 3H), 2.07-1.99 (m, 1H), 1.11- 1.04 (m, 2H), 0.74-0.67 (m, 2H). |
| 27 | 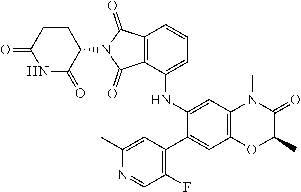 [SMILES: Cc1cc(-c2cc3c(cc2Nc2cccc4c2C(=O)N([C@H]2CCC(=O)NC2=O)C4=O)N(C)C(=O)[C@@H](C)O3)c(F)cn1] | 2-((S)-2,6-dioxopiperidin-3-yl)- 4-(((R)-7-(5-fluoro-2- methylpyridin-4-yl)-2,4- dimethyl-3-oxo-3,4-dihydro-2H- benzo[b][1,4]oxazin-6- yl)amino)isoindoline-1,3-dione | m/z: 558.3 [M + 1]⁺ | 11.10 (s, 1H), 8.38 (s, 1H), 8.10 (s, 1H), 7.51-7.47 (m, 1H), 7.35- 7.34 (m, 2H), 7.17 (s, 1H), 7.13 (d, J = 6.8 Hz, 1H), 6.98 (d, J = 8.4 Hz, 1H), 5.08 (dd, J = 12.8, 5.6 Hz, 1H), 4.86 (q, J = 6.8 Hz, 1H), 3.32 (s, 3H), 2.93-2.84 (m, 1H), 2.62- 2.54 (m, 2H), 2.41 (s, 3H), 2.05- 2.02 (m, 1H), 1.50 (d, J = 6.8 Hz, 3H). |
| 28 | 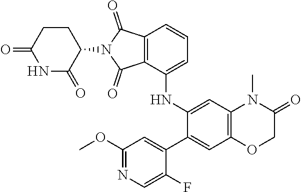 [SMILES: COc1cc(-c2cc3c(cc2Nc2cccc4c2C(=O)N([C@H]2CCC(=O)NC2=O)C4=O)N(C)C(=O)CO3)c(F)cn1] | (S)-2-(2,6-dioxopiperidin-3-yl)- 4-((7-(5-fluoro-2- methoxypyridin-4-yl)-4-methyl- 3-oxo-3,4-dihydro-2H- benzo[b][1,4]oxazin-6- yl)amino)isoindoline-1,3-dione | m/z: 560.3 [M + 1]⁺ | 11.11 (s, 1H), 8.16-8.07 (m, 2H), 7.50 (dd, J = 8.4, 7.2 Hz, 1H), 7.33 (s, 1H), 7.18-7.16 (m, 1H), 7.15-7.12 (m, 1H), 6.96 (d, J = 8.4 Hz, 1H), 6.90 (d, J = 4.8 Hz, 1H), 5.12-5.06 (m, 1H), 4.77 (s, 2H), 3.80 (s, 3H), 3.30 (s, 3H), 2.94-2.83 (m, 1H), 2.62-2.57 (m, 2H), 2.08-2.00 (m, 1H). |
| 29 | 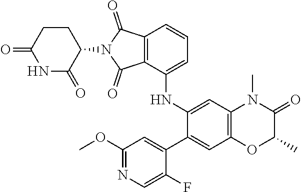 [SMILES: COc1cc(-c2cc3c(cc2Nc2cccc4c2C(=O)N([C@H]2CCC(=O)NC2=O)C4=O)N(C)C(=O)[C@H](C)O3)c(F)cn1] | 2-((S)-2,6-dioxopiperidin-3-yl)- 4-(((S)-7-(5-fluoro-2- methoxypyridin-4-yl)-2,4- dimethyl-3-oxo-3,4-dihydro-2H- benzo[b][1,4]oxazin-6- yl)amino)isoindoline-1,3-dione | m/z: 574.3 [M + 1]⁺ | 11.10 (s, 1H), 8.12 (d, J = 1.2 Hz, 1H), 8.08 (s, 1H), 7.51-7.47 (m, 1H), 7.32 (s, 1H), 7.16 (s, 1H), 7.13 (d, J = 6.8 Hz, 1H), 6.96 (d, J = 8.4 Hz, 1H), 6.89 (d, J = 4.8 Hz, 1H), 5.07 (dd, J = 12.8, 5.6 Hz, 1H), 4.84 (q, J = 6.8 Hz, 1H), 3.79 (s, 3H), 3.30 (s, 3H), 2.93- 2.83 (m, 1H), 2.61-2.53 (m, 2H), 2.05-2.02 (m, 1H), 1.50 (d, J = 6.8 Hz, 3H). |
| 30 | 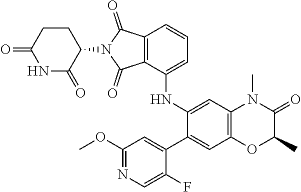 [SMILES: COc1cc(-c2cc3c(cc2Nc2cccc4c2C(=O)N([C@H]2CCC(=O)NC2=O)C4=O)N(C)C(=O)[C@@H](C)O3)c(F)cn1] | 2-((S)-2,6-dioxopiperidin-3-yl)- 4-(((R)-7-(5-fluoro-2- methoxypyridin-4-yl)-2,4- dimethyl-3-oxo-3,4-dihydro-2H- benzo[b][1,4]oxazin-6- yl)amino)isoindoline-1,3-dione | m/z: 574.2 [M + 1]⁺ | 11.12 (s, 1H), 8.13-8.09 (m, 2H), 7.51-7.47 (m, 1H), 7.32 (s, 1H), 7.16 (s, 1H), 7.13 (d, J = 7.2 Hz, 1H), 6.96 (d, J = 8.4 Hz, 1H), 6.89 (d, J = 4.8 Hz, 1H), 5.07 (dd, J = 12.8, 5.2 Hz, 1H), 4.84 (q, J = 6.8 Hz, 1H), 3.79 (s, 3H), 3.29 (s, 3H), 2.92-2.83 (m, 1H), 2.61- 2.53 (m, 2H), 2.04-2.02 (m, 1H), 1.49 (d, J = 6.8 Hz, 3H). |
| 31 | 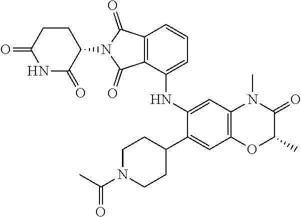 [SMILES: CC(=O)N1CCC(c2cc3c(cc2Nc2cccc4c2C(=O)N([C@H]2CCC(=O)NC2=O)C4=O)N(C)C(=O)[C@H](C)O3)CC1] | 4-(((S)-7-(1-acetylpiperidin-4- yl)-2,4-dimethyl-3-oxo-3,4- dihydro-2H-benzo[b][1,4]oxazin- 6-yl)amino)-2-((S)-2,6- dioxopiperidin-3-yl)isoindoline- 1,3-dione | m/z: 574.3 [M + 1]⁺ | 11.14 (s, 1H), 8.11 (s, 1H), 7.56- 7.52 (m, 1H), 7.17 (d, J = 7.2 Hz, 1H), 7.09 (s, 1H), 7.02 (s, 1H), 6.79 (d, J = 8.8 Hz, 1H), 5.15- 5.11 (m, 1H), 4.76-4.74 (m, 1H), 4.47-4.44 (m, 1H), 3.88-3.81 (m, 1H), 3.22 (s, 3H), 3.00-2.86 (m, 3H), 2.67-2.55 (m, 2H), 2.48- 2.39 (m, 2H), 2.11-2.04 (m, 1H), 1.99 (s, 3H), 1.66-1.63 (m, 3H), 1.45 (d, J = 6.8 Hz, 3H). |
| 32 | 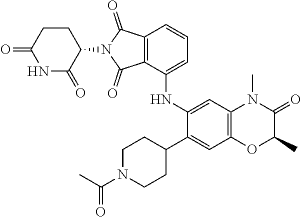 [SMILES: CC(=O)N1CCC(c2cc3c(cc2Nc2cccc4c2C(=O)N([C@H]2CCC(=O)NC2=O)C4=O)N(C)C(=O)[C@@H](C)O3)CC1] | 4-(((R)-7-(1-acetylpiperidin-4- yl)-2,4-dimethyl-3-oxo-3,4- dihydro-2H-benzo[b][1,4]oxazin- 6-yl)amino)-2-((S)-2,6- dioxopiperidin-3-yl)isoindoline- 1,3-dione | m/z: 574.3 [M + 1]⁺ | 11.08 (s, 1H), 8.11 (s, 1H), 7.54 (s, 1H), 7.17 (s, 1H), 7.12-7.00 (m, 2H), 6.79 (s, 1H), 5.20-5.06 (m, 1H), 4.80-4.70 (m, 1H), 4.52- 4.40 (m, 1H), 3.91-3.77 (m, 1H), 3.22 (s, 3H), 3.00-2.84 (m, 3H), 2.64-2.59 (m, 2H), 2.45- 2.25 (m, 2H), 2.12-2.04 (m, 1H), 1.98 (s, 3H), 1.71-1.59 (m, 3H), 1.45 (d, J = 4.8 Hz, 3H). |
| 33 | 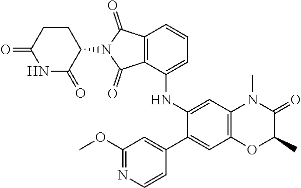 [SMILES: COc1cc(-c2cc3c(cc2Nc2cccc4c2C(=O)N([C@H]2CCC(=O)NC2=O)C4=O)N(C)C(=O)[C@@H](C)O3)ccn1] | 2-((S)-2,6-dioxopiperidin-3-yl)- 4-(((R)-7-(2-methoxypyridin-4- yl)-2,4-dimethyl-3-oxo-3,4- dihydro-2H-benzo[b][1,4]oxazin- 6-yl)amino)isoindoline-1,3-dione | m/z: 556.2 [M + 1]⁺ | 11.12 (s, 1H), 8.25 (s, 1H), 8.06 (d, J = 5.2 Hz, 1H), 7.46-7.42 (m, 1H), 7.26 (s, 1H), 7.15 (s, 1H), 7.09 (d, J = 7.2 Hz, 1H), 7.05-7.03 (m, 1H), 6.87 (s, 1H), 6.82 (d, J = 8.8 Hz, 1H), 5.08 (dd, J = 5.6, 12.8 Hz, 1H), 4.85-4.80 (m, 1H), 3.79 (s, 3H), 3.28 (s, 3H), 2.95-2.83 (m, 1H), 2.69- 2.51 (m, 3H), 1.49-1.48 (m, 3H). |
| 34 | 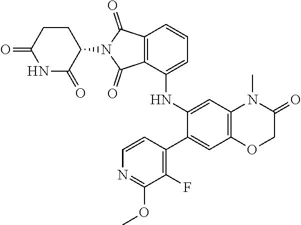 [SMILES: COc1nccc(-c2cc3c(cc2Nc2cccc4c2C(=O)N([C@H]2CCC(=O)NC2=O)C4=O)N(C)C(=O)CO3)c1F] | (S)-2-(2,6-dioxopiperidin-3-yl)- 4-((7-(3-fluoro-2- methoxypyridin-4-yl)-4-methyl- 3-oxo-3,4-dihydro-2H- benzo[b][1,4]oxazin-6- yl)amino)isoindoline-1,3-dione | m/z: 560.2 [M + 1]⁺ | 11.10 (s, 1H), 8.09 (s, 1H), 7.91 (d, J = 5.2 Hz, 1H), 7.51-7.47 (m, 1H), 7.32 (s, 1H), 7.18-7.11 (m, 2H), 7.02-7.00 (m, 1H), 6.94 (d, J = 8.4 Hz, 1H), 5.07 (dd, J = 5.6, 12.8 Hz, 1H), 4.76 (s, 2H), 3.90 (s, 3H), 3.29 (s, 3H), 2.94- 2.82 (m, 1H), 2.65-2.52 (m, 2H), 2.08-1.98 (m, 1H) |
| 35 | 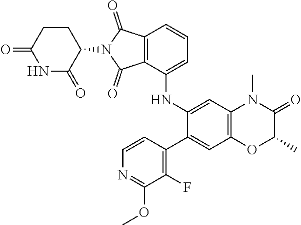 [SMILES: COc1nccc(-c2cc3c(cc2Nc2cccc4c2C(=O)N([C@H]2CCC(=O)NC2=O)C4=O)N(C)C(=O)[C@H](C)O3)c1F] | 2-((S)-2,6-dioxopiperidin-3-yl)- 4-(((S)-7-(3-fluoro-2- methoxypyridin-4-yl)-2,4- dimethyl-3-oxo-3,4-dihydro-2H- benzo[b][1,4]oxazin-6- yl)amino)isoindoline-1,3-dione | m/z: 574.3 [M + 1]⁺ | 11.10 (s, 1H), 8.08 (s, 1H), 7.91 (d, J = 5.2 Hz, 1H), 7.51-7.47 (m, 1H), 7.32 (s, 1H), 7.16-7.11 (m, 2H), 7.01 (t, J = 5.2 Hz, 1H), 6.96 (d, J = 8.8 Hz, 1H), 5.10- 5.03 (m, 1H), 4.85-4.84 (m, 1H), 3.90 (s, 3H), 3.30 (s, 3H), 2.94- 2.81 (m, 1H), 2.61-2.56 (m, 2H), 2.08-1.98 (m, 1H), 1.50 (d, J = 6.8 Hz, 3H). |
| 36 | 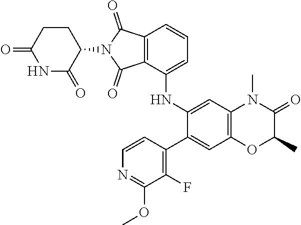 [SMILES: COc1nccc(-c2cc3c(cc2Nc2cccc4c2C(=O)N([C@H]2CCC(=O)NC2=O)C4=O)N(C)C(=O)[C@@H](C)O3)c1F] | 2-((S)-2,6-dioxopiperidin-3-yl)- 4-(((R)-7-(3-fluoro-2- methoxypyridin-4-yl)-2,4- dimethyl-3-oxo-3,4-dihydro-2H- benzo[b][1,4]oxazin-6- yl)amino)isoindoline-1,3-dione | m/z: 574.2 [M + 1]⁺ | 11.11 (s, 1H), 8.08 (s, 1H), 7.91 (d, J = 5.6 Hz, 1H), 7.51-7.47 (m, 1H), 7.32 (s, 1H), 7.16-7.11 (m, 2H), 7.02-7.00 (m, 1H), 6.97- 6.95 (m, 1H), 5.07 (dd, J = 5.6, 12.8 Hz, 1H), 4.87-4.82 (m, 1H), 3.90 (s, 3H), 3.29 (s, 3H), 2.94- 2.82 (m, 1H), 2.65-2.52 (m, 2H), 2.08-1.98 (m, 1H), 1.49 (d, J = 6.8 Hz, 3H). |
| 37 | 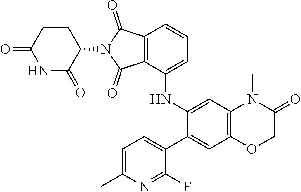 [SMILES: Cc1ccc(-c2cc3c(cc2Nc2cccc4c2C(=O)N([C@H]2CCC(=O)NC2=O)C4=O)N(C)C(=O)CO3)c(F)n1] | (S)-2-(2,6-dioxopiperidin-3-yl)- 4-((7-(2-fluoro-6-methylpyridin- 3-yl)-4-methyl-3-oxo-3,4- dihydro-2H-benzo[b][1,4]oxazin- 6-yl)amino)isoindoline-1,3-dione | m/z: 544.2 [M + 1]⁺ | 11.11 (s, 1H), 8.05 (s, 1H), 7.87- 7.83 (m, 1H), 7.50-7.46 (m, 1H), 7.30 (s, 1H), 7.21-7.19 (m, 1H), 7.14-7.07 (m, 2H), 6.90 (d, J = 8.4 Hz, 1H), 5.07 (dd, J = 5.2, 12.8 Hz, 1H), 4.75 (s, 2H), 3.28 (s, 3H), 2.93-2.81 (m, 1H), 2.58- 2.55 (m, 2H), 2.39 (s, 3H), 2.08- 1.98 (m, 1H) |
| 38 | 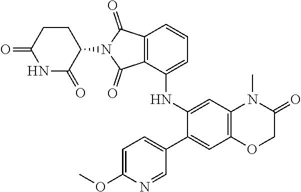 [SMILES: COc1ccc(-c2cc3c(cc2Nc2cccc4c2C(=O)N([C@H]2CCC(=O)NC2=O)C4=O)N(C)C(=O)CO3)cn1] | (S)-2-(2,6-dioxopiperidin-3-yl)- 4-((7-(6-methoxypyridin-3-yl)-4- methyl-3-oxo-3,4-dihydro-2H- benzo[b][1,4]oxazin-6- yl)amino)isoindoline-1,3-dione | m/z: 542.2 [M + 1] | 11.11 (s, 1H), 8.25-8.18 (m, 2H), 7.79 (dd, J = 8.4, 2.4 Hz, 1H), 7.50-7.41 (m, 1H), 7.29-7.23 (m, 1H), 7.13 (s, 1H), 7.11-7.07 (m, 1H), 6.82-6.78 (m, 1H), 6.78- 6.74 (m, 1H), 5.12-5.04 (m, 1H), 4.79-4.72 (m, 2H), 3.85- 3.79 (m, 3H), 3.31-3.27 (m, 3H), 2.95-2.83 (m, 1H), 2.64-2.54 (m, 2H), 2.07-2.00 (m, 1H). |
| 39 | 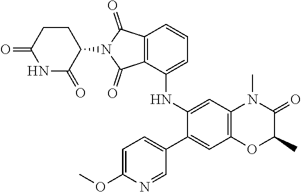 [SMILES: COc1ccc(-c2cc3c(cc2Nc2cccc4c2C(=O)N([C@H]2CCC(=O)NC2=O)C4=O)N(C)C(=O)[C@@H](C)O3)cn1] | 2-((S)-2,6-dioxopiperidin-3-yl)- 4-(((R)-7-(6-methoxypyridin-3- yl)-2,4-dimethyl-3-oxo-3,4- dihydro-2H-benzo[b][1,4]oxazin- 6-yl)amino)isoindoline-1,3-dione | m/z: 556.3 [M + 1]⁺ | 11.10 (s, 1H), 8.22-8.21 (m, 1H), 8.19 (s, 1H), 7.79 (dd, J = 2.4, 8.4 Hz, 1H), 7.47-7.41 (m, 1H), 7.25 (s, 1H), 7.12 (s, 1H), 7.09 (d, J = 7.2 Hz, 1H), 6.80 (d, J = 8.4 Hz, 1H), 6.75 (d, J = 8.4 Hz, 1H), 5.07 (dd, J = 5.6, 12.8 Hz, 1H), 4.83-4.81 (m, 1H), 3.80 (s, 3H), 3.29 (s, 3H), 2.95-2.83 (m, 1H), 2.64-2.52 (m, 2H), 2.07-2.02 (m, 1H), 1.49 (d, J = 6.8 Hz, 3H). |
| 40 | 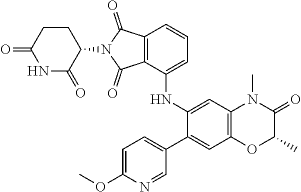 [SMILES: COc1ccc(-c2cc3c(cc2Nc2cccc4c2C(=O)N([C@H]2CCC(=O)NC2=O)C4=O)N(C)C(=O)[C@H](C)O3)cn1] | 2-((S)-2,6-dioxopiperidin-3-yl)- 4-(((S)-7-(6-methoxypyridin-3- yl)-2,4-dimethyl-3-oxo-3,4- dihydro-2H-benzo[b][1,4]oxazin- 6-yl)amino)isoindoline-1,3-dione | m/z: 556.3 [M + 1]⁺ | 11.11 (s, 1H), 8.25-8.17 (m, 2H), 7.79 (dd, J = 2.4, 8.6 Hz, 1H), 7.47-7.40 (m, 1H), 7.25 (s, 1H), 7.15-7.06 (m, 2H), 6.77 (dd, J = 8.4, 18.0 Hz, 2H), 5.07 (dd, J = 5.6, 12.8 Hz, 1H), 4.84-4.80 (m, 1H), 3.80 (s, 3H), 3.29 (s, 3H), 2.94-2.82 (m, 1H), 2.64-2.53 (m, 2H), 2.09-1.99 (m, 1H), 1.49 (d, J = 6.8 Hz, 3H). |
| 41 | 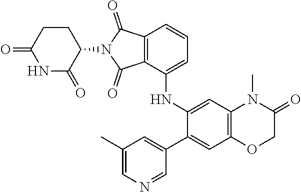 [SMILES: Cc1cncc(-c2cc3c(cc2Nc2cccc4c2C(=O)N([C@H]2CCC(=O)NC2=O)C4=O)N(C)C(=O)CO3)c1] | (S)-2-(2,6-dioxopiperidin-3-yl)- 4-((4-methyl-7-(5-methylpyridin- 3-yl)-3-oxo-3,4-dihydro-2H- benzo[b][1,4]oxazin-6- yl)amino)isoindoline-1,3-dione | m/z: 526.2 [M + 1]⁺ | 11.13-11.08 (m, 1H), 8.41-8.40 (m, 1H), 8.28-8.23 (m, 2H), 7.68 (s, 1H), 7.46-7.39 (m, 1H), 7.30- 7.25 (m, 1H), 7.16 (s, 1H), 7.07 (d, J = 7.2 Hz, 1H), 6.84-6.77 (m, 1H), 5.09-5.05 (m, 1H), 4.78- 4.75 (m, 2H), 3.29 (s, 3H), 2.93- 2.83 (m, 1H), 2.63-2.60 (m, 2H), 2.22 (s, 3H), 2.08-1.98 (m, 1H) |
| 42 | 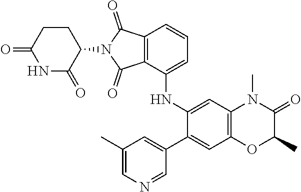 [SMILES: Cc1cncc(-c2cc3c(cc2Nc2cccc4c2C(=O)N([C@H]2CCC(=O)NC2=O)C4=O)N(C)C(=O)[C@@H](C)O3)c1] | 4-(((R)-2,4-dimethyl-7-(5- methylpyridin-3-yl)-3-oxo-3,4- dihydro-2H-benzo[b][1,4]oxazin- 6-yl)amino)-2-((S)-2,6- dioxopiperidin-3-yl)isoindoline- 1,3-dione | m/z: 540.3 [M + 1]⁺ | 11.11 (s, 1H), 8.42-8.41 (m, 1H), 8.26 (s, 2H), 7.68 (s, 1H), 7.44- 7.40 (m, 1H), 7.28 (s, 1H), 7.16 (s, 1H), 7.08-7.07 (m, 1H), 6.81 (d, J = 8.4 Hz, 1H), 5.10-5.05 (m, 1H), 4.86-4.81 (m, 1H), 3.30 (s, 3H), 2.95-2.83 (m, 1H), 2.64- 2.51 (m, 2H), 2.22 (s, 3H), 2.08- 1.99 (m, 1H), 1.50 (d, J = 6.8 Hz, 3H). |
| 43 | 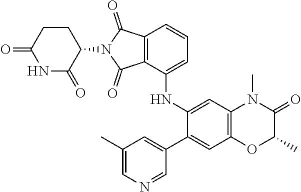 [SMILES: Cc1cncc(-c2cc3c(cc2Nc2cccc4c2C(=O)N([C@H]2CCC(=O)NC2=O)C4=O)N(C)C(=O)[C@H](C)O3)c1] | 4-(((S)-2,4-dimethyl-7-(5- methylpyridin-3-yl)-3-oxo-3,4- dihydro-2H-benzo[b][1,4]oxazin- 6-yl)amino)-2-((S)-2,6- dioxopiperidin-3-yl)isoindoline- 1,3-dione | m/z: 540.2 [M + 1]⁺ | 11.11 (s, 1H), 8.42-8.41 (m, 1H), 8.26 (s, 2H), 7.68 (s, 1H), 7.45- 7.40 (m, 1H), 7.28 (s, 1H), 7.16 (s, 1H), 7.08-7.07 (m, 1H), 6.81 (d, J = 8.4 Hz, 1H), 5.10-5.05 (m, 1H), 4.86-4.81 (m, 1H), 3.30 (s, 3H), 2.94-2.83 (m, 1H), 2.68- 2.51 (m, 2H), 2.22 (s, 3H), 2.08- 1.99 (m, 1H), 1.50 (d, J = 6.4 Hz, 3H). |
| 44 | 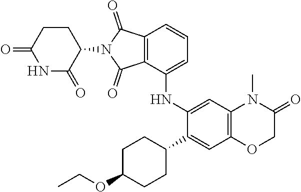 [SMILES: CCO[C@H]1CC[C@H](c2cc3c(cc2Nc2cccc4c2C(=O)N([C@H]2CCC(=O)NC2=O)C4=O)N(C)C(=O)CO3)CC1] | 2-((S)-2,6-dioxopiperidin-3-yl)- 4-((7-((1r,4S)-4- ethoxycyclohexyl)-4-methyl-3- oxo-3,4-dihydro-2H- benzo[b][1,4]oxazin-6- yl)amino)isoindoline-1,3-dione | m/z: 561.3 [M + 1]⁺ | 11.14 (s, 1H), 8.08 (s, 1H), 7.54 (dd, J = 7.2, 8.4 Hz, 1H), 7.16 (d, J = 7.2 Hz, 1H), 7.08 (s, 1H), 7.00 (s, 1H), 6.82 (d, J = 8.4 Hz, 1H), 5.13 (dd, J = 5.2, 12.8 Hz, 1H), 4.67 (s, 2H), 3.43 (q, J = 6.8 Hz, 2H), 3.29-3.24 (m, 1H), 3.22 (s, 3H), 2.97-2.84 (m, 1H), 2.64- 2.54 (m, 3H), 2.14-2.06 (m, 1H), 2.05-1.94 (m, 2H), 1.75-1.62 (m, 2H), 1.55-1.41 (m, 2H), 1.14- 1.04 (m, 5H). |
| 45 | 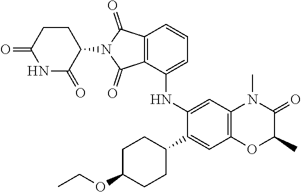 [SMILES: CCO[C@H]1CC[C@H](c2cc3c(cc2Nc2cccc4c2C(=O)N([C@H]2CCC(=O)NC2=O)C4=O)N(C)C(=O)[C@@H](C)O3)CC1] | 2-((S)-2,6-dioxopiperidin-3-yl)- 4-(((R)-7-((1r,4R)-4- ethoxycyclohexyl)-2,4-dimethyl- 3-oxo-3,4-dihydro-2H- benzo[b][1,4]oxazin-6- yl)amino)isoindoline-1,3-dione | m/z: 575.3 [M + 1]+ | 11.14 (s, 1H), 8.11-8.06 (m, 1H), 7.58-7.51 (m, 1H), 7.17 (d, J = 7.2 Hz, 1H), 7.11-7.07 (m, 1H), 7.04-7.00 (m, 1H), 6.88-6.82 (m, 1H), 5.18-5.10 (m, 1H), 4.80- 4.71 (m, 1H), 3.43 (q, J = 7.2 Hz, 2H), 3.29-3.24 (m, 1H), 3.24- 3.21 (m, 3H), 2.97-2.86 (m, 1H), 2.65-2.56 (m, 3H), 2.13- 2.05 (m, 1H), 2.05-1.98 (m, 2H), 1.75-1.68 (m, 2H), 1.52-1.43 (m, 5H), 1.13-1.05 (m, 5H). |
| 46 | 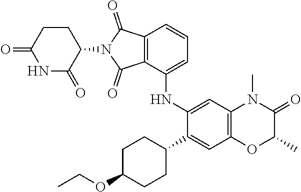 [SMILES: CCO[C@H]1CC[C@H](c2cc3c(cc2Nc2cccc4c2C(=O)N([C@H]2CCC(=O)NC2=O)C4=O)N(C)C(=O)[C@H](C)O3)CC1] | 2-((S)-2,6-dioxopiperidin-3-yl)- 4-(((S)-7-((1r,4S)-4- ethoxycyclohexyl)-2,4-dimethyl- 3-oxo-3,4-dihydro-2H- benzo[b][1,4]oxazin-6- yl)amino)isoindoline-1,3-dione | m/z: 575.3 [M + 1]+ | 11.20 (s, 1H), 8.17-8.13 (m, 1H), 7.63-7.57 (m, 1H), 7.23 (d, J = 7.2 Hz, 1H), 7.16-7.13 (m, 1H), 7.10-7.06 (m, 1H), 6.92-6.87 (m, 1H), 5.23-5.17 (m, 1H), 4.84- 4.78 (m, 1H), 3.49 (q, J = 7.2 Hz, 2H), 3.34-3.27 (m, 4H), 3.03- 2.92 (m, 1H), 2.61 (m, 3H), 2.19- 2.11 (m, 1H), 2.11-2.03 (m, 2H), 1.81-1.73 (m, 2H), 1.58- 1.48 (m, 5H), 1.18-1.08 (m, 5H). |
| 47 | 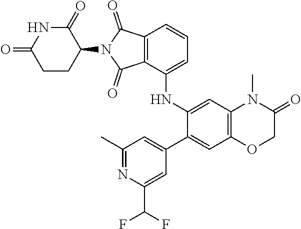 [SMILES: Cc1cc(-c2cc3c(cc2Nc2cccc4c2C(=O)N([C@H]2CCC(=O)NC2=O)C4=O)N(C)C(=O)CO3)cc(C(F)F)n1] | (S)-4-((7-(2-(difluoromethyl)-6- methylpyridin-4-yl)-4-methyl-3- oxo-3,4-dihydro-2H- benzo[b][1,4]oxazin-6- yl)amino)-2-(2,6-dioxopiperidin- 3-yl)isoindoline-1,3-dione | m/z: 576.3 [M + 1]⁺ | 11.11 (s, 1H), 8.37 (s, 1H), 7.50 (s, 2H), 7.40 (dd, J = 7.2, 8.4 Hz, 1H), 7.30 (s, 1H), 7.23 (s, 1H), 7.08 (d, J = 6.8 Hz, 1H), 6.94- 6.60 (m, 2H), 5.08 (dd, J = 5.2, 12.8 Hz, 1H), 4.76 (s, 2H), 3.31 (s, 3H), 2.97-2.81 (m, 1H), 2.64- 2.53 (m, 2H), 2.44 (s, 3H), 2.07- 1.93 (m, 1H). |
| 48 | 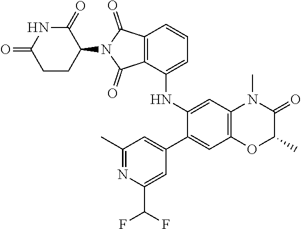 [SMILES: Cc1cc(-c2cc3c(cc2Nc2cccc4c2C(=O)N([C@H]2CCC(=O)NC2=O)C4=O)N(C)C(=O)[C@H](C)O3)cc(C(F)F)n1] | 4-(((S)-7-(2-(difluoromethyl)-6- methylpyridin-4-yl)-2,4- dimethyl-3-oxo-3,4-dihydro-2H- benzo[b][1,4]oxazin-6- yl)amino)-2-((S)-2,6- dioxopiperidin-3-yl)isoindoline- 1,3-dione | m/z: 590.3 [M + 1]⁺ | 11.11 (s, 1H), 8.37 (s, 1H), 7.51 (s, 2H), 7.42-7.38 (m, 1H), 7.30- 7.24 (m, 2H), 7.09-7.07 (m, 1H), 6.93-6.62 (m, 2H), 5.11- 5.06 (m, 1H), 4.87-4.82 (m, 1H), 3.32-3.31 (m, 6H), 2.98-2.82 (m, 1H), 2.68-2.52 (m, 2H), 2.44 (s, 3H), 2.10-1.97 (m, 1H), 1.50 (d, J = 6.8 Hz, 3H). |
| 49 | 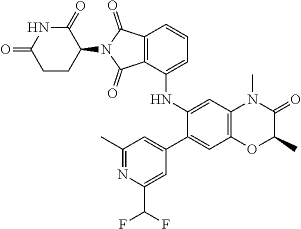 [SMILES: Cc1cc(-c2cc3c(cc2Nc2cccc4c2C(=O)N([C@H]2CCC(=O)NC2=O)C4=O)N(C)C(=O)[C@@H](C)O3)cc(C(F)F)n1] | 4-(((R)-7-(2-(difluoromethyl)-6- methylpyridin-4-yl)-2,4- dimethyl-3-oxo-3,4-dihydro-2H- benzo[b][1,4]oxazin-6- yl)amino)-2-((S)-2,6- dioxopiperidin-3-yl)isoindoline- 1,3-dione | m/z: 590.3 [M + 1]⁺ | 11.11 (s, 1H), 8.37 (s, 1H), 7.51 (s, 2H), 7.44-7.37 (m, 1H), 7.30 (s, 1H), 7.24 (s, 1H), 7.09-707 (m, 1H), 6.92-6.62 (m, 2H), 5.11- 5.06 (m, 1H), 4.86-4.84 (m, 1H), 3.31 (s, 3H), 2.91-2.87 (m, 1H), 2.64-2.52 (m, 2H), 2.44 (s, 3H), 2.06-1.95 (m, 1H), 1.50 (d, J = 6.8 Hz, 3H). |
| 50 | 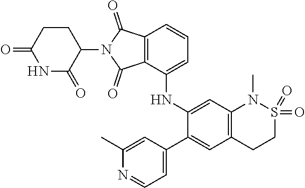 [SMILES: Cc1cc(-c2cc3c(cc2Nc2cccc4c2C(=O)N(C2CCC(=O)NC2=O)C4=O)N(C)S(=O)(=O)CC3)ccn1] | 2-(2,6-dioxopiperidin-3-yl)-4- ((1-methyl-6-(2-methylpyridin-4- yl)-2,2-dioxido-3,4-dihydro-1H- benzo[c][1,2]thiazin-7- yl)amino)isoindoline-1,3-dione | m/z: 560.2 [M + 1]⁺ | 11.12 (s, 1H), 8.28-8.43 (m, 2H), 7.47 (t, J = 8.0 Hz, 1H), 7.40 (s, 1H), 7.32 (s, 1H), 7.23 (br d, J = 5.5 Hz, 1H), 7.20 (s, 1H), 7.14 (d, J = 7.0 Hz, 1H), 6.97 (d, J = 8.5 Hz, 1H), 5.09 (dd, J = 12.5, 5.5 Hz, 1H), 3.61 (t, J = 6.5 Hz, 2H), 3.42 (t, J = 7.5 Hz, 2H), 3.24 (s, 3H), 2.81-2.96 (m, 1H), 2.54-2.65 (m, 2H), 2.41 (s, 3H), 1.97-2.11 (m, 1H) |
| 51 | 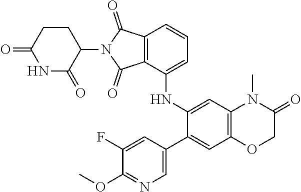 [SMILES: COc1ncc(-c2cc3c(cc2Nc2cccc4c2C(=O)N(C2CCC(=O)NC2=O)C4=O)N(C)C(=O)CO3)cc1F] | 2-(2,6-dioxopiperidin-3-yl)-4- ((7-(5-fluoro-6-methoxypyridin- 3-yl)-4-methyl-3-oxo-3,4- dihydro-2H-benzo[b][1,4]oxazin- 6-yl)amino)isoindoline-1,3-dione | m/z: 560.1 [M + 1]⁺ (558.1) | 11.12 (s, 1H), 8.30 (s, 1H), 8.06 (d, J = 1.9 Hz, 1H), 7.86 (dd, J = 11.6, 1.9 Hz, 1H), 7.44 (dd, J = 8.4, 7.3 Hz, 1H), 7.27 (s, 1H), 7.18 (s, 1H), 7.09 (d, J = 7.0 Hz, 1H), 6.73 (d, J = 8.5 Hz, 1H), 5.09 (dd, J = 12.8, 5.4 Hz, 1H), 4.75 (s, 2H), 3.90 (s, 3H), 3.27-3.31 (m, 3H), 2.83-2.95 (m, 1H), 2.61-2.69 (m, 2H), 2.01-2.11 (m, 1H) |
| 52 | 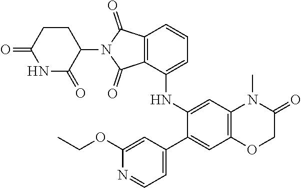 [SMILES: CCOc1cc(-c2cc3c(cc2Nc2cccc4c2C(=O)N(C2CCC(=O)NC2=O)C4=O)N(C)C(=O)CO3)ccn1] | 2-(2,6-dioxopiperidin-3-yl)-4- ((7-(2-ethoxypyridin-4-yl)-4- methyl-3-oxo-3,4-dihydro-2H- benzo[b][1,4]oxazin-6- yl)amino)isoindoline-1,3-dione | m/z: 556.2 [M + 1]⁺ | 11.12 (s, 1H), 8.26 (s, 1H), 8.05 (d, J = 5.8 Hz, 1H), 7.45 (dd, J = 8.4, 7.2 Hz, 1H), 7.27 (s, 1H), 7.15 (s, 1H), 7.10 (d, J = 6.8 Hz, 1H), 7.02 (dd, J = 5.3, 1.4 Hz, 1H), 6.84 (d, J = 0.6 Hz, 1H), 6.82 (d, J = 8.5 Hz, 1H), 5.09 (dd, J = 12.8, 5.4 Hz, 1H), 4.76 (s, 2H), 4.24 (q, J = 7.0 Hz, 2H), 3.27-3.31 (m, 3H), 2.83-2.94 (m, 1H), 2.56-2.65 (m, 2H), 2.00-2.09 (m, 1H), 1.26 (t, J = 7.0 Hz, 3H) |
| 53 | 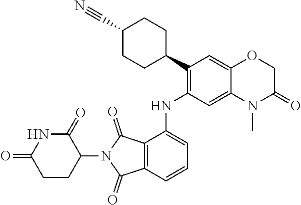 [SMILES: CN1C(=O)COc2cc([C@H]3CC[C@H](C#N)CC3)c(Nc3cccc4c3C(=O)N(C3CCC(=O)NC3=O)C4=O)cc21] | (1r,4r)-4-(6-((2-(2,6- dioxopiperidin-3-yl)-1,3- dioxoisoindolin-4-yl)amino)-4- methyl-3-oxo-3,4-dihydro-2H- benzo[b][1,4]oxazin-7- yl)cyclohexane-1-carbonitrile | m/z: 542.3 [M + 1]⁺ | 11.07 (s, 1H), 8.00 (s, 1H), 7.51- 7.41 (m, 1H), 7.15-7.06 (m, 1H), 7.01 (s, 1H), 6.92 (s, 1H), 6.92 (br dd, J = 19.4, 4.0 Hz, 1H), 6.71 (d, J = 8.3 Hz, 1H), 5.13-5.00 (m, 1H), 4.61 (s, 2H), 3.14 (s, 3H), 2.92-2.78 (m, 1H), 2.70-2.62 (m, 1H), 2.59-2.51 (m, 1H), 2.07- 1.94 (m, 4H), 1.70-1.58 (m, 2H), 1.48-1.36 (m, 4H) |
| 54 | 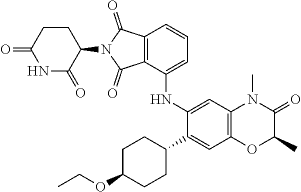 [SMILES: CCO[C@H]1CC[C@H](c2cc3c(cc2Nc2cccc4c2C(=O)N([C@@H]2CCC(=O)NC2=O)C4=O)N(C)C(=O)[C@@H](C)O3)CC1] | 2-((R)-2,6-dioxopiperidin-3-yl)- 4-(((R)-7-((1r,4R)-4- ethoxycyclohexyl)-2,4-dimethyl- 3-oxo-3,4-dihydro-2H- benzo[b][1,4]oxazin-6- yl)amino)isoindoline-1,3-dione | m/z: 575.3 [M + 1]+ | 11.14 (s, 1H), 8.09 (s, 1H), 7.59- 7.51 (m, 1H), 7.19-7.15 (m, 1H), 7.10-7.07 (m, 1H), 7.03-7.00 (m, 1H), 6.87-6.82 (m, 1H), 5.18- 5.10 (m, 1H), 4.80-4.71 (m, 1H), 3.47-3.40 (m, 2H), 3.27- 3.22 (m, 4H), 2.97-2.87 (m, 1H), 2.65-2.57 (m, 3H), 2.14-2.07 (m, 1H), 2.05-1.99 (m, 2H), 1.74- 1.67 (m, 2H), 1.51-1.43 (m, 5H), 1.13-1.04 (m, 5H) |
| 55 | 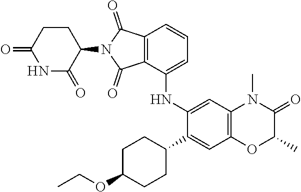 [SMILES: CCO[C@H]1CC[C@H](c2cc3c(cc2Nc2cccc4c2C(=O)N([C@@H]2CCC(=O)NC2=O)C4=O)N(C)C(=O)[C@H](C)O3)CC1] | 2-((R)-2,6-dioxopiperidin-3-yl)- 4-(((S)-7-((1r,4S)-4- ethoxycyclohexyl)-2,4-dimethyl- 3-oxo-3,4-dihydro-2H- benzo[b][1,4]oxazin-6- yl)amino)isoindoline-1,3-dione | m/z: 575.3 [M + 1]+ | 11.14 (s, 1H), 8.11-8.06 (m, 1H), 7.58-7.51 (m, 1H), 7.19-7.15 (m, 1H), 7.11-7.06 (m, 1H), 7.02 (s, 1H), 6.87-6.82 (m, 1H), 5.18- 5.10 (m, 1H), 4.75 (d, J = 6.8 Hz, 1H), 3.43 (q, J = 7.5 Hz, 2H), 3.27-3.21 (m, 4H), 2.97-2.85 (m, 1H), 2.66-2.54 (m, 3H), 2.14- 2.06 (m, 1H), 2.05-1.98 (m, 2H), 1.75-1.66 (m, 2H), 1.52- 1.42 (m, 5H), 1.14-1.04 (m, 5H) |
| 56 | 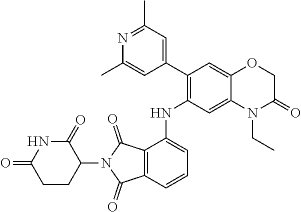 [SMILES: CCN1C(=O)COc2cc(-c3cc(C)nc(C)c3)c(Nc3cccc4c3C(=O)N(C3CCC(=O)NC3=O)C4=O)cc21] | 4-((7-(2,6-dimethylpyridin-4-yl)- 4-ethyl-3-oxo-3,4-dihydro-2H- benzo[b][1,4]oxazin-6- yl)amino)-2-(2,6-dioxopiperidin- 3-yl)isoindoline-1,3-dione | m/z: 554.2 [M + 1]+ | 11.11 (s, 1H), 8.32 (s, 1H), 7.43 (dd, J = 8.5, 7.3 Hz, 1H), 7.31 (s, 1H), 7.14 (s, 1H), 7.06-7.12 (m, 3H), 6.79 (d, J = 8.5 Hz, 1H), 5.10 (dd, J = 12.9, 5.4 Hz, 1H), 4.74 (s, 2H), 3.97 (q, J = 7.0 Hz, 2H), 2.83- 2.97 (m, 1H), 2.53-2.69 (m, 4H), 2.33 (s, 4H), 1.91-2.17 (m, 1H), 1.17 (t, J = 7.1 Hz, 3H) |
| 57 | 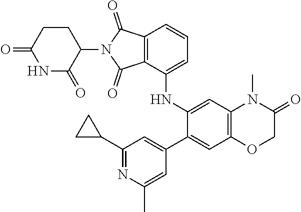 [SMILES: Cc1cc(-c2cc3c(cc2Nc2cccc4c2C(=O)N(C2CCC(=O)NC2=O)C4=O)N(C)C(=O)CO3)cc(C2CC2)n1] | 4-((7-(2-cyclopropyl-6- methylpyridin-4-yl)-4-methyl-3- oxo-3,4-dihydro-2H- benzo[b][1,4]oxazin-6- yl)amino)-2-(2,6-dioxopiperidin- 3-yl)isoindoline-1,3-dione | m/z: 566.1 [M + 1]+ | 11.11 (s, 1H), 8.29 (s, 1H), 7.45 (dd, J = 8.4, 7.3 Hz, 1H), 7.28 (s, 1H), 7.15 (s, 1H), 7.11 (d, J = 6.9 Hz, 1H), 7.04 (s, 2H), 6.84 (d, J = 8.5 Hz, 1H), 5.09 (dd, J = 12.7, 5.4 Hz, 1H), 4.76 (s, 2H), 3.31 (br s, 3H), 2.90 (br s, 1H), 2.56-2.64 (m, 2H), 2.29-2.32 (m, 3H), 2.04 (br d, J = 5.6 Hz, 1H), 1.91-1.98 (m, 1H), 0.74-0.87 (m, 4H) |
| 58 | 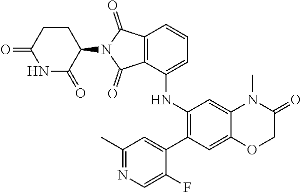 [SMILES: Cc1cc(-c2cc3c(cc2Nc2cccc4c2C(=O)N([C@@H]2CCC(=O)NC2=O)C4=O)N(C)C(=O)CO3)c(F)cn1] | (R)-2-(2,6-dioxopiperidin-3-yl)- 4-((7-(5-fluoro-2-methylpyridin- 4-yl)-4-methyl-3-oxo-3,4- dihydro-2H-benzo[b][1,4]oxazin- 6-yl)amino)isoindoline-1,3-dione | m/z: 544.4 [M + 1]+ | 11.11 (br s, 1H), 8.38 (d, J = 1.6 Hz, 1H), 8.11 (s, 1H), 7.49 (dd, J = 8.4, 7.2 Hz, 1H), 7.37-7.32 (m, 2H), 7.20-7.11 (m, 2H), 6.97 (d, J = 8.4 Hz, 1H), 5.08 (dd, J = 12.8, 5.4 Hz, 1H), 4.77 (s, 2H), 3.31 (s, 3H), 2.89 (ddd, J = 17.0, 13.9, 5.3 Hz, 1H), 2.64-2.57 (m, 2H), 2.41 (s, 3H), 2.10-1.99 (m, 1H) |
| 59 | 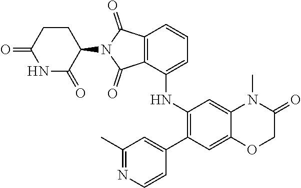 [SMILES: Cc1cc(-c2cc3c(cc2Nc2cccc4c2C(=O)N([C@@H]2CCC(=O)NC2=O)C4=O)N(C)C(=O)CO3)ccn1] | (R)-2-(2,6-dioxopiperidin-3-yl)- 4-((4-methyl-7-(2-methylpyridin- 4-yl)-3-oxo-3,4-dihydro-2H- benzo[b][1,4]oxazin-6- yl)amino)isoindoline-1,3-dione | m/z: 526.3 [M + 1]+ | 11.12 (br s, 1H), 8.34 (d, J = 5.1 Hz, 1H), 8.28 (s, 1H), 7.44 (dd, J = 8.4, 7.2 Hz, 1H), 7.33-7.22 (m, 3H), 7.16 (s, 1H), 7.10 (d, J = 6.6 Hz, 1H), 6.84 (d, J = 8.6 Hz, 1H), 5.08 (dd, J = 12.8, 5.3 Hz, 1H), 4.76 (s, 2H), 3.31 (s, 3H), 2.94-2.83 (m, 1H), 2.64-2.53 (m, 2H), 2.40 (s, 3H), 2.09-2.01 (m, 1H) |
| 60 | 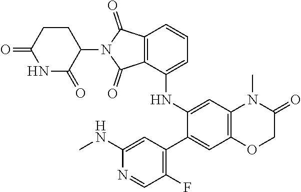 [SMILES: CNc1cc(-c2cc3c(cc2Nc2cccc4c2C(=O)N(C2CCC(=O)NC2=O)C4=O)N(C)C(=O)CO3)c(F)cn1] | 2-(2,6-dioxopiperidin-3-yl)-4- ((7-(5-fluoro-2- (methylamino)pyridin-4-yl)-4- methyl-3-oxo-3,4-dihydro-2H- benzo[b][1,4]oxazin-6- yl)amino)isoindoline-1,3-dione | m/z: 559.2 [M + 1]+ | 11.11 (br d, J = 1.6 Hz, 1H), 8.01 (s, 1H), 7.94 (d, J = 1.8 Hz, 1H), 7.52 (dd, J = 8.4, 7.2 Hz, 1H), 7.32 (s, 1H), 7.15 (d, J = 7.1 Hz, 1H), 7.10 (s, 1H), 7.06 (d, J = 8.5 Hz, 1H), 6.46 (d, J = 4.6 Hz, 1H), 6.38 (d, J = 4.9 Hz, 1H), 5.08 (dd, J = 12.8, 5.4 Hz, 1H), 4.76 (s, 2H), 2.93 (s, 3H), 2.82-2.95 (m, 1H), 2.70 (br d, J = 4.9 Hz, 3H), 2.59- 2.63 (m, 2H), 1.97-2.10 (m, 1H) |
| 61 | 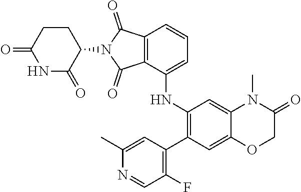 [SMILES: Cc1cc(-c2cc3c(cc2Nc2cccc4c2C(=O)N([C@H]2CCC(=O)NC2=O)C4=O)N(C)C(=O)CO3)c(F)cn1] | (S)-2-(2,6-dioxopiperidin-3-yl)- 4-((7-(5-fluoro-2-methylpyridin- 4-yl)-4-methyl-3-oxo-3,4- dihydro-2H-benzo[b][1,4]oxazin- 6-yl)amino)isoindoline-1,3-dione | m/z: 544.2 [M + 1]+ | 11.23-10.97 (m, 1H), 8.38 (d, J = 1.5 Hz, 1H), 8.11 (s, 1H), 7.49 (dd, J = 8.6, 7.3 Hz, 1H), 7.34 (t, J = 2.9 Hz, 2H), 7.17 (s, 1H), 7.13 (d, J = 7.1 Hz, 1H), 6.97 (d, J = 8.6 Hz, 1H), 5.08 (dd, J = 12.8, 5.5 Hz, 1H), 4.77 (s, 2H), 3.31-3.31 (m, 1H), 3.31 (s, 1H), 3.31-3.30 (m, 1H), 2.94-2.80 (m, 1H), 2.70- 2.52 (m, 2H), 2.41 (s, 3H), 2.06- 2.00 (m, 1H) |
| 62 | 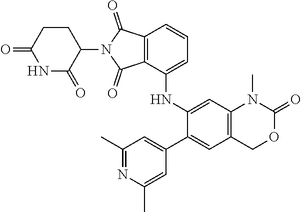 [SMILES: Cc1cc(-c2cc3c(cc2Nc2cccc4c2C(=O)N(C2CCC(=O)NC2=O)C4=O)N(C)C(=O)OC3)cc(C)n1] | 4-((6-(2,6-dimethylpyridin-4-yl)- 1-methyl-2-oxo-1,4-dihydro-2H- benzo[d][1,3]oxazin-7- yl)amino)-2-(2,6-dioxopiperidin- 3-yl)isoindoline-1,3-dione | m/z: 540.2 [M + 1]+ | 11.12 (s, 1H), 8.43 (s, 1H), 7.50 (t, J = 8.0 Hz, 1H), 7.41 (s, 1H), 7.21 (s, 1H), 7.15 (dd, J = 16.3, 7.8 Hz, 2H), 7.08 (s, 2H), 5.32 (s, 2H), 5.09 (br dd, J = 12.5, 5.5 Hz, 1H), 3.31 (s, 3H), 2.79-2.98 (m, 1H), 2.55-2.69 (m, 2H), 2.36 (s, 6H), 1.97-2.11 (m, 1H) |
| 63 | 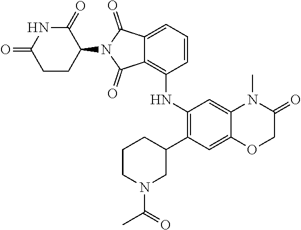 [SMILES: CC(=O)N1CCCC(c2cc3c(cc2Nc2cccc4c2C(=O)N([C@H]2CCC(=O)NC2=O)C4=O)N(C)C(=O)CO3)C1] | 4-((7-(1-acetylpiperidin-3-yl)-4- methyl-3-oxo-3,4-dihydro-2H- benzo[b][1,4]oxazin-6- yl)amino)-2-((S)-2,6- dioxopiperidin-3-yl)isoindoline- 1,3-dione | m/z: 560.7 [M + 1]+ | 11.12 (s, 1H), 8.27-8.04 (m, 1H), 7.53 (s, 1H), 7.18-7.09 (m, 3H), 6.65 (s, 1H), 5.17-5.07 (m, 1H), 4.71 (br s, 2H), 4.43-4.27 (m, 1H), 3.82-3.69 (m, 1H), 3.26- 3.20 (m, 3H), 3.08-2.98 (m, 1H), 2.97-2.85 (m, 1H), 2.82-2.68 (m, 1H), 2.67-2.55 (m, 3H), 2.14- 2.04 (m, 1H), 1.98-1.92 (m, 1H), 1.85-1.76 (m, 2H), 1.76- 1.72 (m, 2H), 1.71-1.59 (m, 1H), 1.45-1.18 (m, 1H) |
| 64 | 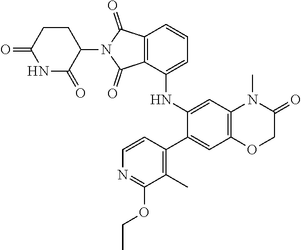 [SMILES: CCOc1nccc(-c2cc3c(cc2Nc2cccc4c2C(=O)N(C2CCC(=O)NC2=O)C4=O)N(C)C(=O)CO3)c1C] | 2-(2,6-dioxopiperidin-3-yl)-4- ((7-(2-ethoxy-3-methylpyridin-4- yl)-4-methyl-3-oxo-3,4-dihydro- 2H-benzo[b][1,4]oxazin-6- yl)amino)isoindoline-1,3-dione | m/z: 570.2 [M + 1]+ | 11.09 (s, 1H), 7.92 (br d, J = 3.9 Hz, 1H), 7.87 (br s, 1H), 7.51 (dd, J = 8.4, 7.4 Hz, 1H), 7.31 (s, 1H), 7.04-7.18 (m, 2H), 6.97 (s, 1H), 6.80 (br s, 1H), 5.05 (dd, J = 12.9, 5.4 Hz, 1H), 4.75 (s, 2H), 4.29 (q, J = 7.0 Hz, 2H), 3.31 (br s, 3H), 2.87 (ddd, J = 17.2, 13.9, 5.5 Hz, 1H), 2.57-2.63 (m, 1H), 2.47 (br d, J = 4.3 Hz, 1H), 1.91-2.06 (m, 4H), 1.29 (t, J = 7.0 Hz, 3H) |
| 65 | 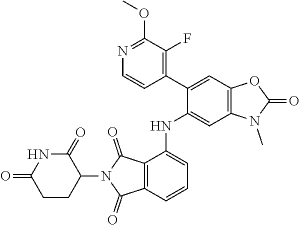 [SMILES: COc1nccc(-c2cc3oc(=O)n(C)c3cc2Nc2cccc3c2C(=O)N(C2CCC(=O)NC2=O)C3=O)c1F] | 2-(2,6-dioxopiperidin-3-yl)-4- ((6-(3-fluoro-2-methoxypyridin- 4-yl)-3-methyl-2-oxo-2,3- dihydrobenzo[d]oxazol-5- yl)amino)isoindoline-1,3-dione | m/z: 546.2 [M + 1]+ | 11.10 (s, 1H), 8.12 (s, 1H), 7.93 (d, J = 5.1 Hz, 1H), 7.47-7.58 (m, 3H), 7.17 (d, J = 6.9 Hz, 1H), 6.98- 7.05 (m, 2H), 5.07 (dd, J = 12.8, 5.4 Hz, 1H), 3.91 (s, 3H), 3.38 (s, 3H), 2.81-2.96 (m, 1H), 2.55-2.65 (m, 2H), 1.99-2.07 (m, 1H) |
| 66 | 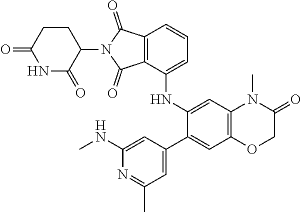 [SMILES: CNc1cc(-c2cc3c(cc2Nc2cccc4c2C(=O)N(C2CCC(=O)NC2=O)C4=O)N(C)C(=O)CO3)cc(C)n1] | 2-(2,6-dioxopiperidin-3-yl)-4- ((4-methyl-7-(2-methyl-6- (methylamino)pyridin-4-yl)-3- oxo-3,4-dihydro-2H- benzo[b][1,4]oxazin-6- yl)amino)isoindoline-1,3-dione | m/z: 555.3 [M + 1]+ | 11.11 (s, 1H), 8.21 (s, 1H), 8.14 (s, 1H), 7.50 (dd, J = 8.4, 7.2 Hz, 1H), 7.27 (s, 1H), 7.14 (d, J = 6.9 Hz, 1H), 7.09 (s, 1H), 6.98 (d, J = 8.5 Hz, 1H), 6.47 (br s, 1H), 6.33 (br s, 1H), 5.09 (dd, J = 12.8, 5.4 Hz, 1H), 4.75 (s, 2H), 3.30 (s, 3H), 2.83-2.97 (m, 1H), 2.66-2.76 (m, 3H), 2.58-2.65 (m, 1H), 2.53- 2.57 (m, 1H), 2.23 (s, 3H), 1.99- 2.09 (m, 1H) |
| 67 | 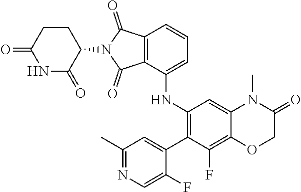 [SMILES: Cc1cc(-c2c(Nc3cccc4c3C(=O)N([C@H]3CCC(=O)NC3=O)C4=O)cc3c(c2F)OCC(=O)N3C)c(F)cn1] | 2-((S)-2,6-dioxopiperidin-3-yl)- 4-((8-fluoro-7-(5-fluoro-2- methylpyridin-4-yl)-4-methyl-3- oxo-3,4-dihydro-2H- benzo[b][1,4]oxazin-6- yl)amino)isoindoline-1,3-dione | m/z: 562.2 [M + 1]+ | (500 MHz, ACETONITRILE-d3) 8.99-8.83 (m, 1H), 8.37 (br d, J = 3.9 Hz, 1H), 7.77 (br s, 1H), 7.55-7.46 (m, 1H), 7.28-7.21 (m, 1H), 7.20-7.15 (m, 1H), 7.15- 7.11 (m, 1H), 7.11-7.04 (m, 1H), 5.02-4.90 (m, 1H), 4.81- 4.71 (m, 2H), 3.34 (s, 3H), 2.81- 2.65 (m, 3H), 2.45 (s, 3H), 2.08 (br d, J = 1.7 Hz, 1H) |
| 68 | 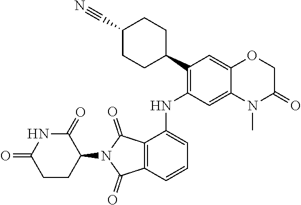 [SMILES: CN1C(=O)COc2cc([C@H]3CC[C@H](C#N)CC3)c(Nc3cccc4c3C(=O)N([C@H]3CCC(=O)NC3=O)C4=O)cc21] | (1S,4r)-4-(6-((2-((S)-2,6- dioxopiperidin-3-yl)-1,3- dioxoisoindolin-4-yl)amino)-4- methyl-3-oxo-3,4-dihydro-2H- benzo[b][1,4]oxazin-7- yl)cyclohexane-1-carbonitrile | m/z: 542.2 [M + 1]+ | (500 MHz, ACETONITRILE-d3) 8.99-8.83 (m, 1H), 8.37 (br d, J = 3.9 Hz, 1H), 7.77 (br s, 1H), 7.55-7.46 (m, 1H), 7.28-7.21 (m, 1H), 7.20-7.15 (m, 1H), 7.15- 7.11 (m, 1H), 7.11-7.04 (m, 1H), 5.02-4.90 (m, 1H), 4.81- 4.71 (m, 2H), 3.34 (s, 3H), 2.81- 2.65 (m, 3H), 2.45 (s, 3H), 2.08 (br d, J = 1.7 Hz, 1H) |
| 69 | 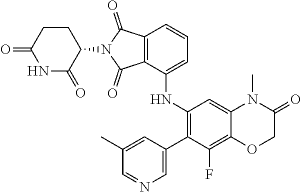 [SMILES: Cc1cncc(-c2c(Nc3cccc4c3C(=O)N([C@H]3CCC(=O)NC3=O)C4=O)cc3c(c2F)OCC(=O)N3C)c1] | (S)-2-(2,6-dioxopiperidin-3-yl)- 4-((8-fluoro-4-methyl-7-(5- methylpyridin-3-yl)-3-oxo-3,4- dihydro-2H-benzo[b][1,4]oxazin- 6-yl)amino)isoindoline-1,3-dione | m/z: 544.0 [M + 1]+ | (500 MHz, DMSO-d6) 11.09 (s, 1H), 8.27-8.39 (m, 2H), 8.18 (s, 1H), 7.65 (s, 1H), 7.46 (dd, J = 8.34, 7.27 Hz, 1H), 7.17 (s, 1H), 7.11 (d, J = 7.03 Hz, 1H), 6.96 (d, J = 8.58 Hz, 1H), 5.05 (dd, J = 12.87, 5.48 Hz, 1H), 4.84 (s, 2H), 3.31 (br s, 3H), 2.87 (ddd, J = 17.14, 13.80, 5.19 Hz, 1H), 2.55-2.66 (m, 2H), 2.24 (s, 3H), 1.98-2.06 (m, 1H) |
| 70 | 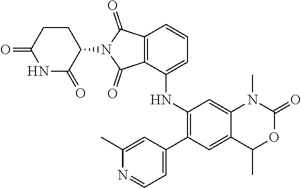 [SMILES: Cc1cc(-c2cc3c(cc2Nc2cccc4c2C(=O)N([C@H]2CCC(=O)NC2=O)C4=O)N(C)C(=O)OC3C)ccn1] | 4-((1,4-dimethyl-6-(2- methylpyridin-4-yl)-2-oxo-1,4- dihydro-2H-benzo[d][1,3]oxazin- 7-yl)amino)-2-((S)-2,6- dioxopiperidin-3-yl)isoindoline- 1,3-dione | m/z: 540.3 [M + 1]+ | (ACETONITRILE-d3) 8.99- 8.84 (m, 1H), 8.39 (d, J = 4.9 Hz, 1H), 7.99 (s, 1H), 7.49 (dd, J = 8.6, 7.1 Hz, 1H), 7.30-7.27 (m, 2H), 7.23-7.15 (m, 4H), 5.51-5.43 (m, 1H), 4.98-4.89 (m, 1H), 3.33 (s, 3H), 2.82-2.59 (m, 4H), 2.45 (s, 3H), 1.68 (d, J = 6.6 Hz, 3H) |
| 71 | 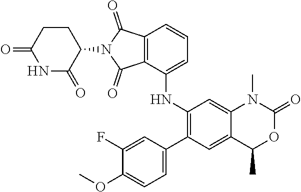 [SMILES: COc1ccc(-c2cc3c(cc2Nc2cccc4c2C(=O)N([C@H]2CCC(=O)NC2=O)C4=O)N(C)C(=O)O[C@H]3C)cc1F] | 2-((S)-2,6-dioxopiperidin-3-yl)- 4-(((S)-6-(3-fluoro-4- methoxyphenyl)-1,4-dimethyl-2- oxo-1,4-dihydro-2H- benzo[d][1,3]oxazin-7- yl)amino)isoindoline-1,3-dione | m/z: 573.6 [M + 1]+ | (ACETONITRILE-d3) 8.90 (br s, 1H), 7.96-7.86 (m, 1H), 7.49 (dd, J = 8.6, 7.1 Hz, 1H), 7.24- 7.15 (m, 5H), 7.14-7.12 (m, 1H), 7.09-7.04 (m, 1H), 5.50-5.36 (m, 1H), 4.93 (dd, J = 12.6, 5.3 Hz, 1H), 3.85 (s, 3H), 3.31 (s, 3H), 2.80-2.60 (m, 3H), 2.10-2.04 (m, 1H), 1.66 (d, J = 6.6 Hz, 3H) |
| 72 | 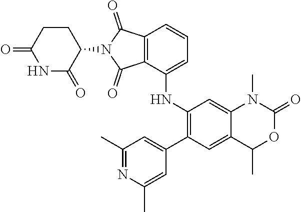 [SMILES: Cc1cc(-c2cc3c(cc2Nc2cccc4c2C(=O)N([C@H]2CCC(=O)NC2=O)C4=O)N(C)C(=O)OC3C)cc(C)n1] | 4-((6-(2,6-dimethylpyridin-4-yl)- 1,4-dimethyl-2-oxo-1,4-dihydro- 2H-benzo[d][1,3]oxazin-7- yl)amino)-2-((S)-2,6- dioxopiperidin-3-yl)isoindoline- 1,3-dione | m/z: 554.3 [M + 1]+ | (ACETONITRILE-d3) 8.90 (br s, 1H), 8.08-8.02 (m, 1H), 7.50 (dd, J = 8.6, 7.1 Hz, 1H), 7.29- 7.22 (m, 2H), 7.20-7.15 (m, 2H), 7.08-7.02 (m, 2H), 5.51-5.40 (m, 1H), 4.98-4.88 (m, 1H), 3.33 (s, 3H), 2.82-2.58 (m, 4H), 2.41- 2.39 (m, 6H), 1.70-1.66 (m, 3H) |
| 73 | 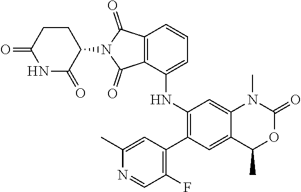 [SMILES: Cc1cc(-c2cc3c(cc2Nc2cccc4c2C(=O)N([C@H]2CCC(=O)NC2=O)C4=O)N(C)C(=O)O[C@H]3C)c(F)cn1] | 2-((S)-2,6-dioxopiperidin-3-yl)- 4-(((S)-6-(5-fluoro-2- methylpyridin-4-yl)-1,4- dimethyl-2-oxo-1,4-dihydro-2H- benzo[d][1,3]oxazin-7- yl)amino)isoindoline-1,3-dione | m/z: 558.3 [M + 1]+ | (ACETONITRILE-d3) 8.36-8.22 (m, 1H), 7.97-7.85 (m, 1H), 7.55- 7.44 (m, 1H), 7.31-7.27 (m, 1H), 7.26-7.19 (m, 3H), 7.17 (d, J = 6.6 Hz, 1H), 5.52-5.40 (m, 1H), 4.98-4.88 (m, 1H), 3.33 (s, 3H), 2.82-2.58 (m, 3H), 2.46 (d, J = 1.0 Hz, 3H), 2.13-2.05 (m, 2H), 1.67 (d, J = 6.6 Hz, 3H) |
| 74 | 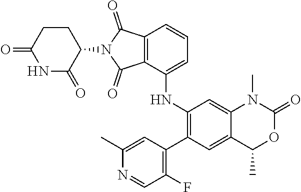 [SMILES: Cc1cc(-c2cc3c(cc2Nc2cccc4c2C(=O)N([C@H]2CCC(=O)NC2=O)C4=O)N(C)C(=O)O[C@@H]3C)c(F)cn1] | 2-((S)-2,6-dioxopiperidin-3-yl)- 4-(((R)-6-(5-fluoro-2- methylpyridin-4-yl)-1,4- dimethyl-2-oxo-1,4-dihydro-2H- benzo[d][1,3]oxazin-7- yl)amino)isoindoline-1,3-dione | m/z: 558.3 [M + 1]+ | (ACETONITRILE-d3) 8.30 (d, J = 1.7 Hz, 1H), 7.93 (s, 1H), 7.50 (dd, J = 8.8, 7.3 Hz, 1H), 7.31- 7.26 (m, 1H), 7.26-7.19 (m, 3H), 7.19-7.16 (m, 1H), 5.47 (d, J = 6.6 Hz, 1H), 4.96-4.91 (m, 1H), 3.36- 3.29 (m, 3H), 2.81-2.63 (m, 3H), 2.46 (d, J = 1.0 Hz, 3H), 2.13- 2.05 (m, 2H), 1.67 (d, J = 6.6 Hz, 3H) |
| 75 | 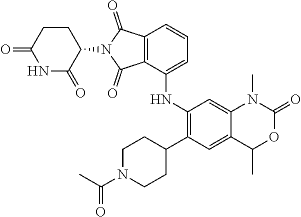 [SMILES: CC(=O)N1CCC(c2cc3c(cc2Nc2cccc4c2C(=O)N([C@H]2CCC(=O)NC2=O)C4=O)N(C)C(=O)OC3C)CC1] | 4-((6-(1-acetylpiperidin-4-yl)- 1,4-dimethyl-2-oxo-1,4-dihydro- 2H-benzo[d][1,3]oxazin-7- yl)amino)-2-((S)-2,6- dioxopiperidin-3-yl)isoindoline- 1,3-dione | m/z: 574.3 [M + 1]+ | (ACETONITRILE-d3) 9.02-8.84 (m, 1H), 8.00-7.86 (m, 1H), 7.58- 7.43 (m, 1H), 7.22-7.14 (m, 2H), 7.04-6.92 (m, 2H), 5.49- 5.32 (m, 1H), 5.08-4.90 (m, 1H), 4.65-4.53 (m, 1H), 3.95-3.85 (m, 1H), 3.29-3.20 (m, 3H), 3.10- 2.96 (m, 2H), 2.84-2.65 (m, 3H), 2.53-2.43 (m, 1H), 2.08 (br d, J = 1.2 Hz, 2H), 2.02 (s, 3H), 1.75-1.66 (m, 2H), 1.65-1.61 (m, 3H), 1.57-1.49 (m, 1H) |
| 76 | 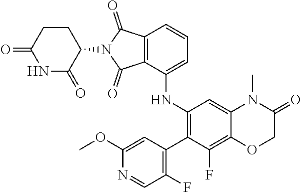 [SMILES: COc1cc(-c2c(Nc3cccc4c3C(=O)N([C@H]3CCC(=O)NC3=O)C4=O)cc3c(c2F)OCC(=O)N3C)c(F)cn1] | 2-((S)-2,6-dioxopiperidin-3-yl)- 4-((8-fluoro-7-(5-fluoro-2- methoxypyridin-4-yl)-4-methyl- 3-oxo-3,4-dihydro-2H- benzo[b][1,4]oxazin-6- yl)amino)isoindoline-1,3-dione | m/z: 578.4 [M + 1]+ | (500 MHz, DMSO-d6) ppm 11.09 (s, 1H), 8.12-8.22 (m, 2H), 7.47- 7.56 (m, 1H), 7.12-7.23 (m, 2H), 6.92-7.04 (m, 2H), 5.06 (dd, J = 12.82, 5.30 Hz, 1H), 4.85 (d, J = 5.84 Hz, 2H), 3.29 (br s, 3H), 2.82-2.94 (m, 1H), 2.55-2.66 (m, 3H), 2.36 (dt, J = 3.58, 1.79 Hz, 2H), 1.98-2.07 (m, 1H) |
| 77 | 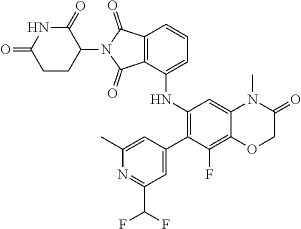 [SMILES: Cc1cc(-c2c(Nc3cccc4c3C(=O)N(C3CCC(=O)NC3=O)C4=O)cc3c(c2F)OCC(=O)N3C)cc(C(F)F)n1] | 4-((7-(2-(difluoromethyl)-6- methylpyridin-4-yl)-8-fluoro-4- methyl-3-oxo-3,4-dihydro-2H- benzo[b][1,4]oxazin-6- yl)amino)-2-(2,6-dioxopiperidin- 3-yl)isoindoline-1,3-dione | m/z: 594.9 [M + 1]+ | 10.93-11.29 (m, 1H), 8.29 (s, 1H), 7.39-7.50 (m, 3H), 7.18 (d, J = 1.22 Hz, 1H), 7.10 (d, J = 7.09 Hz, 1H), 6.89-6.96 (m, 1H), 6.65-6.83 (m, 1H), 5.06 (dd, J = 12.84, 5.38 Hz, 1H), 4.85 (s, 2H), 3.31-3.31 (m, 4H), 2.82- 2.95 (m, 1H), 2.55-2.64 (m, 1H), 2.44 (s, 3H), 1.95-2.05 (m, 1H) |
| 78 | 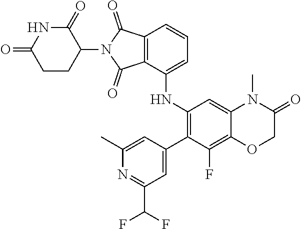 [SMILES: Cc1cc(-c2c(Nc3cccc4c3C(=O)N(C3CCC(=O)NC3=O)C4=O)cc3c(c2F)OCC(=O)N3C)cc(C(F)F)n1] | 4-((7-(2-(difluoromethyl)-6- methylpyridin-4-yl)-8-fluoro-4- methyl-3-oxo-3,4-dihydro-2H- benzo[b][1,4]oxazin-6- yl)amino)-2-(2,6-dioxopiperidin- 3-yl)isoindoline-1,3-dione | m/z: 594.9 [M + 1]+ | 10.85-11.27 (m, 1H), 8.30 (s, 1H), 7.39-7.51 (m, 3H), 7.18 (d, J = 1.34 Hz, 1H), 7.10 (d, J = 6.85 Hz, 1H), 6.89-6.98 (m, 1H), 6.65-6.83 (m, 1H), 5.06 (dd, J = 12.84, 5.38 Hz, 1H), 4.85 (s, 2H), 3.31 (br s, 4H), 2.82-2.96 (m, 1H), 2.55-2.64 (m, 1H), 2.44 (s, 3H), 1.95-2.06 (m, 1H) |
| 79 | 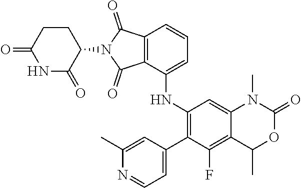 [SMILES: Cc1cc(-c2c(Nc3cccc4c3C(=O)N([C@H]3CCC(=O)NC3=O)C4=O)cc3c(c2F)C(C)OC(=O)N3C)ccn1] | 2-((S)-2,6-dioxopiperidin-3-yl)- 4-((5-fluoro-1,4-dimethyl-6-(2- methylpyridin-4-yl)-2-oxo-1,4- dihydro-2H-benzo[d][1,3]oxazin- 7-yl)amino)isoindoline-1,3-dione | m/z: 558.3 [M + 1]+ | (500 MHz, ACETONITRILE-d3) 9.07-8.85 (m, 1H), 8.51-8.44 (m, 1H), 7.96-7.82 (m, 1H), 7.64- 7.52 (m, 1H), 7.38 (dd, J = 8.5, 1.7 Hz, 1H), 7.27-7.22 (m, 2H), 7.17 (d, J = 5.0 Hz, 1H), 7.08- 7.03 (m, 1H), 5.80-5.65 (m, 1H), 4.98-4.90 (m, 1H), 3.40-3.33 (m, 3H), 2.84-2.60 (m, 3H), 2.50 (s, 3H), 2.08-2.07 (m, 1H), 1.63 (d, J = 6.7 Hz, 3H) |
| 80 | 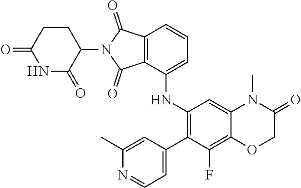 [SMILES: Cc1cc(-c2c(Nc3cccc4c3C(=O)N(C3CCC(=O)NC3=O)C4=O)cc3c(c2F)OCC(=O)N3C)ccn1] | 2-(2,6-dioxopiperidin-3-yl)-4- ((8-fluoro-4-methyl-7-(2- methylpyridin-4-yl)-3-oxo-3,4- dihydro-2H-benzo[b][1,4]oxazin- 6-yl)amino)isoindoline-1,3-dione | m/z: 544.9 [M + 1]+ | 8.40 (d, J = 5.01 Hz, 1H), 8.18 (s, 1H), 7.47 (dd, J = 8.50, 7.15 Hz, 1H), 7.27 (s, 1H), 7.15-7.20 (m, 2H), 7.12 (d, J = 6.97 Hz, 1H), 6.99 (d, J = 8.56 Hz, 1H), 5.06 (dd, J = 12.78, 5.44 Hz, 1H), 4.84 (s, 2H), 3.30 (br s, 3H), 2.81-2.93 (m, 1H), 2.54-2.63 (m, 1H), 2.40 (s, 3H), 1.97-2.07 (m, 1H), 1.78 (br s, 1H), 1.23 (s, 1H) |
| 81 | 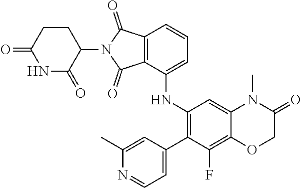 [SMILES: Cc1cc(-c2c(Nc3cccc4c3C(=O)N(C3CCC(=O)NC3=O)C4=O)cc3c(c2F)OCC(=O)N3C)ccn1] | 2-(2,6-dioxopiperidin-3-yl)-4- ((8-fluoro-4-methyl-7-(2- methylpyridin-4-yl)-3-oxo-3,4- dihydro-2H-benzo[b][1,4]oxazin- 6-yl)amino)isoindoline-1,3-dione | m/z: 544.9 [M + 1]+ | 8.40 (d, J = 5.01 Hz, 1H), 8.18 (s, 1H), 7.42-7.52 (m, 1H), 7.27 (s, 1H), 7.10-7.20 (m, 3H), 6.99 (d, J = 8.56 Hz, 1H), 5.06 (dd, J = 12.78, 5.44 Hz, 1H), 4.84 (s, 2H), 3.30 (s, 3H), 2.80-2.94 (m, 1H), 2.55-2.63 (m, 1H), 2.40 (s, 3H), 1.95-2.09 (m, 1H), 1.79 (br s, 2H) |
| 82 | 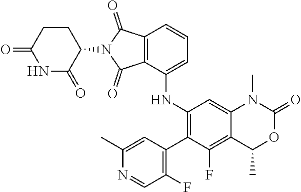 [SMILES: Cc1cc(-c2c(Nc3cccc4c3C(=O)N([C@H]3CCC(=O)NC3=O)C4=O)cc3c(c2F)[C@@H](C)OC(=O)N3C)c(F)cn1] | 2-((S)-2,6-dioxopiperidin-3-yl)- 4-(((4R)-5-fluoro-6-(5-fluoro-2- methylpyridin-4-yl)-1,4- dimethyl-2-oxo-1,4-dihydro-2H- benzo[d][1,3]oxazin-7- yl)amino)isoindoline-1,3-dione | m/z: 576.6 [M + 1]+ | (500 MHz, DMSO-d6) 11.16- 11.05 (m, 1H), 8.50-8.43 (m, 1H), 8.35-8.22 (m, 1H), 7.62- 7.51 (m, 1H), 7.39-7.34 (m, 1H), 7.34-7.27 (m, 1H), 7.26-7.22 (m, 1H), 7.13 (s, 1H), 5.76 (quin, J = 6.6 Hz, 1H), 5.13-4.98 (m, 1H), 3.34 (d, J = 1.8 Hz, 3H), 2.93- 2.82 (m, 1H), 2.66-2.56 (m, 1H), 2.55-2.52 (m, 1H), 2.44- 2.40 (m, 3H), 2.06-1.99 (m, 1H), 1.61-1.52 (m, 3H) |
| 83 | 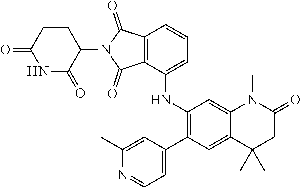 [SMILES: Cc1cc(-c2cc3c(cc2Nc2cccc4c2C(=O)N(C2CCC(=O)NC2=O)C4=O)N(C)C(=O)CC3(C)C)ccn1] | 2-(2,6-dioxopiperidin-3-yl)-4- ((1,4,4-trimethyl-6-(2- methylpyridin-4-yl)-2-oxo- 1,2,3,4-tetrahydroquinolin-7- yl)amino)isoindoline-1,3-dione | m/z: 552.3 [M + 1]+ | 11.13 (s, 1H), 8.60 (brs, 1H), 7.91- 7.89 (m, 1H), 7.80-7.78 (m, 1H), 7.50-7.46 (m, 2H), 7.28 (s, 1H), 7.17 (d, J = 8.0 Hz, 1H), 6.98-6.97 (m, 1H), 5.12-5.08 (m, 1H), 3.31 (s, 3H), 2.98-2.94 (m, 1H), 2.56-2.53 (m, 1H), 2.53- 2.51 (m, 2H), 2.04-2.02 (m, 1H), 1.41 (s, 9H). |
| 84 | 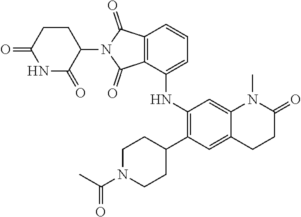 [SMILES: CC(=O)N1CCC(c2cc3c(cc2Nc2cccc4c2C(=O)N(C2CCC(=O)NC2=O)C4=O)N(C)C(=O)CC3)CC1] | 4-((6-(1-acetylpiperidin-4-yl)-1- methyl-2-oxo-1,2,3,4- tetrahydroquinolin-7-yl)amino)- 2-(2,6-dioxopiperidin-3- yl)isoindoline-1,3-dione | m/z: 558.2 [M + 1]⁺ | 11.13 (s, 1H), 8.59 (d, J = 4.4 Hz, 1H), 8.49 (s, 1H), 7.83 (brs, 1H), 7.70 (brs, 1H), 7.49-7.46 (m, 2H), 7.24 (s, 1H), 7.17 (d, J = 7.6 Hz, 1H), 6.97-6.95 (m, 1H), 5.12- 5.08 (m, 1H), 3.28 (s, 3H), 2.98- 2.89 (m, 3H), 2.58-2.53 (m, 1H), 2.51-2.50 (m, 3H), 2.34- 2.30 (m, 1H), 2.04-2.02 (m, 1H), 1.49 (s, 6H). |
| 85 | 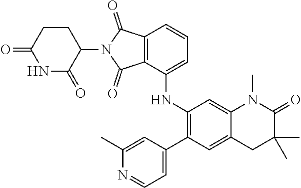 [SMILES: Cc1cc(-c2cc3c(cc2Nc2cccc4c2C(=O)N(C2CCC(=O)NC2=O)C4=O)N(C)C(=O)C(C)(C)C3)ccn1] | 2-(2,6-dioxopiperidin-3-yl)-4- ((1,3,3-trimethyl-6-(2- methylpyridin-4-yl)-2-oxo- 1,2,3,4-tetrahydroquinolin-7- yl)amino)isoindoline-1,3-dione | m/z: 552.3 [M + 1]⁺ | 11.13 (s, 1H), 8.59 (d, J = 4.4 Hz, 1H), 8.49 (s, 1H), 7.83 (brs, 1H), 7.70 (brs, 1H), 7.49-7.46 (m, 2H), 7.24 (s, 1H), 7.17 (d, J = 7.6 Hz, 1H), 6.97-6.95 (m, 1H), 5.12- 5.08 (m, 1H), 3.28 (s, 3H), 2.98- 2.89 (m, 3H), 2.58-2.53 (m, 1H), 2.51-2.50 (m, 3H), 2.34- 2.30 (m, 1H), 2.04-2.02 (m, 1H), 1.49 (s, 6H). |
| 86 | 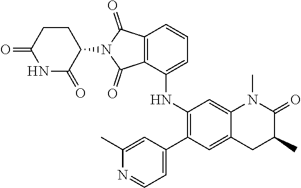 [SMILES: Cc1cc(-c2cc3c(cc2Nc2cccc4c2C(=O)N([C@H]2CCC(=O)NC2=O)C4=O)N(C)C(=O)[C@@H](C)C3)ccn1] | 4-(((S)-1,3-dimethyl-6-(2- methylpyridin-4-yl)-2-oxo- 1,2,3,4-tetrahydroquinolin-7- yl)amino)-2-((S)-2,6- dioxopiperidin-3-yl)isoindoline- 1,3-dione | m/z: 538.3 [M + 1]⁺ | 11.11 (s, 1H), 8.35 (d, J = 5.2 Hz, 1H), 8.32 (s, 1H), 7.49-7.43 (m, 1H), 7.37 (s, 1H), 7.31 (s, 1H), 7.22 (d, J = 4.8 Hz, 1H), 7.19 (s, 1H), 7.13 (d, J = 7.2 Hz, 1H), 7.00 (d, J = 8.8 Hz, 1H), 5.08 (dd, J = 5.4, 12.8 Hz, 1H), 3.28 (s, 3H), 3.02 (m, 1H), 2.94-2.83 (m, 1H), 2.78-2.69 (m, 1H), 2.68- 2.63 (m, 1H), 2.62-2.52 (m, 2H), 2.40 (s, 3H), 2.07-2.00 (m, 1H), 1.18 (d, J = 6.8 Hz, 3H). |
| 87 | 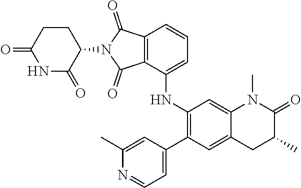 [SMILES: Cc1cc(-c2cc3c(cc2Nc2cccc4c2C(=O)N([C@H]2CCC(=O)NC2=O)C4=O)N(C)C(=O)[C@H](C)C3)ccn1] | 4-(((R)-1,3-dimethyl-6-(2- methylpyridin-4-yl)-2-oxo- 1,2,3,4-tetrahydroquinolin-7- yl)amino)-2-((S)-2,6- dioxopiperidin-3-yl)isoindoline- 1,3-dione | m/z: 538.3 [M + 1]⁺ | 11.10 (s, 1H), 8.35 (d, J = 5.2 Hz, 1H), 8.32 (s, 1H), 7.49-7.43 (m, 1H), 7.37 (s, 1H), 7.31 (s, 1H), 7.22 (d, J = 4.8 Hz, 1H), 7.19 (s, 1H), 7.13 (d, J = 7.2 Hz, 1H), 7.00 (d, J = 8.8 Hz, 1H), 5.08 (dd, J = 5.4, 12.8 Hz, 1H), 3.28 (s, 3H), 3.05-3.00 (m, 1H), 2.94- 2.82 (m, 1H), 2.78-2.69 (m, 1H), 2.67-2.58 (m, 2H), 2.57-2.51 (m, 1H), 2.40 (s, 3H), 2.06-2.00 (m, 1H), 1.18 (d, J = 6.8 Hz, 3H). |
| 88 | 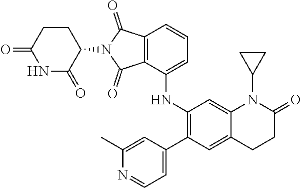 [SMILES: Cc1cc(-c2cc3c(cc2Nc2cccc4c2C(=O)N([C@H]2CCC(=O)NC2=O)C4=O)N(C2CC2)C(=O)CC3)ccn1] | (S)-4-((1-cyclopropyl-6-(2- methylpyridin-4-yl)-2-oxo- 1,2,3,4-tetrahydroquinolin-7- yl)amino)-2-(2,6-dioxopiperidin- 3-yl)isoindoline-1,3-dione | m/z 550.2 [M + 1]⁺. | 11.11 (s, 1H), 8.37 (d, J = 5.2 Hz, 1H), 8.31 (s, 1H), 7.53 (dd, J = 7.2, 8.4 Hz, 1H), 7.39 (s, 1H), 7.34 (s, 1H), 7.32 (s, 1H), 7.24- 7.21 (m, 1H), 7.16 (d, J = 7.2 Hz, 1H), 7.11 (d, J = 8.8 Hz, 1H), 5.08 (dd, J = 5.2, 12.8 Hz, 1H), 2.90-2.82 (m, 3H), 2.75-2.70 (m, 1H), 2.63-2.53 (m, 4H), 2.42 (s, 3H), 2.07-2.00 (m, 1H), 1.07- 1.00 (m, 2H), 0.63-0.58 (m, 2H) |
| 89 | 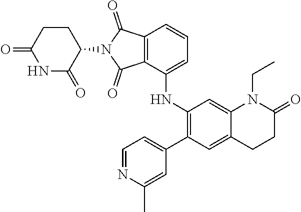 [SMILES: CCN1C(=O)CCc2cc(-c3ccnc(C)c3)c(Nc3cccc4c3C(=O)N([C@H]3CCC(=O)NC3=O)C4=O)cc21] | (S)-2-(2,6-dioxopiperidin-3-yl)- 4-((1-ethyl-6-(2-methylpyridin-4- yl)-2-oxo-1,2,3,4- tetrahydroquinolin-7- yl)amino)isoindoline-1,3-dione | m/z: 538.3 [M + 1]⁺. | 11.12 (s, 1H), 8.37-8.34 (m, 1H), 7.45-7.30 (m, 1H), 7.38 (s, 1H), 7.32 (s, 1H), 7.23-7.22 (m, 2H), 7.12-7.10 (m, 1H), 6.91-6.89 (m, 1H), 5.10 (dd, J = 5.6, 12.8 Hz, 1H), 3.95-3.91 (m, 2H), 2.96- 2.93 (m, 3H), 2.59-2.52 (m, 4H), 2.40 (s, 3H), 2.05-2.04 (m, 1H), 1.14 (t, J = 6.4 Hz, 3H). |
| 90 | 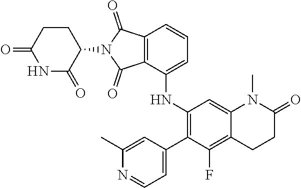 [SMILES: Cc1cc(-c2c(Nc3cccc4c3C(=O)N([C@H]3CCC(=O)NC3=O)C4=O)cc3c(c2F)CCC(=O)N3C)ccn1] | (S)-2-(2,6-dioxopiperidin-3-yl)- 4-((5-fluoro-1-methyl-6-(2- methylpyridin-4-yl)-2-oxo- 1,2,3,4-tetrahydroquinolin-7- yl)amino)isoindoline-1,3-dione | m/z: 542.3 [M + 1]⁺. | 11.11 (s, 1H), 8.43 (s, 1H), 8.23 (s, 1H), 7.54-7.50 (m, 1H), 7.27 (s, 1H), 7.20-7.17 (m, 3H), 7.10 (s, 1H), 5.09-5.04 (m, 1H), 3.28 (s, 3H), 2.93-2.90 (m, 3H), 2.67- 2.65 (m, 3H), 2.45-2.33 (m, 4H), 2.08-2.01 (m, 1H) |
| 91 | 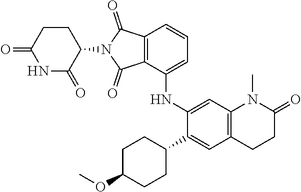 [SMILES: CO[C@H]1CC[C@H](c2cc3c(cc2Nc2cccc4c2C(=O)N([C@H]2CCC(=O)NC2=O)C4=O)N(C)C(=O)CC3)CC1] | 2-((S)-2,6-dioxopiperidin-3-yl)- 4-((6-((1r,4S)-4- methoxycyclohexyl)-1-methyl-2- oxo-1,2,3,4-tetrahydroquinolin- 7-yl)amino)isoindoline-1,3-dione | m/z: 545.1 [M + 1]⁺ | 11.14 (s, 1H), 8.13 (s, 1H), 7.59- 7.53 (m, 1H), 7.23-7.20 (m, 1H), 7.20-7.17 (m, 1H), 7.02-7.00 (m, 1H), 6.99-6.95 (m, 1H), 5.17- 5.11 (m, 1H), 3.30-3.26 (m, 1H), 3.24-3.22 (m, 3H), 3.21- 3.19 (m, 3H), 2.93-2.87 (m, 3H), 2.66 (dd, J = 13.2 Hz, 2H), 2.59 (dd, J = 5.6 Hz, 2H), 2.10-2.06 (m, 1H), 1.97-1.90 (m, 2H), 1.73- 1.64 (m, 2H), 1.52-1.28 (m, 5H) |
| 92 | 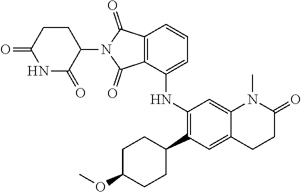 [SMILES: CO[C@H]1CC[C@@H](c2cc3c(cc2Nc2cccc4c2C(=O)N(C2CCC(=O)NC2=O)C4=O)N(C)C(=O)CC3)CC1] | 2-(2,6-dioxopiperidin-3-yl)-4- ((6-((1s,4s)-4- methoxycyclohexyl)-1-methyl-2- oxo-1,2,3,4-tetrahydroquinolin- 7-yl)amino)isoindoline-1,3-dione | m/z: 545.3 [M + 1]⁺ | 11.14 (s, 1H), 8.13 (s, 1H), 7.59- 7.53 (m, 1H), 7.23-7.20 (m, 1H), 7.20-7.17 (m, 1H), 7.02-7.00 (m, 1H), 6.99-6.95 (m, 1H), 5.17- 5.11 (m, 1H), 3.30-3.26 (m, 1H), 3.24-3.22 (m, 3H), 3.21- 3.19 (m, 3H), 2.93-2.87 (m, 3H), 2.66 (dd, J = 13.2 Hz, 2H), 2.59 (dd, J = 5.6 Hz, 2H), 2.10-2.06 (m, 1H), 1.97-1.90 (m, 2H), 1.73- 1.64 (m, 2H), 1.52-1.28 (m, 5H) |
| 93 | 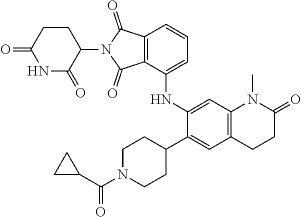 [SMILES: CN1C(=O)CCc2cc(C3CCN(C(=O)C4CC4)CC3)c(Nc3cccc4c3C(=O)N(C3CCC(=O)NC3=O)C4=O)cc21] | 4-((6-(1- (cyclopropanecarbonyl)piperidin- 4-yl)-1-methyl-2-oxo-1,2,3,4- tetrahydroquinolin-7-yl)amino)- 2-(2,6-dioxopiperidin-3- yl)isoindoline-1,3-dione | m/z: 584.4 [M + 1]⁺. | 11.13 (s, 1H), 8.17 (s, 1H), 7.57- 7.53 (m, 1H), 7.29 (s, 1H), 7.19- 7.17 (m, 1H), 7.00 (s, 1H), 6.90- 6.88 (m, 1H), 5.16-5.11 (m, 1H), 4.50-4.32 (m, 2H), 3.19 (s, 3H), 3.06-2.94 (m, 2H), 2.91-2.87 (m, 3H), 2.64-2.54 (m, 4H), 2.10- 1.94 (m, 2H), 1.78-1.69 (m, 2H), 1.61-1.47 (m, 2H), 1.24 (s, 2H) |
| 94 | 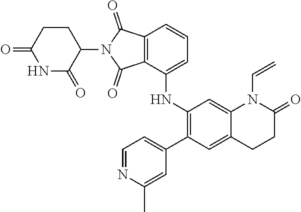 | 2-(2,6-dioxopiperidin-3-yl)-4- ((6-(2-methylpyridin-4-yl)-2- oxo-1-vinyl-1,2,3,4- tetrahydroquinolin-7- yl)amino)isoindoline-1,3-dione | m/z: 525.2 [M + 1]⁺ | 11.12 (s, 1H), 8.54 (s, 1H), 8.39 (d, J = 5.2 Hz, 1H), 7.58 (s, 1H), 7.55-7.52 (m, 1H), 7.50-7.47 (m, 1H), 7.46-7.44 (m, 1H), 7.18 (d, J = 7.2 Hz, 1H), 6.94 (d, J = 8.4 Hz, 1H), 5.14-5.07 (m, 1H), 3.25 (s, 3H), 3.13 (t, J = 7.6 Hz, 2H), 2.95-2.84 (m, 1H), 2.77 (t, J = 7.6 Hz, 2H), 2.65-2.60 (m, 1H), 2.58 (br s, 1H), 2.43 (s, 3H), 2.11-2.01 (m, 1H) |
| 95 | 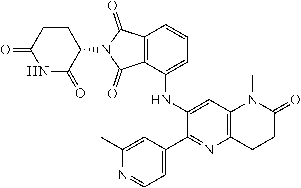 [SMILES: Cc1cc(-c2nc3c(cc2Nc2cccc4c2C(=O)N([C@H]2CCC(=O)NC2=O)C4=O)N(C)C(=O)CC3)ccn1] | (S)-2-(2,6-dioxopiperidin-3-yl)- 4-((5-methyl-2-(2-methylpyridin- 4-yl)-6-oxo-5,6,7,8-tetrahydro- 1,5-naphthyridin-3- yl)amino)isoindoline-1,3-dione | m/z: 525.3 [M + 1]⁺ | 11.12 (s, 1H), 8.55 (s, 1H), 8.40 (d, J = 5.2 Hz, 1H), 7.60-7.55 (m, 2H), 7.52-7.43 (m, 2H), 7.18 (d, J = 7.2 Hz, 1H), 6.94 (d, J = 8.4 Hz, 1H), 5.14-5.06 (m, 1H), 3.25 (s, 3H), 3.13 (t, J = 7.6 Hz, 2H), 2.95-2.84 (m, 1H), 2.77 (t, J = 7.6 Hz, 2H), 2.65-2.61 (m, 1H), 2.60-2.56 (m, 1H), 2.44 (s, 3H), 2.10-2.01 (m, 1H) |
| 96 | 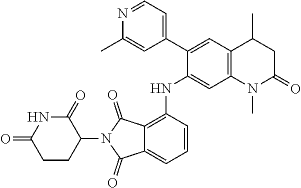 [SMILES: Cc1cc(-c2cc3c(cc2Nc2cccc4c2C(=O)N(C2CCC(=O)NC2=O)C4=O)N(C)C(=O)CC3C)ccn1] | 4-((1,4-dimethyl-6-(2- methylpyridin-4-yl)-2-oxo- 1,2,3,4-tetrahydroquinolin-7- yl)amino)-2-(2,6-dioxopiperidin- 3-yl)isoindoline-1,3-dione | m/z: 538.3 | 11.12 (s, 1H), 8.36 (d, J = 5.5 Hz, 1H), 8.34 (s, 1H), 7.47 (dd, J = 8.3, 7.3 Hz, 1H), 7.37 (s, 1H), 7.33 (s, 1H), 7.21-7.26 (m, 2H), 7.14 (d, J = 7.0 Hz, 1H), 7.01 (d, J = 8.5 Hz, 1H), 5.09 (dd, J = 13.0, 5.5 Hz, 1H), 3.30 (s, 3H), 3.09-3.22 (m, 1H), 2.83-2.95 (m, 1H), 2.76 (dd, J = 16.0, 5.5 Hz, 1H), 2.53- 2.65 (m, 2H), 2.43-2.48 (m, 1H), 2.42 (s, 3H), 1.99-2.09 (m, 1H), 1.25 (d, J = 7.0 Hz, 3H) |

The compounds disclosed herein can be made using conventional organic syntheses and commercially available starting materials. Certain compounds of the disclosure are prepared as described in the examples below:

### Example 1: Synthesis of Compound 18: 2-[(3S)-2,6-dioxo-3-piperidyl]-4-[[4-methyl-7-(2-methyl-4-pyridyl)-3-oxo-1,4-benzoxazin-6-yl]amino]isoindoline-1,3-dione

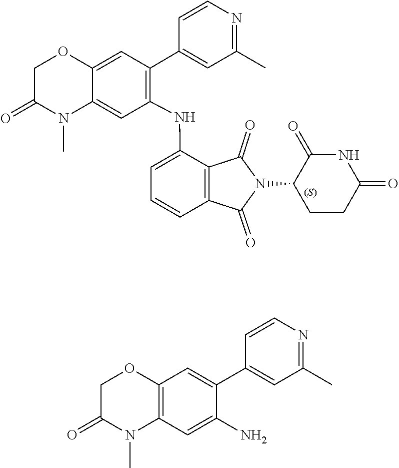

6-amino-4-methyl-7-(2-methyl-4-pyridyl)-1,4-benzoxazin-3-one. To a solution of 6-amino-7-bromo-4-methyl-1,4-benzoxazin-3-one (3.000 g, 11.67 mmol) in 1,4-Dioxane (60 mL) and Water (6 mL) was added 2-methyl-4-(4,4,5,5-tetramethyl-1,3,2-dioxaborolan-2-yl)pyridine (3.068 g, 14 mmol), potassium carbonate (4.831 g, 35.01 mmol) and (1,1′-bis(diphenylphosphino)ferrocene)palladium(II) dichloride (0.976 g, 1.17 mmol). The suspension was degassed under vacuum and purged with nitrogen several times. The mixture was stirred at 110° C. for 12 h. The mixture was filtered and filtrate was concentrated under reduced pressure. The residue was purified by column chromatography on silica gel (ethyl acetate in petroleum ether). Compound 6-amino-4-methyl-7-(2-methyl-4-pyridyl)-1,4-benzoxazin-3-one (2.7 g, 10.1 mmol, 87% yield) was obtained as a brown solid. MS (ESI) m/z 270.0 [M+1]⁺.

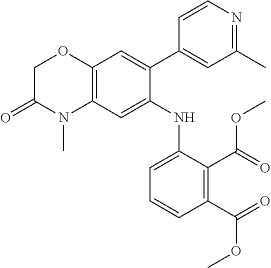 [SMILES: COC(=O)c1cccc(Nc2cc3c(cc2-c2ccnc(C)c2)OCC(=O)N3C)c1C(=O)OC]

Dimethyl-3-[[4-methyl-7-(2-methyl-4-pyridyl)-3-oxo-1,4-benzoxazin-6-yl]amino]benzene-1,2-dicarboxylate. To a solution of 6-amino-4-methyl-7-(2-methyl-4-pyridyl)-1,4-benzoxazin-3-one (2.000 g, 7.43 mmol) in 1,4-Dioxane (40 mL) was added dimethyl 3-bromobenzene-1,2-dicarboxylate (2.231 g, 8.17 mmol), cesium carbonate (7.241 g, 22.28 mmol) and methanesulfonato(2-dicyclohexylphosphino-2′,6′-di-i-propoxy-1,1′-biphenyl)(2′-amino-1,1′-biphenyl-2-yl)palladium(ii) (0.621 g, 0.74 mmol). The suspension was degassed under vacuum and purged with nitrogen several times. The mixture was stirred at 110° C. for 12 h. The mixture was filtered and filtrate was concentrated under reduced pressure. The residue was purified by column chromatography on silica gel (ethyl acetate in petroleum ether). Compound dimethyl 3-[[4-methyl-7-(2-methyl-4-pyridyl)-3-oxo-1,4-benzoxazin-6-yl]amino]benzene-1,2-dicarboxylate (2.8 g, 6.07 mmol, 82% yield) was obtained as a yellow oil. MS (ESI) m/z 462.2 [M+1]⁺.

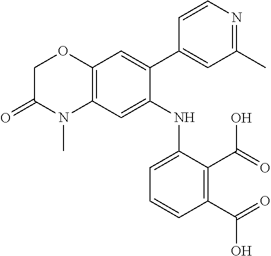 [SMILES: Cc1cc(-c2cc3c(cc2Nc2cccc(C(=O)O)c2C(=O)O)N(C)C(=O)CO3)ccn1]

3-[[4-methyl-7-(2-methyl-4-pyridyl)-3-oxo-1,4-benzoxazin-6-yl]amino]phthalic acid. To a solution of dimethyl 3-[[4-methyl-7-(2-methyl-4-pyridyl)-3-oxo-1,4-benzoxazin-6-yl]amino]benzene-1,2-dicarboxylate (2.800 g, 6.07 mmol) in Methanol (60 mL) and Water (20 mL) was added sodium hydroxide (1.456 g, 36.41 mmol). The mixture was stirred at 80 degree centigrade for 5 h. The mixture was acidified to pH 6-7 with hydrochloric acid (12M). The mixture was concentrated under reduced pressure. The crude product was used directly for next step without purification. Compound 3-[[4-methyl-7-(2-methyl-4-pyridyl)-3-oxo-1,4-benzoxazin-6-yl]amino]phthalic acid (2.5 g, 5.77 mmol, 95% yield) was obtained as a yellow solid. MS (ESI) m/z 434.1 [M+1]⁺.

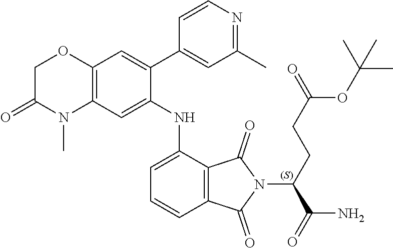

Tert-butyl (4S)-5-amino-4-[4-[[4-methyl-7-(2-methyl-4-pyridyl)-3-oxo-1,4-benzoxazin-6-yl]amino]-1,3-dioxo-isoindolin-2-yl]-5-oxo-pentanoate. To a solution of 3-[[4-methyl-7-(2-methyl-4-pyridyl)-3-oxo-1,4-benzoxazin-6-yl]amino]phthalic acid (2.500 g, 5.77 mmol) in Toluene (130 mL) was added tert-butyl (4S)-4,5-diamino-5-oxo-pentanoate hydrochloride (1.515 g, 6.35 mmol) and triethylamine (10.05 mL, 57.68 mmol). The mixture was stirred at 130° C. for 12 h. The mixture was filtered and then was concentrated under reduced pressure. The residue was purified by column chromatography on silica gel (ethyl acetate in petroleum ether). Compound tert-butyl (4S)-5-amino-4-[4-[[4-methyl-7-(2-methyl-4-pyridyl)-3-oxo-1,4-benzoxazin-6-yl]amino]-1,3-dioxo-isoindolin-2-yl]-5-oxo-pentanoate (2.1 g, 3.50 mmol, 60% yield) was obtained as a yellow solid. MS (ESI) m/z 600.2 [M+1]⁺.

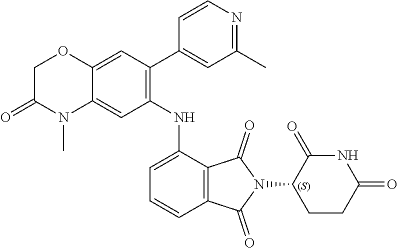

2-[(3S)-2,6-dioxo-3-piperidyl]-4-[[4-methyl-7-(2-methyl-4-pyridyl)-3-oxo-1,4-benzoxazin-6-yl]amino]isoindoline-1,3-dione. To a solution of tert-butyl (4S)-5-amino-4-[4-[[4-methyl-7-(2-methyl-4-pyridyl)-3-oxo-1,4-benzoxazin-6-yl]amino]-1,3-dioxo-isoindolin-2-yl]-5-oxo-pentanoate (2.100 g, 3.5 mmol) in acetonitrile (50 mL) was added benzenesulfonic acid (1.108 g, 7 mmol). The mixture was stirred at 60° C. for 12 h. The mixture was added saturated aqueous sodium bicarbonate and then was extracted with dichloromethane. The combined organic layers were dried over anhydrous sodium sulfate, filtered and concentrated under reduced pressure. The residue was purified by column chromatography on silica gel (methanol in dichloromethane). Compound 2-[(3S)-2,6-dioxo-3-piperidyl]-4-[[4-methyl-7-(2-methyl-4-pyridyl)-3-oxo-1,4-benzoxazin-6-yl]amino]isoindoline-1,3-dione (1.8 g, 3.43 mmol) (92.9% ee) was obtained as a yellow solid.

The 2-[(3S)-2,6-dioxo-3-piperidyl]-4-[[4-methyl-7-(2-methyl-4-pyridyl)-3-oxo-1,4-benzoxazin-6-yl]amino]isoindoline-1,3-dione (0.500 g, 0.95 mmol) was purified by prep-HPLC (acetonitrile+formic acid in water). The mixture was evaporated to remove the organic phase and the water phase was lyophilized. Compound 2-[(3S)-2,6-dioxo-3-piperidyl]-4-[[4-methyl-7-(2-methyl-4-pyridyl)-3-oxo-1,4-benzoxazin-6-yl]amino]isoindoline-1,3-dione (333 mg, 0.6323 mmol, 66% yield) was obtained as a yellow solid. MS (ESI) m/z 526.3 [M+1]^{+ 1}H NMR (400 MHz, DMSO-d₆) δ 11.11 (s, 1H), 8.34 (d, J=5.2 Hz, 1H), 8.27 (s, 1H), 7.48-7.40 (m, 1H), 7.32 (s, 1H), 7.28 (s, 1H), 7.22 (d, J=5.2 Hz, 1H), 7.15 (s, 1H), 7.09 (d, J=7.2 Hz, 1H), 6.83 (d, J=8.8 Hz, 1H), 5.08 (dd, J=5.2, 12.8 Hz, 1H), 4.75 (s, 2H), 3.30 (s, 3H), 2.96-2.81 (m, 1H), 2.64-2.52 (m, 2H), 2.39 (s, 3H), 2.10-1.96 (m, 1H).

### Example 2: Synthesis of Compound 7: 2-[(3S)-2,6-dioxo-3-piperidyl]-4-[[7-(5-fluoro-2-methyl-4-pyridyl)-4-methyl-3-oxo-1,4-benzoxazin-6-yl]amino]isoindoline-1,3-dione

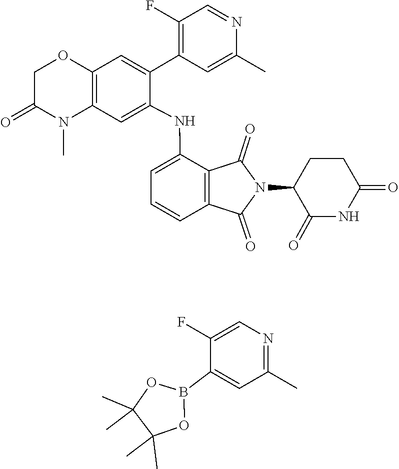

5-fluoro-2-methyl-4-(4,4,5,5-tetramethyl-1,3,2-dioxaborolan-2-yl)pyridine. To a solution of 4-bromo-5-fluoro-2-methyl-pyridine (2.000 g, 10.53 mmol), bis(pinacolato)diboron (4.010 g, 15.79 mmol) in 1,4-Dioxane (15 mL) was added potassium acetate (1.550 g, 15.79 mmol), (1,1′-bis(diphenylphosphino)ferrocene)palladium(II) dichloride (0.860 g, 1.05 mmol) under nitrogen, The solution was stirred at 110° C. for 12 h under nitrogen. The suspension was filtered and the filtrate was concentrated, and the residue was purified by prep-TLC (PE:EA) to afford 5-fluoro-2-methyl-4-(4,4,5,5-tetramethyl-1,3,2-dioxaborolan-2-yl)pyridine (1.5 g, 6.327 mmol, 60% yield) as a yellow oil.

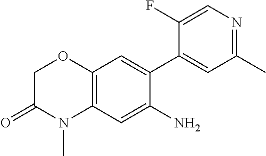 [SMILES: Cc1cc(-c2cc3c(cc2N)N(C)C(=O)CO3)c(F)cn1]

6-amino-7-(5-fluoro-2-methyl-4-pyridyl)-4-methyl-1,4-benzoxazin-3-one. To a solution of 5-fluoro-2-methyl-4-(4,4,5,5-tetramethyl-1,3,2-dioxaborolan-2-yl)pyridine (1.500 g, 6.33 mmol) and 6-amino-7-bromo-4-methyl-1,4-benzoxazin-3-one (1.630 g, 6.33 mmol) in 1,4-Dioxane (50 mL) was added potassium carbonate (2.619 g, 18.98 mmol) in Water (10 mL), and (1,1′-bis(diphenylphosphino)ferrocene)palladium(II) dichloride (0.119 g, 0.82 mmol) under nitrogen. The solution was stirred at 110° C. for 12 h. The reaction mixture was filtered and the filtrate was concentrated under reduce pressure by water pump. The residue was purified by column chromatography on silica gel eluted with petroleum ether/ethyl acetate. The desired 6-amino-7-(5-fluoro-2-methyl-4-pyridyl)-4-methyl-1,4-benzoxazin-3-one (1.3 g, 4.53 mmol, 72% yield) was obtained as a yellow solid. MS (ESI) m/z 288.1 [M+1]⁺.

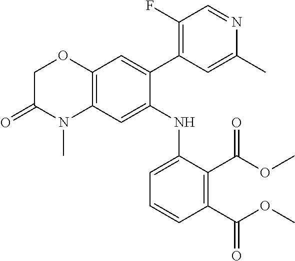 [SMILES: COC(=O)c1cccc(Nc2cc3c(cc2-c2cc(C)ncc2F)OCC(=O)N3C)c1C(=O)OC]

dimethyl 3-[[7-(5-fluoro-2-methyl-4-pyridyl)-4-methyl-3-oxo-1,4-benzoxazin-6-yl]amino]benzene-1,2-dicarboxylate. To a solution of 6-amino-7-(5-fluoro-2-methyl-4-pyridyl)-4-methyl-1,4-benzoxazin-3-one (1.300 g, 4.53 mmol) in 1,4-Dioxane (50 mL) was added dimethyl 3-bromobenzene-1,2-dicarboxylate (1.359 g, 4.98 mmol), cesium carbonate (4.412 g, 13.58 mmol) and methanesulfonato(2-dicyclohexylphosphino-2′,6′-di-i-propoxy-1,1′-biphenyl)(2′-amino-1,1′-biphenyl-2-yl)palladium(ii) (0.378 g, 0.45 mmol). The suspension was degassed under vacuum and purged with nitrogen several times. The mixture was stirred at 110° C. for 12 h. The mixture was filtered and filtrate was concentrated under reduced pressure. The residue was purified by column chromatography on silica gel (ethyl acetate in petroleum ether). Compound dimethyl 3-[[7-(5-fluoro-2-methyl-4-pyridyl)-4-methyl-3-oxo-1,4-benzoxazin-6-yl]amino]benzene-1,2-dicarboxylate (1.7 g, 3.55 mmol, 78% yield) was obtained as a brown oil. ¹H NMR (400 MHz, CDCl₃) δ 8.37 (s, 1H), 8.00 (s, 1H), 7.24 (t, J=7.6 Hz, 1H), 7.07 (d, J=5.6 Hz, 1H), 7.02 (s, 1H), 7.01-6.94 (m, 3H), 4.70 (s, 2H), 3.88 (s, 3H), 3.85 (s, 3H), 3.35 (s, 3H), 2.53 (s, 3H).

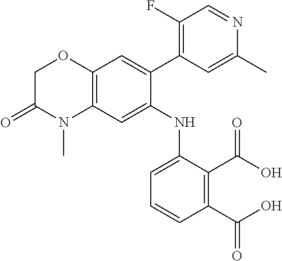 [SMILES: Cc1cc(-c2cc3c(cc2Nc2cccc(C(=O)O)c2C(=O)O)N(C)C(=O)CO3)c(F)cn1]

3-[[7-(5-fluoro-2-methyl-4-pyridyl)-4-methyl-3-oxo-1,4-benzoxazin-6-yl]amino]phthalic acid. To a solution of dimethyl 3-[[7-(5-fluoro-2-methyl-4-pyridyl)-4-methyl-3-oxo-1,4-benzoxazin-6-yl]amino]benzene-1,2-dicarboxylate (1.700 g, 3.55 mmol) in Methanol (60 mL) and Water (20 mL) was added sodium hydroxide (0.851 g, 21.27 mmol). The mixture was stirred at 80° C. for 3 h. The mixture was acidified to pH 6-7 with hydrochloric acid (4M). The mixture was concentrated under reduced pressure. The crude product was used directly for next step without purification. Compound 3-[[7-(5-fluoro-2-methyl-4-pyridyl)-4-methyl-3-oxo-1,4-benzoxazin-6-yl]amino]phthalic acid (1.5 g, 3.32 mmol, 94% yield) was obtained as a yellow solid. MS (ESI) m/z 452.1 [M+1]⁺.

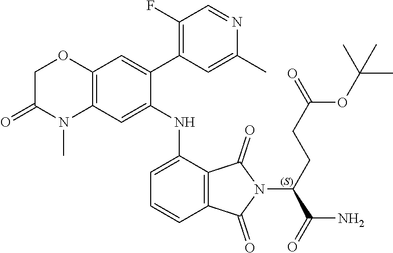

tert-butyl (4S)-5-amino-4-[4-[[7-(5-fluoro-2-methyl-4-pyridyl)-4-methyl-3-oxo-1,4-benzoxazin-6-yl]amino]-1,3-dioxo-isoindolin-2-yl]-5-oxo-pentanoate. To a solution of 3-[[7-(5-fluoro-2-methyl-4-pyridyl)-4-methyl-3-oxo-1,4-benzoxazin-6-yl]amino]phthalic acid (1.500 g, 3.32 mmol) in Toluene (80 mL) was added tert-butyl (4S)-4,5-diamino-5-oxo-pentanoate;hydrochloride (0.873 g, 3.66 mmol) and triethylamine (5.79 mL, 33.23 mmol). The mixture was stirred at 130° C. for 12 h. The mixture was filtered and then was concentrated under reduced pressure. The residue was purified by column chromatography on silica gel (ethyl acetate in petroleum ether). Compound tert-butyl (4S)-5-amino-4-[4-[[7-(5-fluoro-2-methyl-4-pyridyl)-4-methyl-3-oxo-1,4-benzoxazin-6-yl]amino]-1,3-dioxo-isoindolin-2-yl]-5-oxo-pentanoate (1.6 g, 2.59 mmol, 78% yield) was obtained as a yellow solid. MS (ESI) m/z 618.3 [M+1]⁺.

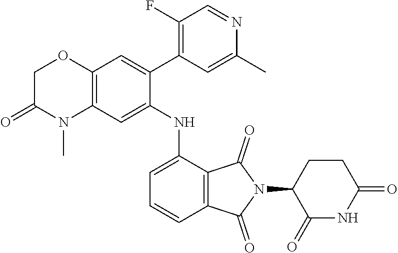 [SMILES: Cc1cc(-c2cc3c(cc2Nc2cccc4c2C(=O)N([C@H]2CCC(=O)NC2=O)C4=O)N(C)C(=O)CO3)c(F)cn1]

2-[(3S)-2,6-dioxo-3-piperidyl]-4-[[7-(5-fluoro-2-methyl-4-pyridyl)-4-methyl-3-oxo-1,4-benzoxazin-6-yl]amino]isoindoline-1,3-dione. To a 85olution of tert-butyl (4S)-5-amino-4-[4-[[7-(5-fluoro-2-methyl-4-pyridyl)-4-methyl-3-oxo-1,4-benzoxazin-6-yl]amino]-1,3-dioxo-isoindolin-2-yl]-5-oxo-pentanoate (1.600 g, 2.59 mmol) in acetonitrile (50 mL) was added benzenesulfonic acid (0.820 g, 5.18 mmol). The mixture was stirred at 60° C. for 12 h. The mixture was added saturated aqueous sodium bicarbonate (50 mL). The water phase was extracted with dichloromethane. The combined organic layers were dried over anhydrous sodium sulfate, filtered and concentrated under reduced pressure. The residue was purified by column chromatography on silica gel (methanol in dichloromethane). Compound 2-[(3S)-2,6-dioxo-3-piperidyl]-4-[[7-(5-fluoro-2-methyl-4-pyridyl)-4-methyl-3-oxo-1,4-benzoxazin-6-yl]amino]isoindoline-1,3-dione (1.2 g, 2.21 mmol, 85% yield) (95.5% ee) was obtained as a yellow solid. MS (ESI) m/z 544.2 [M+1]⁺.

The product 2-[(3S)-2,6-dioxo-3-piperidyl]-4-[[7-(5-fluoro-2-methyl-4-pyridyl)-4-methyl-3-oxo-1,4-benzoxazin-6-yl]amino]isoindoline-1,3-dione (1.200 g, 2.21 mmol) was added acetonitrile (50 mL) and methyl tert-butyl ether (100 mL). The mixture was stirred at 70° C. for 0.5 h. Then the mixture was filtered and filter cake was dried in vacuum. The residue was purified by prep-HPLC (acetonitrile+formic acid in water). The mixture was evaporated to remove the organic phase and the water phase was lyophilized. Compound 2-[(3S)-2,6-dioxo-3-piperidyl]-4-[[7-(5-fluoro-2-methyl-4-pyridyl)-4-methyl-3-oxo-1,4-benzoxazin-6-yl]amino]isoindoline-1,3-dione (735 mg, 1.3525 mmol, 61% yield) was obtained as a yellow solid. MS (ESI) m/z 544.3 [M+1]^{+ 1}H NMR (400 MHz, DMSO-d₆) δ 11.11 (s, 1H), 8.37 (d, J=1.2 Hz, 1H), 8.10 (s, 1H), 7.48 (dd, J=7.6, 8.4 Hz, 1H), 7.35-7.30 (m, 2H), 7.16 (s, 1H), 7.12 (d, J=7.2 Hz, 1H), 6.96 (d, J=8.8 Hz, 1H), 5.07 (dd, J=5.2, 12.8 Hz, 1H), 4.76 (s, 2H), 3.30 (s, 3H), 2.94-2.81 (m, 1H), 2.64-2.51 (m, 2H), 2.40 (s, 3H), 2.07-1.99 (m, 1H).

### Example 3: Synthesis of Compound 19: 2-(2,6-dioxopiperidin-3-yl)-4-((1-methyl-6-(2-methylpyridin-4-yl)-2-thioxo-1,2,3,4-tetrahydroquinolin-7-yl)amino)isoindoline-1,3-dione

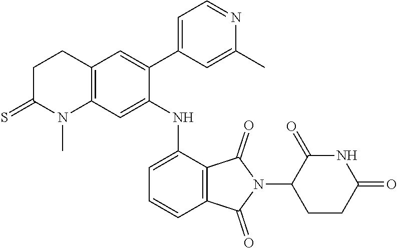 [SMILES: Cc1cc(-c2cc3c(cc2Nc2cccc4c2C(=O)N(C2CCC(=O)NC2=O)C4=O)N(C)C(=S)CC3)ccn1]

To a solution of 2-(2,6-dioxopiperidin-3-yl)-4-((1-methyl-6-(2-methylpyridin-4-yl)-2-oxo-1,2,3,4-tetrahydroquinolin-7-yl)amino)isoindoline-1,3-dione (0.040 g, 0.08 mmol) in toluene (2 mL) was added Lawesson's reagent (0.031 g, 0.08 mmol). The resulting mixture was stirred at 110° C. for 12 hours under nitrogen. The reaction mixture was filtered and the filtrate was concentrated under reduce pressure by water pump. The residue was purified by prep-HPLC (acetonitrile+trifluoroacetic acid in water). After the organic was removed, the aqueous solution was lyophilized to give 2-(2,6-dioxopiperidin-3-yl)-4-((1-methyl-6-(2-methylpyridin-4-yl)-2-thioxo-1,2,3,4-tetrahydroquinolin-7-yl)amino)isoindoline-1,3-dione (9.7 mg, 0.0175 mmol, 23% yield) as a yellow solid. MS (ESI) m/z: 540.3 [M+1]^{+ 1}H NMR (400 MHz, DMSO-d₆) δ 11.12 (s, 1H), 8.58 (s, 1H), 8.50 (s, 1H), 7.82-7.75 (m, 1H), 7.71-7.66 (m, 1H), 7.53 (s, 1H), 7.49-7.45 (m, 2H), 7.17 (d, J=7.2 Hz, 1H), 6.96 (d, J=8.4 Hz, 1H), 5.09 (dd, J=12.8, 5.2 Hz, 1H), 3.85 (s, 3H), 3.19-3.15 (m, 2H), 2.94-2.90 (m, 1H), 2.89-2.86 (m, 2H), 2.62-2.57 (m, 2H), 2.56 (s, 3H), 2.07-2.01 (m, 1H).

### Example 4: Synthesis of Compound 11: 2-[(3S)-2,6-dioxo-3-piperidyl]-4-[[1-methyl-6-(2-methyl-4-pyridyl)-2-oxo-4H-3,1-benzoxazin-7-yl]amino]isoindoline-1,3-dione

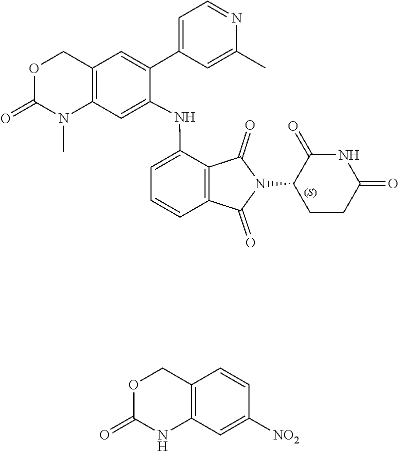

7-nitro-1,4-dihydro-3,1-benzoxazin-2-one. To a solution of (2-amino-4-nitro-phenyl)methanol (10.000 g, 59.47 mmol) in tetrahydrofuran (100 mL) was added slowly a solution of bis(trichloromethyl) carbonate (21.178 g, 71.36 mmol) in tetrahydrofuran (20 mL) at 0° C. The mixture was stirred at 0° C. for 10 min. Then triethylamine (36.25 mL, 208.15 mmol) was added to the mixture and the mixture was stirred at 25° C. for 0.5 h. The mixture was quenched with saturated sodium bicarbonate in aqueous solution. The water phase was extracted with ethyl acetate. The combined organic layers were dried over anhydrous sodium sulfate, filtered and concentrated under reduced pressure. Compound 7-nitro-1,4-dihydro-3,1-benzoxazin-2-one (11.0 g, 56.66 mmol, 95% yield) was obtained as a brown solid. ¹H NMR (400 MHz, DMSO-d₆) δ 10.61 (br s, 1H), 7.87 (d, J=8.4 Hz, 1H), 7.68 (s, 1H), 7.48 (d, J=8.4 Hz, 1H), 5.42 (s, 2H).

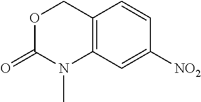

1-methyl-7-nitro-4H-3,1-benzoxazin-2-one. To a solution of 7-nitro-1,4-dihydro-3,1-benzoxazin-2-one (5.000 g, 25.75 mmol) in N,N-dimethylformamide (70 mL) was added sodium hydride (1.545 g, 38.63 mmol) at 0 degree centigrade. The mixture was stirred at 0° C. for 0.5 h. Then methyl iodide (5.483 g, 38.63 mmol) was added to the mixture and the mixture was stirred at 25° C. for 2 h. The mixture was quenched with ice water. The water phase was extracted with ethyl acetate. The combined organic layers were washed with brine and then was dried over anhydrous sodium sulfate, filtered and concentrated under reduced pressure. Compound 1-methyl-7-nitro-4H-3,1-benzoxazin-2-one (4.5 g, 21.62 mmol, 84% yield) was obtained as a yellow solid.

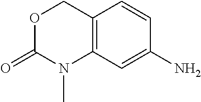

7-amino-1-methyl-4H-3,1-benzoxazin-2-one. To a solution of 1-methyl-7-nitro-4H-3,1-benzoxazin-2-one (4.500 g, 21.62 mmol) in Ethanol (120 mL) and Water (40 mL) was added iron powder (6.036 g, 108.08 mmol) and ammonia hydrochloride (11.563 g, 216.17 mmol). The mixture was stirred at 80° C. for 2 h. The mixture was filtered and filtrate was concentrated under reduced pressure. The residue was purified by column chromatography on silica gel (ethyl acetate in petroleum ether). Compound 7-amino-1-methyl-4H-3,1-benzoxazin-2-one (1.0 g, 5.612 mmol, 26% yield) was obtained as brown solid. ¹H NMR (400 MHz, CDCl₃) δ 6.90 (d, J=8.0 Hz, 1H), 6.38 (dd, J=2.4, 7.6 Hz, 1H), 6.26 (d, J=2.0 Hz, 1H), 5.10 (s, 2H), 3.91-3.69 (m, 2H), 3.33 (s, 3H).

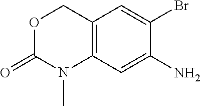

7-amino-6-bromo-1-methyl-4H-3,1-benzoxazin-2-one. To a solution of 7-amino-1-methyl-4H-3,1-benzoxazin-2-one (1.000 g, 5.61 mmol) in dichloromethane (20 mL) and Methanol (5 mL) was added tetra-n-butylammonium tribromide (1.759 g, 3.65 mmol) in portions. The mixture was stirred at 0° C. for 2 h. The mixture was quenched with saturated sodium thiosulfate aqueous solution. The water phase was extracted with dichloromethane. The residue was purified by column chromatography on silica gel (ethyl acetate in petroleum ether). Compound 7-amino-6-bromo-1-methyl-4H-3,1-benzoxazin-2-one (700 mg, 2.72 mmol, 49% yield) was obtained as a brown solid. ¹H NMR (400 MHz, CDCl₃) δ 7.16 (s, 1H), 6.33 (s, 1H), 5.07 (s, 2H), 4.80-3.52 (m, 2H), 3.32 (s, 3H).

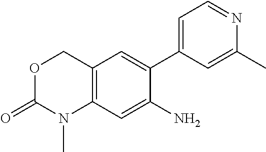 [SMILES: Cc1cc(-c2cc3c(cc2N)N(C)C(=O)OC3)ccn1]

7-amino-1-methyl-6-(2-methyl-4-pyridyl)-4H-3,1-benzoxazin-2-one. To a solution of 7-amino-6-bromo-1-methyl-4H-3,1-benzoxazin-2-one (0.700 g, 2.72 mmol) in 1,4-Dioxane (10 mL) and Water (1 mL) was added 2-methyl-4-(4,4,5,5-tetramethyl-1,3,2-dioxaborolan-2-yl)pyridine (0.716 g, 3.27 mmol), potassium carbonate (1.127 g, 8.17 mmol) and (1,1′-bis(diphenylphosphino)ferrocene)palladium(II) dichloride (0.222 g, 0.27 mmol). The suspension was degassed under vacuum and purged with nitrogen several times. The mixture was stirred at 110° C. for 12 h. The mixture was filtered and filtrate was concentrated under reduced pressure. The residue was purified by column chromatography on silica gel (ethyl acetate in petroleum ether). Compound 7-amino-1-methyl-6-(2-methyl-4-pyridyl)-4H-3,1-benzoxazin-2-one (540 mg, 2.01 mmol, 74% yield) was obtained as a yellow solid. MS (ESI) m/z 270.2 [M+1]⁺.

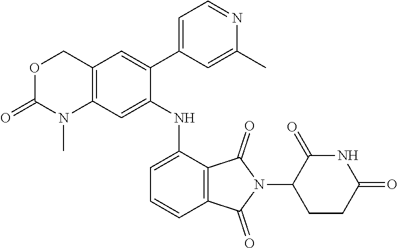 [SMILES: Cc1cc(-c2cc3c(cc2Nc2cccc4c2C(=O)N(C2CCC(=O)NC2=O)C4=O)N(C)C(=O)OC3)ccn1]

2-(2,6-dioxo-3-piperidyl)-4-[[1-methyl-6-(2-methyl-4-pyridyl)-2-oxo-4H-3,1-benzoxazin-7-yl]amino]isoindoline-1,3-dione. To a solution of 7-amino-1-methyl-6-(2-methyl-4-pyridyl)-4H-3,1-benzoxazin-2-one (0.200 g, 0.74 mmol) in 1,4-Dioxane (10 mL) was added 4-bromo-2-(2,6-dioxo-3-piperidyl)isoindoline-1,3-dione (0.300 g, 0.89 mmol), potassium carbonate (0.308 g, 2.23 mmol) and Chloro(2-dicyclohexylphosphino-2′,4′,6′-tri-i-propyl-1,1′-biphenyl)(2′-amino-1,1′-biphenyl-2-yl) palladium(II) (0.058 g, 0.07 mmol). The suspension was degassed under vacuum and purged with nitrogen several times. The mixture was stirred at 110° C. for 12 h. The mixture was filtered and filtrate was concentrated under reduced pressure. The residue was purified by prep-HPLC (acetonitrile+formic acid in water). The mixture was evaporated to remove the organic phase and the water phase was lyophilized. Compound 2-(2,6-dioxo-3-piperidyl)-4-[[1-methyl-6-(2-methyl-4-pyridyl)-2-oxo-4H-3,1-benzoxazin-7-yl]amino]isoindoline-1,3-dione (230 mg, 0.44 mmol, 59% yield) was obtained as a yellow solid. MS (ESI) m/z 526.2 [M+1]⁺.

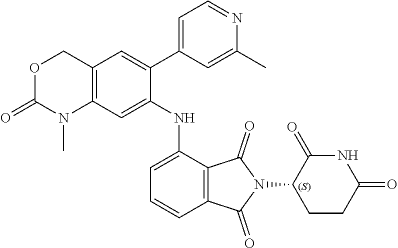

2-[(3S)-2,6-dioxo-3-piperidyl]-4-[[1-methyl-6-(2-methyl-4-pyridyl)-2-oxo-4H-3,1-benzoxazin-7-yl]amino]isoindoline-1,3-dione. The racemic 2-(2,6-dioxo-3-piperidyl)-4-[[1-methyl-6-(2-methyl-4-pyridyl)-2-oxo-4H-3,1-benzoxazin-7-yl]amino]isoindoline-1,3-dione (0.230 g, 0.44 mmol) was separated by SFC. The eluent was evaporated to remove the solvent at 40° C. The mixture was lyophilized. Compound 2-[(3S)-2,6-dioxo-3-piperidyl]-4-[[1-methyl-6-(2-methyl-4-pyridyl)-2-oxo-4H-3,1-benzoxazin-7-yl]amino]isoindoline-1,3-dione (67 mg, 0.1283 mmol, 29% yield) was obtained as a yellow solid. MS (ESI) m/z 526.2 [M+1]^{+ 1}H NMR (400 MHz, DMSO-d₆) δ 11.11 (s, 1H), 8.39 (s, 1H), 8.37 (d, J=5.2 Hz, 1H), 7.48 (dd, J=7.2, 8.4 Hz, 1H), 7.41 (s, 1H), 7.30 (s, 1H), 7.23-7.19 (m, 2H), 7.16 (d, J=7.2 Hz, 1H), 7.08 (d, J=8.4 Hz, 1H), 5.32 (s, 2H), 5.08 (dd, J=5.2, 12.8 Hz, 1H), 3.30 (s, 3H), 2.93-2.83 (m, 1H), 2.64-2.54 (m, 2H), 2.41 (s, 3H), 2.06-1.99 (m, 1H).

### Example 5: Synthesis of Compound 83: 2-(2,6-dioxo-3-piperidyl)-4-[[1,4,4-trimethyl-6-(2-methyl-4-pyridyl)-2-oxo-3H-quinolin-7-yl]amino]isoindoline-1,3-dione

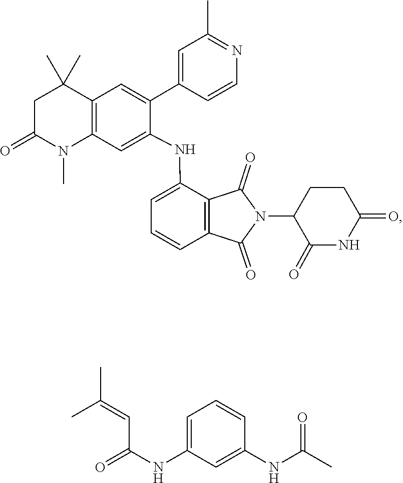 [SMILES: Cc1cc(-c2cc3c(cc2Nc2cccc4c2C(=O)N(C2CCC(=O)NC2=O)C4=O)N(C)C(=O)CC3(C)C)ccn1 ; CC(=O)Nc1cccc(NC(=O)C=C(C)C)c1]

N-(3-acetamidophenyl)-3-methyl-but-2-enamide. To a solution of N-(3-aminophenyl)acetamide (5.5 g, 36.62 mmol), triethylamine (6.7 mL, 38.45 mmol) in dichloromethane (60 mL), THE (30 mL) was added 3-methylbut-2-enoyl chloride (4.34 g, 36.62 mmol) dropwise at 0° C. The solution was stirred at 20° C. for 12 h. LCMS showed this reaction worked. The solution was extracted with dichloromethane. The organic layer was concentrated and the residue was purified by column on silica gel (dichloromethane:methanol) to afford N-(3-acetamidophenyl)-3-methyl-but-2-enamide (7.0 g, 30.13 mmol, 82% yield). MS (ESI) m/z: 233.5 [M+1]⁺.

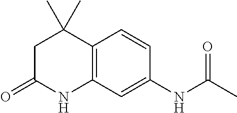 [SMILES: CC(=O)Nc1ccc2c(c1)NC(=O)CC2(C)C]

N-(4,4-dimethyl-2-oxo-1,3-dihydroquinolin-7-yl)acetamide. A mixture N-(3-acetamidophenyl)-3-methyl-but-2-enamide (2.32 g, 9.99 mmol), aluminum chloride (10.00 g) in Chlorobenzene (40 mL) was stirred at 120° C. for 12 h. The solution was poured into ice/water, then it was extracted with dichloromethane. The organic layer was washed with brine, dried over sodium sulfate, filtered and the filtrate was concentrated and the residue was purified by column on silica gel (dichloromethane:methanol) to afford N-(4,4-dimethyl-2-oxo-1,3-dihydroquinolin-7-yl)acetamide (1.2 g, 5.1 mmol, 51% yield). MS (ESI) m/z: 255.3 [M+1]*.

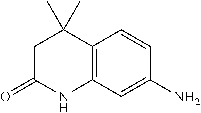

7-amino-4,4-dimethyl-1,3-dihydroquinolin-2-one. To a solution of N-(4,4-dimethyl-2-oxo-1,3-dihydroquinolin-7-yl)acetamide (1.20 g, 5.17 mmol) in Ethanol (30 mL) was added concentrated hydrochloride acid (10 mL, 130.59 mmol). The solution was stirred at 70° C. for 4 h. The solution was poured into ice/water, then adjusted to pH=9 with solid sodium hydroxide, then it was extracted with dichloromethane. The organic layer was washed with brine, dried over sodium sulfate, filtered and the filtrate was concentrated to afford 7-amino-4,4-dimethyl-1,3-dihydroquinolin-2-one (600 mg, 3.15 mmol, 61% yield). MS (ESI) m/z: 191.6 [M+1]⁺.

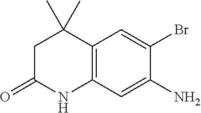

7-amino-6-bromo-4,4-dimethyl-1,3-dihydroquinolin-2-one. To a mixture of 7-amino-4,4-dimethyl-1,3-dihydroquinolin-2-one (0.60 g, 3.15 mmol) suspended in dichloromethane (40 mL), methanol (10 mL) was added tetra-n-butylammonium tribromide (0.99 g, 2.05 mmol). The reaction mixture was stirred at 25° C. for two hours and then was partitioned between dichloromethane and 10% aqueous sodium thiosulfate. The organic layer was dried over MgSO₄, filtered and concentrated under reduced pressure. Purification by column chromatography, eluting with ethyl acetate/hexanes, provided 7-amino-6-bromo-4,4-dimethyl-1,3-dihydroquinolin-2-one (420 mg, 1.560 mmol, 49% yield). MS (ESI) m/z: 269.1 [M+1]⁺.

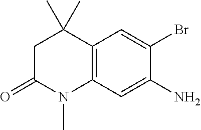 [SMILES: CN1C(=O)CC(C)(C)c2cc(Br)c(N)cc21]

7-amino-6-bromo-1,4,4-trimethyl-3,4-dihydroquinolin-2(1H)-one. 7-amino-6-bromo-4,4-dimethyl-1,3-dihydroquinolin-2-one (0.42 g, 1.56 mmol) was dissolved in THE (15 mL) and cooled to 0° C. Potassium bis(trimethylsilyl)amide (2.1 mL, 2.1 mmol, 1.0M in THF) was then added dropwise, followed by dropwise addition of iodomethane (0.297 g, 2.1 mmol). The mixture was stirred for 12 hours at 20° C. The reaction was quenched with ammonium chloride, and then partitioned between ethyl acetate and brine. The organic layer was dried over sodium sulfate, filtered and concentrated under reduced pressure. The residue was purified by column chromatography, eluting with ethyl acetate/hexanes, provided 7-amino-6-bromo-1,4,4-trimethyl-3H-quinolin-2-one (300 mg, 1.0595 mmol, 68% yield) as a yellow solid ¹H NMR (400 MHz, CDCl₃) δ 7.28 (s, 1H), 6.44 (s, 1H), 4.35-4.31 (m, 2H), 3.34 (s, 3H), 2.48 (s, 6H); MS (ESI) m/z: 283.0 [M+1]⁺.

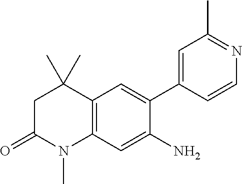 [SMILES: Cc1cc(-c2cc3c(cc2N)N(C)C(=O)CC3(C)C)ccn1]

7-amino-1,4,4-trimethyl-6-(2-methyl-4-pyridyl)-3H-quinolin-2-one. To a solution of 7-amino-6-bromo-1,4,4-trimethyl-3H-quinolin-2-one (0.20 g, 0.71 mmol) in 1,4-Dioxane (10 mL), water (2 mL) was added 2-methyl-4-(4,4,5,5-tetramethyl-1,3,2-dioxaborolan-2-yl)pyridine (0.15 g, 0.71 mmol), potassium carbonate (0.24 g, 1.77 mmol), (1,1′-bis(diphenylphosphino)ferrocene)palladium(II) dichloride (0.057 g, 0.070 mmol) under nitrogen, The solution was stirred at 110° C. for 12 h. The reaction was quenched with water, then extracted with dichloromethane. The organic layer was concentrated and the residue was purified by prep-TLC (Ethyl acetate) to afford 7-amino-1,4,4-trimethyl-6-(2-methyl-4-pyridyl)-3H-quinolin-2-one (170 mg, 0.575 mmol, 81% yield). MS (ESI) m/z: 296.4 [M+1]⁺.

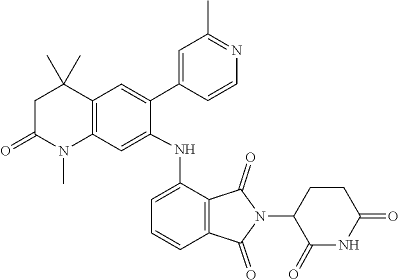 [SMILES: Cc1cc(-c2cc3c(cc2Nc2cccc4c2C(=O)N(C2CCC(=O)NC2=O)C4=O)N(C)C(=O)CC3(C)C)ccn1]

2-(2,6-dioxo-3-piperidyl)-4-[[1,4,4-trimethyl-6-(2-methyl-4-pyridyl)-2-oxo-3H-quinolin-7-yl]amino]isoindoline-1,3-dione. To a solution of 7-amino-1,4,4-trimethyl-6-(2-methyl-4-pyridyl)-3H-quinolin-2-one (0.150 g, 0.510 mmol), 4-bromo-2-(2,6-dioxo-3-piperidyl)isoindoline-1,3-dione (0.171 g, 0.510 mmol) in 1,4-Dioxane (10 mL) was added potassium carbonate (0.175 g, 1.27 mmol), chloro(2-dicyclohexylphosphino-2′,4′,6′-triisopropyl-1,1′-biphenyl)[2-(2′-amino-1,1′-biphenyl)]palladium(II) (0.040 g, 0.050 mmol) under nitrogen. Then the solution was stirred at 110° C. for 12 h. The solution was filtered and the filtrate was concentrated. The residue was purified by prep-HPLC (acetonitrile/water) to afford 2-(2,6-dioxo-3-piperidyl)-4-[[1,4,4-trimethyl-6-(2-methyl-4-pyridyl)-2-oxo-3H-quinolin-7-yl]amino]isoindoline-1,3-dione (43 mg, 0.079 mmol, 15% yield). ¹H NMR (400 MHz, DMSO-d₆) δ=11.13 (s, 1H), 8.60 (brs, 1H), 7.91-7.89 (m, 1H), 7.80-7.78 (m, 1H), 7.50-7.46 (m, 2H), 7.28 (s, 1H), 7.17 (d, J=8.0 Hz, 1H), 6.98-6.97 (m, 1H), 5.12-5.08 (m, 1H), 3.31 (s, 3H), 2.98-2.94 (m, 1H), 2.56-2.53 (m, 1H), 2.53-2.51 (m, 2H), 2.04-2.02 (m, 1H), 1.41 (s, 9H). MS (ESI) m/z: 552.3 [M+1]⁺.

### Example 6: Synthesis of Compound 1: 2-(2,6-dioxopiperidin-3-yl)-4-((4-methyl-7-(2-methylpyridin-4-yl)-3,4-dihydro-2H-benzo[b][1,4]oxazin-6-yl)amino)isoindoline-1,3-dione

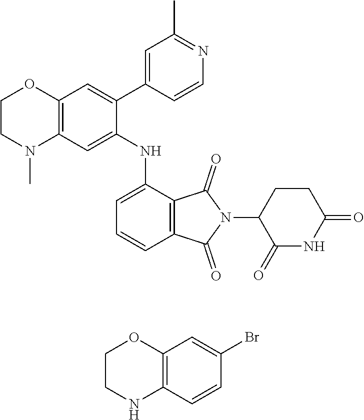 [SMILES: Cc1cc(-c2cc3c(cc2Nc2cccc4c2C(=O)N(C2CCC(=O)NC2=O)C4=O)N(C)CCO3)ccn1 ; Brc1ccc2c(c1)OCCN2]

7-bromo-3,4-dihydro-2H-1,4-benzoxazine. To a solution of 2-amino-5-bromo-phenol (2.000. g, 10.64 mmol) and 1,2-dibromoethane (2.397 g, 12.76 mmol) in N,N-dimethylformamide (20 mL) was added potassium carbonate (4.404. g, 31.91 mmol), the mixture was stirred at 120° C. for 12 hours. The mixture was cooled to room temperature. The reaction mixture was diluted with aqueous Sodium chloride (50 mL) and extracted with ethyl acetate. The combined organic layers were washed with aqueous Sodium chloride, dried over sodium sulfate, filtered and evaporated to give the crude which was purified by silica gel column chromatography (ethyl acetate in petroleum ether) to give 7-bromo-3,4-dihydro-2H-1,4-benzoxazine (0.800 g, 3.74 mmol, 35% yield) as grey oil. MS (ESI) m/z: 215.9 [M+1]⁺.

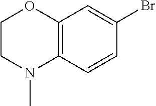

7-bromo-4-methyl-3,4-dihydro-2H-benzo[b][1,4]oxazine. To a solution of 7-bromo-3,4-dihydro-2H-1,4-benzoxazine (0.400. g, 1.87 mmol) in tetrahydrofuran (5 mL) was added sodium hydride (0.063. g, 2.62 mmol) stirred at 0° C. for 0.5 hours, then was added methyl iodide (0.450. g, 3.17 mmol) and stirred at 50° C. for 12 hours. The reaction mixture was filtered and the filtrate was concentrated under reduce pressure by water pump. The residue was purified column chromatography on silica gel eluted with petroleum ether:ethyl acetate=10:1 to 5:1 to afford 7-bromo-4-methyl-3,4-dihydro-2H-benzo[b][1,4]oxazine (160 mg, 0.70 mmol, 38% yield) as grey oil. MS (ESI) m/z: 266.0 [M+1]⁺.

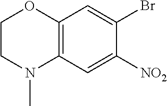

7-bromo-4-methyl-6-nitro-3,4-dihydro-2H-benzo[b][1,4]oxazine. To a solution of 7-bromo-4-methyl-2,3-dihydro-1,4-benzoxazine (0.160. g, 0.70 mmol) in concentrated sulfuric acid (2 mL, 37.55 mmol) was added nitrate of potash (0.071. g, 0.70 mmol) at 0° C. and the mixture was stirred at 0° C. for 2 hours. TLC (petroleum ether:ethyl acetate=3:1) showed the starting material was consumed completely and new spot formed. Quench the reaction mixture by pouring it into ice water. Then filtered to obtain the product 7-bromo-4-methyl-6-nitro-3,4-dihydro-2H-benzo[b][1,4]oxazine (40 mg, 0.15 mmol, 21% yield) as yellow solid. ¹H NMR (400 MHz, CHCl₃) δ 7.29-7.28 (m, 1H), 7.05-7.04 (m, 1H), 4.37-4.34 (m, 2H), 3.34-3.31 (m, 2H), 2.95-2.94 (m, 3H).

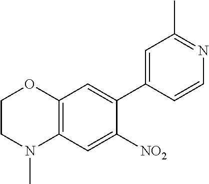 [SMILES: Cc1cc(-c2cc3c(cc2[N+](=O)[O-])N(C)CCO3)ccn1]

4-methyl-7-(2-methylpyridin-4-yl)-6-nitro-3,4-dihydro-2H-benzo[b][1,4]oxazine. To a solution of 7-bromo-4-methyl-6-nitro-2,3-dihydro-1,4-benzoxazine (0.040. g, 0.15 mmol) and 2-methyl-4-(4,4,5,5-tetramethyl-1,3,2-dioxaborolan-2-yl)pyridine (0.035. g, 0.16 mmol) in 1,4-Dioxane (2 mL) was added potassium carbonate (0.061. g, 0.44 mmol), the mixture was replaced gas of nitrogen for 3 times and added (1,1′-bis(diphenylphosphino)ferrocene)palladium(II) dichloride (0.012. g, 0.01 mmol), the mixture was stirred at 110° C. for 12 hours. The suspension was filtered and the filtrate was concentrated. The residue was purified by prep-TLC (petroleum ether:ethyl acetate) to afford 4-methyl-7-(2-methylpyridin-4-yl)-6-nitro-3,4-dihydro-2H-benzo[b][1,4]oxazine (30 mg, 0.11 mmol, 72% yield) as yellow solid. MS (ESI) m/z: 286.4 [M+1]⁺.

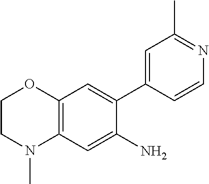 [SMILES: Cc1cc(-c2cc3c(cc2N)N(C)CCO3)ccn1]

4-methyl-7-(2-methylpyridin-4-yl)-3,4-dihydro-2H-benzo[b][1,4]oxazin-6-amine. To a solution of 4-methyl-7-(2-methylpyridin-4-yl)-6-nitro-3,4-dihydro-2H-benzo[b][1,4]oxazine (0.035 g, 0.12 mmol) in Ethanol (1 mL) and Water (0.300 mL) was added ferrous powder (0.070. g, 1.25 mmol) and ammonium chloride (0.070. g, 1.31 mmol), the mixture was stirred at 70° C. for 2 hours. The combined organic layers were washed with brine, dried with anhydrous sodium sulfate, filtered and concentrated in vacuum to give the residue. The suspension was filtered and the filtrate was concentrated to obtain the product 4-methyl-7-(2-methylpyridin-4-yl)-3,4-dihydro-2H-benzo[b][1,4]oxazin-6-amine (30 mg, 0.12 mmol, 96% yield) as yellow solid. MS (ESI) m/z: 256.5 [M+1]⁺.

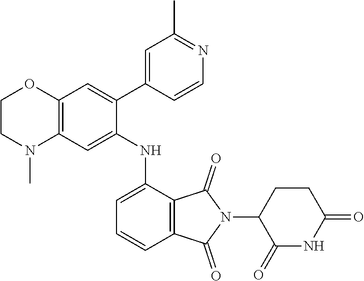 [SMILES: Cc1cc(-c2cc3c(cc2Nc2cccc4c2C(=O)N(C2CCC(=O)NC2=O)C4=O)N(C)CCO3)ccn1]

2-(2,6-dioxopiperidin-3-yl)-4-((4-methyl-7-(2-methylpyridin-4-yl)-3,4-dihydro-2H-benzo[b][1,4]oxazin-6-yl)amino)isoindoline-1,3-dione. To a solution of 4-methyl-7-(2-methyl-4-pyridyl)-2,3-dihydro-1,4-benzoxazin-6-amine (0.030. g, 0.12 mmol) and 4-bromo-2-(2,6-dioxo-3-piperidyl)isoindoline-1,3-dione (0.042. g, 0.12 mmol) in 1,4-Dioxane (1 mL) was added potassium carbonate (0.060. g, 0.43 mmol), then the mixture was replaced gas of nitrogen for 3 times and added chloro(2-dicyclohexylphosphino-2′,4′,6′-triisopropyl-1,1′-biphenyl)[2-(2′-amino-1,1′-biphenyl)]palladium(II) (0.009. g, 0.01 mmol), the mixture was stirred at 110° C. for 12 hours. The reaction mixture was filtered and the filtrate was concentrated under reduce pressure by water pump. The crude product was purified by prep-HPLC. The purified solution was lyophilized to give 2-(2,6-dioxopiperidin-3-yl)-4-((4-methyl-7-(2-methylpyridin-4-yl)-3,4-dihydro-2H-benzo[b][1,4]oxazin-6-yl)amino)isoindoline-1,3-dione (24 mg, 0.0469 mmol, 40% yield) as yellow solid. MS (ESI) m/z: 512.2 [M+1]⁺. ¹H NMR (400 MHz, DMSO-d6) δ 11.16-11.08 (m, 1H), 8.58-8.48 (m, 1H), 8.38-8.32 (m, 1H), 7.94-7.87 (m, 1H), 7.86-7.79 (m, 1H), 7.46-7.39 (m, 1H), 7.07 (s, 2H), 6.80-6.75 (m, 1H), 6.72-6.68 (m, 1H), 5.13-5.07 (m, 1H), 4.33-4.27 (m, 2H), 3.43-3.39 (m, 2H), 2.95-2.92 (m, 3H), 2.80-2.70 (m, 1H), 2.65-2.63 (m, 1H), 2.60-2.59 (m, 1H), 2.57 (s, 3H), 2.08-2.03 (m, 1H).

### Example 7: Synthesis of Compound 84: 4-((6-(1-Acetylpiperidin-4-yl)-1-methyl-2-oxo-1,2,3,4-tetrahydroquinolin-7-yl)amino)-2-(2,6-dioxopiperidin-3-yl)isoindoline-1,3-dione

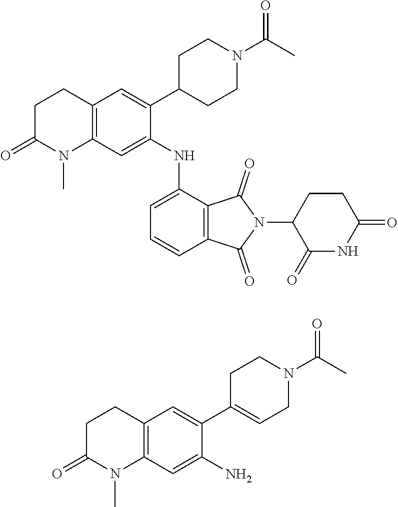 [SMILES: CC(=O)N1CCC(c2cc3c(cc2Nc2cccc4c2C(=O)N(C2CCC(=O)NC2=O)C4=O)N(C)C(=O)CC3)CC1 ; CC(=O)N1CC=C(c2cc3c(cc2N)N(C)C(=O)CC3)CC1]

6-(1-Acetyl-1,2,3,6-tetrahydropyridin-4-yl)-7-amino-1-methyl-3,4-dihydroquinolin 2(1H)-one. To a stirred solution of 7-amino-6-bromo-1-methyl-3,4-dihydroquinolin-2(1H)-one (150 mg, 0.583 mmol) and 1-(4-(4,4,5,5-tetramethyl-1,3,2-dioxaborolan-2-yl)-3,6-dihydropyridin-1(2H)-yl)ethan-1-one (220 mg, 0.875 mmol) in 1,4-dioxane (9 mL) and H₂O (1 mL) was added K₂CO₃ (195 mg, 1.411 mmol). The mixture was purged with nitrogen gas for 10 min, followed by addition of PdCl₂(dppf)-CH₂Cl₂ adduct (38.4 mg, 0.047 mmol). The reaction mixture was warmed to 90° C. and stirred for 16 h. The reaction mixture was quenched with water and extracted with Ethyl acetate. The combined organic extracts were dried over sodium sulfate, filtered, and concentrated under reduced pressure. The resulting crude residue was purified by silica gel column chromatography to afford 6-(1-Acetyl-1,2,3,6-tetrahydropyridin-4-yl)-7-amino-1-methyl-3,4-dihydroquinolin 2(1H)-one (122 mg, 0.335 mmol, 57% yield). MS (ESI) m z: 300 [M+H]⁺.

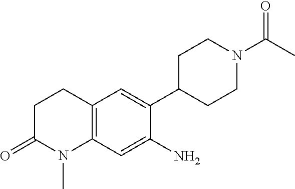 [SMILES: CC(=O)N1CCC(c2cc3c(cc2N)N(C)C(=O)CC3)CC1]

6-(1-acetylpiperidin-4-yl)-7-amino-1-methyl-3,4-dihydroquinolin-2(1H)-one. To a stirred solution of 6-(1-acetyl-1,2,3,6-tetrahydropyridin-4-yl)-7-amino-1-methyl-3,4-dihydroquinolin-2(1H)-one (70 mg, 0.192 mmol) in THE (3 mL) under nitrogen atmosphere was added palladium on carbon (20.43 mg, 0.019 mmol). The reaction mixture was stirred under H₂ atmosphere at 25° C. for 10 h. The reaction mixture was filtered through celite and washed with THF. The filtrate was concentrated under reduced pressure to yield 6-(1-acetylpiperidin-4-yl)-7-amino-1-methyl-3,4-dihydroquinolin-2(1H)-one (70 mg, 0.155 mmol, 81% yield). MS (ESI) m z: 302 [M+H]⁺.

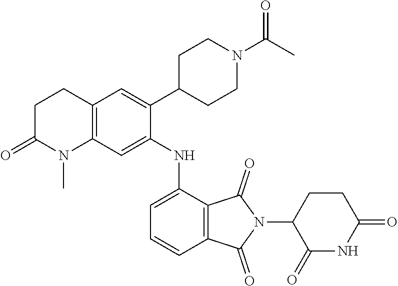 [SMILES: CC(=O)N1CCC(c2cc3c(cc2Nc2cccc4c2C(=O)N(C2CCC(=O)NC2=O)C4=O)N(C)C(=O)CC3)CC1]

4-((6-(1-acetylpiperidin-4-yl)-1-methyl-2-oxo-1,2,3,4-tetrahydroquinolin-7-yl)amino)-2-(2,6 dioxopiperidin-3 yl)isoindoline-1,3-dione. To a solution of 6-(1-acetylpiperidin-4-yl)-7-amino-1-methyl-3,4-dihydroquinolin-2(1H)-one (70 mg, 0.231 mmol) and 4-bromo-2-(2,6-dioxopiperidin-3-yl)isoindoline-1,3-dione (94 mg, 0.278 mmol) in 1,4-dioxane (15 mL) was added cesium carbonate (226 mg, 0.694 mmol). The reaction mixture was degassed with nitrogen, followed by addition of RuPhos Pd G3 (19.38 mg, 0.023 mmol). The reaction mixture was stirred in MW at 110° C. for 4 h. The reaction was monitored by UPLC-MS. The reaction mixture was filtered through celite, and washed with hot dioxane (20 mL) and THE (20 mL). The filtrate was concentrated under reduced pressure and the resulting crude residue was purified by prep-HPLC (ACN/H₂O (5 mM Ammonium formate)) and lyophilized to give 4-((6-(1-acetylpiperidin-4-yl)-1-methyl-2-oxo-1,2,3,4-tetrahydroquinolin-7-yl)amino)-2-(2,6 dioxopiperidin-3 yl)isoindoline-1,3-dione (21 mg, 0.037 mmol, 16% yield). ¹H NMR (400 MHz, DMSO-d6): δ 11.14 (s, 1H), 8.17 (s, 1H), 7.56 (dd, J=7.20, 8.40 Hz, 1H), 7.28 (s, 1H), 7.19 (d, J=6.80 Hz, 1H), 7.01 (s, 1H), 6.90 (d, J=8.40 Hz, 1H), 5.14 (dd, J=5.20, 12.80 Hz, 1H), 4.47 (m, 1H), 3.86 (m, 1H), 3.19 (s, 3H), 3.08-2.82 (m, 5H), 2.70-2.52 (m, 4H), 2.08 (m, 1H), 2.00 (s, 3H), 1.72-1.42 (m, 4H). MS (ESI) m z: 558 [M+H]⁺.

### Example 8: Synthesis of Compound 85: 2-(2,6-dioxopiperidin-3-yl)-4-((1,3,3-trimethyl-6-(2-methylpyridin-4-yl)-2-oxo-1,2,3,4-tetrahydroquinolin-7-yl)amino)isoindoline-1,3-dione

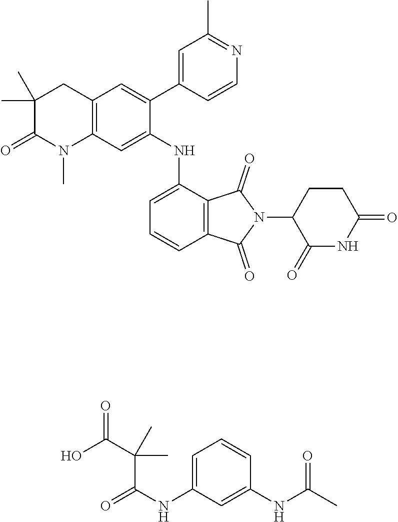 [SMILES: Cc1cc(-c2cc3c(cc2Nc2cccc4c2C(=O)N(C2CCC(=O)NC2=O)C4=O)N(C)C(=O)C(C)(C)C3)ccn1 ; CC(=O)Nc1cccc(NC(=O)C(C)(C)C(=O)O)c1]

3-(3-acetamidoanilino)-2,2-dimethyl-3-oxo-propanoic acid. To a solution of N-(3-aminophenyl)acetamide (5.33 g, 35.5 mmol) in THE (20 mL) was added triethylamine (6.2 mL, 35.5 mmol). Then the solution was cooled to 0° C. 2,2-dimethylpropanedioyl dichloride (6.0 g, 35.5 mmol) in THE (5 mL) was added into the reaction solution dropwise at 0° C. The solution was stirred at 0° C. for 2 h. The reaction was quenched with Water, then extracted with dichloromethane. The organic layer was concentrated and the residue was triturated with Ethyl acetate to afford 3-(3-acetamidoanilino)-2,2-dimethyl-3-oxo-propanoic acid (7.0 g, 26.5 mmol, 75% yield). MS (ESI) m/z: 221.7[M+1]⁺.

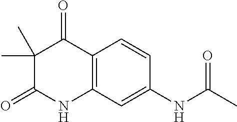 [SMILES: CC(=O)Nc1ccc2c(c1)NC(=O)C(C)(C)C2=O]

N-(3,3-dimethyl-2,4-dioxo-1H-quinolin-7-yl)acetamide. A mixture 3-(3-acetamidoanilino)-2,2-dimethyl-3-oxo-propanoic acid (2.7 g, 10.2 mmol), polyphosphoric acid (20.0 g, 59.1 mmol) in was stirred at 70° C. for 12 h. The solution was poured into ice/water, then it was extracted with dichloromethane. The organic layer was washed with brine, dried over sodium sulfate, filtered and the filtrate was concentrated and the residue was purified by column on silica gel (dichloromethane:Methanol) to afford N-(3,3-dimethyl-2,4-dioxo-1H-quinolin-7-yl)acetamide (1.2 g, 4.8 mmol, 48% yield). MS (ESI) m/z: 247.4 [M+1]⁺.

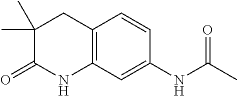 [SMILES: CC(=O)Nc1ccc2c(c1)NC(=O)C(C)(C)C2]

N-(3,3-dimethyl-2-oxo-1,4-dihydroquinolin-7-yl)acetamide. To a solution of N-(3,3-dimethyl-2,4-dioxo-1H-quinolin-7-yl)acetamide (0.80 g, 3.25 mmol) in Acetic acid (80 mL) was added acetic anhydride (0.37 mL, 3.9 mmol), concentrated sulfuric acid (0.10 g, 3.25 mmol), palladium on carbon (0.345 g, 3.25 mmol) under nitrogen, The solution was degassed under nitrogen. Then the solution was stirred at 25° C. under hydrogenation (30 psi) for 12 h. The solution was filtered. After the addition of sodium bicarbonate, the filtrate was concentrated and extracted with dichloromethane. The extract was washed with water, dried, and concentrated to afford N-(3,3-dimethyl-2-oxo-1,4-dihydroquinolin-7-yl)acetamide (260 mg, 1.11 mmol, 34% yield). MS (ESI) m/z: 233.3 [M+1]⁺.

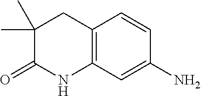

7-amino-3,3-dimethyl-1,4-dihydroquinolin-2-one. To solution of N-(3,3-dimethyl-2-oxo-1,4-dihydroquinolin-7-yl)acetamide (260 mg, 1.12 mmol) in Ethanol (5 mL) was added concentrated hydrochloride acid (0.09 mL, 1.12 mmol). The solution was stirred at 70° C. for 12 h. The solution was extracted with Ethyl acetate. The water layer was adjusted to pH=8 with sodium hydroxide (1N) then extracted with dichloromethane. The organic layer was washed with brine, dried over sodium sulfate, filtered and the filtrate was concentrated to afford 7-amino-3,3-dimethyl-1,4-dihydroquinolin-2-one (130 mg, 0.683 mmol, 61% yield). MS (ESI) m/z: 191.6 [M+1]⁺.

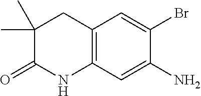

7-amino-6-bromo-3,3-dimethyl-1,4-dihydroquinolin-2-one. To a mixture of 7-amino-3,3-dimethyl-1,4-dihydroquinolin-2-one (0.2 g, 1.05 mmol) suspended in dichloromethane (10 mL), methanol (2.5 mL) was added tetra-n-butylammonium tribromide (0.33 g, 0.68 mmol). The reaction mixture was stirred at 25° C. for two hours and then was partitioned between dichloromethane and 10% aqueous sodium thiosulfate. The organic layer was dried over sodium sulfate, filtered and concentrated under reduced pressure. Purification by column chromatography, eluting with ethyl acetate/hexanes, provided 7-amino-6-bromo-3,3-dimethyl-1,4-dihydroquinolin-2-one (150 mg, 0.557 mmol, 53% yield). MS (ESI) m/z: 269.3 [M+1]⁺.

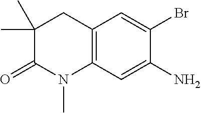 [SMILES: CN1C(=O)C(C)(C)Cc2cc(Br)c(N)cc21]

7-amino-6-bromo-1,3,3-trimethyl-4H-quinolin-2-one. 7-amino-6-bromo-3,3-dimethyl-1,4-dihydroquinolin-2-one (0.150 g, 0.560 mmol) was dissolved in THE (5 mL) and cooled to 0° C. Potassium bis(trimethylsilyl)amide (0.8 mL, 0.800 mmol, 1.0M in THF) then was added dropwise, followed by dropwise addition of iodomethane (0.087 g, 0.6100 mmol). The mixture was stirred for 12 hours at 20° C. The reaction was quenched with ammonium chloride, and then partitioned between ethyl acetate and brine. The organic layer was dried over sodium sulfate, filtered and concentrated under reduced pressure. The residue was purified by prep-TLC (ethyl acetate/hexanes) to provided 7-amino-6-bromo-1,3,3-trimethyl-4H-quinolin-2-one (50 mg, 0.176 mmol, 31% yield). MS (ESI) m/z: 285.4 [M+1]⁺.

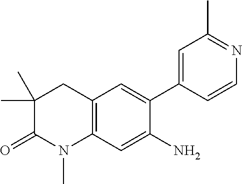 [SMILES: Cc1cc(-c2cc3c(cc2N)N(C)C(=O)C(C)(C)C3)ccn1]

7-amino-1,3,3-trimethyl-6-(2-methyl-4-pyridyl)-4H-quinolin-2-one. To a solution of 7-amino-6-bromo-1,3,3-trimethyl-4H-quinolin-2-one (0.050 g, 0.180 mmol) 2-methyl-4-(4,4,5,5-tetramethyl-1,3,2-dioxaborolan-2-yl)pyridine (0.046 g, 0.210 mmol) in, 1,4-Dioxane (5 mL) Water (1 mL) was added potassium carbonate (0.060 g, 0.440 mmol), (1,1′-bis(diphenylphosphino)ferrocene)palladium(II) dichloride (0.014 g, 0.020 mmol) under nitrogen. The solution was stirred at 110° C. for 12 h under nitrogen. The solution was concentrated and the residue was purified by prep-TLC (dichloromethane:methanol) to afford 7-amino-1,3,3-trimethyl-6-(2-methyl-4-pyridyl)-4H-quinolin-2-one (35 mg, 0.118 mmol, 67% yield). MS (ESI) m/z: 296.5 [M+1]⁺.

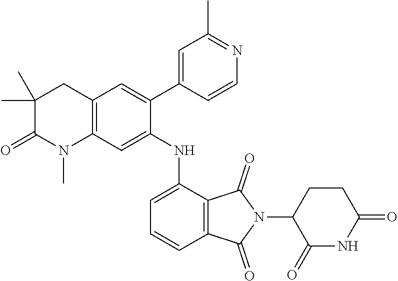 [SMILES: Cc1cc(-c2cc3c(cc2Nc2cccc4c2C(=O)N(C2CCC(=O)NC2=O)C4=O)N(C)C(=O)C(C)(C)C3)ccn1]

2-(2,6-dioxo-3-piperidyl)-4-[[1,3,3-trimethyl-6-(2-methyl-4-pyridyl)-2-oxo-4H-quinolin-7-yl]amino]isoindoline-1,3-dione. To a solution of 7-amino-1,3,3-trimethyl-6-(2-methyl-4-pyridyl)-4H-quinolin-2-one (0.035 g, 0.120 mmol), 4-bromo-2-(2,6-dioxo-3-piperidyl)isoindoline-1,3-dione (0.039 g, 0.120 mmol) in 1,4-Dioxane (2 mL) was added potassium carbonate (0.040 g, 0.300 mmol), chloro(2-dicyclohexylphosphino-2′,4′,6′-triisopropyl-1,1′-biphenyl)[2-(2′-amino-1,1′-biphenyl)]palladium(II) (0.010 g, 0.010 mmol) under nitrogen. The solution was degassed under nitrogen for 5 times. Then the solution was stirred at 110° C. for 12 h. The solution was filtered and the filtrate was concentrated. The residue was purified by prep-HPLC (acetonitrile/water) to afford 2-(2,6-dioxo-3-piperidyl)-4-[[1,3,3-trimethyl-6-(2-methyl-4-pyridyl)-2-oxo-4H-quinolin-7-yl]amino]isoindoline-1,3-dione (19 mg, 0.034 mmol, 29% yield). ¹H NMR (400 MHz, DMSO-d₆) δ 11.13 (s, 1H), 8.59 (d, J=4.4 Hz, 1H), 8.49 (s, 1H), 7.83 (brs, 1H), 7.70 (brs, 1H), 7.49-7.46 (m, 2H), 7.24 (s, 1H), 7.17 (d, J=7.6 Hz, 1H), 6.97-6.95 (m, 1H), 5.12-5.08 (m, 1H), 3.28 (s, 3H), 2.98-2.89 (m, 3H), 2.58-2.53 (m, 1H), 2.51-2.50 (m, 3H), 2.34-2.30 (m, 1H), 2.04-2.02 (m, 1H), 1.49 (s, 6H). MS (ESI) m/z: 552.3 [M+1]⁺.

### Example 9: Synthesis of Compound 86: 4-(((S)-1,3-dimethyl-6-(2-methylpyridin-4-yl)-2-oxo-1,2,3,4-tetrahydroquinolin-7-yl)amino)-2-((S)-2,6-dioxopiperidin-3-yl)isoindoline-1,3-dione

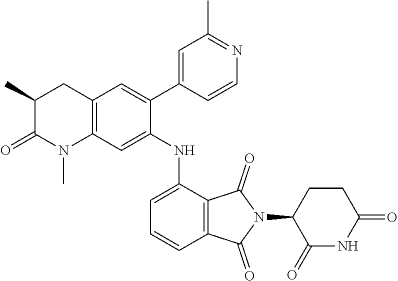 [SMILES: Cc1cc(-c2cc3c(cc2Nc2cccc4c2C(=O)N([C@H]2CCC(=O)NC2=O)C4=O)N(C)C(=O)[C@@H](C)C3)ccn1]

3-(2,4-dinitrophenyl)-2-methylpropanoic acid. 2-met yl-3-phenyl-propanoic acid (2.00 g, 12.18 mmol) was dissolved in concentrated sulfuric acid (10 mL) and cooled to about −15° C. Nitric acid (1.36 mL, 30.45 mmol) was added dropwise via syringe maintaining the temperature below −15° C. The mixture was then allowed to warm to 25° C. and was stirred for about 2 hours. The reaction mixture was quenched with water and then extracted with ethyl acetate. The combined organic phase was washed with brine, dried with anhydrous sodium sulfate, filtered and concentrated in vacuum to afford 3-(2,4-dinitrophenyl)-2-methylpropanoic acid (1.50 g, 5.90 mmol, 48% yield).

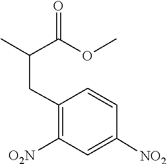 [SMILES: COC(=O)C(C)Cc1ccc([N+](=O)[O-])cc1[N+](=O)[O-]]

methyl 3-(2,4-dinitrophenyl)-2-methyl-propanoate. To a solution of 3-(2,4-dinitrophenyl)-2-methylpropanoic acid (1.50 g, 5.90 mmol) in methanol (20 mL) was added [€-diazomethyl]-trimethylsilane (6.730 g, 59.01 mmol) in toluene (20 mL) was stirred at 25° C. The reaction mixture was filtered and the filtrate was concentrated under reduce pressure by water pump. The suspension was filtered and the filtrate was concentrated. The residue was purified by column chromatography on silica gel (petroleum ether:ethyl acetate) to afford methyl 3-(2,4-dinitrophenyl)-2-methyl-propanoate (1.50 g, 5.59 mmol, 95% yield).

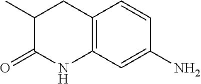

7-amino-3-methyl-3,4-dihydroquinolin-2(1H)-one. To a solution of methyl 3-(2,4-dinitrophenyl)-2-methyl-propanoate (1.50 g, 5.59 mmol) in tert-butanol (20 mL) was added palladium on activated carbon (0.300 g, 2.82 mmol). The reaction mixture is stirred at 50° C. for 15 hours, with hydrogen (50 psi) atmosphere. The reaction mixture was filtered and the filtrate was concentrated to afforded 7-amino-3-methyl-3,4-dihydroquinolin-2(1H)-one (0.800 g, 4.54 mmol, 81% yield). MS (ESI) m/z: 296.5 [M+1]⁺.

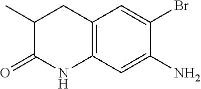

7-amino-6-bromo-3-methyl-3,4-dihydroquinolin-2(1H)-one. To a mixture of 7-amino-3-methyl-3,4-dihydroquinolin-2(1H)-one (0.800 g, 4.54 mmol) suspended in dichloromethane (8 mL) and methanol (2 mL) was added tetra-n-butylammonium tribromide (1.313 g, 2.72 mmol) at 0° C. The reaction mixture was stirred at 25° C. for two hours. The solution was partitioned between dichloromethane and 10% aqueous sodium thiosulfate. The organic layer was dried over sodium sulfate, filtered and concentrated under reduced pressure. Purification by column chromatography, eluting with ethyl acetate/hexanes, provided 7-amino-6-bromo-3-methyl-3,4-dihydroquinolin-2(1H)-one (400 mg, 1.57 mmol, 35% yield).

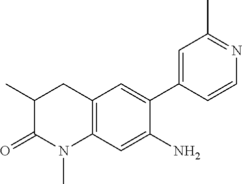 [SMILES: Cc1cc(-c2cc3c(cc2N)N(C)C(=O)C(C)C3)ccn1]

7-amino-1,3-dimethyl-6-(2-methyl-4-pyridyl)-3,4-dihydroquinolin-2-one. To a solution of the mixture of 7-amino-6-bromo-1,3-dimethyl-3,4-dihydroquinolin-2-one (0.300 g, 1.11 mmol) in 1,4-dioxane (10 mL) and water (1 mL) was added 2-methyl-4-(4,4,5,5-tetramethyl-1,3,2-dioxaborolan-2-yl)pyridine (0.269 g, 1.23 mmol), (1,1′-bis(diphenylphosphino)ferrocene)palladium(II) dichloride (0.046 g, 0.060 mmol) and potassium carbonate (0.461 g, 3.34 mmol). The suspension was degassed under vacuum and purged with nitrogen several times. The mixture was stirred at 110° C. for 12 hours. The reaction was concentrated in vacuum, and it was purified by silica gel column chromatography(methanol/dichloromethane) to afford 7-amino-1,3-dimethyl-6-(2-methyl-4-pyridyl)-3,4-dihydroquinolin-2-one (260 mg, 0.92 mmol, 83% yield). MS (ESI) m/z: 282.1 [M+1]⁺.

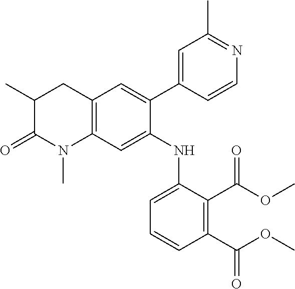 [SMILES: COC(=O)c1cccc(Nc2cc3c(cc2-c2ccnc(C)c2)CC(C)C(=O)N3C)c1C(=O)OC]

Dimethyl 3-((1,3-dimethyl-6-(2-methylpyridin-4-yl)-2-oxo-1,2,3,4-tetrahydroquinolin-7-yl)amino)phthalate. To a solution of 7-amino-1,3-dimethyl-6-(2-methylpyridin-4-yl)-3,4-dihydroquinolin-2(1H)-one (0.260 g, 0.92 mmol) and dimethyl 3-bromophthalate (0.303 g, 1.11 mmol) in 1,4-dioxane (5 mL) was added cesium carbonate (0.901 g, 2.77 mmol) and Methanesulfonato(2-dicyclohexylphosphino-2,6-di-i-propoxy-1,1-biphenyl)(2-amino-1,1-biphenyl-2-yl)palladium(II) (0.077 g, 0.09 mmol). The mixture was replaced gas of nitrogen for 3 min. The resulting mixture was stirred at 110° C. for 12 hours under nitrogen. The reaction mixture was filtered and the filtrate was concentrated under reduce pressure by water pump. The residue was purified by column chromatography on silica gel eluted with petroleum ether/ethyl acetate. The desired dimethyl 3-((1,3-dimethyl-6-(2-methylpyridin-4-yl)-2-oxo-1,2,3,4-tetrahydroquinolin-7-yl)amino)phthalate (350 mg, 0.74 mmol, 80% yield). MS (ESI) m/z: 474.1 [M+1]⁺.

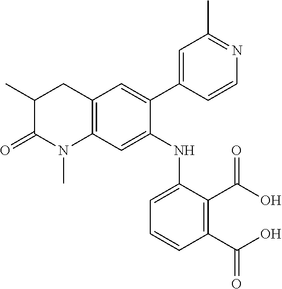 [SMILES: Cc1cc(-c2cc3c(cc2Nc2cccc(C(=O)O)c2C(=O)O)N(C)C(=O)C(C)C3)ccn1]

3-((1,3-dimethyl-6-(2-methylpyridin-4-yl)-2-oxo-1,2,3,4-tetrahydroquinolin-7-yl)amino)phthalic acid. To a solution of dimethyl 3-((1,3-dimethyl-6-(2-methylpyridin-4-yl)-2-oxo-1,2,3,4-tetrahydroquinolin-7-yl)amino)phthalate (0.350 g, 0.74 mmol) in methanol (6 mL) was added the solution of sodium hydroxide (0.300 g, 7.39 mmol) in water (20 mL). The mixture was stirred at 80° C. for 12 hours. The reaction mixture was added water (50 mL) and then adjusted pH=6, the solid was formed, the mixture was filtered, the filter cake was concentrated under reduce pressure by water pump. The filtrate was extracted with DCM and water, the combined organic layer was dried with sodium sulfate and filtered, the filtrate was concentrated under reduce pressure by water pump. The desired 3-((1,3-dimethyl-6-(2-methylpyridin-4-yl)-2-oxo-1,2,3,4-tetrahydroquinolin-7-yl)amino)phthalic acid (250 mg, 0.56 mmol, 76% yield). MS (ESI) m/z: 446.2 [M+1]⁺.

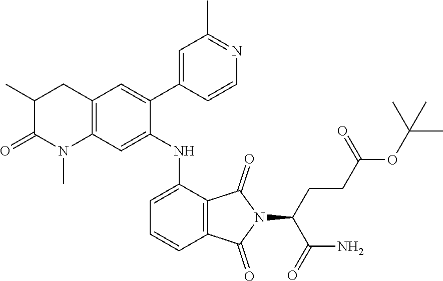 [SMILES: Cc1cc(-c2cc3c(cc2Nc2cccc4c2C(=O)N([C@@H](CCC(=O)OC(C)(C)C)C(N)=O)C4=O)N(C)C(=O)C(C)C3)ccn1]

Tert-butyl (4S)-5-amino-4-(4-((1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate. To a solution of 3-((6-(2-methoxy-6-methylpyridin-4-yl)-1-methyl-2-oxo-1,2,3,4-tetrahydroquinolin-7-yl)amino)phthalic acid (0.300 g, 0.67 mmol) and tert-butyl (S)-4,5-diamino-5-oxopentanoate (0.136 mg, 0.67 mmol) in toluene (3 mL) was added triethylamine (1.17 mL, 2.6 mmol). The resulting mixture was stirred at 130° C. for 12 hours. The reaction mixture was concentrated under reduce pressure by water pump. The residue was purified by column chromatography on silica gel eluted with petroleum ether/ethyl acetate. The desired tert-butyl (4S)-5-amino-4-(4-((1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate (350 mg, 0.57 mmol, 85% yield). MS (ESI) m/z: 621.3 [M+1]⁺.

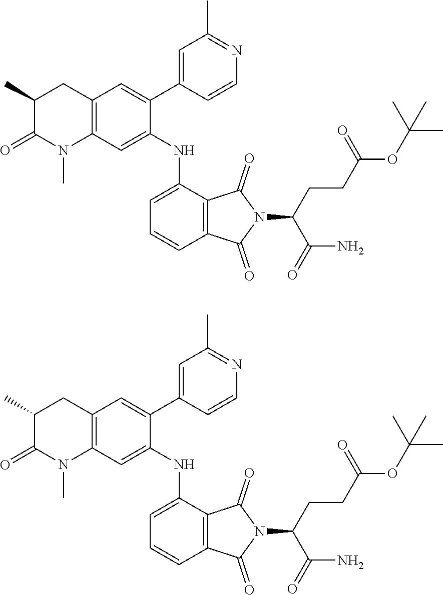 [SMILES: Cc1cc(-c2cc3c(cc2Nc2cccc4c2C(=O)N([C@@H](CCC(=O)OC(C)(C)C)C(N)=O)C4=O)N(C)C(=O)[C@@H](C)C3)ccn1 ; Cc1cc(-c2cc3c(cc2Nc2cccc4c2C(=O)N([C@@H](CCC(=O)OC(C)(C)C)C(N)=O)C4=O)N(C)C(=O)[C@H](C)C3)ccn1]

tert-butyl (S)-5-amino-4-(4-(((S)-1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate and tert-butyl (S)-5-amino-4-(4-(((R)-1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate. The tert-butyl (4S)-5-amino-4-(4-((1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate (0.350 g, 0.57 mmol) was separated by prep-SFC to give tert-butyl (S)-5-amino-4-(4-(((S)-1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate (120 mg, 0.20 mmol, 34% yield) and tert-butyl (S)-5-amino-4-(4-(((R)-1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate (150 mg, 0.26 mmol, 43% yield).

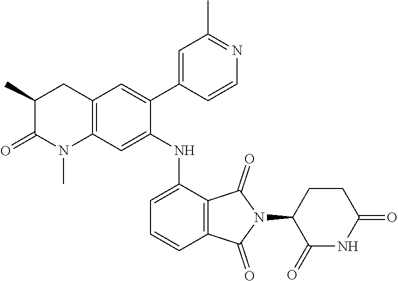 [SMILES: Cc1cc(-c2cc3c(cc2Nc2cccc4c2C(=O)N([C@H]2CCC(=O)NC2=O)C4=O)N(C)C(=O)[C@@H](C)C3)ccn1]

4-(((S)-1,3-dimethyl-6-(2-methylpyridin-4-yl)-2-oxo-1,2,3,4-tetrahydroquinolin-7-yl)amino)-2-((S)-2,6-dioxopiperidin-3-yl)isoindoline-1,3-dione. To a solution of tert-butyl (S)-5-amino-4-(4-(((S)-1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate (0.120 g, 0.20 mmol) in acetonitrile (4 mL) was added p-toluenesulfonic acid (0.338 g, 1.96 mmol). The resulting mixture was stirred at 80° C. for 2 hours under nitrogen. The reaction mixture was quenched with dichloromethane and sodium bicarbonate, and then extracted with dichloromethane. The combined organic layer was dried with anhydrous sodium sulfate and then filtered, the filtrate was concentrated under reduce pressure by water pump. Then the collected fraction was concentrated to remove the dichloromethane. It was purified by silica gel column chromatography (methanol in dichloromethane) to afford 4-(((S)-1,3-dimethyl-6-(2-methylpyridin-4-yl)-2-oxo-1,2,3,4-tetrahydroquinolin-7-yl)amino)-2-((S)-2,6-dioxopiperidin-3-yl)isoindoline-1,3-dione (70 mg, 0.130 mmol, 67% yield). ¹H NMR (400 MHz, DMSO-d₆) δ 11.11 (s, 1H), 8.35 (d, J=5.2 Hz, 1H), 8.32 (s, 1H), 7.49-7.43 (m, 1H), 7.37 (s, 1H), 7.31 (s, 1H), 7.22 (d, J=4.8 Hz, 1H), 7.19 (s, 1H), 7.13 (d, J=7.2 Hz, 1H), 7.00 (d, J=8.8 Hz, 1H), 5.08 (dd, J=5.4, 12.8 Hz, 1H), 3.28 (s, 3H), 3.02 (m, 1H), 2.94-2.83 (m, 1H), 2.78-2.69 (m, 1H), 2.68-2.63 (m, 1H), 2.62-2.52 (m, 2H), 2.40 (s, 3H), 2.07-2.00 (m, 1H), 1.18 (d, J=6.8 Hz, 3H). MS (ESI) m/z: 538.3 [M+1]⁺.

### Example 10: Synthesis of Compound 87: 4-(((R)-1,3-dimethyl-6-(2-methylpyridin-4-yl)-2-oxo-1,2,3,4-tetrahydroquinolin-7-yl)amino)-2-((S)-2,6-dioxopiperidin-3-yl)isoindoline-1,3-dione

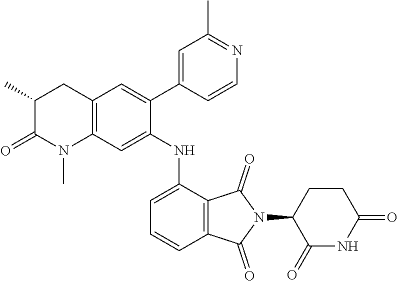 [SMILES: Cc1cc(-c2cc3c(cc2Nc2cccc4c2C(=O)N([C@H]2CCC(=O)NC2=O)C4=O)N(C)C(=O)[C@H](C)C3)ccn1]

4-(((R)-1,3-dimethyl-6-(2-methylpyridin-4-yl)-2-oxo-1,2,3,4-tetrahydroquinolin-7-yl)amino)-2-((S)-2,6-dioxopiperidin-3-yl)isoindoline-1,3-dione. To a solution of tert-butyl (S)-5-amino-4-(4-(((R)-1,3-dimethyl-6-(2-methylpyridin-4-yl)-2-oxo-1,2,3,4-tetrahydroquinolin-7-yl)amino)-1,3-dioxoisoindolin-2-yl)-5-oxopentanoate (0.06 g, 0.20 mmol) in acetonitrile (3 mL) was added p-toluenesulfonic acid (0.338 g, 1.96 mmol). The resulting mixture was stirred at 80° C. for 2 hours under nitrogen. The reaction mixture was quenched with dichloromethane and sodium bicarbonate, and then extracted with dichloromethane. The combined organic layer was dried with anhydrous sodium sulfate and then filtered, the filtrate was concentrated under reduce pressure by water pump. Then the collected fraction was concentrated to remove the dichloromethane. It was purified by silica gel column chromatography (methanol in dichloromethane). Further purification ion using prep-SFC gave 4-(((R)-1,3-dimethyl-6-(2-methylpyridin-4-yl)-2-oxo-1,2,3,4-tetrahydroquinolin-7-yl)amino)-2-((S)-2,6-dioxopiperidin-3-yl)isoindoline-1,3-dione (29 mg, 0.054 mmol, 73% yield). ¹H NMR (400 MHz, DMSO-d₆) δ 11.10 (s, 1H), 8.35 (d, J=5.2 Hz, 1H), 8.32 (s, 1H), 7.-9-7.43 (m, 1H), 7.37 (s, 1H), 7.31 (s, 1H), 7.22 (d, J=4.8 Hz, 1H), 7.19 (s, 1H), 7.13 (d, J=7.2 Hz, 1H), 7.00 (d, J=8.8 Hz, 1H), 5.08 (dd, J=5.4, 12.8 Hz, 1H), 3.28 (s, 3H), 3.-5-3.00 (m, 1H), 2.-4-2.82 (m, 1H), 2.-8-2.69 (m, 1H), 2.-7-2.58 (m, 2H), 2.-7-2.51 (m, 1H), 2.40 (s, 3H), 2.-6-2.00 (m, 1H), 1.18 (d, J=6.8 Hz, 3H). MS (ESI) m/z: 538.3 [M+1]⁺.

### Example 11: Synthesis of Compound 88: 4-[[1-cyclopropyl-6-(2-methyl-4-pyridyl)-2-oxo-3,4-dihydroquinolin-7-yl]amino]-2-[(3S)-2,6-dioxo-3-piperidyl]isoindoline-1,3-dione

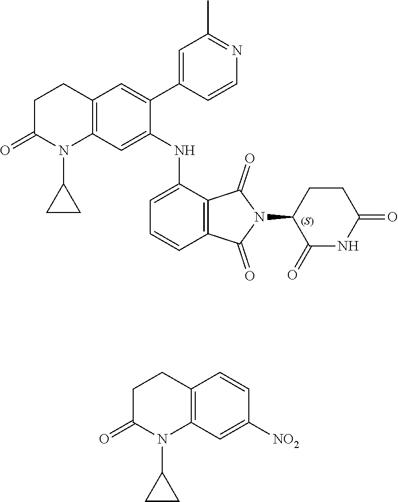

1-cyclopropyl-7-nitro-3,4-dihydroquinolin-2-one. To a solution of 7-nitro-3,4-dihydro-1H-quinolin-2-one (0.450 g, 2.34 mmol) in Toluene (5 mL) was added cyclopropylboronic acid (0.402 g, 4.68 mmol), cesium carbonate (0.761 g, 2.34 mmol), pyridine (0.58 mL, 7.03 mmol) and copper diacetate (0.372 g, 2.34 mmol). The mixture was stirred at 95° C. for 12 h under air. The mixture was concentrated under reduced pressure. The residue was diluted with ethyl acetate. The mixture was filtered and filtrate was concentrated under reduced pressure. The residue was purified by prep-TLC (ethyl acetate/petroleum ether). 1-cyclopropyl-7-nitro-3,4-dihydroquinolin-2-one (450 mg, 1.94 mmol, 82% yield) was obtained as a white solid. ¹H NMR (400 MHz, DMSO-d₆) δ 8.05 (d, J=2.4 Hz, 1H), 7.88 (dd, J=2.4, 8.4 Hz, 1H), 7.48 (d, J=8.4 Hz, 1H), 2.92 (t, J=7.2 Hz, 2H), 2.86-2.79 (m, 1H), 2.58-2.54 (m, 2H), 1.13-1.07 (m, 2H), 0.58-0.52 (m, 2H); MS (ESI) m/z 233.2 [M+1]*.

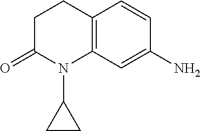 [SMILES: Nc1ccc2c(c1)N(C1CC1)C(=O)CC2]

7-amino-1-cyclopropyl-3,4-dihydroquinolin-2-one. To a solution of 1-cyclopropyl-7-nitro-3,4-dihydroquinolin-2-one (0.450 g, 1.94 mmol) in Ethanol (15 mL) and Water (5 mL) was added iron powder (0.541 g, 9.69 mmol) and ammonia hydrochloride (1.046 g, 19.38 mmol). The mixture was stirred at 80° C. for 2 h. The mixture was filtered and filtrate was concentrated under reduced pressure. The residue was purified by column chromatography on silica gel (ethyl acetate/petroleum ether). Compound 7-amino-1-cyclopropyl-3,4-dihydroquinolin-2-one (300 mg, 1.48 mmol, 76% yield). ¹H NMR (400 MHz, DMSO-d₆) δ 6.78 (d, J=7.6 Hz, 1H), 6.57 (d, J=2.0 Hz, 1H), 6.18 (dd, J=2.0, 7.6 Hz, 1H), 5.07-4.92 (m, 2H), 2.62-2.57 (m, 1H), 2.55 (d, J=7.2 Hz, 2H), 2.43-2.35 (m, 2H), 1.06-0.92 (m, 2H), 0.51-0.38 (m, 2H); MS (ESI) m/z 203.1 [M+1]⁺.

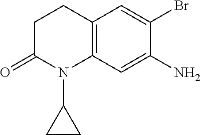 [SMILES: Nc1cc2c(cc1Br)CCC(=O)N2C1CC1]

7-amino-6-bromo-1-cyclopropyl-3,4-dihydroquinolin-2-one. To a solution of 7-amino-1-cyclopropyl-3,4-dihydroquinolin-2-one (0.300 g, 1.48 mmol) in dichloromethane (10 mL) and methanol (2.5 mL) was added tetra-n-butylammonium tribromide (0.465 g, 0.96 mmol). The mixture was stirred at 15° C. for 2 h. The mixture was concentrated under reduced pressure. The residue was purified by column chromatography on silica gel (ethyl acetate/petroleum ether). 7-amino-6-bromo-1-cyclopropyl-3,4-dihydroquinolin-2-one (330 mg, 1.17 mmol, 79% yield). ¹H NMR (400 MHz, CDCl₃) δ 7.15 (s, 1H), 6.74 (s, 1H), 4.07 (br s, 2H), 2.72-2.66 (m, 3H), 2.59-2.54 (m, 2H), 1.15-1.07 (m, 2H), 0.71-0.62 (m, 2H); MS (ESI) m/z 281.1 [M+1]⁺.

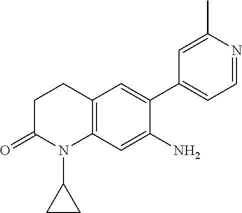 [SMILES: Cc1cc(-c2cc3c(cc2N)N(C2CC2)C(=O)CC3)ccn1]

7-amino-1-cyclopropyl-6-(2-methyl-4-pyridyl)-3,4-dihydroquinolin-2-one. To a solution of 7-amino-6-bromo-1-cyclopropyl-3,4-dihydroquinolin-2-one (0.330 g, 1.17 mmol) in 1,4-Dioxane (10 mL) and Water (1 mL) was added 2-methyl-4-(4,4,5,5-tetramethyl-1,3,2-dioxaborolan-2-yl)pyridine (0.309 g, 1.41 mmol), potassium carbonate (0.486 g, 3.52 mmol) and (1,1′-bis(diphenylphosphino)ferrocene)palladium(II) dichloride (0.096 g, 0.12 mmol). The suspension was degassed under vacuum and purged with nitrogen several times. The mixture was stirred at 110° C. for 12 h. The mixture was filtered and filtrate was concentrated under reduced pressured. The residue was purified by prep-HPLC. Compound 7-amino-1-cyclopropyl-6-(2-methyl-4-pyridyl)-3,4-dihydroquinolin-2-one (210 mg, 0.72 mmol, 61% yield) was obtained as a brown solid. ¹H NMR (400 MHz, CDCl₃) δ 8.55 (d, J=5.2 Hz, 1H), 7.27 (s, 1H), 7.22 (dd, J=1.2, 5.2 Hz, 1H), 6.90 (s, 1H), 6.75 (s, 1H), 3.84 (br s, 2H), 2.77-2.70 (m, 3H), 2.64-2.60 (m, 5H), 1.19-1.13 (m, 2H), 0.75-0.69 (m, 2H); MS (ESI) m/z 294.1 [M+1]⁺.

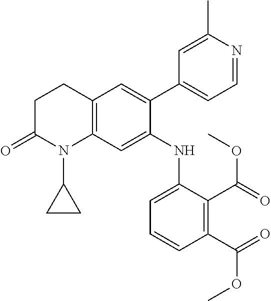 [SMILES: COC(=O)c1cccc(Nc2cc3c(cc2-c2ccnc(C)c2)CCC(=O)N3C2CC2)c1C(=O)OC]

3-[[1-cyclopropyl-6-(2-methyl-4-pyridyl)-2-oxo-3,4-dihydroquinolin-7-yl]amino]benzene-1,2-dicarboxylate. To a solution of 7-amino-1-cyclopropyl-6-(2-methyl-4-pyridyl)-3,4-dihydroquinolin-2-one (0.210 g, 0.72 mmol) in 1,4-Dioxane (10 mL) was added dimethyl 3-bromobenzene-1,2-dicarboxylate (0.235 g, 0.86 mmol), cesium carbonate (0.699 g, 2.15 mmol) and methanesulfonato(2-dicyclohexylphosphino-2′,6′-di-i-propoxy-1,1′-biphenyl)(2′-amino-1,1′-biphenyl-2-yl)palladium(ii) (0.060 g, 0.07 mmol). The suspension was degassed under vacuum and purged with nitrogen several times. The mixture was stirred at 110° C. for 12 h. The mixture was filtered and filtrate was concentrated under reduced pressure. The residue was purified by prep-TLC (ethyl acetate) to provide dimethyl 3-[[1-cyclopropyl-6-(2-methyl-4-pyridyl)-2-oxo-3,4-dihydroquinolin-7-yl]amino]benzene-1,2-dicarboxylate (280 mg, 0.58 mmol, 81% yield). ¹H NMR (400 MHz, CDCl₃) δ 8.50 (d, J=5.2 Hz, 1H), 8.02 (s, 1H), 7.35-7.31 (m, 1H), 7.31 (s, 1H), 7.28 (s, 1H), 7.19 (s, 1H), 7.15-7.11 (m, 2H), 7.09 (dd, J=3.2, 5.6 Hz, 1H), 3.88 (s, 3H), 3.80 (s, 3H), 2.90-2.80 (m, 2H), 2.72-2.63 (m, 3H), 2.58 (s, 3H), 1.16-1.06 (m, 2H), 0.78-0.65 (m, 2H); MS (ESI) m/z 486.3 [M+1]*.

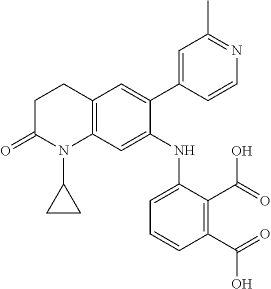 [SMILES: Cc1cc(-c2cc3c(cc2Nc2cccc(C(=O)O)c2C(=O)O)N(C2CC2)C(=O)CC3)ccn1]

3-[[1-cyclopropyl-6-(2-methyl-4-pyridyl)-2-oxo-3,4-dihydroquinolin-7-yl]amino]phthalic acid. To a solution of dimethyl 3-[[1-cyclopropyl-6-(2-methyl-4-pyridyl)-2-oxo-3,4-dihydroquinolin-7-yl]amino]benzene-1,2-dicarboxylate (0.280 g, 0.58 mmol) in Methanol (5 mL) and Water (3 mL) was added sodium hydroxide (0.069 g, 1.73 mmol). The mixture was stirred at 80° C. for 12 h. The mixture was concentrated under reduced pressure. The mixture was diluted with water (20 mL) and was acidified to pH 6-7 with hydrochloric acid (1M). The mixture was concentrated under reduced pressure. The mixture was dried over anhydrous sodium sulfate, filtered and concentrated under reduced pressure to provide 3-[[1-cyclopropyl-6-(2-methyl-4-pyridyl)-2-oxo-3,4-dihydroquinolin-7-yl]amino]phthalic acid (240 mg, 0.52 mmol, 91% yield). MS (ESI) m/z 458.2 [M+1]⁺.

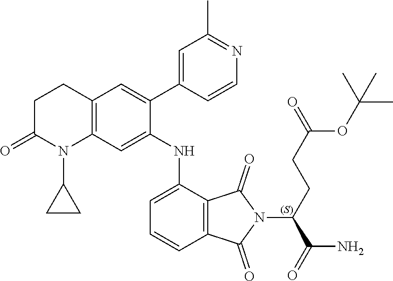

tert-butyl (S)-5-amino-4-(4-((1-cyclopropyl-6-(2-methylpyridin-4-yl)-2-oxo-1,2,3,4-tetrahydroquinolin-7-yl)amino)-1,3-dioxoisoindolin-2-yl)-5-oxopentanoate. To a solution of 3-[[1-cyclopropyl-6-(2-methyl-4-pyridyl)-2-oxo-3,4-dihydroquinolin-7-yl]amino]phthalic acid (0.220 g, 0.48 mmol) in Toluene (30 mL) was added tert-butyl (4S)-4,5-diamino-5-oxo-pentanoate (0.107 g, 0.53 mmol) and triethylamine (0.84 mL, 4.81 mmol). The mixture was stirred at 130° C. for 12 h. The mixture was concentrated under reduced pressure. The residue was purified by prep-TLC (methanol in dichloromethane). Compound tert-butyl (S)-5-amino-4-(4-((1-cyclopropyl-6-(2-methylpyridin-4-yl)-2-oxo-1,2,3,4-tetrahydroquinolin-7-yl)amino)-1,3-dioxoisoindolin-2-yl)-5-oxopentanoate (100 mg, 0.16 mmol, 34% yield) was obtained as a yellow solid. MS (ESI) m/z 624.3 [M+1]⁺.

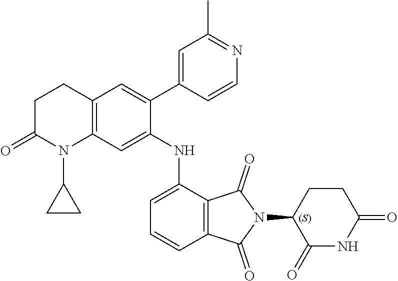

4-[[I-cyclopropyl-6-(2-methyl-4-pyridyl)-2-oxo-3,4-dihydroquinolin-7-yl]amino]-2-[(3S)-2,6-dioxo-3-piperidyl]isoindoline-1,3-dione. To a solution of tert-butyl (4S)-5-amino-4-[4-[[1-cyclopropyl-6-(2-methyl-4-pyridyl)-2-oxo-3,4-dihydroquinolin-7-yl]amino]-1,3-dioxo-isoindolin-2-yl]-5-oxo-pentanoate (0.100 g, 0.16 mmol) in acetonitrile (5 mL) was added benzenesulfonic acid (0.051 g, 0.32 mmol). The mixture was stirred at 60° C. for 12 h. The residue was purified by prep-TLC (methanol in dichloromethane) to provide 4-[[1-cyclopropyl-6-(2-methyl-4-pyridyl)-2-oxo-3,4-dihydroquinolin-7-yl]amino]-2-[(3S)-2,6-dioxo-3-piperidyl]isoindoline-1,3-dione (45 mg, 0.0812 mmol, 51% yield). ¹H NMR (400 MHz, DMSO-d₆) δ 11.11 (s, 1H), 8.37 (d, J=5.2 Hz, 1H), 8.31 (s, 1H), 7.53 (dd, J=7.2, 8.4 Hz, 1H), 7.39 (s, 1H), 7.34 (s, 1H), 7.32 (s, 1H), 7.24-7.21 (m, 1H), 7.16 (d, J=7.2 Hz, 1H), 7.11 (d, J=8.8 Hz, 1H), 5.08 (dd, J=5.2, 12.8 Hz, 1H), 2.90-2.82 (m, 3H), 2.75-2.70 (m, 1H), 2.63-2.53 (m, 4H), 2.42 (s, 3H), 2.07-2.00 (m, 1H), 1.07-1.00 (m, 2H), 0.63-0.58 (m, 2H); MS (ESI) m/z 550.2 [M+1]⁺.

### Example 12: Synthesis of Compound 89: (S)-2-(2,6-dioxopiperidin-3-yl)-4-((1-ethyl-6-(2-methylpyridin-4-yl)-2-oxo-1,2,3,4-tetrahydroquinolin-7-yl)amino)isoindoline-1,3-dione

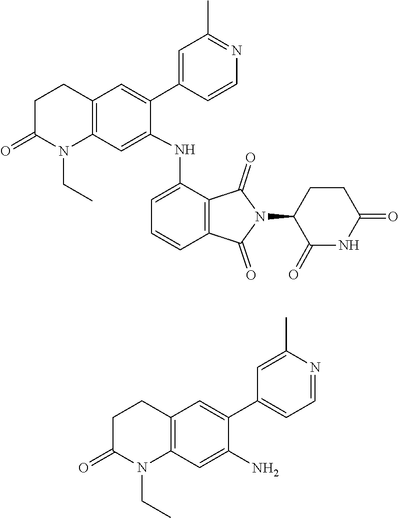

7-amino-6-bromo-1-ethyl-3,4-dihydroquinolin-2(1H)-one. 7-amino-6-bromo-3,4-dihydro-1H-quinolin-2-one (0.50 g, 2.07 mmol) was dissolved in THE (15 mL) and cooled to 0° C. Potassium bis(trimethylsilyl)amide (1M, 2.1 mL, 2.1 mmol) was then added dropwise, followed by dropwise addition of iodoethane (0.323 g, 2.28 mmol). The mixture was stirred for 12 hours at 20° C. The reaction was quenched with saturated ammonium chloride solution, and then partitioned between ethyl acetate and brine. The organic layer was dried over magnesium sulfate filtered and concentrated under reduced pressure. The crude residue was purified by column chromatography, eluting with ethyl acetate/hexanes, provided 7-amino-6-bromo-1-ethyl-3,4-dihydroquinolin-2(1H)-one (200 mg, 0.7431 mmol, 36% yield) as a colorless oil. MS (ESI) m/z: 269.1 [M+1]⁺.

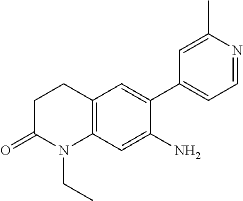 [SMILES: CCN1C(=O)CCc2cc(-c3ccnc(C)c3)c(N)cc21]

7-amino-1-ethyl-6-(2-methylpyridin-4-yl)-3,4-dihydroquinolin-2(1H)-one. To a solution of 7-amino-1-ethyl-6-(2-methylpyridin-4-yl)-3,4-dihydroquinolin-2(1H)-one (0.20 g, 0.740 mmol)2-methyl-4-(4,4,5,5-tetramethyl-1,3,2-dioxaborolan-2-yl)pyridine (0.195 g, 0.890 mmol) in, 1,4-Dioxane (5 mL) Water (1 mL) was added potassium carbonate (0.256 g, 1.86 mmol), (1,1′-bis(diphenylphosphino)ferrocene)palladium(II) dichloride (0.060 g, 0.074 mmol) under nitrogen. The solution was stirred at 110° C. for 12 h under nitrogen. The solution was concentrated and the residue was purified by prep-TLC (dichloromethane:Methanol) to provide 7-amino-1-ethyl-6-(2-methylpyridin-4-yl)-3,4-dihydroquinolin-2(1H)-one (150 mg, 0.533 mmol, 72% yield). MS (ESI) m/z: 282.4 [M+1]⁺.

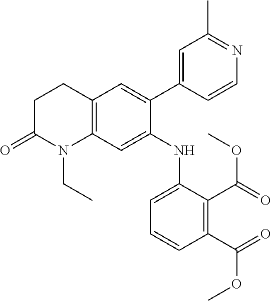 [SMILES: CCN1C(=O)CCc2cc(-c3ccnc(C)c3)c(Nc3cccc(C(=O)OC)c3C(=O)OC)cc21]

Dimethyl 3-((1-ethyl-6-(2-methylpyridin-4-yl)-2-oxo-1,2,3,4-tetrahydroquinolin-7-yl)amino)phthalate. To a solution of 7-amino-1-ethyl-6-(2-methylpyridin-4-yl)-3,4-dihydroquinolin-2(1H)-one (0.150 g, 0.530 mmol) and dimethyl 3-bromobenzene-1,2-dicarboxylate (0.160 g, 0.590 mmol) in 1,4-Dioxane (10 mL) was added cesium carbonate (0.52 g, 1.6 mmol) and Methanesulfonato(2-dicyclohexylphosphino-2,6-di-i-propoxy-1,1-biphenyl)(2-amino-1,1-biphenyl-2-yl)palladium(II) (0.044 g, 0.050 mmol). The resulting mixture was stirred at 110° C. for 12 hours under nitrogen. The reaction mixture was filtered and the filtrate was concentrated under reduce pressure by water pump and the residue was purified by prep-TLC (dichloromethane:Methanol) to afford dimethyl 3-((1-ethyl-6-(2-methylpyridin-4-yl)-2-oxo-1,2,3,4-tetrahydroquinolin-7-yl)amino)phthalate (150 mg, 0.316 mmol, 59% yield). MS (ESI) m/z: 474.3 [M+1]⁺.

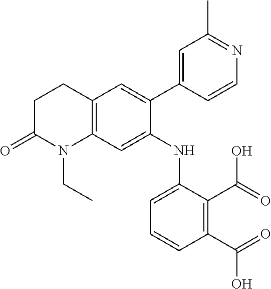 [SMILES: CCN1C(=O)CCc2cc(-c3ccnc(C)c3)c(Nc3cccc(C(=O)O)c3C(=O)O)cc21]

3-((1-ethyl-6-(2-methylpyridin-4-yl)-2-oxo-1,2,3,4-tetrahydroquinolin-7-yl)amino)phthalic acid. To a solution of dimethyl 3-((1-ethyl-6-(2-methylpyridin-4-yl)-2-oxo-1,2,3,4-tetrahydroquinolin-7-yl)amino)phthalate (0.140 g, 0.300 mmol) in Methanol (5 mL), Water (1 mL) was added sodium hydroxide (0.024 g, 0.600 mmol). The solution was stirred at 80° C. for 12 h. The solution was adjusted to pH=3 with hydrochloride acid, then extracted with dichloromethane. The organic layer was washed with brine, dried over sodium sulfate, filtered and filtrate was concentrated to afford 3-((1-ethyl-6-(2-methylpyridin-4-yl)-2-oxo-1,2,3,4-tetrahydroquinolin-7-yl)amino)phthalic acid (100 mg, 0.224 mmol, 76% yield). MS (ESI) m/z: 462.1 [M+1]⁺.

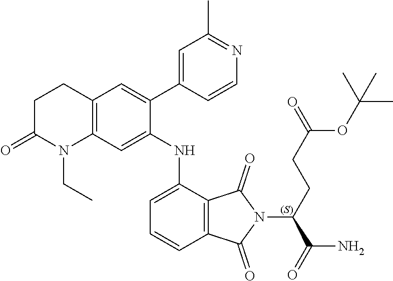

tert-butyl (S)-5-amino-4-(4-((1-ethyl-6-(2-methylpyridin-4-yl)-2-oxo-1,2,3,4-tetrahydroquinolin-7-yl)amino)-1,3-dioxoisoindolin-2-yl)-5-oxopentanoate. To a solution of 3-((1-ethyl-6-(2-methylpyridin-4-yl)-2-oxo-1,2,3,4-tetrahydroquinolin-7-yl)amino)phthalic acid (0.10 g, 0.220 mmol) in Toluene (3 mL) was added diisopropylethylamine (0.2 mL, 1.12 mmol), tert-butyl rac-(4S)-4,5-diamino-5-oxo-pentanoate (45.4 mg, 0.220 mmol). The solution was stirred at 130° C. for 12 h. LCMS showed this reaction worked well. The solution was concentrated and the residue was purified by prep-TLC (dichloromethane:methanol) to afford tert-butyl (S)-5-amino-4-(4-((1-ethyl-6-(2-methylpyridin-4-yl)-2-oxo-1,2,3,4-tetrahydroquinolin-7-yl)amino)-1,3-dioxoisoindolin-2-yl)-5-oxopentanoate (50 mg, 0.081 mmol, 36% yield). MS (ESI) m/z: 612.5 [M+1]⁺.

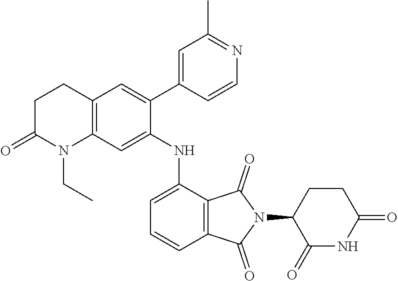 [SMILES: CCN1C(=O)CCc2cc(-c3ccnc(C)c3)c(Nc3cccc4c3C(=O)N([C@H]3CCC(=O)NC3=O)C4=O)cc21]

(S)-2-(2,6-dioxopiperidin-3-yl)-4-((1-ethyl-6-(2-methylpyridin-4-yl)-2-oxo-1,2,3,4-tetrahydroquinolin-7-yl)amino)isoindoline-1,3-dione. To a solution of tert-butyl (S)-5-amino-4-(4-((1-ethyl-6-(2-methylpyridin-4-yl)-2-oxo-1,2,3,4-tetrahydroquinolin-7-yl)amino)-1,3-dioxoisoindolin-2-yl)-5-oxopentanoate (0.050 g, 0.080 mmol) in acetonitrile (5 mL) was added benzenesulfonic acid (0.025 g, 0.160 mmol). The solution was stirred at 60° C. for 12 h. The reaction mixture was quenched with dichloromethane and sodium bicarbonate, and then extracted with dichloromethane. The combined organic layer was dried with anhydrous sodium sulfate and then filtered, the filtrate was concentrated under reduce pressure by water pump. The crude product was purified by prep-HPLC (acetonitrile/water TFA to give 27 mg of (S)-2-(2,6-dioxopiperidin-3-yl)-4-((1-ethyl-6-(2-methylpyridin-4-yl)-2-oxo-1,2,3,4-tetrahydroquinolin-7-yl)amino)isoindoline-1,3-dione (27 mg, 0.050 mmol, 63% yield). ¹H NMR (400 MHz, DMSO-d₆) δ 11.12 (s, 1H), 8.37-8.34 (m, 1H), 7.45-7.30 (m, 1H), 7.38 (s, 1H), 7.32 (s, 1H), 7.23-7.22 (m, 2H), 7.12-7.10 (m, 1H), 6.91-6.89 (m, 1H), 5.10 (dd, J=5.6, 12.8 Hz, 1H), 3.95-3.91 (m, 2H), 2.96-2.93 (m, 3H), 2.59-2.52 (m, 4H), 2.40 (s, 3H), 2.05-2.04 (m, 1H), 1.14 (t, J=6.4 Hz, 3H). MS (ESI) m/z: 538.3 [M+1]⁺.

### Example 13: Synthesis of Compound 90: (S)-2-(2,6-dioxopiperidin-3-yl)-4-((5-fluoro-1-methyl-6-(2-methylpyridin-4-yl)-2-oxo-1,2,3,4-tetrahydroquinolin-7-yl)amino)isoindoline-1,3-dione

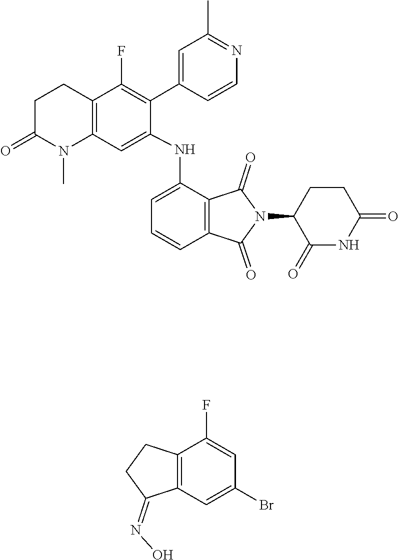 [SMILES: Cc1cc(-c2c(Nc3cccc4c3C(=O)N([C@H]3CCC(=O)NC3=O)C4=O)cc3c(c2F)CCC(=O)N3C)ccn1 ; O/N=C1/CCc2c(F)cc(Br)cc21]

(Z)-6-bromo-4-fluoro-2,3-dihydro-1H-inden-1-one oxime. To a solution of 6-bromo-4-fluoro-indan-1-one (9. g, 39.29 mmol) in Methanol (50 mL) was added hydroxylamine;hydrochloride (4.1 g, 58.94 mmol), followed by potassium hydroxide (6.6 g, 117.88 mmol) in Water (10 mL). The solution was stirred at 90° C. for 3 h. The mixture was cooled down to room temperature and acidified with 1N HCl. The resulting precipitate was collected by filtration and washed with water to give (Z)-6-bromo-4-fluoro-2,3-dihydro-1H-inden-1-one oxime (9.0 g, 36.8 mmol, 94% yield) as a white solid. MS (ESI) m/z: 244.1 [M+1]⁺.

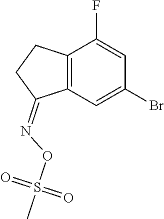 [SMILES: CS(=O)(=O)O/N=C1/CCc2c(F)cc(Br)cc21]

(Z)-6-bromo-4-fluoro-2,3-dihydro-1H-inden-1-one O-methylsulfonyl oxime. To a solution of 6-bromo-4-fluoro-indan-1-one oxime (9.0 g, 36.8 mmol) in dichloromethane (100 mL) was added triethylamine (12.8 mL, 73.7 mmol). The Ms₂O (7.7 g, 44.2 mmol) was added to the solution at −10° C. The solution was stirred at −10° C. for 2 h. The reaction was quenched with water, then extracted with dichloromethane. The organic layer was concentrated and the residue was purified by column on silica gel (petroleum ether:ethyl acetate) to afford (Z)-6-bromo-4-fluoro-2,3-dihydro-1H-inden-1-one O-methylsulfonyl oxime (5.0 g, 15.5 mmol, 42% yield) as a white solid. MS (ESI) m/z: 286.4 [M+1]⁺.

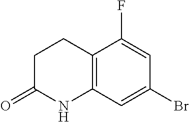 [SMILES: O=C1CCc2c(F)cc(Br)cc2N1]

7-bromo-5-fluoro-3,4-dihydroquinolin-2(1H)-one. To a solution of [(Z)-(6-bromo-4-fluoro-indan-1-ylidene)amino]methanesulfonate (8.5 g, 26.3 mmol) in dichloromethane (800 mL) was added aluminum chloride (7.0 g, 52.7 mmol). The solution was stirred at 25° C. for 12 h. The reaction solution was poured into ice/water, then extracted with dichloromethane. The combined organic layer was concentrated and the residue was purified by column on silica gel (petroleum ether:ethyl acetate) to afford crude 7-bromo-5-fluoro-3,4-dihydroquinolin-2(1H)-one (1.6 g, 6.5 mmol, 25% yield). ¹H NMR (400 MHz, CDCl₃) δ 8.05 (s, 1H), 7.65-7.63 (m, 1H), 6.95-6.93 (m, 1H), 4.08-4.04 (m, 2H), 3.14-3.12 (m, 2H); MS (ESI) m/z: 244.1 [M+1]⁺.

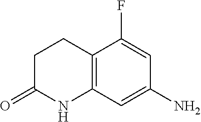 [SMILES: Nc1cc(F)c2c(c1)NC(=O)CC2]

7-amino-5-fluoro-3,4-dihydroquinolin-2(1H)-one. To a solution of 7-bromo-5-fluoro-3,4-dihydroquinolin-2(1H)-one (0.8 g, 3.28 mmol), diphenylmethanimine (0.71 g, 3.93 mmol), 2,2′-Bis(diphenylphosphino)-1,1′-binaphthyl (0.2 g, 0.3300 mmol) in Toluene (20 mL) was added sodium tert-butoxide (10. mL, 9.83 mmol, 1.0 M in THF), Pd₂(dba)₃ (0.30 g, 0.3300 mmol) under nitrogen for 12 h. The reaction was quenched water, then extracted with dichloromethane. The organic layer was washed with brine, dried over sodium sulfate, filtered and the filtrate was concentrated to afford crude 7-(benzhydrylideneamino)-5-fluoro-3,4-dihydro-1H-quinolin-2-one (800 mg, 2.32 mmol, 71% yield) as a yellow oil, which was used into next step without purification. To a solution of 7-(benzhydrylideneamino)-5-fluoro-3,4-dihydro-1H-quinolin-2-one (1.0 g, 2.9 mmol) in THE (20 mL) was added hydrochloride acid (1.67 mL, 21.7 mmol, 3M). The solution was stirred at 20° C. for 12 h. The solution was extracted with dichloromethane. The organic layer was washed with brine, dried over sodium sulfate, filtered and the filtrate was concentrated to afford 7-amino-5-fluoro-3,4-dihydroquinolin-2(1H)-one (800 mg, 4.44 mmol, 51% yield). MS (ESI) m/z: 182.3 [M+1]⁺.

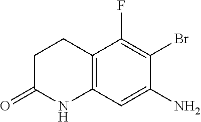 [SMILES: Nc1cc2c(c(F)c1Br)CCC(=O)N2]

7-amino-6-bromo-5-fluoro-3,4-dihydroquinolin-2(1H)-one. To a mixture of 7-amino-5-fluoro-3,4-dihydroquinolin-2(1H)-one (400. mg, 2.22 mmol) suspended in dichloromethane (10 mL), Methanol (2.5 mL) was added tetra-n-butylammonium tribromide (695 mg, 1.44 mmol) at 0° C. The reaction mixture was stirred at 25° C. for two hours. TLC showed this reaction worked well. Then it was partitioned between dichloromethane and 10% aqueous sodium thiosulfate. The organic layer was dried over sodium sulfate, filtered and concentrated under reduced pressure. Purification by column chromatography, eluting with ethyl acetate/hexanes, provided 7-amino-6-bromo-5-fluoro-3,4-dihydroquinolin-2(1H)-one (300 mg, 1.15 mmol, 52% yield). ¹H NMR (400 MHz, CDCl₃) δ 7.81 (brs, 1H), 6.01 (s, 1H), 4.14 (brs, 2H), 2.95-2.91 (m, 2H), 2.63-2.59 (m, 2H).

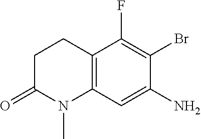

7-amino-6-bromo-5-fluoro-1-methyl-3,4-dihydroquinolin-2(1H)-one. To a solution of 7-amino-6-bromo-5-fluoro-3,4-dihydroquinolin-2(1H)-one (0.30 g, 1.16 mmol) in THE (5 mL) was added Potassium bis(trimethylsilyl)amide (1M) (1.2 mL, 1.27 mmol) at 0° C. Then iodomethane (180 mg, 1.27 mmol) was added to the solution. The solution was stirred at 20° C. for 12 h. The reaction was quenched with saturated ammonium chloride solution, then extracted with dichloromethane. The organic layer was concentrated and the residue was purified by prep-TLC (Ethyl acetate) to afford 7-amino-6-bromo-5-fluoro-1-methyl-3,4-dihydroquinolin-2(1H)-one (250 mg, 0.915 mmol, 79% yield) as a white solid. MS (ESI) m/z: 273.1 [M+1]⁺.

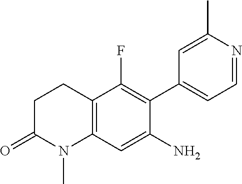 [SMILES: Cc1cc(-c2c(N)cc3c(c2F)CCC(=O)N3C)ccn1]

7-amino-5-fluoro-1-methyl-6-(2-methylpyridin-4-yl)-3,4-dihydroquinolin-2(1H)-one. To a solution of 7-amino-6-bromo-5-fluoro-1-methyl-3,4-dihydroquinolin-2(1H)-one (0.170 g, 0.620 mmol), 2-methyl-4-(4,4,5,5-tetramethyl-1,3,2-dioxaborolan-2-yl)pyridine (0.136 g, 0.620 mmol), potassium carbonate (0.215 g, 1.56 mmol) in 1,4-Dioxane (10 mL), Water (3 mL) was added (1,1′-bis(diphenylphosphino)ferrocene)palladium(II) dichloride (0.030 g, 0.620 mmol) under nitrogen. The solution was stirred at 110° C. for 12 h. The reaction mixture was filtered and the filtrate was concentrated under reduce pressure by water pump. The residue was purified by prep-TLC (dichloromethane:methanol) to afford 7-amino-5-fluoro-1-methyl-6-(2-methylpyridin-4-yl)-3,4-dihydroquinolin-2(1H)-one (150 mg, 0.525 mmol, 84% yield). MS (ESI) m/z: 286.4 [M+1]⁺.

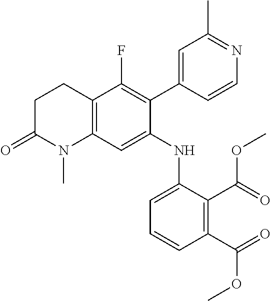 [SMILES: COC(=O)c1cccc(Nc2cc3c(c(F)c2-c2ccnc(C)c2)CCC(=O)N3C)c1C(=O)OC]

Dimethyl 3-((5-fluoro-1-methyl-6-(2-methylpyridin-4-yl)-2-oxo-1,2,3,4-tetrahydroquinolin-7-yl)amino)phthalate. To a solution of 7-amino-5-fluoro-1-methyl-6-(2-methylpyridin-4-yl)-3,4-dihydroquinolin-2(1H)-one (0.150 g, 0.530 mmol) and dimethyl 3-bromobenzene-1,2-dicarboxylate (0.157 g, 0.580 mmol) in 1,4-Dioxane (10 mL) was added cesium carbonate (0.51 g, 1.58 mmol) and Methanesulfonato(2-dicyclohexylphosphino-2,6-di-i-propoxy-1,1-biphenyl)(2-amino-1,1-biphenyl-2-yl)palladium(II) (0.043 g, 0.050 mmol). The resulting mixture was stirred at 110° C. for 12 hours under nitrogen. The reaction mixture was filtered and the filtrate was concentrated under reduce pressure by water pump. The residue was purified by prep-TLC (dichloromethane:methanol) to give dimethyl 3-((5-fluoro-1-methyl-6-(2-methylpyridin-4-yl)-2-oxo-1,2,3,4-tetrahydroquinolin-7-yl)amino)phthalate (150 mg, 0.314 mmol, 59% yield). MS (ESI) m/z: 478.2 [M+1]⁺.

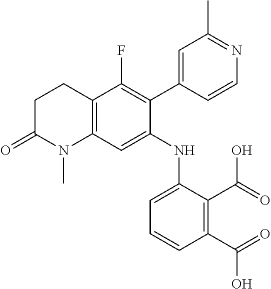 [SMILES: Cc1cc(-c2c(Nc3cccc(C(=O)O)c3C(=O)O)cc3c(c2F)CCC(=O)N3C)ccn1]

3-((5-fluoro-1-methyl-6-(2-methylpyridin-4-yl)-2-oxo-1,2,3,4-tetrahydroquinolin-7-yl)amino)phthalic acid. To a solution of dimethyl 3-((5-fluoro-1-methyl-6-(2-methylpyridin-4-yl)-2-oxo-1,2,3,4-tetrahydroquinolin-7-yl)amino)phthalate (0.150 g, 0.310 mmol) in Methanol (10 mL) was added sodium hydroxide (0.037 g, 0.940 mmol) in Water (2 mL). The solution was stirred at 85° C. for 12 h. The solution was adjusted to pH=3 with hydrochloride acid (1M), then extracted with dichloromethane. The organic layer was washed with brine, dried over sodium sulfate, filtered and filtrate was concentrated to afford 3-((5-fluoro-1-methyl-6-(2-methylpyridin-4-yl)-2-oxo-1,2,3,4-tetrahydroquinolin-7-yl)amino)phthalic acid (140 mg, 0.311 mmol, 99% yield). MS (ESI) m/z: 450.3 [M+1]⁺.

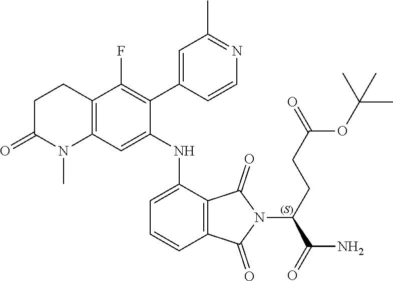

tert-butyl (S)-5-amino-4-(4-((5-fluoro-1-methyl-6-(2-methylpyridin-4-yl)-2-oxo-1,2,3,4-tetrahydroquinolin-7-yl)amino)-1,3-dioxoisoindolin-2-yl)-5-oxopentanoate. To a solution of 3-[[5-fluoro-1-methyl-6-(2-methyl-4-pyridyl)-2-oxo-3,4-dihydroquinolin-7-yl]amino]phthalic acid (0.140 g, 0.3100 mmol), tert-butyl (4S)-4,5-diamino-5-oxo-pentanoate;hydrochloride (0.074 g, 0.3100 mmol) in Toluene (5 mL) was added diisopropylethylamine (0.27 mL, 1.56 mmol). The solution was stirred at 135° C. for 12 h. The solution was concentrated and the residue was purified by column on silica gel (petroleum ether:ethyl acetate=1:1 to dichloromethane:methanol=10:1) to afford tert-butyl (S)-5-amino-4-(4-((5-fluoro-1-methyl-6-(2-methylpyridin-4-yl)-2-oxo-1,2,3,4-tetrahydroquinolin-7-yl)amino)-1,3-dioxoisoindolin-2-yl)-5-oxopentanoate (150 mg, 0.2436 mmol, 78% yield) as a yellow solid. ¹H NMR (400 MHz, CDCl3) δ 8.57 (s, 1H), 7.90 (s, 1H), 7.53-7.50 (m, 1H), 7.29-7.26 (m, 2H), 7.14-7.05 (m, 1H), 6.95 (s, 1H), 6.25 (brs, 1H), 5.50 (brs, 1H), 4.80-4.77 (m, 1H), 3.37 (s, 3H), 3.02-3.00 (m, 2H), 2.77-2.75 (m, 2H), 2.63 (s, 3H), 2.48-2.46 (m, 2H), 2.32-2.30 (m, 2H), 1.43 (s, 9H); MS (ESI) m/z: 616.2 [M+1]⁺.

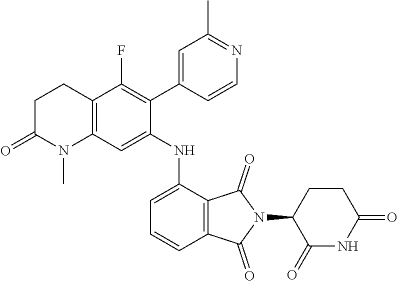 [SMILES: Cc1cc(-c2c(Nc3cccc4c3C(=O)N([C@H]3CCC(=O)NC3=O)C4=O)cc3c(c2F)CCC(=O)N3C)ccn1]

(S)-2-(2,6-dioxopiperidin-3-yl)-4-((5-fluoro-1-methyl-6-(2-methylpyridin-4-yl)-2-oxo-1,2,3,4-tetrahydroquinolin-7-yl)amino)isoindoline-1,3-dione. A solution of tert-butyl (S)-5-amino-4-(4-((5-fluoro-1-methyl-6-(2-methylpyridin-4-yl)-2-oxo-1,2,3,4-tetrahydroquinolin-7-yl)amino)-1,3-dioxoisoindolin-2-yl)-5-oxopentanoate (0.060 g, 0.1000 mmol), benzenesulfonic acid (0.030 g, 0.190 mmol) in acetonitrile (1 mL) was stirred at 60° C. for 12 h. The reaction was distilled with dichloromethane and sodium bicarbonate, then extracted with dichloromethane. The organic layer was washed with brine, dried over anhydrous sodium sulfate, filtered and the filtrate was concentrated. The residue was purified by prep-TLC (dichloromethane:methanol) to afford (S)-2-(2,6-dioxopiperidin-3-yl)-4-((5-fluoro-1-methyl-6-(2-methylpyridin-4-yl)-2-oxo-1,2,3,4-tetrahydroquinolin-7-yl)amino)isoindoline-1,3-dione (50 mg, 0.092 mmol, 57% yield). The (S)-2-(2,6-dioxopiperidin-3-yl)-4-((5-fluoro-1-methyl-6-(2-methylpyridin-4-yl)-2-oxo-1,2,3,4-tetrahydroquinolin-7-yl)amino)isoindoline-1,3-dione (0.050 g, 0.0900 mmol) was separated by SFC. After the separation, the major peak was concentrated under reduced pressure to afford (S)-2-(2,6-dioxopiperidin-3-yl)-4-((5-fluoro-1-methyl-6-(2-methylpyridin-4-yl)-2-oxo-1,2,3,4-tetrahydroquinolin-7-yl)amino)isoindoline-1,3-dione (30 mg, 0.0561 mmol, 61% yield) was obtained as a yellow solid. ¹H NMR (400 MHz, DMSO-d₆) ¹H NMR (400 MHz, DMSO-d₆) δ 11.11 (s, 1H), 8.43 (s, 1H), 8.23 (s, 1H), 7.54-7.50 (m, 1H), 7.27 (s, 1H), 7.20-7.17 (m, 3H), 7.10 (s, 1H), 5.09-5.04 (m, 1H), 3.28 (s, 3H), 2.93-2.90 (m, 3H), 2.67-2.65 (m, 3H), 2.45-2.33 (m, 4H), 2.08-2.01 (m, 1H); MS (ESI) m/z: 542.3 [M+1]⁺.

### Example 14: Synthesis of Compound 91: 2-(2,6-dioxopiperidin-3-yl)-4-((6-((1r,4r)-4-methoxycyclohexyl)-1-methyl-2-oxo-1,2,3,4-tetrahydroquinolin-7-yl)amino)isoindoline-1,3-dione

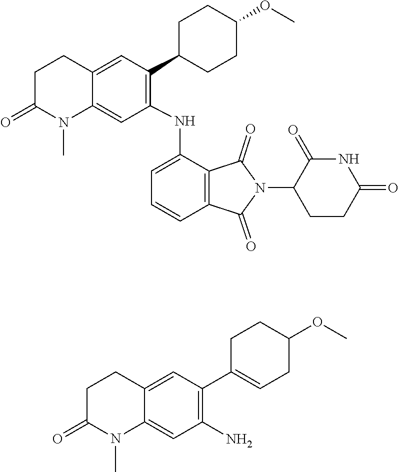 [SMILES: CO[C@H]1CC[C@H](c2cc3c(cc2Nc2cccc4c2C(=O)N(C2CCC(=O)NC2=O)C4=O)N(C)C(=O)CC3)CC1 ; COC1CC=C(c2cc3c(cc2N)N(C)C(=O)CC3)CC1]

7-amino-6-(4-methoxycyclohex-1-en-1-yl)-1-methyl-3,4-dihydroquinolin-2(1H)-one. To a solution of 7-amino-6-bromo-1-methyl-3,4-dihydroquinolin-2-one (0.150. g, 0.59 mmol) and 2-(4-methoxycyclohexen-1-yl)-4,4,5,5-tetramethyl-1,3,2-dioxaborolane (0.169. g, 0.71 mmol) in 1,4-Dioxane (2 mL) and was added potassium carbonate (0.253 g, 1.83 mmol), then the mixture replaced gas of nitrogen for 3 times was added (1,1′-bis(diphenylphosphino)ferrocene)palladium(II) dichloride (0.048 g, 0.06 mmol), the mixture was stirred at 110° C. for 12 hours under the nitrogen. The suspension was filtered and the filtrate was concentrated. The residue was purified by column chromatography on silica gel (petroleum ether:ethyl acetate) to afford 7-amino-6-(4-methoxycyclohex-1-en-1-yl)-1-methyl-3,4-dihydroquinolin-2(1H)-one (140 mg, 0.49 mmol, 83% yield). MS (ESI) m/z: 287.1 [M+1]⁺.

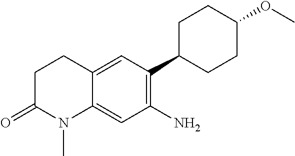 [SMILES: CO[C@H]1CC[C@H](c2cc3c(cc2N)N(C)C(=O)CC3)CC1]

7-amino-6-((1r,4r)-4-methoxycyclohexyl)-1-methyl-3,4-dihydroquinolin-2(1H)-one. To a solution of 7-amino-6-(4-methoxycyclohex-1-en-1-yl)-1-methyl-3,4-dihydroquinolin-2(1H)-one (0.140 g, 0.49 mmol) in Methanol (2 mL) was added wet palladium carbon (0.050 g, 0.47 mmol), the mixture was stirred at 25° C. under the hydrogen for 12 hours. The suspension was filtered and the filtrate was concentrated. The residue was purified by prep-TLC (petroleum ether:ethyl acetate) to afford 7-amino-6-((1r,4r)-4-methoxycyclohexyl)-1-methyl-3,4-dihydroquinolin-2(1H)-one (40 mg, 0.14 mmol, 28% yield) as purple solid. ¹H NMR (400 MHz, CDCl₃) δ 6.87 (s, 1H), 6.42-6.39 (m, 1H), 3.39 (s, 3H), 3.32-3.30 (m, 3H), 3.26-3.20 (m, 1H), 2.82-2.77 (m, 2H), 2.64-2.60 (m, 2H), 2.51-2.42 (m, 1H), 2.26-2.19 (m, 2H), 2.00-1.93 (m, 2H), 1.49-1.42 (m, 2H), 1.41-1.34 (m, 2H); MS (ESI) m/z: 289.2 [M+1]⁺.

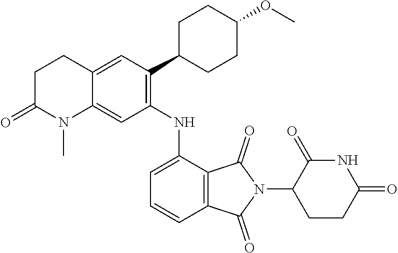 [SMILES: CO[C@H]1CC[C@H](c2cc3c(cc2Nc2cccc4c2C(=O)N(C2CCC(=O)NC2=O)C4=O)N(C)C(=O)CC3)CC1]

2-(2,6-dioxopiperidin-3-yl)-4-((6-((1r,4r)-4-methoxycyclohexyl)-1-methyl-2-oxo-1,2,3,4-tetrahydroquinolin-7-yl)amino)isoindoline-1,3-dione. To a solution of 7-amino-6-((1r,4r)-4-methoxycyclohexyl)-1-methyl-3,4-dihydroquinolin-2(1H)-one (0.040 g, 0.14 mmol) in 1,4-Dioxane (2 mL) was added 4-bromo-2-(2,6-dioxo-3-piperidyl)isoindoline-1,3-dione (0.056 g, 0.17 mmol) and potassium carbonate (0.067 g, 0.48 mmol) and chloro(2-dicyclohexylphosphino-2′,4′,6′-triisopropyl-1,1′-biphenyl)[2-(2′-amino-1,1′-biphenyl)]palladium(II) (0.011. g, 0.01 mmol), the mixture was stirred at 110° C. for 12 hours under the nitrogen. The reaction mixture was filtered and the filtrate was concentrated under reduce pressure. The residue was purified by prep-TLC (ethyl acetate) to afford the crude. The crude product was purified by prep-HPLC (acetonitrile/formic acid/water]) to obtain the product 2-(2,6-dioxopiperidin-3-yl)-4-((6-((1r,4r)-4-methoxycyclohexyl)-1-methyl-2-oxo-1,2,3,4-tetrahydroquinolin-7-yl)amino)isoindoline-1,3-dione (24 mg, 0.044 mmol, 32% yield). ¹H NMR (400 MHz, DMSO-d₆) δ 11.13 (s, 1H), 8.17-8.12 (m, 1H), 7.59-7.54 (m, 1H), 7.27-7.23 (m, 1H), 7.21-7.17 (m, 1H), 7.02-7.00 (m, 1H), 6.99-6.95 (m, 1H), 5.17-5.12 (m, 1H), 3.24-3.22 (m, 3H), 3.21-3.19 (m, 3H), 3.17-3.10 (m, 1H), 2.92-2.87 (m, 3H), 2.66-2.62 (m, 2H), 2.59 (br s, 2H), 2.12-2.07 (m, 2H), 2.07-2.04 (m, 1H), 1.78-1.71 (m, 2H), 1.56-1.46 (m, 2H), 1.30-1.20 (m, 1H), 1.16-1.06 (m, 2H); MS (ESI) m/z: 545.3 [M+1]⁺.

### Example 15: Synthesis of Compound 92: 2-(2,6-dioxopiperidin-3-yl)-4-((6-((1s,4s)-4-methoxycyclohexyl)-1-methyl-2-oxo-1,2,3,4-tetrahydroquinolin-7-yl)amino)isoindoline-1,3-dione

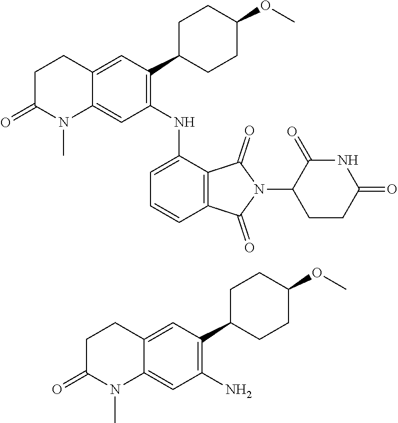

7-amino-6-((1s,4s)-4-methoxycyclohexyl)-1-methyl-3,4-dihydroquinolin-2(1H)-one. To a solution of 7-amino-6-(4-methoxycyclohex-1-en-1-yl)-1-methyl-3,4-dihydroquinolin-2(1H)-one (0.140. g, 0.49 mmol) in Methanol (2 mL) was added wet palladium carbon (0.050. g, 0.47 mmol), the mixture was stirred at 25° C. under the hydrogen for 12 hours. The suspension was filtered and the filtrate was concentrated. The residue was purified by prep-TLC (petroleum ether:ethyl acetate=1:1) to afford 7-amino-6-((1s,4s)-4-methoxycyclohexyl)-1-methyl-3,4-dihydroquinolin-2(1H)-one (70 mg, 0.20 mmol, 49.7% yield) as purple solid. ¹H NMR (400 MHz, CDCl₃) δ 6.96 (s, 1H), 6.39-6.37 (m, 1H), 3.57-3.52 (m, 1H), 3.36 (s, 3H), 3.32-3.30 (m, 3H), 2.82-2.77 (m, 2H), 2.64-2.60 (m, 2H), 2.55-2.45 (m, 1H), 2.15-2.06 (m, 2H), 1.84-1.72 (m, 2H), 1.66-1.61 (m, 2H), 1.55-1.49 (m, 2H); MS (ESI) m/z: MS (ESI) m/z: 289.2 [M+1]⁺.

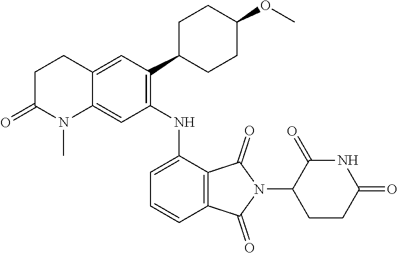 [SMILES: CO[C@H]1CC[C@@H](c2cc3c(cc2Nc2cccc4c2C(=O)N(C2CCC(=O)NC2=O)C4=O)N(C)C(=O)CC3)CC1]

2-(2,6-dioxopiperidin-3-yl)-4-((6-((1s,4s)-4-methoxycyclohexyl)-1-methyl-2-oxo-1,2,3,4-tetrahydroquinolin-7-yl)amino)isoindoline-1,3-dione. To a solution of 7-amino-6-((1s,4s)-4-methoxycyclohexyl)-1-methyl-3,4-dihydroquinolin-2(1H)-one (0.070 g, 0.24 mmol) in 1,4-Dioxane (2 mL) was added potassium carbonate (0.118 mg, 0.86 mmol), 4-bromo-2-(2,6-dioxo-3-piperidyl)isoindoline-1,3-dione (0.090 g, 0.27 mmol) and chloro(2-dicyclohexylphosphino-2′,4′,6′-triisopropyl-1,1′-biphenyl)[2-(2′-amino-1,1′-biphenyl)]palladium(II) (0.019 g, 0.02 mmol), the mixture was stirred at 125° C. for 12 hours under the nitrogen. LCMS (WXW-CRO-202766-32-P1A) showed the desired mass was checked. The reaction mixture was filtered and the filtrate was concentrated under reduce pressure by water pump. The residue was purified by prep-TLC (ethyl acetate) to afford the crude. The crude product was purified by prep-HPLC (Column: Phenomenex C18 75*30 mm*3 μm, [38%-68% acetonitrile+0.1% formic acid in water], 7 min). To obtained the product 2-(2,6-dioxopiperidin-3-yl)-4-((6-((1s,4s)-4-methoxycyclohexyl)-1-methyl-2-oxo-1,2,3,4-tetrahydroquinolin-7-yl)amino)isoindoline-1,3-dione (61.44 mg, 0.1109 mmol, 46% yield) as yellow solid. ¹H NMR (400 MHz, DMSO-d₆) δ 11.14 (s, 1H), 8.13 (s, 1H), 7.59-7.53 (m, 1H), 7.23-7.20 (m, 1H), 7.20-7.17 (m, 1H), 7.02-7.00 (m, 1H), 6.99-6.95 (m, 1H), 5.17-5.11 (m, 1H), 3.30-3.26 (m, 1H), 3.24-3.22 (m, 3H), 3.21-3.19 (m, 3H), 2.93-2.87 (m, 3H), 2.66 (dd, J=13.2 Hz, 2H), 2.59 (dd, J=5.6 Hz, 2H), 2.10-2.06 (m, 1H), 1.97-1.90 (m, 2H), 1.73-1.64 (m, 2H), 1.52-1.28 (m, 5H); MS (ESI) m/z: 545.1 [M+1]⁺.

### Example 16: Synthesis of Compound 93: 4-((6-(1-(cyclopropanecarbonyl)piperidin-4-yl)-1-methyl-2-oxo-1,2,3,4-tetrahydroquinolin-7-yl)amino)-2-(2,6-dioxopiperidin-3-yl)isoindoline-1,3-dione

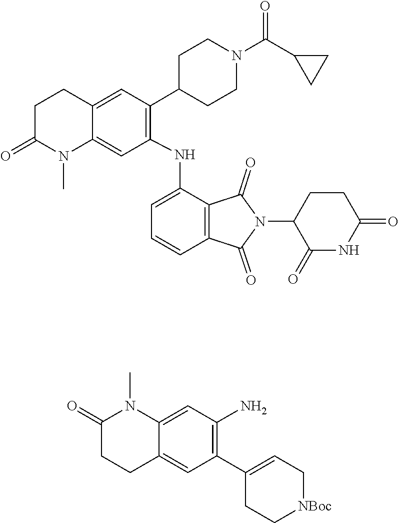 [SMILES: CN1C(=O)CCc2cc(C3CCN(C(=O)C4CC4)CC3)c(Nc3cccc4c3C(=O)N(C3CCC(=O)NC3=O)C4=O)cc21 ; CN1C(=O)CCc2cc(C3=CCN(C(=O)OC(C)(C)C)CC3)c(N)cc21]

tert-butyl 4-(7-amino-1-methyl-2-oxo-1,2,3,4-tetrahydroquinolin-6-yl)-3,6-dihydropyridine-1(2H)-carboxylate. To a solution of 7-amino-6-bromo-1-methyl-3,4-124ioxaborolanel24ol-2(1H)-one (0.300 g, 1.18 mmol) in 1,4-Dioxane (10 mL) and Water (1 mL) were added tert-butyl 4-(4,4,5,5-tetramethyl-1,3,2-dioxaborolan-2-yl)-3,6-dihydropyridine-1(2H)-carboxylate (0.364 g, 1.18 mmol), (1,1′-bis(diphenylphosphino)ferrocene)palladium(II) dichloride (0.043 g, 0.06 mmol) and potassium carbonate (0.195 g, 1.41 mmol), then the reaction was stirred at 110° C. for 12 hours under nitrogen. The reaction mixture was poured in water and extracted with EtOAc. The organic layer was filtrated and the filtrate was concentrated. The residue was purified by silica column chromatography on silica gel using Petroleum ether and Ethyl acetate to afford tert-butyl 4-(7-amino-1-methyl-2-oxo-1,2,3,4-tetrahydroquinolin-6-yl)-3,6-dihydropyridine-1(2H)-carboxylate (400 mg, 1.12 mmol, 95% yield) was obtained as a yellow oil. ¹H NMR 400 MHz, DMSO-d₆) δ 6.72 (s, 1H), 6.42 (s, 1H), 5.64 (s, 1H), 4.79 (s, 1H), 3.94 (s, 2H), 3.54-3.52 (m, 2H), 3.32 (s, 3H), 2.64-2.67 (m, 2H), 2.47-2.44 (m, 2H), 1.42 (s, 9H); MS (ESI) m/z: 358.2 [M+1]⁺.

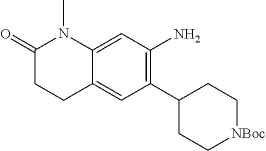 [SMILES: CN1C(=O)CCc2cc(C3CCN(C(=O)OC(C)(C)C)CC3)c(N)cc21]

tert-butyl 4-(7-amino-1-methyl-2-oxo-1,2,3,4-tetrahydroquinolin-6-yl)piperidine-1-carboxylate. To a solution of tert-butyl 4-(7-amino-1-methyl-2-oxo-1,2,3,4-tetrahydroquinolin-6-yl)-3,6-dihydropyridine-1(2H)-carboxylate (0.400 g, 1.12 mmol) in tetrahydrofuran (5 mL) was added palladium active/carbon catalyst (0.363 g, 3.41 mmol), the mixture was stirred under hydrogen atmosphere at 25° C. for 12 hours. A desired mass was observed by LCMS. The mixture was filtered and the filtrate was concentrated to afford tert-butyl 4-(7-amino-1-methyl-2-oxo-1,2,3,4-tetrahydroquinolin-6-yl)piperidine-1-carboxylate (400 mg, 1.11 mmol, 99.4% yield) was obtained as a colorless oil. MS (ESI) m/z: 360.2 [M+1]⁺.

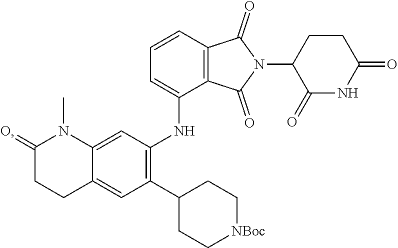 [SMILES: CN1C(=O)CCc2cc(C3CCN(C(=O)OC(C)(C)C)CC3)c(Nc3cccc4c3C(=O)N(C3CCC(=O)NC3=O)C4=O)cc21]

tert-butyl 4-(7-((2-(2,6-dioxopiperidin-3-yl)-1,3-dioxoisoindolin-4-yl)amino)-1-methyl-2-oxo-1,2,3,4-tetrahydroquinolin-6-yl)piperidine-1-carboxylate. To a solution of tert-butyl 4-(7-amino-1-methyl-2-oxo-1,2,3,4-tetrahydroquinolin-6-yl)piperidine-1-carboxylate (0.400 g, 1.11 mmol) in 1,4-Dioxane (6 mL) was added 4-bromo-2-(2,6-dioxopiperidin-3-yl)isoindoline-1,3-dione (0.413 g, 1.22 mmol), potassium carbonate (0.461 g, 3.34 mmol), Chloro(2-dicyclohexylphosphino-2,4,6-triisopropyl-1,1-biphenyl)[2-(2-amino-1,1-biphenyl)]palladium(II) (0.044 g, 0.06 mmol), the mixture was stirred under nitrogen at 110° C. for 12 hours. The organic layer was filtrated and the filtrate was concentrated. The residue was purified by silica column chromatography on silica gel (Petroleum ether:Ethyl acetate) to afford tert-butyl 4-(7-((2-(2,6-dioxopiperidin-3-yl)-1,3-dioxoisoindolin-4-yl)amino)-1-methyl-2-oxo-1,2,3,4-tetrahydroquinolin-6-yl)piperidine-1-carboxylate (500 mg, 0.81 mmol, 73% yield) was obtained as a yellow solid. ¹H NMR (400 MHz, DMSO-d₆) δ 11.14 (s, 1H), 8.15 (s, 1H), 7.57-7.53 (m, 1H), 7.28 (s, 1H), 7.19-7.17 (m, 1H), 7.00 (s, 1H), 6.91-6.89 (m, 1H), 5.15-5.11 (m, 1H), 3.19 (s, 3H), 2.91-2.83 (m, 4H), 2.64 (s, 2H), 2.59-2.54 (m, 4H), 1.99 (s, 3H), 1.68-1.65 (m, 2H), 1.52-1.49 (m, 2H), 1.39 (s, 9H); MS (ESI) m/z: 516.3 [M+1]⁺.

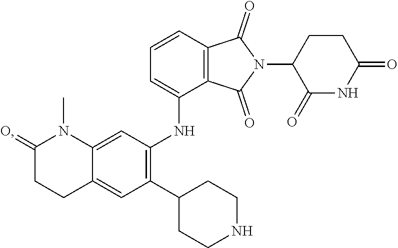 [SMILES: CN1C(=O)CCc2cc(C3CCNCC3)c(Nc3cccc4c3C(=O)N(C3CCC(=O)NC3=O)C4=O)cc21]

2-(2,6-dioxopiperidin-3-yl)-4-((1-methyl-2-oxo-6-(piperidin-4-yl)-1,2,3,4-tetrahydroquinolin-7-yl)amino)isoindoline-1,3-dione. To a solution of tert-butyl 4-(7-((2-(2,6-dioxopiperidin-3-yl)-1,3-dioxoisoindolin-4-yl)amino)-1-methyl-2-oxo-1,2,3,4-126ioxaborolane126oline-6-yl)piperidine-1-carboxylate (0.50 g, 0.81 mmol) in dichloromethane (1 mL) was added trifluoroacetic acid (0.12 mL, 1.62 mmol). The mixture was stirred at 25° C. for 12 hours. The organic layer was filtrated and the filtrate was concentrated to give 2-(2,6-dioxopiperidin-3-yl)-4-((1-methyl-2-oxo-6-(piperidin-4-yl)-1,2,3,4-tetrahydroquinolin-7-yl)amino)isoindoline-1,3-dione (400 mg, 0.78 mmol, 96% yield). MS (ESI) m/z: 516.2 [M+1]⁺.

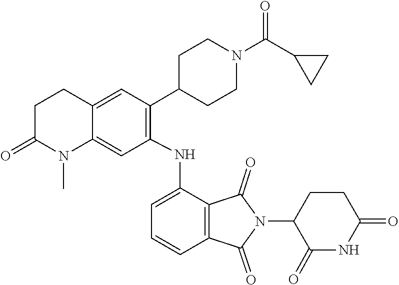 [SMILES: CN1C(=O)CCc2cc(C3CCN(C(=O)C4CC4)CC3)c(Nc3cccc4c3C(=O)N(C3CCC(=O)NC3=O)C4=O)cc21]

4-((6-(1-(cyclopropanecarbonyl)piperidin-4-yl)-1-methyl-2-oxo-1,2,3,4-tetrahydroquinolin-7-yl)amino)-2-(2,6-dioxopiperidin-3-yl)isoindoline-1,3-dione. To a mixture of 2-(2,6-dioxopiperidin-3-yl)-4-((1-methyl-2-oxo-6-(piperidin-4-yl)-1,2,3,4-tetrahydroquinolin-7-yl)amino)isoindoline-1,3-dione (0.080 g, 0.16 mmol) in dichloromethane (1 mL) was added N-ethyl-N-isopropylpropan-2-amine (0.29 mL, 1.67 mmol) and cyclopropanecarbonyl chloride (0.008 g, 0.08 mmol) The mixture was stirred at 25° C. for 2 hours. A desired mass was observed by LCMS. The mixture was filtered and the filtrate was concentrated. The residue was purified by prep-HPLC (acetonitrile/formic acid/water) to afford 4-((6-(1-(cyclopropanecarbonyl)piperidin-4-yl)-1-methyl-2-oxo-1,2,3,4-tetrahydroquinolin-7-yl)amino)-2-(2,6-dioxopiperidin-3-yl)isoindoline-1,3-dione (12 mg, 0.021 mmol, 14% yield). ¹H NMR (400 MHz, DMSO-d₆) δ 11.13 (s, 1H), 8.17 (s, 1H), 7.57-7.53 (m, 1H), 7.29 (s, 1H), 7.19-7.17 (m, 1H), 7.00 (s, 1H), 6.90-6.88 (m, 1H), 5.16-5.11 (m, 1H), 4.50-4.32 (m, 2H), 3.19 (s, 3H), 3.06-2.94 (m, 2H), 2.91-2.87 (m, 3H), 2.64-2.54 (m, 4H), 2.10-1.94 (m, 2H), 1.78-1.69 (m, 2H), 1.61-1.47 (m, 2H), 1.24 (s, 2H); MS (ESI) m/z: 584.4 [M+1]⁺.

### Example 17: Synthesis of Compound 94: 2-(2,6-dioxo-3-piperidyl)-4-[[6-(2-methyl-4-pyridyl)-2-oxo-1-vinyl-3,4-dihydroquinolin-7-yl]amino]isoindoline-1,3-dione

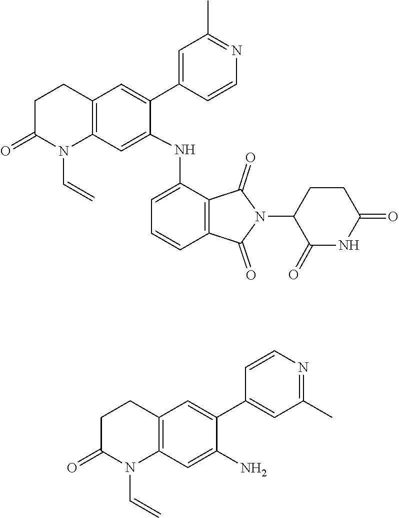 [SMILES: C=CN1C(=O)CCc2cc(-c3ccnc(C)c3)c(Nc3cccc4c3C(=O)N(C3CCC(=O)NC3=O)C4=O)cc21 ; C=CN1C(=O)CCc2cc(-c3ccnc(C)c3)c(N)cc21]

7-amino-6-(2-methyl-4-pyridyl)-1-vinyl-3,4-dihydroquinolin-2-one. To a solution of 7-amino-6-bromo-1-vinyl-3,4-dihydroquinolin-2-one (0.050 g, 0.19 mmol) in 1,4-Dioxane (10 mL) and Water (1 mL) was added 2-methyl-4-(4,4,5,5-tetramethyl-1,3,2-127ioxaborolane-2-yl)pyridine (0.049 g, 0.22 mmol), potassium carbonate (0.077 g, 0.56 mmol) and (1,1′-bis(diphenylphosphino)ferrocene)palladium(II) dichloride (0.015 g, 0.02 mmol). The suspension was degassed under vacuum and purged with nitrogen several times. The mixture was stirred at 110° C. for 12 h. The mixture was filtered and filtrate was concentrated under reduced pressure. The residue was purified by prep-TLC (ethyl acetate) to give 7-amino-6-(2-methyl-4-pyridyl)-1-vinyl-3,4-dihydroquinolin-2-one (40 mg, 0.14 mmol, 77% yield). ¹H NMR (400 MHz, CDCl₃) δ 8.56 (d, J=5.2 Hz, 1H), 7.29 (s, 1H), 7.24 (d, J=5.2 Hz, 1H), 6.97 (s, 1H), 6.80 (dd, J=8.8, 16.0 Hz, 1H), 6.72 (s, 1H), 5.46 (d, J=16.4 Hz, 1H), 5.25 (d, J=9.2 Hz, 1H), 3.81 (br s, 2H), 2.87-2.81 (m, 2H), 2.70-2.66 (m, 2H), 2.63 (s, 3H); MS (ESI) m/z: 280.1 [M+1]⁺.

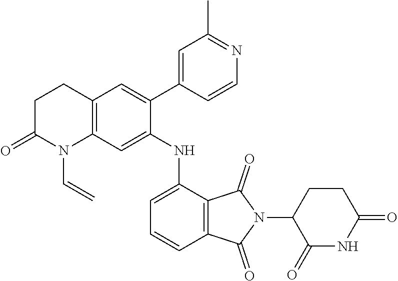 [SMILES: C=CN1C(=O)CCc2cc(-c3ccnc(C)c3)c(Nc3cccc4c3C(=O)N(C3CCC(=O)NC3=O)C4=O)cc21]

2-(2,6-dioxo-3-piperidyl)-4-[[6-(2-methyl-4-pyridyl)-2-oxo-1-vinyl-3,4-dihydroquinolin-7-yl]amino]isoindoline-1,3-dione. To a solution of 7-amino-6-(2-methyl-4-pyridyl)-1-vinyl-3,4-dihydroquinolin-2-one (0.040 g, 0.14 mmol) in 1,4-Dioxane (5 mL) was added 4-bromo-2-(2,6-dioxo-3-piperidyl)isoindoline-1,3-dione (0.058 g, 0.17 mmol), potassium carbonate (0.059 g, 0.43 mmol) and chloro(2-dicyclohexylphosphino-2′,4′,6′-tri-i-propyl-1,1′-biphenyl)(2′-amino-1,1′-biphenyl-2-yl) palladium(II) (0.011 g, 0.01 mmol). The suspension was degassed under vacuum and purged with nitrogen several times. The mixture was stirred at 110° C. for 12 h. The residue was purified by prep-HPLC (acetonitrile/formic acid/water). The mixture was evaporated to remove the organic phase and the water phase was lyophilized. Then the residue was purified by prep-TLC (methanol in dichloromethane) to provide 2-(2,6-dioxo-3-piperidyl)-4-[[6-(2-methyl-4-pyridyl)-2-oxo-1-vinyl-3,4-dihydroquinolin-7-yl]amino]isoindoline-1,3-dione (31 mg, 0.057 mmol, 40% yield). ¹H NMR (400 MHz, DMSO-d₆) δ 11.11 (s, 1H), 8.37 (d, J=5.2 Hz, 1H), 8.34 (s, 1H), 7.50-7.45 (m, 1H), 7.45 (s, 1H), 7.33 (s, 2H), 7.24 (d, J=4.8 Hz, 1H), 7.16-7.10 (m, 1H), 7.01-6.93 (m, 1H), 6.83 (dd, J=9.2, 16.4 Hz, 1H), 5.43 (d, J=16.0 Hz, 1H), 5.19 (d, J=9.2 Hz, 1H), 5.08 (dd, J=5.2, 12.8 Hz, 1H), 3.00-2.92 (m, 2H), 2.91-2.82 (m, 1H), 2.69-2.64 (m, 2H), 2.63-2.56 (m, 2H), 2.41 (s, 3H), 2.07-1.97 (m, 1H); MS (ESI) m/z: 536.1 [M+1]⁺.

### Example 18: Synthesis of Compound 95: (S)-2-(2,6-dioxopiperidin-3-yl)-4-((5-methyl-2-(2-methylpyridin-4-yl)-6-oxo-5,6,7,8-tetrahydro-1,5-naphthyridin-3-yl)amino)isoindoline-1,3-dione

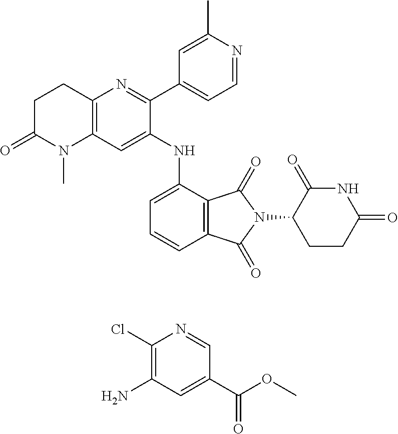 [SMILES: Cc1cc(-c2nc3c(cc2Nc2cccc4c2C(=O)N([C@H]2CCC(=O)NC2=O)C4=O)N(C)C(=O)CC3)ccn1 ; COC(=O)c1cnc(Cl)c(N)c1]

5-amino-6-chloro-pyridine-3-carboxylate. To a solution of methyl 6-chloro-5-nitronicotinate (0.50 g, 2.31 mmol) in ethanol (30 mL) and water (10 mL) were added ammonium chloride (1.25 g, 23.09 mmol) and ferrous powder (0.64 g, 11.54 mmol). The reaction was warmed to 80° C. and stirred for 12 hours. The reaction mixture was filtered and the filtrate was concentrated to afford methyl 5-amino-6-chloro-pyridine-3-carboxylate (400 mg, 2.14 mmol, 93% yield). ¹H NMR (400 MHz, DMSO-d₆) δ 8.07 (d, J=2.0 Hz, 1H), 7.65 (d, J=2.0 Hz, 1H), 5.93 (brs, 2H), 3.85 (s, 3H); MS (ESI) m/z: 187.0 [M+1]⁺.

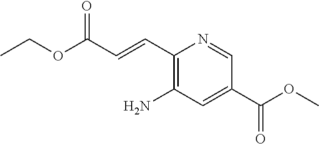 [SMILES: CCOC(=O)/C=C/c1ncc(C(=O)OC)cc1N]

methyl (E)-5-amino-6-(3-ethoxy-3-oxoprop-1-en-1-yl)nicotinate. To a solution of methyl 5-amino-6-chloronicotinate (2.0 g, 10.7 mmol) in N,N-dimethylformamide (30 mL) and water (3 mL) was added sodium carbonate (3.4 g, 32.1 mmol), eth€(E)-3-(4,4,5,5-tetramethyl-1,3,2-dioxaborolan-2-yl)acrylate (2.9 g, 12.8 mmol) an′ (1,1′-bis(diphenylphosphino)ferrocene)palladium(II) dichloride (0.44 g, 0.54 mmol), the mixture was stirred at 125° C. for 2 hours under the nitrogen. The reaction mixture was filtered and the filtrate was concentrated under reduce pressure by water pump. The residue was purified by column chromatography on silica gel (petroleum ether:ethyl acetate). The desired m€yl (E)-5-amino-6-(3-ethoxy-3-oxoprop-1-en-1-yl)nicotinate (2.200 g, 8.79 mmol, 82% yield). ¹H NMR (400 MHz, DMSO-d₆) δ 8.30 (d, J=2.0 Hz, 1H), 7.97 (d, J=15.2 Hz, 1H), 7.63 (d, J=2.0 Hz, 1H), 6.83 (d, J=15.2 Hz, 1H), 6.17 (s, 2-), 4.25-4.15 (m, 2H), 3.85 (s, 3H), 1.26 (t, J=7.2 Hz, 3H); MS (ESI) m/z: 251.1[M+1]⁺.

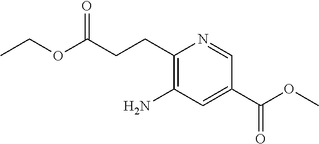 [SMILES: CCOC(=O)CCc1ncc(C(=O)OC)cc1N]

Methyl 5-amino-6-(3-ethoxy-3-oxopropyl)nicotinate. To a solution o€ethyl (E)-5-amino-6-(3-ethoxy-3-oxoprop-1-en-1-yl)nicotinate (0.500 g, 2 mmol) in ethanol (10 mL) was added dried palladium on activated carbon (0.100 g, 0.20 mmol). The reaction mixture is stirred at 25° C. for 12 hours, with hydrogen (50 psi) atmosphere. The reaction mixture was filtered and the filtrate was concentrated to afforded methyl 5-amino-6-(3-ethoxy-3-oxopropyl)nicotinate (400 mg, 1.49 mmol, 75% yield). ¹H NMR (400 MHz, DMSO-d6) δ 8.20 (d, J=2.0 Hz, 1H), 7.45 (d, J=2.0 Hz, 1H), 5.46 (s, 2H), 4.08-4.00 (m, 2H), 3.82 (s, 3H), 2.90-2.84 (m, 2H), 2.76-2.71 (m, 2H), 1.16 (t, J=7.2 Hz, 3H).

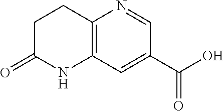 [SMILES: O=C1CCc2ncc(C(=O)O)cc2N1]

6-oxo-5,6,7,8-tetrahydro-1,5-naphthyridine-3-carboxylic acid. To a solution of methyl 5-amino-6-(3-ethoxy-3-oxopropyl)nicotinate (0.300 g, 1.19 mmol) in tetrahydrofuran (3 mL) was added potassium 2-methylpropan-2-olate (0.28 mL, 1.78 mmol). The reaction mixture was stirred at 25° C. for 1 hour, then diluted with water. The aqueous layer was extracted with ethyl acetate. The combined organic phase was dried over sodium sulfate and concentrated under reduced pressure to give 6-oxo-5,6,7,8-tetrahydro-1,5-naphthyridine-3-carboxylic acid (160 mg, 0.83 mmol, 70% yield). ¹H NMR (400 MHz, DMSO-d6) δ 10.29 (s, 1H), 8.55 (d, J=1.2 Hz, 1H), 7.65 (d, J=1.6 Hz, 1H), 3.08-3.04 (m, 2H), 2.62-2.58 (m, 2H); MS (ESI) m/z: 193.0 [M+1]⁺.

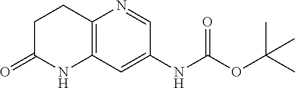 [SMILES: CC(C)(C)OC(=O)Nc1cnc2c(c1)NC(=O)CC2]

tert-butyl (6-oxo-5,6,7,8-tetrahydro-1,5-naphthyridin-3-yl)carbamate. A suspension of 6-oxo-5,6,7,8-tetrahydro-1,5-naphthyridine-3-carboxylic acid (0.100 g, 0.52 mmol) in toluene (3 mL) was treated with triethylamine (0.27 mL, 1.56 mmol), 2-methylpropan-2-ol (0.386 g, 5.20 mmol), and diphenyl phosphoryl azide (0.17 mL, 0.78 mmol). The reaction was warmed to 100° C. and stirred under argon for 12 hours. The reaction mixture was concentrated under reduce pressure and the resulting residue was purified by silica gel column chromatography (petroleum ether/ethyl acetate) to give tert-butyl (6-oxo-5,6,7,8-tetrahydro-1,5-naphthyridin-3-yl)carbamate (50 mg, 0.19 mmol, 37% yield). MS (ESI) m/z: 264.1 [M+1]⁺.

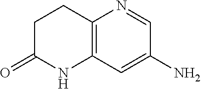 [SMILES: Nc1cnc2c(c1)NC(=O)CC2]

7-amino-3,4-dihydro-1H-1,5-naphthyridin-2-one. To a solution of tert-butyl (6-oxo-5,6,7,8-tetrahydro-1,5-naphthyridin-3-yl)carbamate (0.050 g, 0.19 mmol) in ethyl acetate (2 mL) was added HCl (0.15 mL, 1.9 mmol). The resulting mixture was stirred at 25° C. for 12 hours under nitrogen. The reaction mixture was concentrated under reduced pressure. The resulting residue was suspended in acetonitrile and water and then lyophilized to afford 7-amino-3,4-dihydro-1H-1,5-naphthyridin-2-one HCl (20 mg, 0.12 mmol, 65% yield). MS (ESI) m/z: 164.0 [M+1].

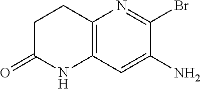 [SMILES: Nc1cc2c(nc1Br)CCC(=O)N2]

7-amino-6-bromo-3,4-dihydro-1,5-naphthyridin-2(1H)-one. To a solution of 7-amino-3,4-dihydro-1H-1,5-naphthyridin-2-one HCl (0.020 g, 0.12 mmol) in dichloromethane (1 mL) at 0° C. was added 1-bromopyrrolidine-2,5-dione (0.017 g, 0.10 mmol). The resulting mixture was stirred at 0° C. for 1 hour. The reaction mixture was filtered and the filtrate was concentrated under reduce pressure. The resulting residue was purified by silica gel column chromatography (petroleum ether:ethyl acetate) to give 7-amino-6-bromo-3,4-dihydro-1,5-naphthyridin-2(1H)-one (10 mg, 0.04 mmol, 34% yield). ¹H NMR (400 MHz, DMSO-d6) δ 10.08 (s, 1H), 6.61 (s, 1H), 5.39 (s, 2H), 2.81 (t, J=7.6 Hz, 2H), 2.52-2.49 (m, 2H).

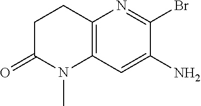

7-amino-6-bromo-1-methyl-3,4-dihydro-1,5-naphthyridin-2(1H)-one. 7-amino-6-bromo-3,4-dihydro-1,5-naphthyridin-2(1H)-one (0.010 g, 0.04 mmol) was dissolved in tetrahydrofuran (1 mL) and cooled to 0° C. Potassium bis(trimethylsilyl)amide (0.008 g, 0.04 mmol) was then added dropwise, followed by dropwise addition of iodomethane (0.006 g, 0.04 mmol). The mixture was stirred for 12 hours at 25° C. The reaction was quenched with water, and then partitioned between ethyl acetate and brine. The organic layer was dried over sodium sulfate, filtered and concentrated under reduced pressure. Purification by silica gel column chromatography (ethyl acetate/hexanes) provided 7-amino-6-bromo-1-methyl-3,4-dihydro-1,5-naphthyridin-2(1H)-one (5 mg, 0.02 mmol, 47% yield). ¹H NMR (400 MHz, DMSO-d6) δ 6.84 (s, 1H), 5.40 (s, 2H), 3.14 (s, 3H), 2.85-2.79 (m, 2H), 2.63-2.58 (m, 2H); MS (ESI) m/z: 258.0 [M+1]⁺.

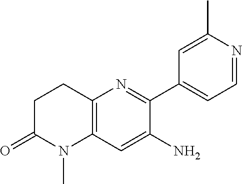 [SMILES: Cc1cc(-c2nc3c(cc2N)N(C)C(=O)CC3)ccn1]

7-amino-1-methyl-6-(2-methylpyridin-4-yl)-3,4-dihydro-1,5-naphthyridin-2(1H)-one. To a solution of 7-amino-6-bromo-1-methyl-3,4-dihydro-1,5-naphthyridin-2(1H)-one (70 mg, 0.27 mmol) in 1,4-dioxane (3 mL) and water (0.3 mL) was added 2-methyl-4-(4,4,5,5-tetramethyl-1,3,2-dioxaborolan-2-yl)pyridine (90 mg, 0.41 mmol), potassium carbonate (0.113 g, 0.82 mmol) and (1,1′-bis(diphenylphosphino)ferrocene)palladium(II) dichloride (21 mg, 0.028 mmol). The suspension was degassed under vacuum and purged with nitrogen. The mixture was stirred at 110° C. for 12 hours. The reaction mixture was concentrated in vacuo, and the resulting residue was purified by silica gel column chromatography (methanol in dichloromethane) to afford 7-amino-1-methyl-6-(2-methylpyridin-4-yl)-3,4-dihydro-1,5-naphthyridin-2(1H)-one (60 mg, 0.22 mmol, 82% yield). MS (ESI) m/z: 269.1 [M+1]⁺.

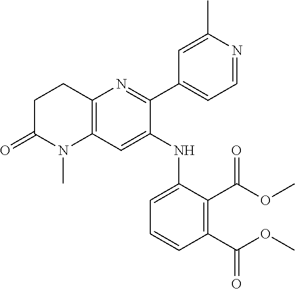 [SMILES: COC(=O)c1cccc(Nc2cc3c(nc2-c2ccnc(C)c2)CCC(=O)N3C)c1C(=O)OC]

dimethyl 3-((5-methyl-2-(2-methylpyridin-4-yl)-6-oxo-5,6,7,8-tetrahydro-1,5-naphthyridin-3-yl)amino)phthalate. To a solution of 7-amino-1-methyl-6-(2-methylpyridin-4-yl)-3,4-dihydro-1,5-naphthyridin-2(1H)-one (60 mg, 0.22 mmol) in 1,4-dioxane (3 mL) was added dimethyl 3-bromophthalate (73 mg, 0.27 mmol), cesium carbonate (0.218 g, 0.67 mmol) and methanesulfonato(2-dicyclohexylphosphino-2,6-di-i-propoxy-1,1-biphenyl)(2-amino-1,1-biphenyl-2-yl)palladium(II) (10 mg, 0.02 mmol). The suspension was degassed under vacuum and purged with nitrogen several times. The mixture was stirred at 110° C. for 12 hours. The reaction was filtered and the filtrate was concentrated under reduced pressure. The resulting crude residue was purified by silica gel column chromatography (petroleum ether/ethyl acetate) to give dimethyl 3-((5-methyl-2-(2-methylpyridin-4-yl)-6-oxo-5,6,7,8-tetrahydro-1,5-naphthyridin-3-yl)amino)phthalate (70 mg, 0.15 mmol, 68% yield). ¹H NMR (400 MHz, CDCl3) δ 8.46 (d, J=5.2 Hz, 1H), 7.96 (s, 1H), 7.40 (s, 1H), 7.26 (d, J=8.0 Hz, 1H), 7.15-7.07 (m, 4H), 3.82 (s, 3H), 3.75 (s, 3H), 3.22 (s, 3H), 3.11 (t, J=7.6 Hz, 2H), 2.76 (t, J=7.6 Hz, 2H), 2.52 (s, 3H); MS (ESI) m/z: 461.3 [M+1]⁺.

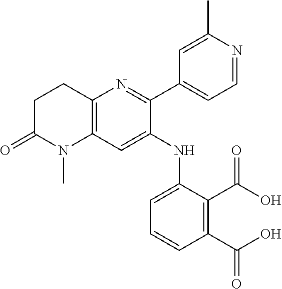 [SMILES: Cc1cc(-c2nc3c(cc2Nc2cccc(C(=O)O)c2C(=O)O)N(C)C(=O)CC3)ccn1]

3-((5-methyl-2-(2-methylpyridin-4-yl)-6-oxo-5,6,7,8-tetrahydro-1,5-naphthyridin-3-yl)amino)phthalic acid. To a solution of dimethyl 3-((5-methyl-2-(2-methylpyridin-4-yl)-6-oxo-5,6,7,8-tetrahydro-1,5-naphthyridin-3-yl)amino)phthalate (70 mg, 0.15 mmol) in methanol (3 mL) was added sodium hydroxide (52 mg, 1.30 mmol) in water (2 mL). The mixture was warmed to 80° C. and stirred for 12 hours. The reaction mixture was added to water and acidified to pH=6, followed by formation of a precipitate. The mixture was filtered, and the filtrate was extracted with DCM and washed with water. The combined organic phase was dried with sodium sulfate and filtered. The filtrate was concentrated under reduced pressure to give 3-((5-methyl-2-(2-methylpyridin-4-yl)-6-oxo-5,6,7,8-tetrahydro-1,5-naphthyridin-3-yl)amino)phthalic acid (45 mg, 0.10 mmol, 80% yield). MS (ESI) m/z: 433.2 [M+1]⁺.

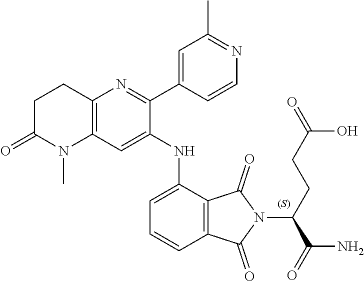

(S)-5-amino-4-(4-((5-methyl-2-(2-methylpyridin-4-yl)-6-oxo-5,6,7,8-tetrahydro-1,5-naphthyridin-3-yl)amino)-1,3-dioxoisoindolin-2-yl)-5-oxopentanoic acid. To a solution of 3-((5-methyl-2-(2-methylpyridin-4-yl)-6-oxo-5,6,7,8-tetrahydro-1,5-naphthyridin-3-yl)amino)phthalic acid (0.045 g, 0.10 mmol) and tert-butyl (S)-4,5-diamino-5-oxopentanoate (0.025 g, 0.12 mmol) in toluene (3 mL) was added triethylamine (0.112 g, 1.11 mmol). The resulting mixture was stirred at 130° C. for 12 hours. The reaction mixture was concentrated under reduce pressure to give (S)-5-amino-4-(4-((5-methyl-2-(2-methylpyridin-4-yl)-6-oxo-5,6,7,8-tetrahydro-1,5-naphthyridin-3-yl)amino)-1,3-dioxoisoindolin-2-yl)-5-oxopentanoic acid (0.050 g, 0.09 mmol, 89% yield). MS (ESI) m/z: 543.2 [M+1]⁺.

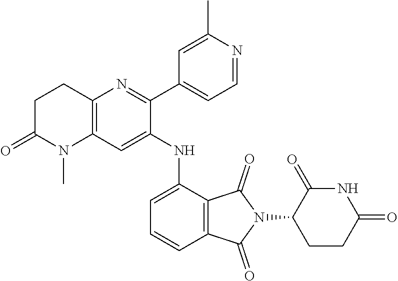 [SMILES: Cc1cc(-c2nc3c(cc2Nc2cccc4c2C(=O)N([C@H]2CCC(=O)NC2=O)C4=O)N(C)C(=O)CC3)ccn1]

(S)-2-(2,6-dioxopiperidin-3-yl)-4-((5-methyl-2-(2-methylpyridin-4-yl)-6-oxo-5,6,7,8-tetrahydro-1,5-naphthyridin-3-yl)amino)isoindoline-1,3-dione. To a solution of (S)-5-amino-4-(4-((5-methyl-2-(2-methylpyridin-4-yl)-6-oxo-5,6,7,8-tetrahydro-1,5-naphthyridin-3-yl)amino)-1,3-dioxoisoindolin-2-yl)-5-oxopentanoic acid (0.050 g, 0.09 mmol) in acetonitrile (3 mL) was added p-toluenesulfonic acid (0.144 g, 0.84 mmol). The resulting mixture was stirred at 80° C. for 2 hours under nitrogen. The reaction mixture was quenched with dichloromethane and aqueous sodium bicarbonate and extracted with dichloromethane. The combined organic phase was dried over anhydrous sodium sulfate and filtered. The filtrate was concentrated under reduce pressure, and the resulting crude material was purified by silica gel column chromatography (methanol in dichloromethane) followed by preparative chiral SFC (isopropyl alcohol/acetonitrile]) and concentrated under reduced pressure to afford (S)-2-(2,6-dioxopiperidin-3-yl)-4-((5-methyl-2-(2-methylpyridin-4-yl)-6-oxo-5,6,7,8-tetrahydro-1,5-naphthyridin-3-yl)amino)isoindoline-1,3-dione (9.6 mg, 0.018 mmol, 32% yield). ¹H NMR (400 MHz, DMSO-d6) δ 11.12 (s, 1H), 8.54 (s, 1H), 8.39 (d, J=5.2 Hz, 1H), 7.58 (s, 1H), 7.55-7.52 (m, 1H), 7.50-7.47 (m, 1H), 7.46-7.44 (m, 1H), 7.18 (d, J=7.2 Hz, 1H), 6.94 (d, J=8.4 Hz, 1H), 5.14-5.07 (m, 1H), 3.25 (s, 3H), 3.13 (t, J=7.6 Hz, 2H), 2.95-2.84 (m, 1H), 2.77 (t, J=7.6 Hz, 2H), 2.65-2.60 (m, 1H), 2.58 (br s, 1H), 2.43 (s, 3H), 2.11-2.01 (m, 1H); MS (ESI) m/z: 525.2 [M+1]⁺.

### Example 19: Synthesis of Compound 50: 2-(2,6-dioxopiperidin-3-yl)-4-((1-methyl-6-(2-methylpyridin-4-yl)-2,2-dioxido-3,4-dihydro-1H-benzo[c][1,2]thiazin-7-yl)amino)isoindoline-1,3-dione

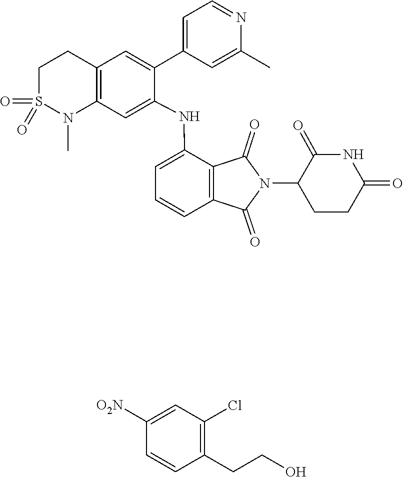 [SMILES: Cc1cc(-c2cc3c(cc2Nc2cccc4c2C(=O)N(C2CCC(=O)NC2=O)C4=O)N(C)S(=O)(=O)CC3)ccn1 ; O=[N+]([O-])c1ccc(CCO)c(Cl)c1]

2-(2-chloro-4-nitrophenyl)ethan-1-ol. To solution of 2-(2-chloro-4-nitrophenyl)acetic acid (5 g, 23.19 mmol) in Tetrahydrofuran (60 mL) at 0° C. was added BH₃·DMS (5.80 mL, 58.0 mmol) and stirred for 30 min at room temperature. Reaction mixture was heated at 65° C. and stirred for overnight. Reaction mixture was quenched with MeOH and heated at 70° C. and stirred overnight. Reaction mixture was concentrated under reduced pressure. The crude compound was purified by column chromatography on silica gel using EtOAc in pet ether to obtain 2-(2-chloro-4-nitrophenyl)ethan-1-ol (3.8 g, 18.47 mmol, 80% yield) as pale yellow solid.

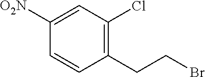 [SMILES: O=[N+]([O-])c1ccc(CCBr)c(Cl)c1]

1-(2-bromoethyl)-2-chloro-4-nitrobenzene. To a solution of 2-(2-chloro-4-nitrophenyl)ethan-1-ol (3.8 g, 18.85 mmol) and triphenylphosphine (8.16 g, 31.1 mmol) in DCM (10 mL) at 0° C. and stored for 15 min was added CBr₄ (7.81 g, 23.56 mmol) and stirred for overnight at room temperature. Solvent was evaporated to get crude compound. The crude compound was purified by column chromatography on silica gel using EtOAc in pet ether, concentrated compound fraction to obtain 1-(2-bromoethyl)-2-chloro-4-nitrobenzene (4 g, 14.97 mmol, 79% yield) as pale yellow liquid.

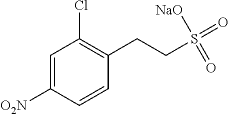

sodium 2-(2-chloro-4-nitrophenyl)ethane-1-sulfonate. To a solution of 1-(2-bromoethyl)-2-chloro-4-nitrobenzene (3.6 g, 13.61 mmol) in Ethanol (20 mL) and Water (31 mL), was added sodium sulfite (1.715 g, 13.61 mmol). Reaction mixture was heated at 100° C., and stirred for overnight. Reaction mixture was concentrated under reduced pressure to get crude compound. To the crude compound was added EtOH:H₂O mixture (1:1.5) and the mixture was kept in fridge overnight. Solid was precipitate collected by filteration and dried over 48 h to obtain sodium 2-(2-chloro-4-nitrophenyl)ethane-1-sulfonate (2.6 g, 8.41 mmol, 62% yield) as an offwhite solid. MS (ESI) m/z: 264.0 [M−Na]⁻.

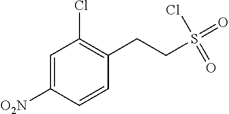 [SMILES: O=[N+]([O-])c1ccc(CCS(=O)(=O)Cl)c(Cl)c1]

2-(2-chloro-4-nitrophenyl)ethane-1-sulfonyl chloride. To a solution of sodium 2-(2-chloro-4-nitrophenyl)ethane-1-sulfonate (2.6 g, 9.04 mmol) in Toluene (20 mL) at room temperature was added SOCl₂ (1.319 mL, 18.08 mmol) and DMF (0.070 mL, 0.904 mmol). The reaction mixture was heated at 85° C. and stirred for overnight. Reaction mixture was concentrated under reduced pressure to get 2-(2-chloro-4-nitrophenyl)ethane-1-sulfonyl chloride (2.8 g, 93% yield) as pale yellow gum. The crude compound was used directly in next step.

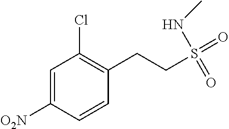 [SMILES: CNS(=O)(=O)CCc1ccc([N+](=O)[O-])cc1Cl]

2-(2-chloro-4-nitrophenyl)-N-methylethane-1-sulfonamide. To solution of 2-(2-chloro-4-nitrophenyl)ethane-1-sulfonyl chloride (2.6 g, 9.15 mmol) in DCM (20 mL) at 0° C. was added methanamine in THE (13.73 mL, 27.5 mmol) and stirred for overnight at room temperature. Reaction mixture was diluted with DCM and washed with water. Organic layer was dried over Na₂SO₄ and solvent was evaporated to obtained crude compound. The crude compound was purified by column chromatography on silica gel using Acetone in pet ether to obtained 2-(2-chloro-4-nitrophenyl)-N-methylethane-1-sulfonamide (1400 mg, 4.67 mmol, 51% yield) as offwhite solid. MS (ESI) m/z: 277.0 [M−H]⁻.

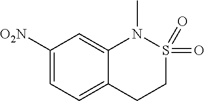 [SMILES: CN1c2cc([N+](=O)[O-])ccc2CCS1(=O)=O]

1-methyl-7-nitro-3,4-dihydro-1H-benzo[c][1,2]thiazine 2,2-dioxide. To a solution of 2-(2-chloro-4-nitrophenyl)-N-methylethane-1-sulfonamide (900 mg, 3.23 mmol) in 1,4-Dioxane (6 mL) was added cesium carbonate (3156 mg, 9.69 mmol). Reaction mixture was de-gassed with N₂ for 10 min. Then Xphos Pd-G2 (254 mg, 0.323 mmol) was added. Reaction mixture was stirred for 1 h at 125° C. under N₂. Reaction mixture was diluted with EtOAc and washed with water. Organic layer was dried over Na₂SO₄, and concentrated under reduced pressure to get crude compound. The crude compound was purified by column chromatography on silica gel using EtOAc in pet ether, concentrated compound fraction to obtain 1-methyl-7-nitro-3,4-dihydro-1H-benzo[c][1,2]thiazine 2,2-dioxide (720 mg, 2.97 mmol, 92% yield) as pale brick solid.

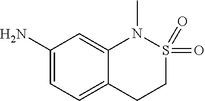

7-amino-1-methyl-3,4-dihydro-1H-benzo[c][1,2]thiazine 2,2-dioxide. To a suspension of 1-methyl-7-nitro-3,4-dihydro-1H-benzo[c][1,2]thiazine 2,2-dioxide (620 mg, 2.56 mmol)) in Ethanol (24 mL) and Water (6 mL) was added iron (715 mg, 12.80 mmol) and ammonium chloride (821 mg, 15.36 mmol). Reaction mixture was heated to 80° C. and stirred for 2 h. Reaction mixture was filtered through celite bed and washed with MeOH. Filter solvent was concentrated under reduced pressure and diluted with EtOAc and washed with 10% NaHCO₃. Organic layer was dried over Na₂SO₄ and solvent was evaporated to get 7-amino-1-methyl-3,4-dihydro-1H-benzo[c][1,2]thiazine 2,2-dioxide (500 mg, 1.790 mmol, 70% yield) as an off white solid. MS (ESI) m/z: 213.1 [M+1]⁺.

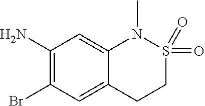

7-amino-6-bromo-1-methyl-3,4-dihydro-1H-benzo[c][1,2]thiazine 2,2-dioxide. To a solution of 7-amino-1-methyl-3,4-dihydro-1H-benzo[c][1,2]thiazine 2,2-dioxide (250 mg, 1.178 mmol) in DCM (9 mL) and MeOH (1 mL) at 0° C. was added mono(tetrabutylammonium) tribromide (568 mg, 1.178 mmol) and stirred for 2 h. The reaction mixture was diluted with DCM and washed with NaHCO₃ solution. Organic layer was dried over Na₂SO₄. Solvent was evaporated to get crude compound. The crude compound was purified by column chromatography on silica gel using EtOAc in PE. Compound fraction was concentrated to obtain 7-amino-6-bromo-1-methyl-3,4-dihydro-1H-benzo[c][1,2]thiazine 2,2-dioxide (380 mg, 1.266 mmol, 100% yield) as pale brick solid. MS (ESI) m/z: 292.9 [M+1]⁺.

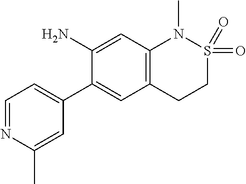 [SMILES: Cc1cc(-c2cc3c(cc2N)N(C)S(=O)(=O)CC3)ccn1]

7-amino-1-methyl-6-(2-methylpyridin-4-yl)-3,4-dihydro-1H-benzo[c][1,2]thiazine 2,2-dioxide. To a solution of 7-amino-6-bromo-1-methyl-3,4-dihydro-1H-benzo[c][1,2]thiazine 2,2-dioxide (180 mg, 0.618 mmol) and (2-methylpyridin-4-yl)boronic acid (102 mg, 0.742 mmol) in 1,2-Dimethoxyethane (8 mL) and 1,2-Dimethoxyethane (8 mL) was added Na₂CO₃ (3.71 mL, 1.855 mmol). The suspension was degassed with nitrogen for 10 min. To the reaction mixture as added Pd(Ph₃P)₄ (35.7 mg, 0.031 mmol). The reaction mixture was heated to 90° C. and stirred overnight. Reaction mixture was diluted with EtOAc and washed with water. The organic layer was dried over Na₂SO₄ and concentrated under reduced pressure to get crude compound. The crude compound was purified by prep HPLC to obtain 7-amino-1-methyl-6-(2-methylpyridin-4-yl)-3,4-dihydro-1H-benzo[c][1,2]thiazine 2,2-dioxide (100 mg, 0.303 mmol, 49% yield) as pale yellow gum. MS (ESI) m/z: 304.1 [M+1]⁺.

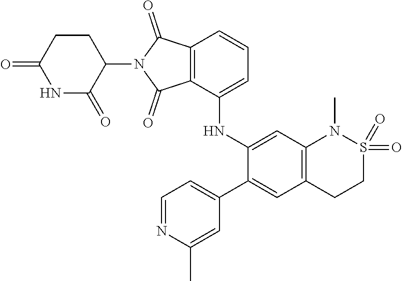 [SMILES: Cc1cc(-c2cc3c(cc2Nc2cccc4c2C(=O)N(C2CCC(=O)NC2=O)C4=O)N(C)S(=O)(=O)CC3)ccn1]

2-(2,6-dioxopiperidin-3-yl)-4-((1-methyl-6-(2-methylpyridin-4-yl)-2,2-dioxido-3,4-dihydro-1H-benzo[c][1,2]thiazin-7-yl)amino)isoindoline-1,3-dione. To a solution of 7-amino-1-methyl-6-(2-methylpyridin-4-yl)-3,4-dihydro-1H-benzo[c][1,2]thiazine 2,2-dioxide (80 mg, 0.264 mmol) in 1,4-Dioxane (4 mL) was added 4-bromo-2-(2,6-dioxopiperidin-3-yl)isoindoline-1,3-dione (116 mg, 0.343 mmol) and K₂CO₃ (109 mg, 0.791 mmol). Reaction mixture was degassed with N₂ for 15 min and was added XPhos Pd G2 (41.5 mg, 0.053 mmol). Reaction mixture was heated at 115° C. and stirred for overnight. Reaction mixture was diluted with EtOAc and washed with water. Organic layer was dried over Na₂SO₄ and solvent was evaporated to get crude compound. The crude compound was purified by prep HPLC to get 2-(2,6-dioxopiperidin-3-yl)-4-((1-methyl-6-(2-methylpyridin-4-yl)-2,2-dioxido-3,4-dihydro-1H-benzo[c][1,2]thiazin-7-yl)amino)isoindoline-1,3-dione (52 mg, 0.091 mmol, 35% yield) as yellow solid. MS (ESI) m/z: 560.2 [M+1]⁺. ¹H NMR (500 MHz, DMSO-d6) δ 11.11 (s, 1H), 8.38-8.33 (m, 2H), 7.47 (t, J=7.8 Hz, 1H), 7.40 (s, 1H), 7.32 (s, 1H), 7.26-7.22 (m, 1H), 7.21-7.19 (m, 1H), 7.14 (d, J=7.2 Hz, 1H), 6.98 (d, J=8.5 Hz, 1H), 5.14-5.04 (m, 1H), 3.61 (t, J=6.8 Hz, 2H), 3.42 (br t, J=6.9 Hz, 2H), 3.24 (s, 3H), 2.94-2.84 (m, 1H), 2.66-2.53 (m, 2H), 2.41 (s, 3H), 2.08-2.00 (m, 1H).

### Example 20: Synthesis of Compound 65: 2-(2,6-dioxopiperidin-3-yl)-4-((6-(3-fluoro-2-methoxypyridin-4-yl)-3-methyl-2-oxo-2,3-dihydrobenzo[d]oxazol-5-yl)amino)isoindoline-1,3-dione

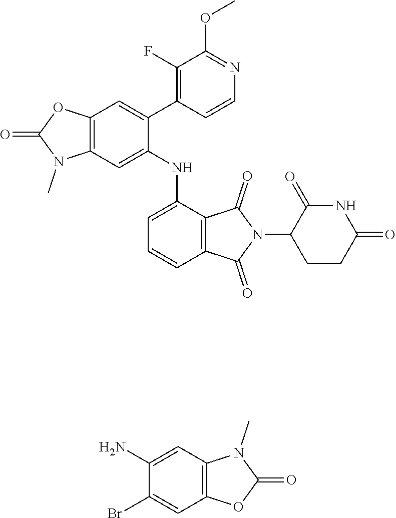 [SMILES: COc1nccc(-c2cc3oc(=O)n(C)c3cc2Nc2cccc3c2C(=O)N(C2CCC(=O)NC2=O)C3=O)c1F ; Cn1c(=O)oc2cc(Br)c(N)cc21]

5-amino-6-bromo-3-methylbenzo[d]oxazol-2(3H)-one. To a suspension of 5-amino-3-methylbenzo[d]oxazol-2(3H)-one (1 g, 6.09 mmol) in MeOH (2 mL) and DCM (18 mL) at 0° C. was added tetra-n-butylammonium tribromide (2.94 g, 6.09 mmol) and stirred for 2 h. Reaction mixture was diluted with DCM and washed with NaHCO₃. Organic layer was dried over Na₂SO₄ and concentrated to give crude compound. The crude compound was purified by column 139hromatography on silica gel using EtOAc in pet ether to obtained 5-amino-6-bromo-3-methylbenzo[d]oxazol-2(3H)-one (900 mg, 3.67 mmol, 60% yield) as pale yellow solid. MS (ESI) m/z: 244.1 [M+1]⁺.

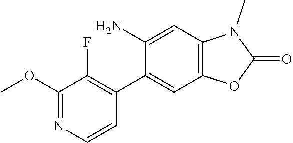 [SMILES: COc1nccc(-c2cc3oc(=O)n(C)c3cc2N)c1F]

5-amino-6-(3-fluoro-2-methoxypyridin-4-yl)-3-methylbenzo[d]oxazol-2(3H)-one. To a stirred solution of 5-amino-6-bromo-3-methylbenzo[d]oxazol-2(3H)-one (150 mg, 0.617 mmol) in 1,4-Dioxane (5 mL) and H₂O (1 mL) was added (3-fluoro-2-methoxypyridin-4-yl)boronic acid (211 mg, 1.234 mmol) and potassium carbonate (213 mg, 1.543 mmol)). The reaction mixture was purged with nitrogen for 20 min and was added Pd(dppf)Cl2·CH2Cl2 (50.4 mg, 0.062 mmol). The reaction mixture was stirred at 80° C. for 16 h. After completion of the reaction mixture was filtered through celite pad and wash with Ethyl Acetate and filtration was distilled under reduce pressure. Crude was purified by Flash column chromatography eluting EtOAc to afford 5-amino-6-(3-fluoro-2-methoxypyridin-4-yl)-3-methylbenzo[d]oxazol-2(3H)-one (60 mg, 0.124 mmol, 20% yield). MS (ESI) m/z: 290.2 [M+1]⁺.

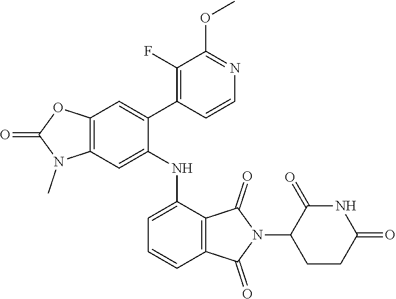 [SMILES: COc1nccc(-c2cc3oc(=O)n(C)c3cc2Nc2cccc3c2C(=O)N(C2CCC(=O)NC2=O)C3=O)c1F]

2-(2,6-dioxopiperidin-3-yl)-4-((6-(3-fluoro-2-methoxypyridin-4-yl)-3-methyl-2-oxo-2,3-dihydrobenzo[d]oxazol-5-yl)amino)isoindoline-1,3-dione. To a stirred solution of 5-amino-6-(3-fluoro-2-methoxypyridin-4-yl)-3-methylbenzo[d]oxazol-2(3H)-one (60 mg, 0.124 mmol) in Dioxane (3 mL) in a 50 ml sealed tube was added 4-bromo-2-(2,6-dioxopiperidin-3-yl)isoindoline-1,3-dione (88 mg, 0.261 mmol) and potassium carbonate (51.6 mg, 0.373 mmol). The reaction mixture was purged with nitrogen for 20 min and Xphos Pd G2 (25.4 mg, 0.031 mmol) was added. The reaction mixture was stirred in sealed tube at 115° C. for 16 h. After completion of Reaction mixture was filter through celite pad and wash with Ethyl Acetate and filtration was distilled under reduce pressure. Crude compound was purified by prep-HPLC purification to afford 2-(2,6-dioxopiperidin-3-yl)-4-((6-(3-fluoro-2-methoxypyridin-4-yl)-3-methyl-2-oxo-2,3-dihydrobenzo[d]oxazol-5-yl)amino)isoindoline-1,3-dione (17.93 mg, 0.033 mmol, 26% yield) as yellow solid. MS (ESI) m/z: 546.2 [M+1]⁺. ¹H NMR (500 MHz, DMSO-d6) δ 11.10 (s, 1H), 8.12 (s, 1H), 7.93 (d, J=5.1 Hz, 1H), 7.56-7.48 (m, 3H), 7.17-7.16 (m, 1H), 7.04-6.98 (m, 2H), 5.07 (dd, J=12.9, 5.4 Hz, 1H), 3.91 (s, 3H), 3.38 (s, 3H), 2.88 (br s, 1H), 2.65-2.53 (m, 2H), 2.06-1.98 (m, 1H).

### In Vitro Assays

WIZ Assay and ZBTB7A EC50 Assays. The HiBiT degradation assays for WIZ and ZBTB7A in HUDEP-2 cells were performed as described below. HUDEP-2 cells engineered to express a HiBiT tag in either the WIZ (Widely interspaced zinc finger) or ZBTB7A (Zinc Finger And BTB Domain Containing 7A) proteins were maintained in low attachment flasks in StemSpan SFEM II media supplemented with Penicillin-Streptomycin (50 U/mL), rhSCF (50 ng/mL), rhEPO (3 IU/mL), dexamethasone (0.4 μg/mL) and doxycycline (1 μg/mL). Prior to start of the assay, cell count and viability were measured by trypan blue exclusion using the Vi-cell XR cell viability analyzer.

For the HUDEP-2 WIZ HiBiT assay, cells were transferred to a 50 mL conical and centrifuged at 500 g for five minutes. Cells were resuspended in fresh StemSpan SFEM II media supplemented with Penicillin-Streptomycin (50 U/mL), rhSCF (50 ng/mL), rhEPO (3 IU/mL), dexamethasone (0.4 μg/mL) and doxycycline (1 μg/mL) at a density of 1.0×10⁶ cells/ml. Forty microliters of cell suspension was dispensed into 384-well Low Flange White Flat Bottom Polystyrene TC-treated Microplates containing pre-dispensed compounds, using a VIAFLO 384 liquid hander, and placed in a 37° C. incubator with 5% CO₂. Each compound was dispensed in duplicate and had a final DMSO concentration of 0.1%. After 24 hours of treatment, 40 μL Nano-Glo HiBiT Lytic Detection System reagent was dispensed into each well using a VIAFLO 384 liquid hander. Plates were incubated for 25 minutes at room temperature and the luminescence was read as relative luminescent units using the EnVision plate reader. EC50 and Y-min values were calculated using curves calculated from dotmatics software after the normalization to the DMSO control of 10 different concentrations: 10, 3.33, 1.11, 0.37, 0.12, 0.04 0.0137, 0.0046, 0.0015 and 0.0005 M respectively.

For the HUDEP-2 ZBTB7A HiBiT assay, cell count and viability were measured by trypan blue exclusion using the Vi-cell XR cell viability analyzer and the cells were transferred to a 50 mL conical and centrifuged at 500 g for five minutes. Cells were resuspended in fresh IMDM media supplemented with L-Glutamine (1×), Penicillin-Streptomycin (50 U/mL), holo-transferrin (330 μg/mL), heparin (2 IU/mL), r-human Insulin (10 μg/mL), rhEPO (3 IU/mL), rhSCF (100 ng/mL)+Dox (1 μg/mL) and 5% human plasma in low attachment flasks, which were placed in a 37° C. incubator with 5% CO₂. After 48 hours, cell count and viability were measured by trypan blue exclusion using the Vi-cell XR cell viability analyzer. The HUDEP-2 ZBTB7A cells were transferred to a 50 mL conical, centrifuged at 500 g for five minutes and resuspended at a density of 1.0×10⁶ cells/ml in IMDM media supplemented with L-Glutamine (1×), Penicillin-Streptomycin (50 U/mL), holo-transferrin (330 μg/mL), heparin (2 IU/mL), r-human Insulin (10 μg/mL), rhEPO (3 IU/mL), rhSCF (100 ng/mL)+Dox (1 μg/mL) and 5% human plasma. Forty microliters of cell suspension was dispensed into 384-well Low Flange White Flat Bottom Polystyrene TC-treated Microplates containing pre-dispensed compounds, using a VIAFLO 384 liquid hander, and placed in a 37° C. incubator with 5% CO₂. Each compound was dispensed in duplicate and had a final DMSO concentration of 0.1%. After 24 hours of treatment, 40 μL Nano-Glo HiBiT Lytic Detection System reagent was dispensed into each well using a VIAFLO 384 liquid hander. Plates were incubated for 25 minutes at room temperature and the luminescence was read as relative luminescent units using the EnVision plate reader. EC50 and Y-min values were calculated using curves calculated from dotmatics software after the normalization to the DMSO control of 10 different concentrations: 10, 3.33, 1.11, 0.37, 0.12, 0.04 0.0137, 0.0046, 0.0015 and 0.0005 M respectively.

Results from the HiBiT degradation assays and EC50 values for WIZ and ZBTB7A degradation in HUDEP-2 cells are reported in Table 2.

HbF HiBiT induction assay. The HiBiT induction assay for HbF in HUDEP-2 cells was performed as described below. HUDEP-2 cells engineered to express a HiBiT tag in HBG1/2 were maintained at a density of 0.1-1×10⁶ cells/ml in StemSpan SFEM II supplemented with 1% penicillin/streptomycin, hSCF (50 ng/mL), erythropoietin (EPO; 3 IU/mL), dexamethasone (DEX; 0.4 μg/mL), and doxycycline hyclate (DOX; 1 μg/mL). The cells were cultured in low adherence flasks, in a humidified incubator, set at 37° C. with 5% CO₂. Cell counts and viability were measured by trypan blue exclusion using the Vi-CELL XR cell viability analyzer and the cell growth was consistently maintained in log phase.

Prior to the start of experiments, HbF HiBiT-tagged HUDEP-2 cells were transferred to a 50 mL conical and centrifuged at 500×g for 5 minutes, the supernatant removed, and the pellet washed in 5 mL of 1×PBS. The washed cells were centrifuged again at 500×g for 5 minutes and resuspended in IMDM containing; 1×glutamine, 1×penicillin/streptomycin, holo-transferrin (330 μg/mL), heparin (2 IU/mL), r-human insulin (10 μg/mL), EPO (3 IU/mL), 5% human plasma, hSCF (100 ng/mL) and DOX (1 μg/mL) to a final density of 0.3×10⁶ cells/mL. Forty microliters of cell suspension were dispensed into 384-well Low Flange White Flat Bottom Polystyrene TC-treated Microplates containing pre-dispensed compounds, using a VIAFLO 384 liquid hander, and placed in a humidified incubator at 37° C. with 5% CO₂. Each compound was dispensed in duplicate and had a final DMSO concentration of 0.1%. After 72 hours of treatment, 40 μL of Nano-Glo HiBiT Lytic Solution (prepared according to manufacturer's instructions) were dispensed into each well using a VIAFLO 384 liquid handler, and the plates were shaken for 20 seconds. Plates were incubated for 20 minutes at room temperature, and the luminescence was read as relative luminescent units using an EnVision plate reader.

Dose response curves were generated, and EC₅₀, Yₘₐₓ, and then area under the curve (AUC) values were calculated. HbF induction was measured using the calculated area under the curve (AUC) value, with higher AUC values indicative of greater HbF induction relative to lower AUC values.

Results from the HbF HiBiT induction assay are reported in Table 2.

TABLE 2

| | | | HBF AUC |
|---|---|---|---|
| | | | (+ = 15 ≤ AUC < 100 |
| Compound | ZBTB7A | WIZ | ++ = 100 ≤ AUC < 150 |
| No. | EC₅₀ (μM) | EC₅₀ (μM) | +++ = AUC ≥ 150) |
| 1 | 0.018 | 0.021 | + |
| 2 | 0.011 | 0.006 | +++ |
| 3 | 0.029 | 0.017 | +++ |
| 4 | 0.026 | 0.083 | +++ |
| 5 | 0.019 | 0.015 | + |
| 6 | 0.049 | 0.009 | + |
| 7 | 0.009 | 0.007 | +++ |
| 8 | 0.021 | 0.015 | +++ |
| 9 | 0.032 | 0.024 | + |
| 10 | 0.020 | 0.012 | ++ |
| 11 | 0.017 | 0.021 | ++ |
| 12 | 0.034 | 0.037 | + |
| 13 | 0.038 | 0.050 | + |
| 14 | 0.017 | 0.012 | + |
| 15 | 0.022 | 0.007 | + |
| 16 | 0.073 | 0.032 | + |
| 17 | 0.018 | 0.013 | +++ |
| 18 | 0.011 | 0.011 | +++ |
| 19 | 0.021 | 0.028 | ++ |
| 20 | 0.012 | 0.014 | + |
| 21 | 0.012 | 0.017 | + |
| 22 | 0.013 | 0.017 | +++ |
| 23 | 0.015 | 0.011 | ++ |
| 24 | 0.026 | 0.014 | +++ |
| 25 | 0.024 | 0.018 | + |
| 26 | 0.098 | 0.013 | ++ |
| 27 | 0.011 | 0.006 | + |
| 28 | 0.013 | 0.011 | +++ |
| 29 | 0.023 | 0.005 | + |
| 30 | 0.019 | 0.011 | ++ |
| 31 | 0.035 | 0.006 | +++ |
| 32 | 0.014 | 0.005 | +++ |
| 33 | 0.024 | 0.021 | + |
| 34 | 0.011 | 0.011 | +++ |
| 35 | 0.008 | 0.011 | + |
| 36 | 0.019 | 0.015 | + |
| 37 | 0.013 | 0.007 | + |
| 38 | 0.013 | 0.014 | ++ |
| 39 | 0.016 | 0.013 | + |
| 40 | 0.016 | 0.012 | + |
| 41 | 0.017 | 0.010 | +++ |
| 42 | 0.018 | 0.009 | + |
| 43 | 0.024 | 0.015 | ++ |
| 44 | 0.006 | 0.004 | ++ |
| 45 | 0.010 | 0.006 | ++ |
| 46 | 0.007 | 0.002 | ++ |
| 47 | 0.049 | 0.043 | +++ |
| 48 | 0.052 | 0.031 | ++ |
| 49 | 0.071 | 0.039 | ++ |
| 50 | 0.028 | 0.018 | + |
| 51 | 0.030 | 0.037 | ++ |
| 52 | 0.143 | 0.012 | + |
| 53 | 0.010 | 0.004 | ++ |
| 54 | 0.014 | 0.008 | ++ |
| 55 | 0.009 | 0.006 | ++ |
| 56 | 0.043 | 0.020 | ++ |
| 57 | 0.054 | 0.018 | +++ |
| 58 | 0.010 | 0.009 | ++ |
| 59 | 0.010 | 0.008 | +++ |
| 60 | 0.011 | 0.009 | +++ |
| 61 | 0.011 | 0.007 | ++ |
| 62 | 0.019 | 0.017 | ++ |
| 63 | 0.014 | 0.016 | ++ |
| 64 | 0.053 | 0.012 | ++ |
| 65 | 0.096 | 0.041 | + |
| 66 | 0.188 | 0.015 | ++ |
| 67 | 0.025 | 0.017 | ++ |
| 68 | 0.006 | 0.003 | +++ |
| 69 | 0.054 | 0.020 | + |
| 70 | 0.015 | 0.019 | ++ |
| 71 | 0.010 | 0.016 | ++ |
| 72 | 0.021 | 0.014 | +++ |
| 73 | 0.011 | 0.012 | ++ |
| 74 | 0.010 | 0.011 | ++ |
| 75 | 0.075 | 0.037 | ++ |
| 76 | 0.065 | 0.022 | + |
| 77 | 0.102 | 0.112 | + |
| 78 | 0.070 | 0.076 | ++ |
| 79 | 0.028 | 0.027 | + |
| 80 | 0.038 | 0.029 | ++ |
| 81 | 0.028 | 0.024 | ++ |
| 82 | 0.019 | 0.054 | ++ |
| 83 | 0.013 | 0.008 | + |
| 84 | 0.019 | 0.007 | +++ |
| 85 | 0.043 | 0.026 | ND |
| 86 | 0.048 | 0.009 | + |
| 87 | 0.016 | 0.012 | + |
| 88 | 0.060 | 0.008 | + |
| 89 | 0.013 | 0.014 | + |
| 90 | 0.024 | 0.014 | ++ |
| 91 | 0.004 | 0.004 | ++ |
| 92 | 0.003 | 0.002 | + |
| 93 | 0.015 | 0.004 | ++ |
| 94 | 0.028 | 0.017 | + |
| 95 | 0.256 | 0.089 | + |
| 96 | 0.013 | 0.011 | ++ |

ND means no data.

A number of references have been cited, the disclosures of which are incorporated herein by reference in their entireties.

1. A compound of the following structural formula: 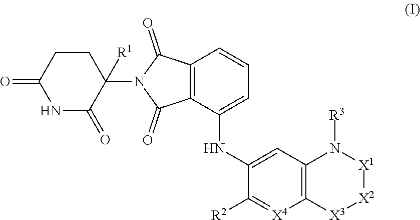
or a pharmaceutically acceptable salt, tautomer, isotopologue, and/or stereoisomer thereof, wherein:
X¹ is C(═O), C(═S), S(═O)₂, or C(R¹⁰)₂;
each R¹⁰ is independently H or (C₁-C₃)alkyl;
X² is C(R²⁰)₂ and X³ is O or C(R³⁰)₂; or X² is O and X³ is C(R³⁰)₂ or absent;
each R²⁰ is independently H or (C₁-C₃)alkyl;
each R³⁰ is independently H or (C₁-C₃)alkyl;
X⁴ is C(R⁴⁰) or N;
R⁴⁰ is H or F;
R¹ is H or methyl;
R² is five- to ten-membered heteroaryl, (C₆-C₁₀)aryl, three- to eight-membered heterocyclyl, or (C₃-C₈)cycloalkyl, and is optionally substituted with (R⁴)ₓ;
R³ is (C₁-C₃)alkyl, (C₁-C₃)alkenyl, or (C₃-C₆)cycloalkyl;
each R⁴ is independently cyano, halo, (C₁-C₃)alkyl, halo(C₁-C₃)alkyl, —O—(C₁-C₃)alkyl, (C₃-C₆)cycloalkyl, —C(O)(C₁-C₃)alkyl, —C(O)(C₃-C₆)cycloalkyl, —NH₂, —N(H)(C₁-C₃)alkyl, or —N((C₁-C₃)alkyl)₂; and
x is 1, 2, 3, or 4;
provided the compound is not

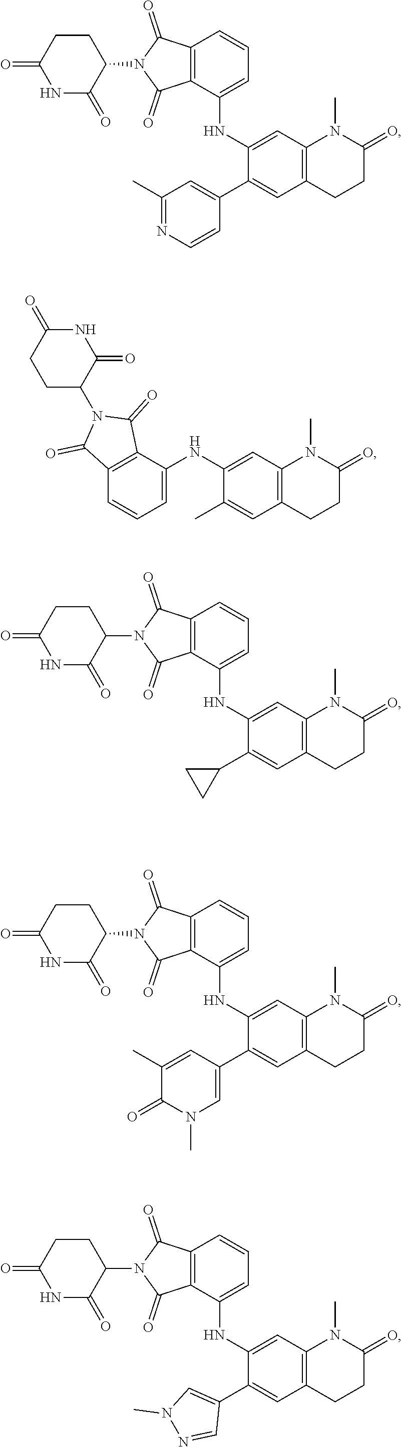 [SMILES: Cc1cc(-c2cc3c(cc2Nc2cccc4c2C(=O)N([C@H]2CCC(=O)NC2=O)C4=O)N(C)C(=O)CC3)ccn1 ; Cc1cc2c(cc1Nc1cccc3c1C(=O)N(C1CCC(=O)NC1=O)C3=O)N(C)C(=O)CC2 ; CN1C(=O)CCc2cc(C3CC3)c(Nc3cccc4c3C(=O)N(C3CCC(=O)NC3=O)C4=O)cc21 ; Cc1cc(-c2cc3c(cc2Nc2cccc4c2C(=O)N([C@H]2CCC(=O)NC2=O)C4=O)N(C)C(=O)CC3)cn(C)c1=O ; CN1C(=O)CCc2cc(-c3cnn(C)c3)c(Nc3cccc4c3C(=O)N(C3CCC(=O)NC3=O)C4=O)cc21]

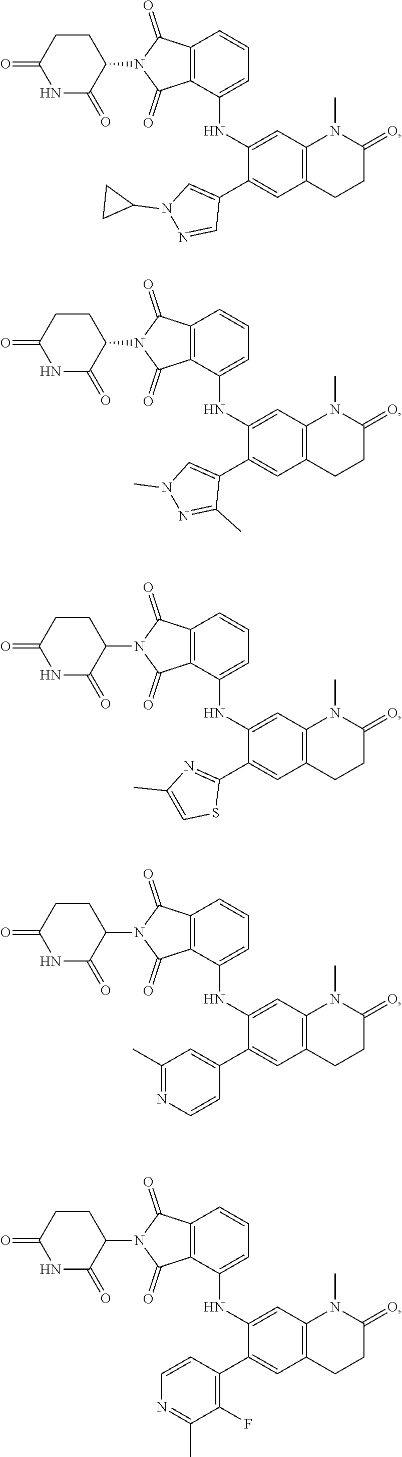 [SMILES: CN1C(=O)CCc2cc(-c3cnn(C4CC4)c3)c(Nc3cccc4c3C(=O)N([C@H]3CCC(=O)NC3=O)C4=O)cc21 ; Cc1nn(C)cc1-c1cc2c(cc1Nc1cccc3c1C(=O)N([C@H]1CCC(=O)NC1=O)C3=O)N(C)C(=O)CC2 ; Cc1csc(-c2cc3c(cc2Nc2cccc4c2C(=O)N(C2CCC(=O)NC2=O)C4=O)N(C)C(=O)CC3)n1 ; Cc1cc(-c2cc3c(cc2Nc2cccc4c2C(=O)N(C2CCC(=O)NC2=O)C4=O)N(C)C(=O)CC3)ccn1 ; Cc1nccc(-c2cc3c(cc2Nc2cccc4c2C(=O)N(C2CCC(=O)NC2=O)C4=O)N(C)C(=O)CC3)c1F]

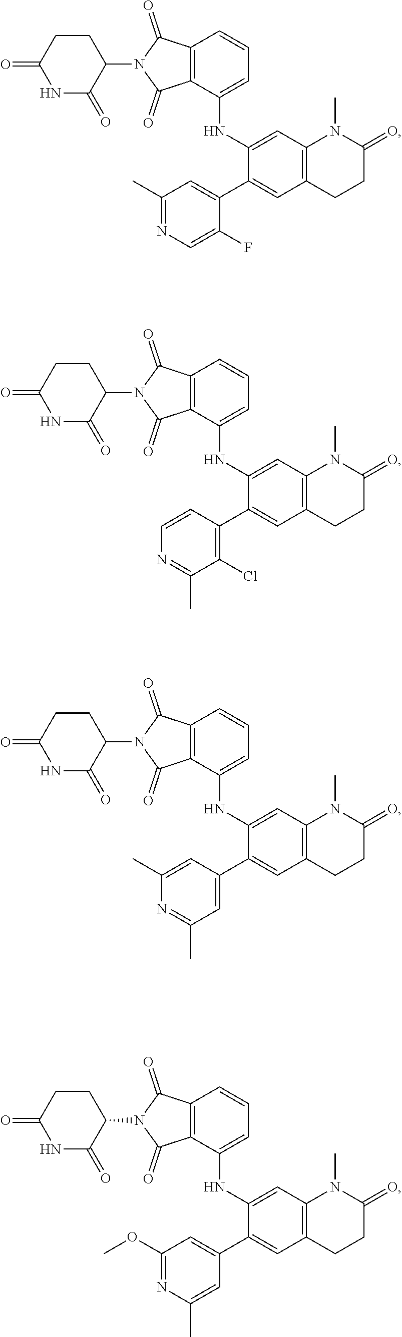 [SMILES: Cc1cc(-c2cc3c(cc2Nc2cccc4c2C(=O)N(C2CCC(=O)NC2=O)C4=O)N(C)C(=O)CC3)c(F)cn1 ; Cc1nccc(-c2cc3c(cc2Nc2cccc4c2C(=O)N(C2CCC(=O)NC2=O)C4=O)N(C)C(=O)CC3)c1Cl ; Cc1cc(-c2cc3c(cc2Nc2cccc4c2C(=O)N(C2CCC(=O)NC2=O)C4=O)N(C)C(=O)CC3)cc(C)n1 ; COc1cc(-c2cc3c(cc2Nc2cccc4c2C(=O)N([C@H]2CCC(=O)NC2=O)C4=O)N(C)C(=O)CC3)cc(C)n1]

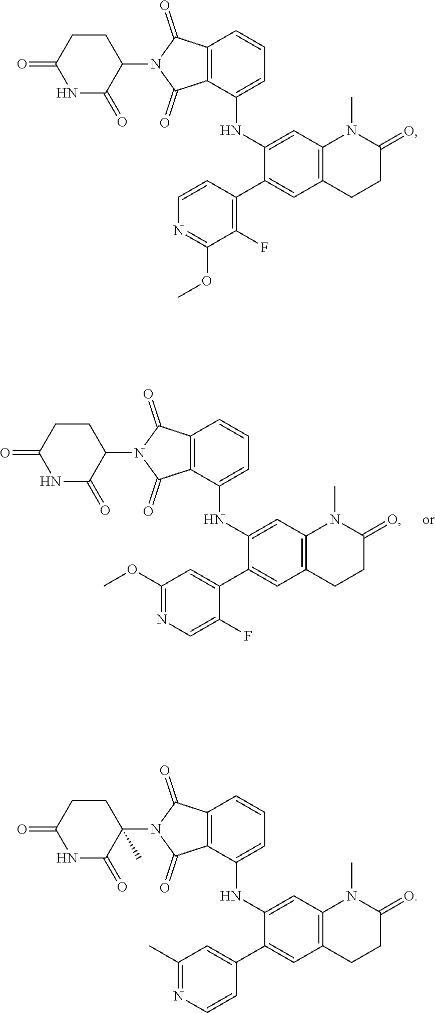 [SMILES: COc1nccc(-c2cc3c(cc2Nc2cccc4c2C(=O)N(C2CCC(=O)NC2=O)C4=O)N(C)C(=O)CC3)c1F ; COc1cc(-c2cc3c(cc2Nc2cccc4c2C(=O)N(C2CCC(=O)NC2=O)C4=O)N(C)C(=O)CC3)c(F)cn1 ; Cc1cc(-c2cc3c(cc2Nc2cccc4c2C(=O)N([C@]2(C)CCC(=O)NC2=O)C4=O)N(C)C(=O)CC3)ccn1]

2. (canceled)

3. (canceled)

4. (canceled)

5. The compound of claim 1, wherein X² is C(R²⁰)₂ and X³ is O or C(R³⁰)₂.

6. (canceled)

7. (canceled)

8. (canceled)

9. The compound of claim 1, wherein X² is O, and X³ is C(R³⁰)₂ or absent.

10. (canceled)

11. (canceled)

12. The compound of claim 1, wherein R¹ is H.

13. (canceled)

14. The compound of claim 1, wherein R² is pyridinyl, er phenyl, piperidinyl, or cyclohexyl, and is optionally substituted with (R⁴)ₓ.

15. (canceled)

16. (canceled)

17. The compound of claim 1, wherein each R⁴ is independently cyano, fluoro, methyl, difluoromethyl, methoxy, ethoxy, cyclopropyl, acetyl, cyclopropanecarbonyl, —NH₂, —N(H)CH₃, or —N(CH₃)₂.

18. The compound of claim 1, wherein x is 1, 2, or 3.

19. (canceled)

20. The compound of claim 1, wherein R² is

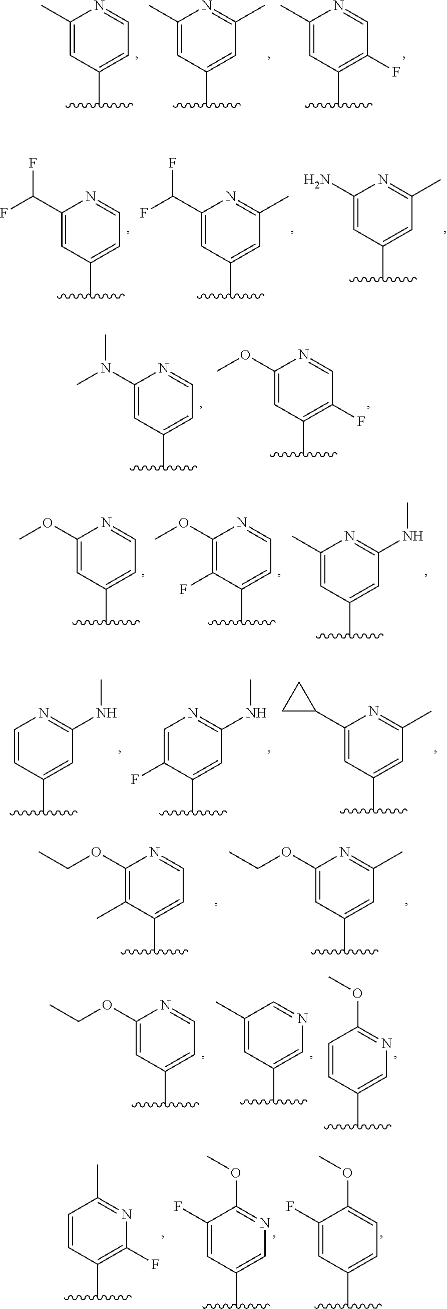

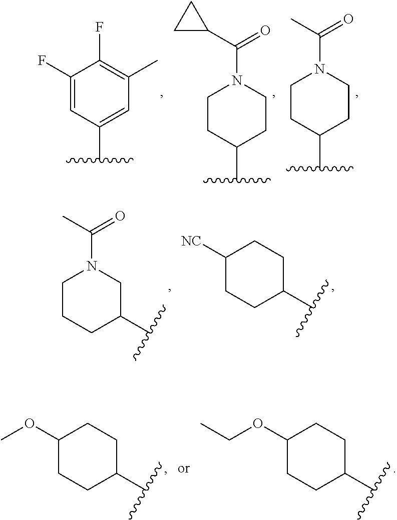

21. (canceled)

22. The compound of claim 1, wherein R³ is methyl, ethyl, vinyl, or cyclopropyl.

23. (canceled)

24. The compound of claim 1, of the following structural formula: 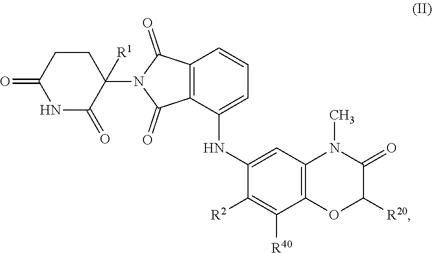
or a pharmaceutically acceptable salt, tautomer, isotopologue, and/or stereoisomer thereof.

25. The compound of claim 1, of the following structural formula: 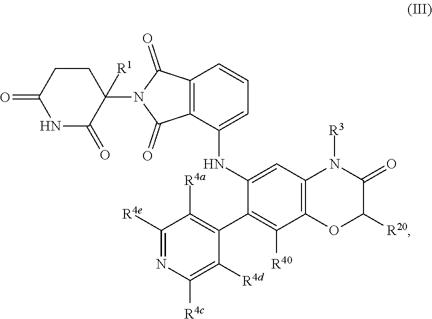
or a pharmaceutically acceptable salt, tautomer, isotopologue, and/or stereoisomer thereof, wherein:
R^{4a}, R^{4b}, R^{4c}, and R^{4d} are each independently H, cyano, halo, (C₁-C₃)alkyl, halo(C₁-C₃)alkyl, —O—(C₁-C₃)alkyl, (C₃-C₆)cycloalkyl, —C(O)(C₁-C₃)alkyl, —C(O)(C₃-C₆)cycloalkyl, —NH₂, —N(H)(C₁-C₃)alkyl, or —N((C₁-C₃)alkyl)₂.

26-31. (canceled)

32. The compound of claim 1, of the following structural formula: 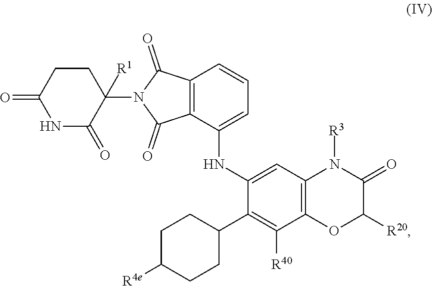
or a pharmaceutically acceptable salt, tautomer, isotopologue, and/or stereoisomer thereof, wherein:
R^{4e} is H, cyano, halo, (C₁-C₃)alkyl, halo(C₁-C₃)alkyl, —O—(C₁-C₃)alkyl, (C₃-C₆)cycloalkyl, —C(O)(C₁-C₃)alkyl, —C(O)(C₃-C₆)cycloalkyl, —NH₂, —N(H)(C₁-C₃)alkyl, or —N((C₁-C₃)alkyl)₂.

33. (canceled)

34. (canceled)

35. A compound of one of the following structural formulas, or a pharmaceutically acceptable salt, tautomer, isotopologue, and/or stereoisomer thereof;

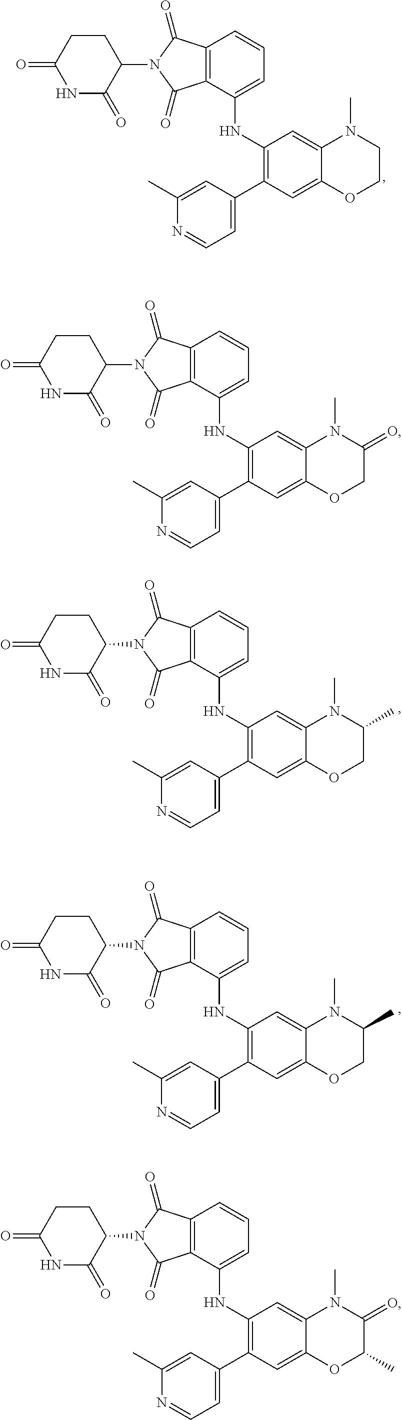 [SMILES: Cc1cc(-c2cc3c(cc2Nc2cccc4c2C(=O)N(C2CCC(=O)NC2=O)C4=O)N(C)CCO3)ccn1 ; Cc1cc(-c2cc3c(cc2Nc2cccc4c2C(=O)N(C2CCC(=O)NC2=O)C4=O)N(C)C(=O)CO3)ccn1 ; Cc1cc(-c2cc3c(cc2Nc2cccc4c2C(=O)N([C@H]2CCC(=O)NC2=O)C4=O)N(C)[C@H](C)CO3)ccn1 ; Cc1cc(-c2cc3c(cc2Nc2cccc4c2C(=O)N([C@H]2CCC(=O)NC2=O)C4=O)N(C)[C@@H](C)CO3)ccn1 ; Cc1cc(-c2cc3c(cc2Nc2cccc4c2C(=O)N([C@H]2CCC(=O)NC2=O)C4=O)N(C)C(=O)[C@H](C)O3)ccn1]

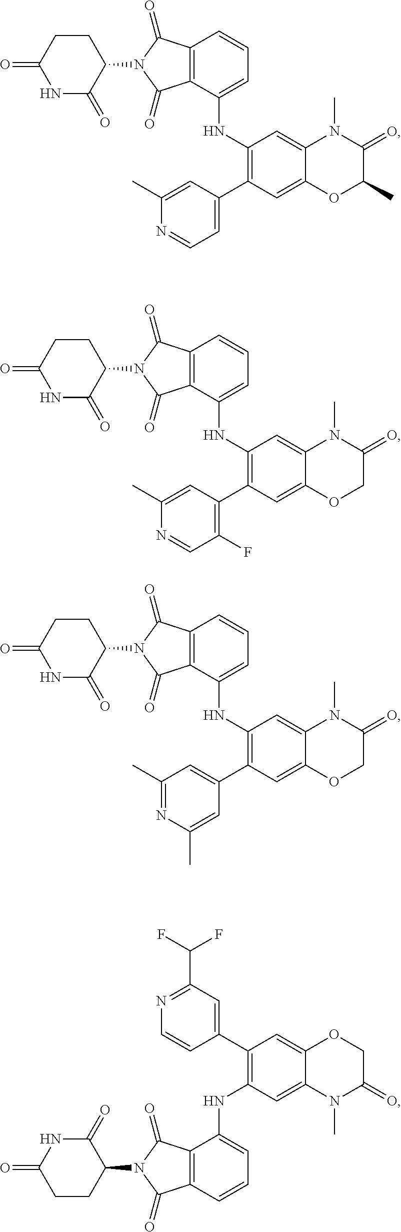 [SMILES: Cc1cc(-c2cc3c(cc2Nc2cccc4c2C(=O)N([C@H]2CCC(=O)NC2=O)C4=O)N(C)C(=O)[C@@H](C)O3)ccn1 ; Cc1cc(-c2cc3c(cc2Nc2cccc4c2C(=O)N([C@H]2CCC(=O)NC2=O)C4=O)N(C)C(=O)CO3)c(F)cn1 ; Cc1cc(-c2cc3c(cc2Nc2cccc4c2C(=O)N([C@H]2CCC(=O)NC2=O)C4=O)N(C)C(=O)CO3)cc(C)n1 ; CN1C(=O)COc2cc(-c3ccnc(C(F)F)c3)c(Nc3cccc4c3C(=O)N([C@H]3CCC(=O)NC3=O)C4=O)cc21]

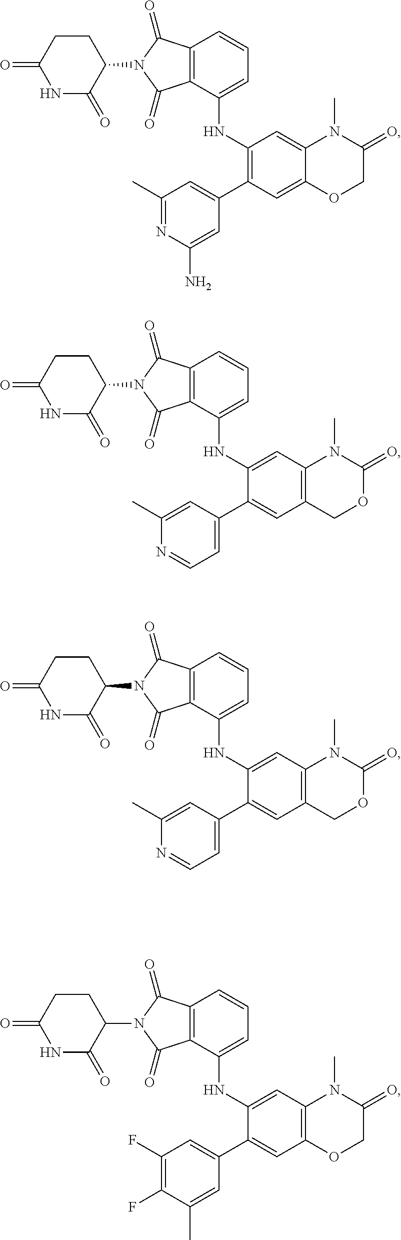 [SMILES: Cc1cc(-c2cc3c(cc2Nc2cccc4c2C(=O)N([C@H]2CCC(=O)NC2=O)C4=O)N(C)C(=O)CO3)cc(N)n1 ; Cc1cc(-c2cc3c(cc2Nc2cccc4c2C(=O)N([C@H]2CCC(=O)NC2=O)C4=O)N(C)C(=O)OC3)ccn1 ; Cc1cc(-c2cc3c(cc2Nc2cccc4c2C(=O)N([C@@H]2CCC(=O)NC2=O)C4=O)N(C)C(=O)OC3)ccn1 ; Cc1cc(-c2cc3c(cc2Nc2cccc4c2C(=O)N(C2CCC(=O)NC2=O)C4=O)N(C)C(=O)CO3)cc(F)c1F]

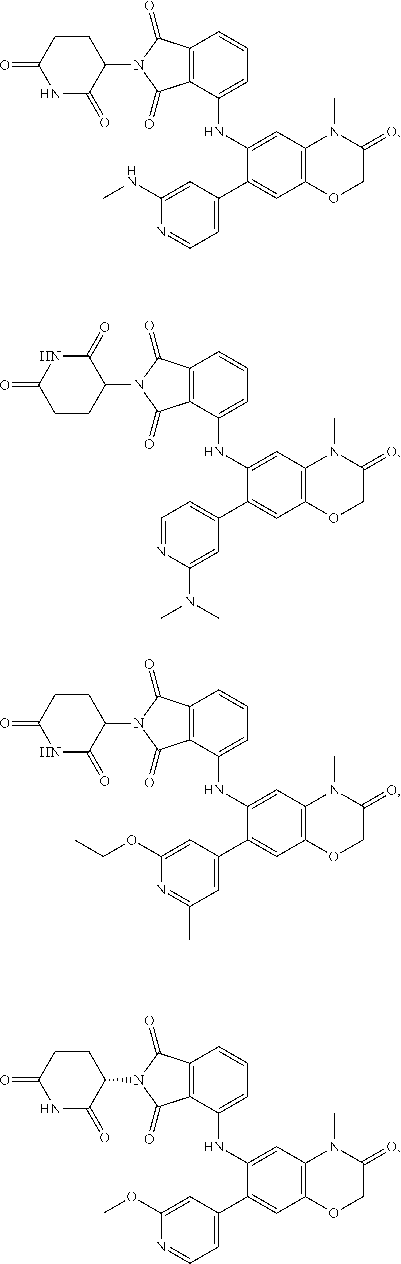 [SMILES: CNc1cc(-c2cc3c(cc2Nc2cccc4c2C(=O)N(C2CCC(=O)NC2=O)C4=O)N(C)C(=O)CO3)ccn1 ; CN(C)c1cc(-c2cc3c(cc2Nc2cccc4c2C(=O)N(C2CCC(=O)NC2=O)C4=O)N(C)C(=O)CO3)ccn1 ; CCOc1cc(-c2cc3c(cc2Nc2cccc4c2C(=O)N(C2CCC(=O)NC2=O)C4=O)N(C)C(=O)CO3)cc(C)n1 ; COc1cc(-c2cc3c(cc2Nc2cccc4c2C(=O)N([C@H]2CCC(=O)NC2=O)C4=O)N(C)C(=O)CO3)ccn1]

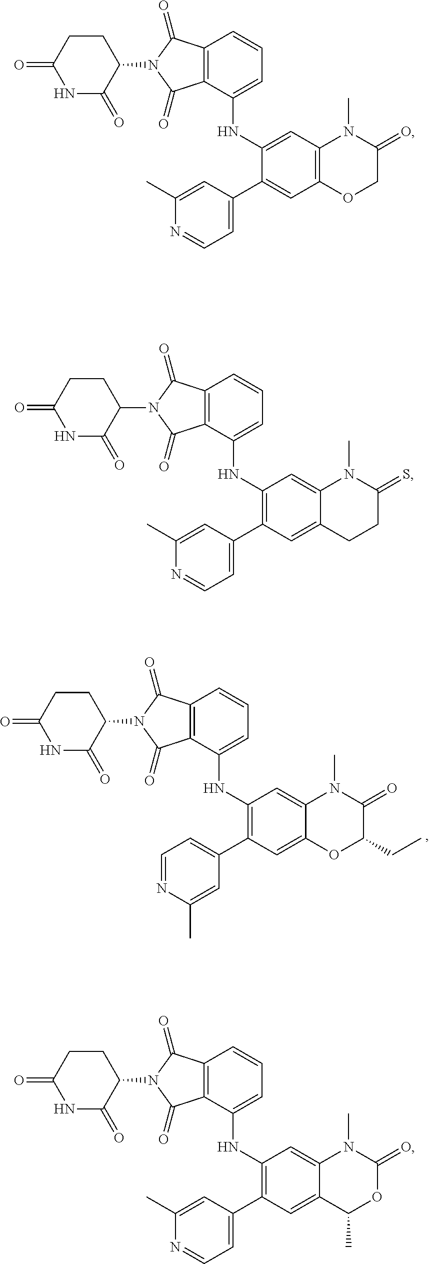 [SMILES: Cc1cc(-c2cc3c(cc2Nc2cccc4c2C(=O)N([C@H]2CCC(=O)NC2=O)C4=O)N(C)C(=O)CO3)ccn1 ; Cc1cc(-c2cc3c(cc2Nc2cccc4c2C(=O)N(C2CCC(=O)NC2=O)C4=O)N(C)C(=S)CC3)ccn1 ; CC[C@@H]1Oc2cc(-c3ccnc(C)c3)c(Nc3cccc4c3C(=O)N([C@H]3CCC(=O)NC3=O)C4=O)cc2N(C)C1=O ; Cc1cc(-c2cc3c(cc2Nc2cccc4c2C(=O)N([C@H]2CCC(=O)NC2=O)C4=O)N(C)C(=O)O[C@@H]3C)ccn1]

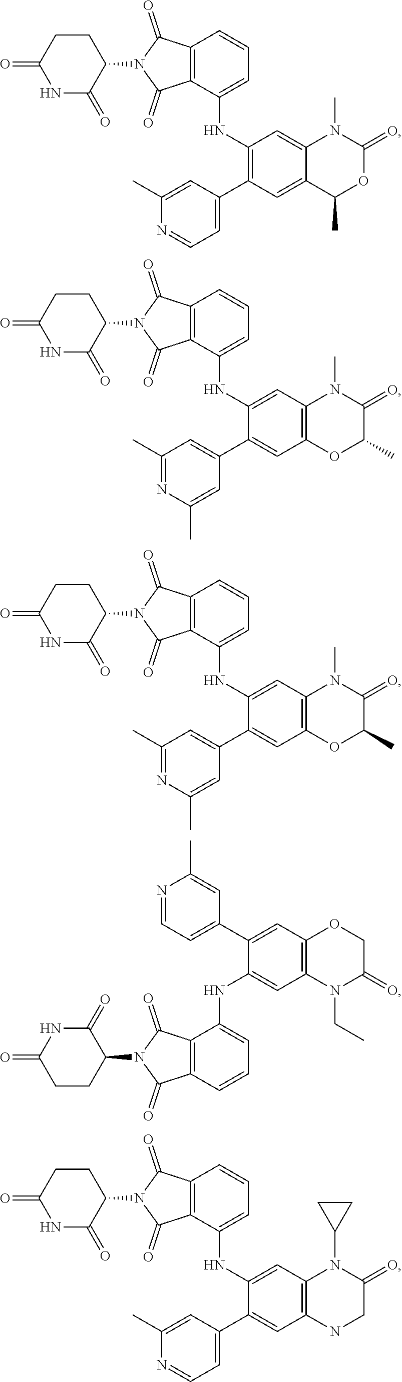

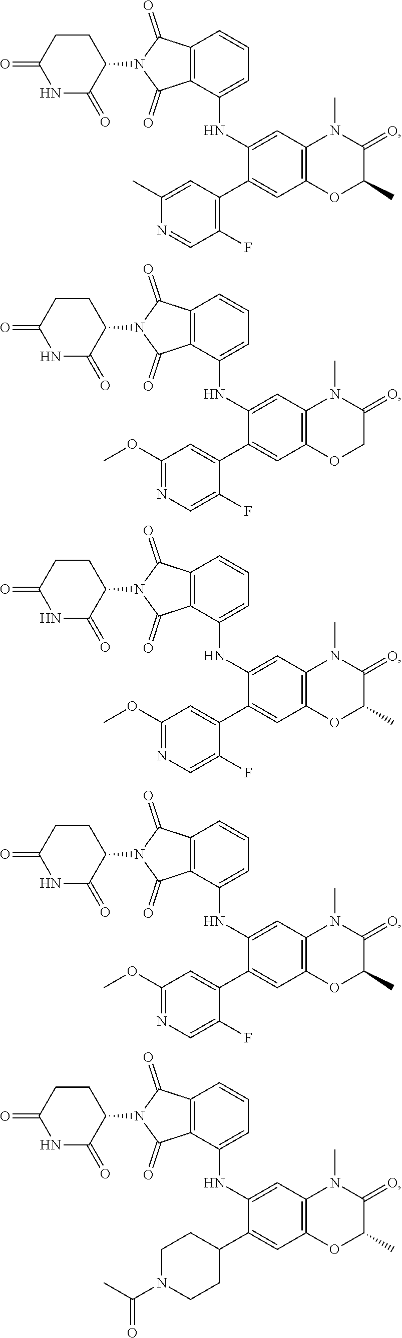

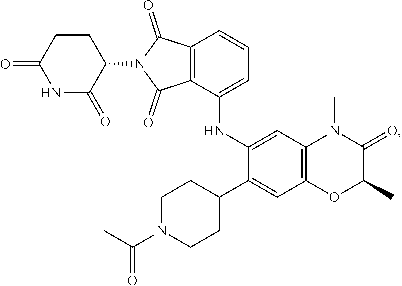 [SMILES: CC(=O)N1CCC(c2cc3c(cc2Nc2cccc4c2C(=O)N([C@H]2CCC(=O)NC2=O)C4=O)N(C)C(=O)[C@@H](C)O3)CC1]

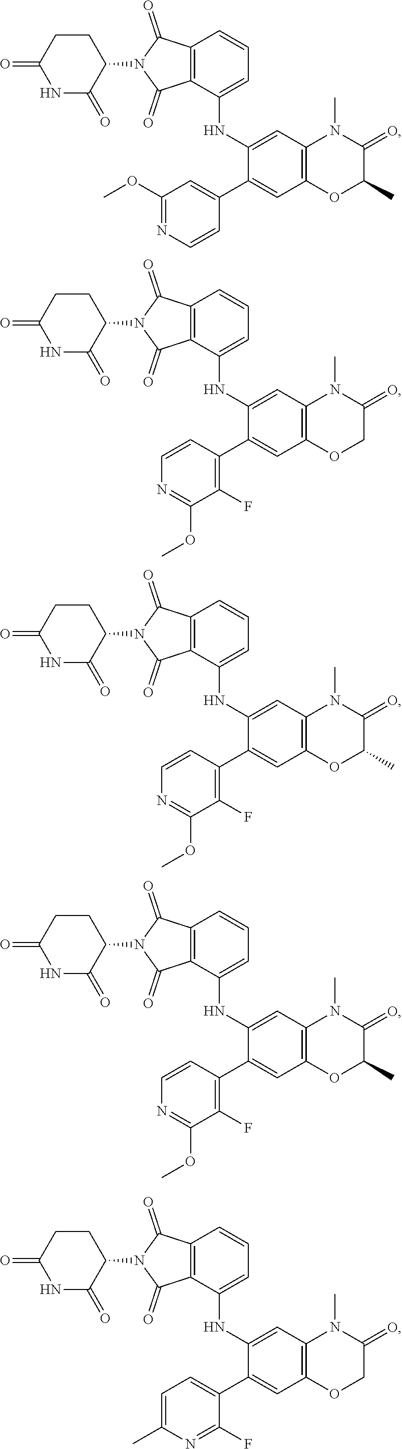

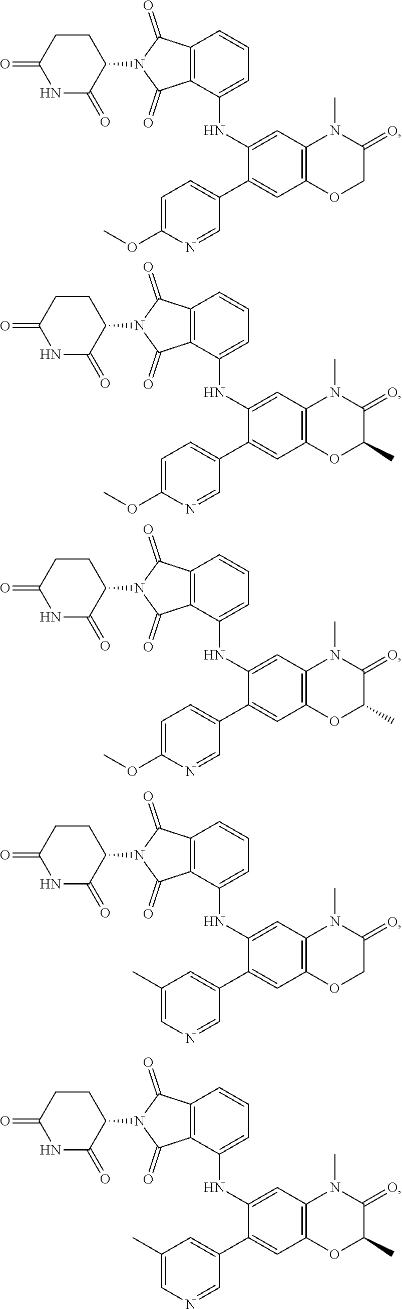

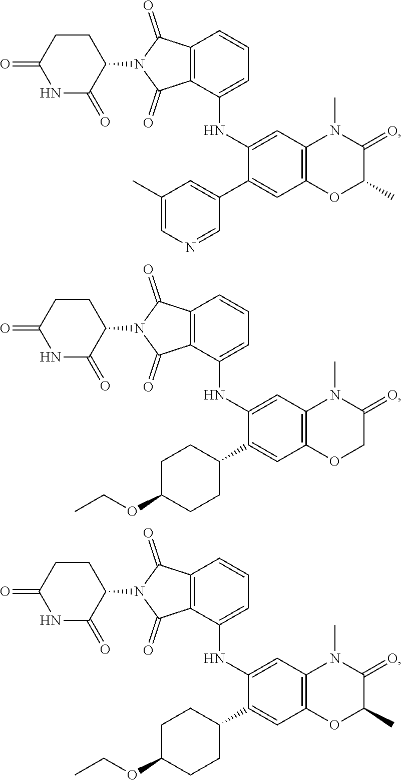

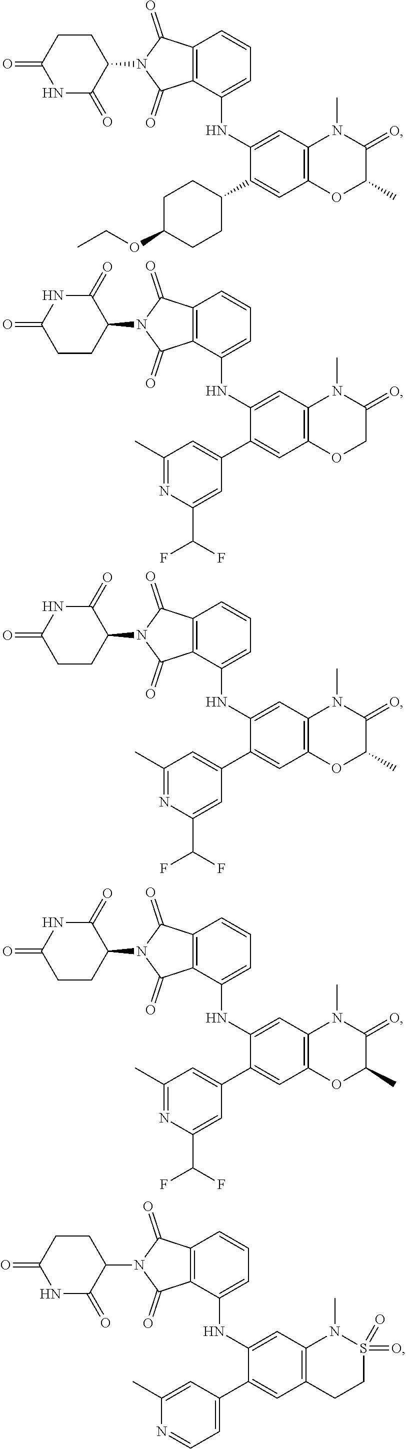

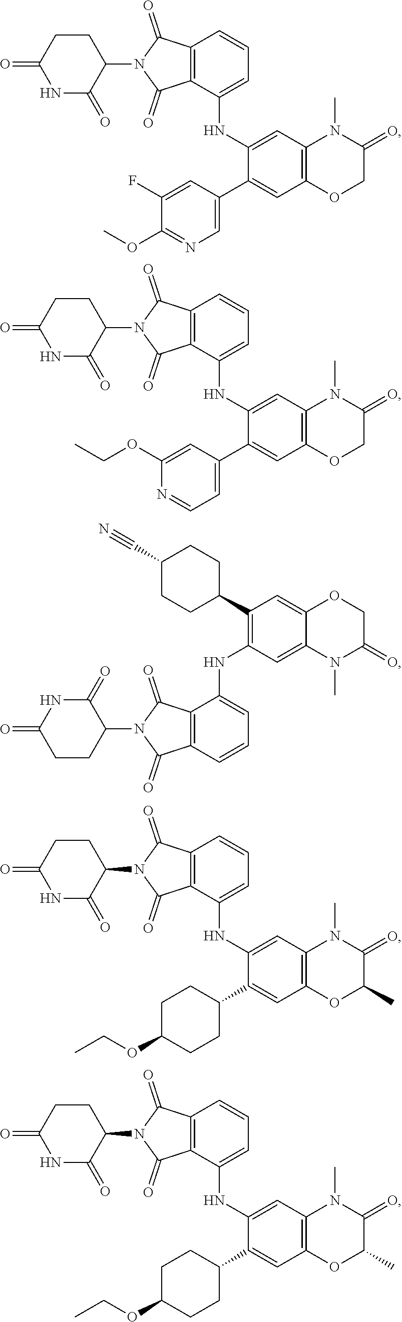

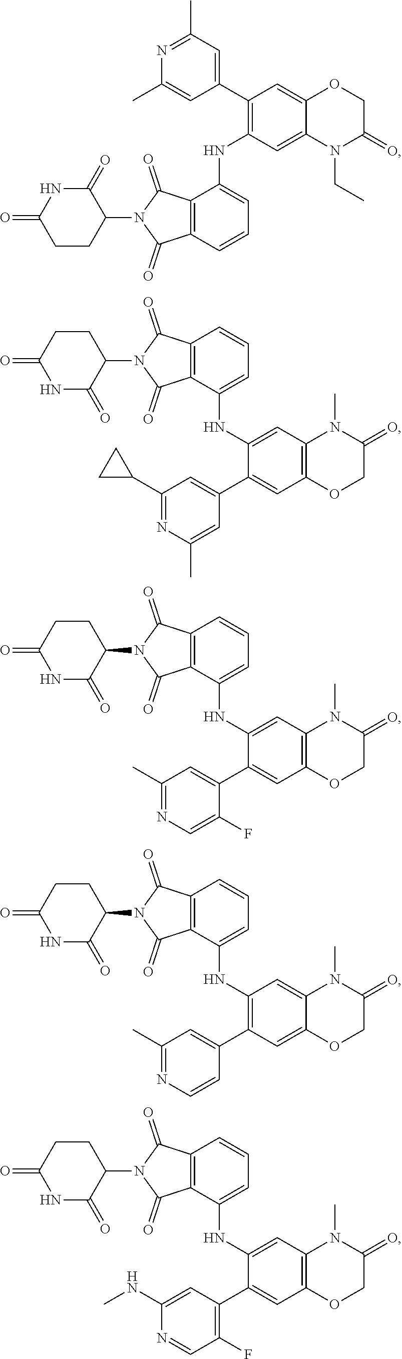

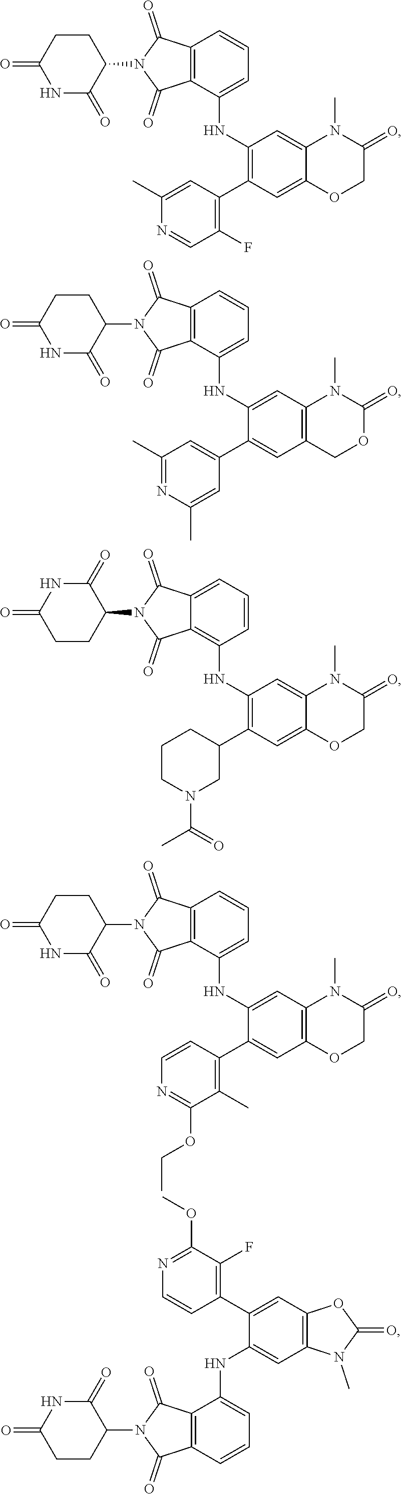

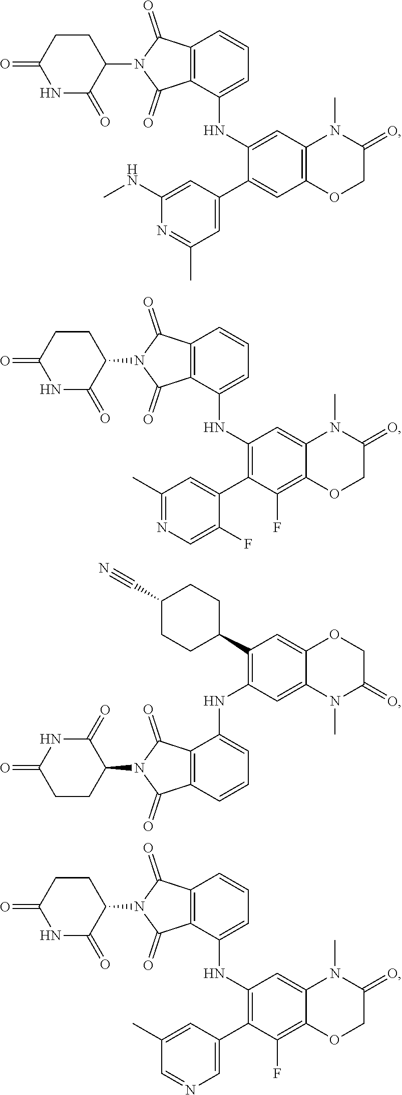

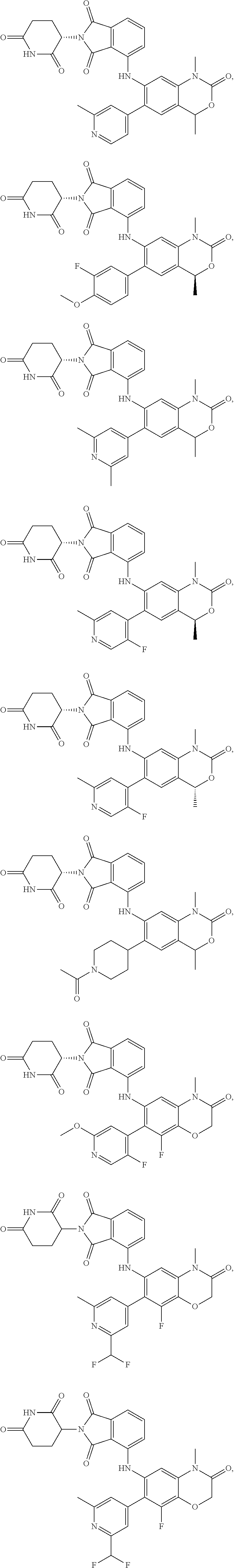

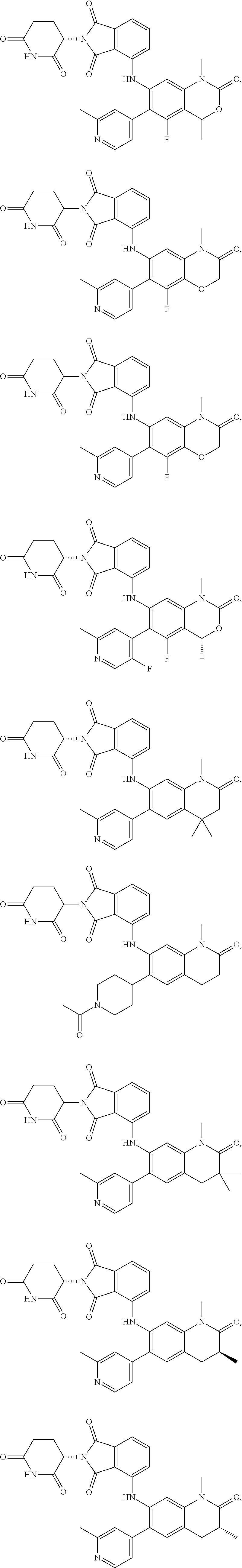

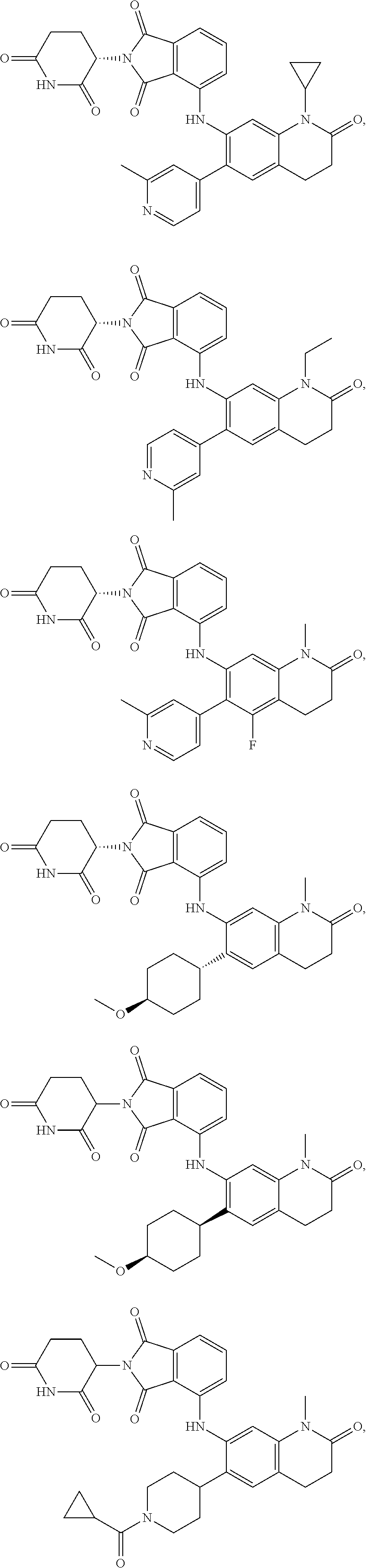

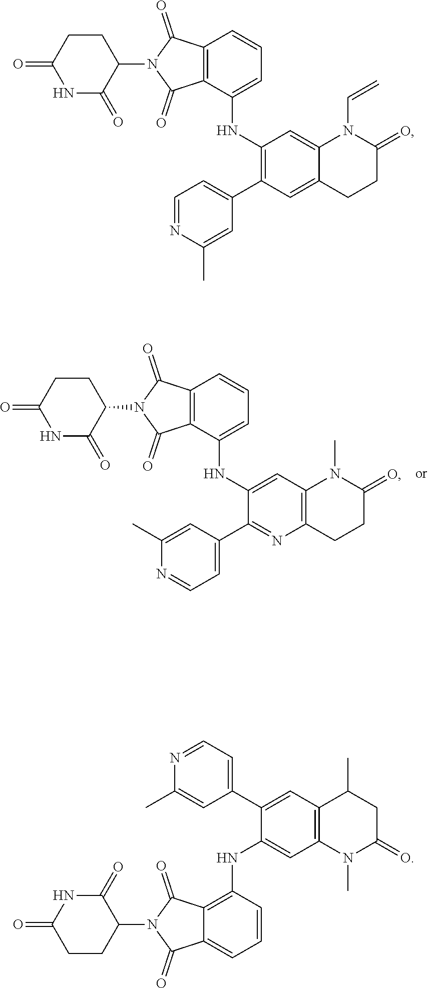 [SMILES: C=CN1C(=O)CCc2cc(-c3ccnc(C)c3)c(Nc3cccc4c3C(=O)N(C3CCC(=O)NC3=O)C4=O)cc21 ; Cc1cc(-c2nc3c(cc2Nc2cccc4c2C(=O)N([C@H]2CCC(=O)NC2=O)C4=O)N(C)C(=O)CC3)ccn1 ; Cc1cc(-c2cc3c(cc2Nc2cccc4c2C(=O)N(C2CCC(=O)NC2=O)C4=O)N(C)C(=O)CC3C)ccn1]

36. The compound of claim 35, of the following structural formula: 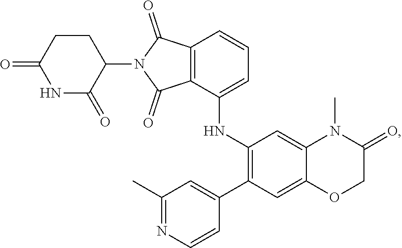 [SMILES: Cc1cc(-c2cc3c(cc2Nc2cccc4c2C(=O)N(C2CCC(=O)NC2=O)C4=O)N(C)C(=O)CO3)ccn1]
or a pharmaceutically acceptable salt, tautomer, isotopologue, and/or stereoisomer thereof.

37. The compound of claim 35, of the following structural formula: 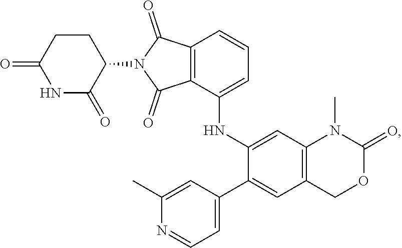 [SMILES: Cc1cc(-c2cc3c(cc2Nc2cccc4c2C(=O)N([C@H]2CCC(=O)NC2=O)C4=O)N(C)C(=O)OC3)ccn1]
or a pharmaceutically acceptable salt, tautomer, isotopologue, and/or stereoisomer thereof.

38. The compound of claim 35, of the following structural formula: 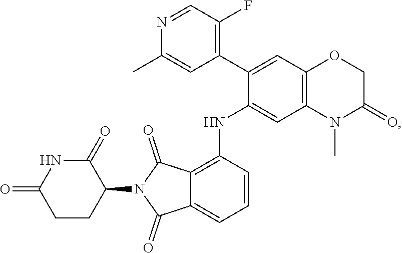 [SMILES: Cc1cc(-c2cc3c(cc2Nc2cccc4c2C(=O)N([C@H]2CCC(=O)NC2=O)C4=O)N(C)C(=O)CO3)c(F)cn1]
or a pharmaceutically acceptable salt, tautomer, isotopologue, and/or stereoisomer thereof.

39. The compound of claim 35, of the following structural formula: 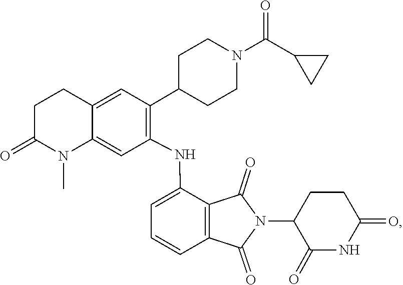 [SMILES: CN1C(=O)CCc2cc(C3CCN(C(=O)C4CC4)CC3)c(Nc3cccc4c3C(=O)N(C3CCC(=O)NC3=O)C4=O)cc21]
or a pharmaceutically acceptable salt, tautomer, isotopologue, and/or stereoisomer thereof.

40. A pharmaceutical composition comprising a compound or a pharmaceutically acceptable salt, tautomer, isotopologue, or stereoisomer thereof of claim 1, and a pharmaceutically acceptable carrier, excipient or vehicle.

41. (canceled)

42. (canceled)

43. (canceled)

44. A method of inducing HbF expression in a cell, and/or decreasing ZBTB7A expression in a cell expressing ZBTB7A, and/or decreasing WIZ expression in a cell expressing WIZ, comprising contacting the cell with a compound or a pharmaceutically acceptable salt, tautomer, isotopologue, or stereoisomer thereof of claim 1.

45. A method of treating a hemoglobinopathy in a subject in need thereof, comprising administering to the subject an effective amount of a compound or pharmaceutically acceptable salt, tautomer, isotopologue, or stereoisomer thereof of claim 1.

46-65. (canceled)
